(19) United States
(12) Patent Application Publication
(10) Pub. No.: US 2019/0024173 A1
(43) Pub. Date: Jan. 24, 2019

(54) Computer System And Methods For Harnessing Synthetic Rescues And Applications Thereof

(71) Applicants: UNIVERSITY OF MARYLAND, COLLEGE PARK, College Park, MD (US); RAMOT AT TEL AVIV UNIVERSITY LTD., Tel Aviv (IL)

(72) Inventors: Joo Sang LEE, (US); Avinash DAS, College Park, MD (US); Eytan RUPPIN, College Park, MD (US)

(73) Assignees: UNIVERSITY OF MARYLAND, COLLEGE PARK, College Park, MD (US); RAMOT AT TEL AVIV UNIVERSITY LTD., Tel Aviv (IL)

(21) Appl. No.: 15/756,371

(22) PCT Filed: Sep. 14, 2016
(86) PCT No.: PCT/IB2016/001427

§ 371 Date: Feb. 28, 2018

Related U.S. Application Data
(60) Provisional application No. 62/211,518, filed on Aug. 28, 2015.

(51) Int. Cl. C12Q 1/6883; G06F 19/20; G06F 17/18; G16H 50/20; G16H 50/30

(52) U.S. Cl. CPC C12Q 1/6883; G06F 19/20; G06F 17/18; C12Q 2600/158; G16H 50/30; C12Q 2600/156; G16H 50/20

(57) ABSTRACT

The disclosure comprises methods for predicting survival rates in subjects or populations of subject affected by a disease or disorder. The disclosure relates to methods of predicting the likely effect of and/or likely resistance developed from a treatments or combination of treatments. Software so execute the steps disclosed here and computer-implemented methods are also disclosed.

### CROSS-REFERENCE TO RELATED APPLICATIONS

This application is a PCT application claiming priority to a United States Provisional Application, U.S. Application No. 62/211,528, filed Aug. 28, 2015, which is incorporated by reference in its entirety.

### FIELD

The disclosure relates to methods and a system for predicting components of genetic interactions, or interrelated genes, the expression and/or activity levels of such genes, which are used to establish a prognosis for a subject, predict the likelihood of a subject to respond to a therapy for treatment of a disease or disorder, and/or predict improved therapies for treatment of as disease or disorder. In some embodiments, the disease or disorder is cancer, and, in some cases, breast cancer.

### BACKGROUND

The frequent emergence of resistance to anti-cancer therapies remains one of the most challenging problems in fighting cancer. Many recent clinical and experimental studies have aimed to address this challenge by characterizing drug and tumor-specific molecular signatures of emerging resistance through DNA or RNA sequencing¹⁻⁵. Such studies involve human cost, requiring collection and assessment of pre and post treatment data for every specific treatment and cancer type in dedicated clinical studies which can last for years. Moreover, clinical trials cannot be conducted for investigational drugs during early stages of their development.

Recent advances have led to significant improvements in targeted cancer therapy, however, quite frequently resistance emerges and cancer relapses. Here we rigorously define and comprehensively study a new class of cellular reprogramming termed synthetic rescues (SR). We develop INCISOR, a data-driven framework for inferring genome-wide SR networks in cancer. We find that SR reprogramming is widespread across cancer types and of significant clinical importance. We show that SR networks provide a universal framework for predicting and providing molecular insights into the response of many different cancers to a variety of treatments, and specifically, to the emergence of resistance to cancer therapies.

### SUMMARY OF EMBODIMENTS

The present disclosure relates to in-silico identification of molecular determinants of resistance, which can dramatically advance efforts of designing more efficient anti-cancer precision therapies. The present disclosure also relates to a method of mining large-scale cancer genomic data to identify molecular events which can be attributed to a class of genetic interactions termed synthetic rescues (SR) (and also synthetic lethality (SL) and synthetic dosage lethality (SDL)). An SR denotes a functional interaction between two genes or nucleic acid sequences in which a change in the activity of a vulnerable gene (which may be a target of a cancer drug) is lethal, but the subsequent altered activity of its partner (rescuer gene) restores cell viability. The method mines a large collection of cancer patients' data (TCGA)⁶ to identify the first genome-wide SR networks, composed of SR interactions common to many cancer types. INCISOR accurately recapitulates known and experimentally verified SR interactions. Analyzing genome-wide shRNA and drug response dataset, we demonstrate in vitro and in vivo emergence of synthetic rescue by shRNA or drug inhibition of INCISOR predicted rescuer genes, providing large-scale validations of the SR network. We then further test and validate a subset of these interactions involving key cancer genes in a set of new experiments. We show that SRs can be utilized to predict successfully patients' survival, response to the majority of current cancer drugs and an emergence of resistance. Finally, by in vitro and in vivo analyses, including our experiments, we show targeting particular rescuer gene of a drug re-sensitizes a resistant cell to the drug, revealing the therapeutic opportunities of SR network. Our analysis puts forward a new genome-wide approach for enhancing the effectiveness of existing cancer therapies by counteracting resistance pathways.

The present disclosure relates to in-silico identification of molecular determinants of resistance, which can dramatically advance efforts of designing more efficient anti-cancer precision therapies.

The present disclosure also relates to a method of mining large-scale cancer genomic data to identify molecular events which can be attributed to a class of genetic interactions termed synthetic rescues (SR). An SR denotes a functional interaction between two genes or nucleic acid sequences in which a change in the activity of a vulnerable gene (which may be a target of a cancer drug) is lethal, but the subsequent altered activity of its partner (rescuer gene) restores cell viability. mines a large collection of cancer patients' data (TCGA)⁶ to identify the first genome-wide SR networks, composed of SR interactions common to many cancer types. INCISOR accurately recapitulates known and experimentally verified SR interactions. Analyzing genome-wide shRNA and drug response dataset, we demonstrate in vitro and in vivo emergence of synthetic rescue by shRNA or drug inhibition of INCISOR predicted rescuer genes, providing large-scale validations of the SR network. We then further test and validate a subset of these interactions involving key cancer genes in a set of new experiments. We show that SRs can be utilized to predict successfully patients' survival, response to the majority of current cancer drugs and an emergence of resistance. Finally, by in vitro and in vivo analyses, including our experiments, we show targeting particular rescuer gene of a drug re-sensitizes a resistant cell to the drug, revealing the therapeutic opportunities of SR network. Our analysis puts forward a new genome-wide approach for enhancing the effectiveness of existing cancer therapies by counteracting resistance pathways.

The present disclosure further relates to a method of identifying a genetic interaction in a subject or population of subjects. The method can first perform the step of selecting at least a first pair of nucleic acids having a first and second nucleic acid from a dataset of a subject or population of subjects. The expression or somatic copy number alteration (SCNA) of the first nucleic acid can contribute to susceptibility of a disease or disorder and expression or SCNA of the second nucleic acid at least partially modulates or reverses the susceptibility caused by expression of the first nucleic acid. Alternatively, expression or somatic copy number alteration (SCNA) of both the first and second nucleic acids can contribute to susceptibility of a disease or disorder greater than expression or SCNA in a control subject or control population of subjects. The method can then perform the step of correlating expression of the first pair of genes with a survival rate associated with a disease or disorder in the subject or the population of subjects. The method can further perform the step of assigning a probability score to the first pair of genes based upon the survival rate. Finally, the method can perform the step of identifying the first pair of nucleic acid sequences as being in a genetic interaction if the probability score of the prior step is about or within the top twenty percent of a set of pairs of nucleic acid sequences correlated in the prior step.

The present disclosure also relates to a method of predicting responsiveness of a subject or population of subjects to a therapy. The method can first perform the step of selecting, from the subject or the population on the therapy, at least a first pair of nucleic acid sequences having a first and second sequence. The first nucleic acid sequence can be targeted by the therapy and expression of the second nucleic acid sequence which at least partially contributes to the development of the resistance or at least partially enhances the responsiveness of the therapy targeting the first gene. The method can then perform the step of correlating expression of the first pair of nucleic acid sequences with a survival rate associated with a disease or disorder in the subject or the population of subjects. The method can further perform the step of assigning a probability score to the first pair of nucleic acid sequences based upon the survival rate. Finally, the method can perform the step of predicting the subject or population's responsiveness to a therapy based upon expression of the second nucleic acid sequence if the probability score of the prior step is about or within the top twenty percent of a set of pairs of nucleic acid sequences correlated in the prior step.

The present disclosure also relates to a method of predicting a likelihood of a subject or population of subjects develops a resistance to a therapy. The method can first perform the step of selecting, from the subject or the population of subjects administered the therapy, at least a first pair of nucleic acid sequences having a first and second nucleic acid sequence. The first nucleic acid sequence can be targeted by the therapy and alteration in the expression of the second nucleic acid sequence which at least partially contributes to the emergence of resistance reducing the effectiveness of the therapy targeting the first nucleic acid sequence. The method can then perform the step of correlating expression of the first pair of nucleic acid sequences with a survival rate associated with a disease or disorder in the subject or the population of subjects. The method can then perform the step of assigning a probability score to the first pair of nucleic acid sequences based upon the survival rate. Finally, the method performs the step of predicting the subject or population's likelihood of developing resistance to a therapy based upon expression of the second nucleic acid sequence if the probability score of the prior step is about or within the top twenty percent of a set of pairs of nucleic acid sequences correlated in the prior step.

The present disclosure also relates to a method of predicting a prognosis and/or a clinical outcome of a subject or population of subjects suffering from a disease or disorder. The method first perform the step of selecting at least a first pair of nucleic acids having a first and second nucleic acid. Expression or SCNA of the first nucleic acid can contribute to severity of a disease or disorder and expression of the second nucleic acid at least partially modulates the severity of the disease or disorder caused by expression of the first nucleic acid. Alternatively, expression or SCNA of both the nucleic acids can contribute to susceptibility of a disease or disorder greater than a control subjects or population. The method can then perform the step of correlating expression of the first pair of nucleic acid sequences with a survival rate associated with a disease or disorder in the subject or the population of subjects. The method can then perform the step of assigning a probability score to the first pair of nucleic acid sequences based upon the survival rate. Finally, the method can perform the step of prognosing the clinical outcome of the subject or the population of subjects based upon the expression of the first pair of nucleic acid sequences if the probability score of the prior step is about or within the top twenty percent of a set of pairs of nucleic acid sequences correlated in the prior step.

The present disclosure also relates to a method of selecting or optimizing a therapy for treatment of a disease or disorder in a subject or population of subjects. The method can first perform the step of analyzing information from a subject or population of subjects associated with a disease or disorder and selecting at least a first pair of nucleic acids having a first and second nucleic acid. Expression of the first nucleic acid can contribute to severity of a disease or disorder and expression of the second nucleic acid which at least partially modulates the severity of the disease or disorder caused by expression of the first nucleic acid. Alternatively, expression of both nucleic acid can contribute at least partially to severity of a disease or disorder and this has greater than control subject or control population. The method can then perform the step of comparing expression of the first pair of nucleic acid sequences with a survival rate associated with a disease or disorder in a control population of subjects. The method can then perform the step of assigning a probability score to the expression of the first pair of nucleic acid sequences based upon the survival rate of the subject or population of subjects associated with a disease or disorder. Finally, the method can perform the step of selecting a therapy useful for treatment of the disease or disorder based upon the expression of the first pair of nucleic acid sequences.

The present disclosure also relates to a computer program product encoded on a computer-readable storage medium having instructions for analyzing information from a subject or population of subjects associated with a disease or disorder and selecting at least a first pair of nucleic acids having a first and second nucleic acid. Expression of the first nucleic acid contributes to severity of a disease or disorder and expression of the second nucleic acid at least partially modulates the severity of the disease or disorder caused by expression of the first nucleic acid. The computer readable medium also has instructions for comparing expression of the first pair of nucleic acid sequences with a survival rate associated with a disease or disorder in a control population of subjects. The computer readable medium also has instructions for assigning a probability score to the expression of the first pair of nucleic acid sequences based upon the survival rate of the subject or population of subjects associated with a disease or disorder.

The present disclosure also relates to a method of identifying a genetic interaction in a subject or population of subjects. The method can first perform the step of classifying one or a plurality of nucleic acid sequences into an active state or inactive state. The method can then perform the step of identifying at least a first pair of nucleic acid sequences, the first pair of nucleic acid sequences having a gene in an active state and a gene in an inactive state. The identifying step can predict that the expression of one of the nucleic acid sequences affects the expression of the other gene. The method can then perform the step of correlating expression of the first pair of nucleic acid sequences with a survival rate associated with a disease or disorder in the subject or the population of subjects and comparing expression of the first pair of nucleic acid sequences in a subject or population of subjects with the disease or disorder with expression of the first pair of nucleic acid sequences in a control subject or control population of subjects. The method can then perform the step of calculating an essentiality value associated with the first pair of nucleic acid sequences in an expression dataset excluding short hairpin RNA (shRNA) dataset. The method can then perform the step of correlating the essentiality value with a likelihood that the first pair of nucleic acid sequences is associated with the disease or disorder. The method can then perform the step of conducting a phylogenetic analysis across one or a plurality of expression data associated with a species unlike a species of the subject or population of the subjects. The method can then perform the step of assigning a probability score to the first pair of nucleic acid sequences based upon the phylogenetic analysis. Finally, the method can perform the step of identifying the first pair of nucleic acid sequences as being in a genetic interaction if the probability score of in the prior step is about or within the top five, six, seven, eight, nine or ten percent of those pairs of nucleic acid sequences analyzed in step of conducting a phylogenetic analysis.

### BRIEF DESCRIPTION OF THE DRAWINGS

FIG. 1. The INCISOR pipeline: The figure shows the four statistical screens composing it, and the datasets analyzed. The resulting output is a network of SR interactions of a specific type—the one displayed is of the SR type (red denotes vulnerable genes and green rescuer genes; the size of the nodes is proportional to the number of interactions they have. Synthetic Rescue functional truth tables: (a) (DU): the down-regulation of vulnerable gene is lethal but the cancer cell is rescued by the up-regulation of its rescuer partner. (b-d): Analogous functional truth tables for the three other SR types, (DD, UD, and UU). Red denotes lethal, green is viable, and blue is rescued. In difference, in SL (e) the down-regulation of each gene is viable but the down-regulation of both genes is lethal. (f,g): The SR (DU-type) network identified by INCISOR is composed of two large disconnected components: (f). A Growth factor subnetwork including 483 SR interactions between 225 vulnerable genes (red nodes) and 168 rescuers (green nodes), and (g), a DNA-damage subnetwork includes 451 SR interactions between 181 vulnerable genes and 111 rescuers. Names of the rescuer and vulnerable genes hubs are provided.

FIG. 2. Validation of INCISOR predicted SR interactions: (a-d) Using four gold standard datasets reported in five recent publications identifying rescuers of four drugs (a) ABT-737⁷, (b) Vorinostat⁸, (c) Lapatinib 9. and (d) BET-inhibitors^{1,2}. Prediction accuracy is assessed using Receiver operator curves (ROC). The results are displayed for SRs inferred using each screen of INCISOR individually and in combination. (e) in vitro and in vivo validation of predicted DD-SR interaction employing shRNA knockdowns¹⁰ and drug inhibitors: (e-g): The X axis shows the general effect on cell proliferation of DD-rescuer knockdowns (either by shRNA knockdown or by drug inhibitors) across all cell lines without a copy number loss of their corresponding vulnerable gene. The Y axis shows the conditional effect on proliferation of the knockdown of DD-rescuer genes only in the cell lines with a copy number loss of the corresponding vulnerable genes (and the DD-rescue is hence predicted to take place). A rescue effect is defined as the increase of proliferation in the conditional cases (Y axis) over that of general case (X-axis). Its significance is determined using a Wilcoxon rank sum test comparing the proliferation observed in the conditional vs. general cases. Red denotes predicted DD-rescuers and blue denotes random, control pairs. Circles denote pairs that have a significant rescue effect (Wilcox P-value <0.01) and crosses denote pairs insignificant rescue effects. As evident, a much larger fraction of the predicted rescuers shows a significant rescue effect (in all cases in vivo and in-vitro Wilcoxon P-value <2.2 E−16). Cell proliferation is measured in (e) as cell line growth rate post shRNA knockdown in large number of cell lines, in (f) normalized IC50 (Methods) of drug treatment in large number of cell lines, in (g) as cumulative percentage increase in tumor size following treatment with 38 drugs in 375 mice xenograft. (h,i) Experimental shRNA screening validates the predicted DD-SR rescue interactions involving mTOR in a head and neck cancer cell line: Predicted DD-SR pairs involving mTOR both as (h) a rescuer gene and as (i) a vulnerable gene were tested (Methods). The vertical axis shows the cell count fold change in Rapamycin-treated vs. untreated (i.e., in the rescued versus the non-rescued state), and the significance was quantified using one-sided Wilcoxon rank-sum test for three technical replicates with at least two independent shRNAs per each gene in each condition. Several sets of control genes (5 genes in each set that is the total of 25 genes) that are not predicted as SR partners of mTOR were additionally knocked down and screened for comparison. These control sets include proteins known to physically interact with mTOR, computationally predicted SL and SDL partners of mTOR, predicted DD-SR vulnerable partners of non-mTOR genes, and DD-SR predicted rescuer partners of non-mTOR genes. The black horizontal line indicates the median effect of Rapamycin treatment in these controls as a reference point. Experiments were carried with at least two independent shRNAs for each gene of interest and controls.

FIG. 3. The SR networks successfully predict cancer patient's survival and drug response. (a-d) A Kaplan-Meier (KM) analysis comparing the survival of patients whose tumors have many rescued SRs (top 10 percentile (N=800), rescued) to those with a few (bottom ten percentile (N=800), non-rescued). The difference in the areas under the curve between rescued (blue) and non-rescued (red) samples (ΔAUC) and their log rank p-values are denoted. (e) Patients with tumors having a large fraction of vulnerable genes that are not down-regulated (termed viable, green curve) have only intermediate levels of survival, less than those patients whose tumors are highly rescued. (f) Survival prediction by integrating both SL and SR networks. The subset of non-rescued patients in FIG. 3a that also have many functionally active SLs (top 10 percentile (N=87); Supplementary Information) show remarkably better survival than the subset of rescued patients that also have few functionally active SLs (bottom ten percentile (N=158)). (g) The SR network successfully predicts the response to cancer drug treatments. (g) We present the increase in hazard rates for patients with many over-expressed drug-specific rescuer genes compared to patients with few, as estimated via a Cox regression (KM plots for each drug are provided in Extended Data FIG. 3). (h) Rescuers of drugs over-expressed in tumors of non-responders. The fraction of predicted rescuers of drugs over-expressed in responders and non-responders (annotated based on post-treatment tumor reduction) for 19 drugs. Non-responders show a significantly higher fraction of rescuers over-expressed (Wilcox P<0.05) for 13 out 19 targeted drugs marked in red. SR network successfully predicts the response to cancer drug treatments. (a) The CDSRN includes 170 interactions between 36 vulnerable genes (red) the target of drug (violet) and 103 rescuers (green). (b) The predictive power (logrank p-value) of the CDSRN in classifying responder vs. non-responder patients for 36 different drugs, in descending order. (c) The increase in post to pretreatment expression of the rescuer genes (vertical axis) of the 4 drug targets, in resistant (red) vs sensitive tumors (blue). The rescuers of 3 targets show a significant increase (ranksum p-value<0.01). (d) The increase in expression of 5 rescuers of the gene target BCL2 in resistant vs sensitive samples (ranksum p-value<1E−3). (e) The correlation between the survival predictive power of the rescuers' interactions (measured over BC data) and their increased differential expression in resistant vs sensitive tumors (Spearman correlation 0.54 with p-value<1E−3). (f) The accuracy of SVM prediction of treatment response by Receiver Operator Curve (ROC) (Area Under Curve (AUC)=0.71).

FIG. 4. SR-based predictions of emerging resistance: (a) The DU-SR network identifies key molecular alterations associated with tumor relapse after Taxane treatment. Post-treatment expression of the predicted rescuer genes in the relapsed tumors (red) compared to their activation level in pre-treatment primary tumors (green). Significantly altered genes (10 out of 14, all in the predicted direction) are marked by stars (one-sided Wilcoxon rank-sum P<0.05). (b) The likelihood of developing drug SR-mediated resistance following current cancer treatments. (c) The predicted clinical impact of rescuer gene down-regulation: Key rescuer genes and their corresponding drugs are listed on the vertical axis, and the survival increase associated with rescuer inhibition is presented on the horizontal axis. (b,c) are generated via an SR-mediated data-driven analysis of the TCGA collection. (d-e) in-vitro and in vivo validation of SR-predicted anti-cancer combinational therapies. (d) INCISOR performance in identifying drugs that mitigate resistance to EGFR or ALK inhibitors¹¹ presenting the association of INCISOR scores (Y-axis) and the experimentally observed anti-resistance effectiveness of drugs (X-axis). (e) INCISOR performance in identifying synergistic drugs combination in the SAGE dataset (f-h) Experimental validation of PREDICTED drug combinations of KIT and PIK3CA inhibitors (from FIG. 4b). (f): Cell viability post treatment with various concentration combinations of KIT and PIK3CA inhibitors in head and neck cancer Detroit-562 cell lines. (g): Fa-CI (TC-Chou) plot of drug synergism between KIT and PIK3CA: The X-axis denotes the fraction of cells affected by drug combination (i.e. fraction of cell died due to drug treatments). The Y axis denotes the combination index (CI) of the inhibitor pair¹², where CI=1 denotes the inhibitor are additive, CI<1 denotes the inhibitor are synergistic and CI>one denotes the inhibitors are antagonistic. (h): Re-sensitization of Cal33 to KIT inhibitor Dasatinib by siRNA knockdown of it rescuer gene PIK3CA: The cell line response to Dasatinib regarding cell viability (Y axis) at different concentrations of Dasatinib treatment (X axis) in Cal33. The Dasatinib response is shown for two different PIK3CA siRNA and a non-targeting control. (a) The data includes gene expression, SCNA, and mutations of primary (N=81) and relapsed tumors (N=11). The primary tumors are classified as refractory (N=12), resistant (N=37), and sensitive (N=32). We compared the rescuers activation in pre-treatment vs posttreatment relapsed samples (b) and their pre-treatment activation in non-responders vs. responders (c), and built a binary classifier to predict which patient will eventually relapse among the 32 initial responders ((d) ROC plot comparing the accuracy obtained based on the rescuers genes (blue line, AUC=0.75) compared to that obtained with 11 random genes (red line, AUC=0.51)). (e) The expected clinical impact of the rescuer knockdown: Key rescuer genes and their corresponding drugs are listed on the vertical axis, and the expected clinical benefit of the rescuer knockdown is presented in the horizontal axis The clinical impact was measured by comparing the survival of drug-treated patients with and without the corresponding over-active rescuer (f) The likelihood of developing drug resistance: The probability of developing SR mediated resistance is estimated by the fraction of samples that have non-zero over-activation of rescuers.

FIG. 5: A block diagram is provided which illustrates an example embodiment of the system of the present application. Also provided are flowcharts illustrating the processing logic of the INCISOR and ISLE algorithms.

FIG. 6: The functional activity states of the DU-SR interaction types. Each state denotes the cell viability states—viable (green), non-rescued (i.e., lethal—red), and rescued (blue)—as a function of the activity state of each of the SR pair genes (down-regulated, wild-type and up-regulated). The states are enumerated as state 1 to state 9.

FIG. 7. (a) Pan-cancer clinical significance of SR network. X axis shows 23 different cancer types, and Y axis shows the fraction of significant pan-cancer SR in each cancer type. Pan-cancer TCGA dataset was divided into two halves. DU-SR network was identified by applying INCISOR using one half of the data, and clinical significance was determined in the other half of the data. (b) Clinical predictive power of pancancer DU-SR pairs in an independent ovarian cancer dataset. The KM plot compared the survival of rescued (top 5-percentile; blue) vs non-rescued (bottom 5-percentile; red) ovarian cancer samples (N=92). The rescued samples show worse patient survival (logrank p-value<0.017, ΔAUC=0.4). (c-e) Rescuer activation associated with the vulnerable gene inactivation due to somatic mutations. (c) Rescuer activation per each vulnerable gene. The horizontal axis lists vulnerable genes with somatic mutations in TCGA samples, and the vertical axis denotes the significance of rescuer gene-activity between samples with vs. without vulnerable gene mutations. (d) Rescuer activation per each rescuer. The horizontal axis lists rescuer genes with somatic mutations in TCGA samples and the vertical axis denotes the significance of rescuer gene-activity between samples with vs. without vulnerable gene mutations. (e) The KM plot depicts the aggregate clinical predictive power of rescuers of CDH11 gene, among patient with CDH11 mutation. (f) Predictive power of SR when they are treated as SL. In this predictor an activation of SR as defined as when a rescuer expression is wild type and vulnerable gene is inactive Specifically, for each patients we count number of rescuer activity is wild-type, patients with the higher count (top 10 percentile) were considered as non-responder and lower count (bottom 10 percentile) were considered as non-responder. (g) GO-term enrichment analysis with rescuers of the drug targets. Rescuers are enriched with lipid storage/transport, thioester/fatty acid metabolism, and drug efflux transporters.

FIG. 8. (a,c) Synthetic rescue interaction in ovarian cancer dataset: (a) Rescuers are up regulated in non-responders: We compared activation of 18 rescuer genes (of the treatment drug's 3 targets) in non-responders (blue) vs. responders (red) before primary treatments. Ranksum p-values denote significant non-responder vs. responder expression differences. Significant genes are marked by stars (ranksum p-value<0.05). (b) A binary classifier based on pre-treatment rescuer gene expression predicts patient relapse among 32 initial responders (AUC=0.77 (blue), vs. AUC=0.53 (red) for an 18-gene random classifier). (c) Pre-treatment SL partners' expression is insufficient to predict future relapse among initial responders in ovarian cancer. An ROC plot showing the prediction accuracy obtained by a linear SVM based on 18 SL partners (AUC=0.52) compared to the accuracy obtained based on 18 random genes (red line, AUC=0.52) in ovarian cancer. (d) Pre-treatment rescuers expression successfully predicts future relapse among initial responders in breast cancer. An ROC plot in breast cancer shows the prediction accuracy obtained by a linear SVM (AUC=0.74) compared to the accuracy obtained based on 13 random genes (red line, AUC=0.57). (e) Clinical significance of SL pairs identified by INCISOR Patients were scored based on number of functionally active SL pairs. Kaplan-Meier analysis shows the survival of patients who belong to top 10 percentile (SL+) is better than the survival of those belonging to bottom 10 percentile (SL−). (f-g) Experimental shRNA screening validates (DD) rescue effects of mTOR. (f) Summary of pooled shRNA experiment. Time points, treated and control samples are explained in the figure. (g) 19 predicted vulnerable partners for mTOR are knocked down using shRNA. Next, Rapamycin is used to inhibit mTOR. The vertical axes show fold change in cell counts after versus before Rapamycin treatment (i.e., in the non-rescued versus the rescued state). SR partners of mTOR are compared to several control genes that are not in SR pairs with mTOR.

FIG. 9. TCGA drug response. Drug response of top 15 anti-cancer drugs using drug-DU-SR in TCGA data. Each subplot represents a KM analysis of responder (red) v/s non-responders (blue) for a drug. The name of drug, log-rank p-value and ΔAUC is indicated in each subplot.

FIG. 10. (a-d) Clinical significance of 4 types of SR interactions in breast cancer: The Kaplan Meier (KM) plot depicts the difference in clinical prognosis between patients with rescued tumors (>90-percentile of number of functionally active SR pairs, blue) vs patients with non-rescued (<10-percentile of number of functionally active SR, red) samples. As predicted, a large number of functionally active rescuer pairs renders significantly marked worse survival based on all four different SR networks: (a) DD, (b) DU (c) UD and (d) UU. The logrank p-values and ΔAUC are marked, and DU shows the strongest clinical significance. (e) Illustration of effect of non-rescued, viable and rescued states on survival due to SR interaction between FGF10 (vulnerable gene) and EEA1 (rescuer gene) SR interaction. Patients were divided based on state of FGF10/EEA1 SR interaction: i) in viable state EEA1 was WT in patients, ii) in non-rescued state EEA1 was inactive and FGF10 was not over-active, and iii) in rescued stated EEA1 was inactive and FGF10 was over-active. (f) Rescue effect of SR network is due to interaction: Shuffling the vulnerable genes in SR network and KM analysis similar to FIG. 3e. (g-h) The functional activity of SR increases as cancer progresses. (g) The number of functionally active SRs (green) and random gene pairs (red) as cancer progresses. (h) The number of rescued inactive vulnerable genes with varying number of active rescuers (from single rescuer with darkest blue line to five rescuers with the lightest blue line) as cancer progresses. (i-l) The breast cancer SR-DU network predicts drug response in cell lines and cancer patients. (i) The rescuer activity profiles of individual cell-lines predict drug response of 9 out of 24 drugs. We compared the experimentally measured drug response (IC50 values) between predicted rescued vs. non-rescued cell lines using a ranksum test. The horizontal axis represents the 24 drugs in CCLE database, and the vertical axis denotes the ranksum p-values. (j) The rescuer activity profiles successfully predict the survival of patients whose tumors are rescued vs. those whose tumors are non-rescued (the latter patients have better survival) for 15 out of 37 drugs as quantified by a logrank test. The horizontal axis lists the 37 drugs in TCGA BC dataset, and the vertical axis represents the logrank p-values examining the separation between predicted rescued and non-rescued tumors. (k) The expected clinical impact of rescuer genes' knockdown: Key rescuer genes and their corresponding drugs (in parenthesis) are listed on the vertical axis, and the expected clinical benefit of the rescuer knockdown is presented in the horizontal axis. The clinical impact was measured by comparing the survival of drug-treated patients with and without the corresponding over-active rescuer (l) The likelihood of developing drug resistance: The probability of developing SR mediated resistance (vertical axis) for each drug (horizontal axis) is estimated by the fraction of samples that have non-zero over-activation of rescuers.

FIG. 11. (a-e) Synthetic rescues functional truth tables: The truth tables of the four SR and SL interaction types. Each truth table denotes the cell viability states—viable (green), non-rescued (i.e., lethal—red), and rescued (blue)—as a function of the activity state of each of the SR pair genes (down regulated, wild-type and up-regulated). The states are enumerated as state 1 to state 9: (a) (DU-SR): Down-regulation of a vulnerable gene is lethal but the cancer cell is rescued (retains viability) by the up-regulation of its rescuer partner; (b-d): Analogous functional truth tables for (DD, UD, and UU) SR types. (e) In an SL interaction, in difference, the down-regulation of either gene alone is viable but the down-regulation of both genes together is lethal. (f) Overview of INCISOR. INICISOR takes inputs as expression, somatic copy number of alternations (SCNA) and survival of patients sample as input and output SR pairs. It composes of 4 steps: SoF performs 4 Wilcoxon test to compare expression between groups highlighted in red and black (and similar 4 wilcox test for SCNA). Next three step survival data uses survival data and perform KM analyses to compare survival between the groups highlighted in red and black. (g-i) DU-type SR network and functional characterization. (f) Pairwise gene enrichment analysis: The figure shows relationship between vulnerable gene biological processes (red) and rescuer gene biological processes. Edges between a vulnerable process and rescuer process represents enrichment of the vulnerable process in vulnerable gene partner of rescuer process genes. (g) SR-DU network of metabolic genes and functional characterization. The figure depicts synthetic rescues network with 152 vulnerable genes (green) and 210 rescuer genes (red) of 131 metabolic genes (diamond) encompassing 258 interactions. The size of nodes indicates their degree in the network as in (c).

FIG. 12. (a-d) SR network successfully predicts the response to cancer drug treatments in breast cancer. (a) Expression fold change (pre- versus post-drug treatment) is shown for the rescuer genes of the four vulnerable genes that are targeted by a drug cocktail in a cohort of 25 clinical breast cancer patients (i.e., from the BC25 dataset). Box plots aggregate rescuer expression changes for all rescuers of a given vulnerable target across patients that are clinical responders (blue) and non-responders (red). Ranksum p-values denote differences in overall rescuer fold change between these responder groups for each target gene. (b) Expression fold changes are shown for clinical responders and non-responders of BC25 for the 5 rescuers of the gene target BCL2. In (a) and (b) significant genes are marked by stars (ranksum p-value<0.05). (c) The 20 DU gene pairs active in the BC25 dataset are ranked by degree of potency (i.e., by the ranksum p-value denoting differential responder- versus non-responder pre- to post-drug fold change) (y-axis), and also ranked by their rescue effect (as calculated using the BC-DU-SR network as in step 2 of INCISOR) (x-axis). These measures correlate (Spearman ρ=−0.54, p<1e−3). (d) Receiver Operating Characteristic (ROC) curve for an SVM predictor of patient treatment response, trained on the BC25 dataset. Area under the curve (AUC) is 0.71 for the predictor (blue), as compared to 0.54 for a random predictor (red). (e-k) SR network successfully predicts the response to cancer drug treatments in gastric cancer (e) The bar plot shows the significance of over-expression of 15 rescuers of THYMS in the tumors of patients who acquired resistance to Cisplatin and Fluorouracil compared to the patients who did not acquire resistance. (f,g) The KM plots depict the clinical significance of rescuer over-expression in patient tumors in terms of progression free survival (f) and overall survival (g). The patients with highly rescued tumors (>90 percentile) have significantly worse survival compared the patients with lowly rescued tumors (<10 percentile). The KM plot compares the difference in survival rates between “rescued” patients with many rescuers over-expressed (top 10 percentile) and “non-rescued” patients with fewer rescue events (bottom 10 percentile) for random chosen rescuer genes (h) for over-all survival and (i) progression-free survival. Both figures show no statistical significance. (j) The contribution of the 4 steps of INCISOR in predicting over-activation of rescuers. The rescuers identified by combining 4 steps of INCISOR show the highest significance, and this is followed by significances of rescuers' over-expression identified with each of the step separately: robust rescue effect (step 3), oncogene rescuer screening (step 4), molecular survival of the fittest (step 1), vulnerable gene screening (step 2), and random control. (k) The clinical significance of the rescuer up-regulation (rescue effect) of the 4 steps of INCISOR (estimated in ΔAUC). The rescuers identified by all 4 steps of INCISOR have the most significant clinical impact, and this is followed by those identified by robust rescue effect (step 3), molecular survival of the fittest (step 1), oncogene rescuer screening (step 4), and vulnerable gene screening (step 2).

FIG. 13. (a-b) Characterization of rSR and bSR. (a) We identified rSR by selecting SR pairs whose rescuer activation (green) consistently drives the functional activation of SR (blue) as cancer progresses. (b) We identified bSR pairs by selecting SR pairs whose vulnerable gene inactivation (red) drives the functional activation. (c-j) Clinical impact of rSR and bSR (c,d) The KM plots depict the patients with highly rescued tumors (red; >90 percentile) have worse survival than the patients with lowly rescued tumors (blue; <10 percentile). The rSR shows more significant clinical rescue effect (logrank p-value<1E−300) than bSR (logrank p-value<1E−8) in comparison to rescuer controls (g) and (h). (e,f) The KM plots depict the difference in the survival between two groups of patients whose tumors are highly vulnerable (red; >90 percentile) vs. lowly vulnerable (blue; <10 percentile) given over-activation of rescuer genes. The rSR shows more significant impact (logrank p-value<1E−300) than bSR (logrank p-value<1E−8) in comparison to vulnerable controls (i) and (j).

FIG. 14. Clinical significance of SR network in breast cancer subtypes The KM plot depicting the differences in clinical prognosis between rescued (>90-percentile of number of functionally active SR, blue) vs non-rescued (<10-percentile of number of functionally active SR, red) samples in her2 subtype (first row), triple-negative (second row), luminalA (third row), and luminalB (fourth row). The high fraction of rescue renders worse survival in all 4 different types of SR: DD (first column), DU (second column), UD (third column), and UU (fourth column). Their logrank p-values and the ΔAUC are represented.

FIG. 15. The DU-SR network identifies key molecular alterations associated with tumor relapse after Taxane treatment. (a) The OC81 dataset includes gene expression, copy number, and mutational information for primary (N=81) and relapsed (N=11) tumors. The tumors were classified as refractory (N=12), resistant (N=37), and sensitive (N=32). (b) Post-treatment activation in the relapsed tumors (blue) of rescuer genes compared to their activation level in pre-treatment primary tumors (red) of the 11 patients. Significant genes are marked by stars (one-sided Wilcoxon rank-sum P<0.05). (c) SR—(blue) and MDR—(red) mediated responses co-vary in the patients developing resistance to Taxane treatment in the 11 patients: The horizontal axis denotes the extent (−log 10(one-sided Wilcoxon rank-sum P)) of post-treatment increase in MDR genes activation and the vertical axis represents the extent of post-treatment increase in the predicted rescuers' activation (−log 10(one-sided Wilcoxon rank-sum P)).

FIG. 16. (a,b): Experimental shRNA screening validates the predicted DD-SR rescue interactions involving mTOR in a head and neck cancer cell-line: Predicted DD-SR pairs involving mTOR both as (a) a rescuer gene and as (b) a vulnerable gene were tested. The vertical axis shows the cell count fold change in Rapamycin treated vs. untreated (i.e., in the rescued versus the non-rescued state), and the significance was quantified using one-sided Wilcoxon rank-sum test for three technical replicates with at least 2 independent shRNAs per each gene in each condition. Several sets of control genes (5 genes in each set that is total of 25 genes) that are not predicted as SR partners of mTOR were additionally knocked down and screened for comparison. These control sets include proteins known to physically interact with mTOR, computationally predicted SL and SDL partners of mTOR, predicted DD-SR vulnerable partners of non-mTOR genes, and DD-SR predicted rescuer partners of non-mTOR genes. The horizontal black line indicates the median effect of Rapamycin treatment in these controls as a reference point. Experiments were carried with at least 2 independent shRNAs for each gene of interest and controls. (c-e) The SR network successfully predicts the response to cancer drug treatments. (c) The SR network of a few cancer drugs whose resistance mechanisms were recently published (see text). The network includes the drug targets (red) and their rescuers (green). The rescuers are involved in Wnt signaling (diamond), and hepatocyte growth factor receptor and actin cytoskeleton (box).

FIG. 17. Pan-cancer DU-type SR network. (a) Pan-cancer DU-type synthetic rescues network with 686 rescuer genes (green) and 1,513 vulnerable genes (red) encompassing 2,033 interactions. The size of nodes indicates their degree in the network. (b,c): Gene Ontology enrichment of vulnerable and rescuer genes. (b) The vulnerable genes are enriched with cell adhesion, protein modification, metabolism and deubiquitination. (c) The rescuer genes are enriched with mitotic cell cycle phase transition, chromatid segregation, cell migration and RNA transport. Only significant pathways (one-sided hypergeometric FDR adjusted P<0.05) are shown in the figure.

### DETAILED DESCRIPTION OF EMBODIMENTS

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The terms “amino acid” refer to a molecule containing both an amino group and a carboxyl group bound to a carbon which is designated the a-carbon. Suitable amino acids include, without limitation, both the D- and L-isomers of the naturally-occurring amino acids, as well as non-naturally occurring amino acids prepared by organic synthesis or other metabolic routes. In some embodiments, a single “amino acid” might have multiple sidechain moieties, as available per an extended aliphatic or aromatic backbone scaffold. Unless the context specifically indicates otherwise, the term amino acid, as used herein, is intended to include amino acid analogs including non-natural analogs.

As used herein, the terms “biopsy” means a cell sample, collection of cells, or bodily fluid removed from a subject or patient for analysis. In some embodiments, the biopsy is a bone marrow biopsy, punch biopsy, endoscopic biopsy, needle biopsy, shave biopsy, incisional biopsy, excisional biopsy, or surgical resection.

As used herein, the terms “bodily fluid” means any fluid from isolated from a subject including, but not necessarily limited to, blood sample, serum sample, urine sample, mucus sample, saliva sample, and sweat sample. The sample may be obtained from a subject by any means such as intravenous puncture, biopsy, swab, capillary draw, lancet, needle aspiration, collection by simple capture of excreted fluid.

The terms “comprise(s),” “include(s),” “having,” “has,” “can,” “contain(s),” and variants thereof, as used herein, are intended to be open-ended transitional phrases, terms, or words that do not preclude the possibility of additional acts or structures.

As used herein the terms “disease or disorder” is any one of a group of ailments capable of causing an negative health in a subject by: (i) expression of one or a plurality of mutated nucleic acid sequences in one or a plurality of amino acids; or (ii) aberrant expression of one or a plurality of nucleic acid sequences in one or a plurality of amino acids, in each case, in an amount that causes an abnormal biological affect that negatively affects the health of the subject. In some embodiments, the disease or disorder is chosen from: cancer of the adrenal gland, bladder, bone, bone marrow, brain, spine, breast, cervix, gall bladder, ganglia, gastrointestinal tract, stomach, colon, heart, kidney, liver, lung, muscle, ovary, pancreas, parathyroid, penis, prostate, salivary glands, skin, spleen, testis, thymus, thyroid, or uterus. In some embodiments, a disease or disorder is a hyperproliferative disease. The term hyperproliferative disease means a cancer chosen from: lung cancer, bone cancer, CMML, pancreatic cancer, skin cancer, cancer of the head and neck, cutaneous or intraocular melanoma, uterine cancer, ovarian cancer, rectal cancer, cancer of the anal region, stomach cancer, colon cancer, breast cancer, testicular, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas).

As used herein the terms “electronic medium” mean any physical storage employing electronic technology for access, including a hard disk, ROM, EEPROM, RAM, flash memory, nonvolatile memory, or any substantially and functionally equivalent medium. In some embodiments, the software storage may be co-located with the processor implementing an embodiment of the invention, or at least a portion of the software storage may be remotely located but accessible when needed.

As used herein, the terms “information associated with the disease or disorder” means any information related to a disease or disorder necessary to perform the method described herein or to run the software identified herein. In some embodiments, the information associated with a disease or disorder is any information from a subject that can be used or is used as a parameter or variable in the input of any analytical function performed in the course of performing any method disclosed herein. In some embodiments, the information associated with the disease or disorder is selected from: DNA or RNA expression levels of a subject or population of subjects, amino acid expression levels of a subject or population of subjects, whether or not the subject or population is taking a therapy for a condition, the age of a subject or population of subjects, the gender of a subject or population of subjects, the; or whether and, if so, how much or how long a subject or population of subjects has been exposed to an environmental condition, drug or biologic.

As used herein, “inhibitors” or “antagonists” of a given protein refer to modulatory molecules or compounds that, e.g., bind to, partially or totally block activity, decrease, prevent, delay activation, inactivate, desensitize, or down regulate the activity or expression of the given protein, or downstream molecules regulated by such a protein. Inhibitors can include siRNA or antisense RNA, genetically modified versions of the protein, e.g., versions with altered activity, as well as naturally occurring and synthetic antagonists, antibodies, small chemical molecules and the like. Assays for identifying other inhibitors can be performed in vitro or in vivo, e.g., in cells, or cell membranes, by applying test inhibitor compounds, and then determining the functional effects on activity.

The term “nucleic acid” refers to a molecule comprising two or more linked nucleotides. “Nucleic acid” and “nucleic acid molecule” are used interchangeably and refer to oligoribonucleotides as well as oligodeoxyribonucleotides. The terms also include polynucleosides (i.e., a polynucleotide minus a phosphate) and any other organic base containing nucleic acid. The organic bases include adenine, uracil, guanine, thymine, cytosine and inosine. The nucleic acids may be single or double stranded. The nucleic acid may be naturally or non-naturally occurring. Nucleic acids can be obtained from natural sources, or can be synthesized using a nucleic acid synthesizer (i.e., synthetic). Isolation of nucleic acids are routinely performed in the art and suitable methods can be found in standard molecular biology textbooks. (See, for example, Maniatis' Handbook of Molecular Biology.) The nucleic acid may be DNA or RNA, such as genomic DNA, mitochondrial DNA, mRNA, cDNA, rRNA, miRNA, PNA or LNA, or a combination thereof, as described herein. In some embodiments, the term nucleic acid sequence is used to refer to expression of genes with all or part of their regulatory sequences operably linked to the expressible components of the gene. In some embodiments, the expression of genes is analyzed for genetic interactions. In other embodiments, genetic interactions are analyzed by identifying pairs of a first gene and a second gene whose expression or activity contributes to the modulation of the lethality or likelihood of a subject from which the information associated with a disease or disorder is obtained. In some embodiments, the nucleic acid pair (comprising a first and second nucleic acid) is a pair of microRNAs, shRNAs, amino acids or nucleic acid sequences defined with presence of only partial regulatory sequences operably linked to the expressible components of a gene.

For purposes of this disclosure nucleic acid pairs may be identified as an SR or SL. SRs or synthetic rescues may be identified by the methods provided herein, wherein any one gene of the pair may contribute to at least partially controlling the likelihood of a negative impact of its expression or activity on the health of a subject and the other pair may rescue the likelihood of the negative impact. There are four kinds of SRs: (a) DU, where the Downregulation of vulnerable gene is rescued by Upregulation of rescuer gene; (b) DD, where the Downregulation of vulnerable gene is rescued by the Downregulation of rescuer gene; (c) UU and (d) UD are analogous to DU and DD respectively, but the initial stress event is the upregulation of vulnerable gene. In some embodiments, any of the methods may be performed to identify a DU and/or DD that correlates with inhibition of their drug targets of the first nucleic acid sequence in the pair.

Some aspects of this invention relate to the use of nucleic acid derivatives or synthetic sequences. The use of certain nucleic acid derivatives or synthetic sequences may enable complementarity as between natural expression products (such as mRNA) and the synthetic sequences to block protein translation of products for validation of software analysis and corroboration with biological assays. As used herein, a nucleic acid derivative is a non-naturally occurring nucleic acid or a unit thereof. Nucleic acid derivatives may contain non-naturally occurring elements such as non-naturally occurring nucleotides and non-naturally occurring backbone linkages. Nucleic acid derivatives according to some aspects of this invention may contain backbone modifications such as but not limited to phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof. The backbone composition of the nucleic acids may be homogeneous or heterogeneous. Nucleic acid derivatives according to some aspects of this invention may contain substitutions or modifications in the sugars and/or bases. For example, some nucleic acid derivatives may include nucleic acids having backbone sugars which are covalently attached to low molecular weight organic groups other than a hydroxyl group at the 3′ position and other than a phosphate group at the 5′ position (e.g., an 2′-O-alkylated ribose group). Nucleic acid derivatives may include non-ribose sugars such as arabinose. Nucleic acid derivatives may contain substituted purines and pyrimidines such as C-5 propyne modified bases, 5-methylcytosine, 2-aminopurine, 2-amino-6-chloropurine, 2,6-diaminopurine, hypoxanthine, 2-thiouracil and pseudoisocytosine. In some embodiments, a nucleic acid may comprise a peptide nucleic acid (PNA), a locked nucleic acid (LNA), DNA, RNA, or a co-nucleic acids of the above such as DNA-LNA co-nucleic acid.

As used herein, the term “probability score” refers to a quantitative value given to the output of any one or series of algorithms that are disclosed herein. In some embodiments, the probability score is determined by application of one or plurality of algorithm disclosed herein by: setting, by the at least one processor, a predetermined value, stored in the memory, that corresponds to a threshold value above which the first pair of nucleic acid sequence is correlated to an interaction event, the ineffectiveness or effectiveness of a therapy, the resistance of a therapy, and/or the prognosis of the subject or population of subjects suffering from a disease or disorder; calculating, by the at least one processor, the probability score, wherein calculating the probability score comprises: (i) analyzing information associated with a disease or disorder of the subject or the population of subjects; and

(ii) conducting one or a plurality of statistical tests from the information associated with a disease or disorder; and (iii) assigning a probability score related to an interaction event, the ineffectiveness or effectiveness of a therapy, the resistance of a therapy, and/or the prognosis of the subject or population of subjects suffering from a disease or disorder based upon a comparison of outcomes from the operation of statistical tests and the threshold value.

As used herein, the term “prognosing” means determining the probable course and/or clinical outcome of a disease.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises bodily fluid. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. in some embodiments, the methods disclosed herein do not comprise a processed sample. Representative biological samples include, but are not limited to: blood, a component of blood, a portion of a tumor, plasma, serum, saliva, sputum, urine, cerebral spinal fluid, cells, a cellular extract, a tissue specimen, a tissue biopsy, or a stool specimen. In some embodiments a biological sample is whole blood and this whole blood is used to obtain measurements for a biomarker profile. In some embodiments a biological sample is tumor biopsy and this tumor biopsy is used to obtain measurements for a biomarker profile. In some embodiments a biological sample is some component of whole blood. For example, in some embodiments some portion of the mixture of proteins, nucleic acid, and/or other molecules (e.g., metabolites) within a cellular fraction or within a liquid (e.g., plasma or serum fraction) of the blood. In some embodiments, the biological sample is whole blood but the biomarker profile is resolved from biomarkers expressed or otherwise found in monocytes that are isolated from the whole blood. In some embodiments, the biological sample is whole blood but the biomarker profile is resolved from biomarkers expressed or otherwise found in red blood cells that are isolated from the whole blood. In some embodiments, the biological sample is whole blood but the biomarker profile is resolved from biomarkers expressed or otherwise found in platelets that are isolated from the whole blood. In some embodiments, the biological sample is whole blood but the biomarker profile is resolved from biomarkers expressed or otherwise found in neutrophils that are isolated from the whole blood. In some embodiments, the biological sample is whole blood but the biomarker profile is resolved from biomarkers expressed or otherwise found in eosinophils that are isolated from the whole blood. In some embodiments, the biological sample is whole blood but the biomarker profile is resolved from biomarkers expressed or otherwise found in basophils that are isolated from the whole blood. In some embodiments, the biological sample is whole blood but the biomarker profile is resolved from biomarkers expressed or otherwise found in lymphocytes that are isolated from the whole blood. In some embodiments, the biological sample is whole blood but the biomarker profile is resolved from biomarkers expressed or otherwise found in monocytes that are isolated from the whole blood. In some embodiments, the biological sample is whole blood but the biomarker profile is resolved from one, two, three, four, five, six, or seven cell types from the group of cells types consisting of red blood cells, platelets, neutrophils, eosinophils, basophils, lymphocytes, and monocytes. In some embodiments, a biological sample is a tumor that is surgically removed from the patient, grossly dissected, and snap frozen in liquid nitrogen within twenty minutes of surgical resection.

The term “subject” is used throughout the specification to describe an animal from which a sample is taken. In some embodiment, the animal is a human. For diagnosis of those conditions which are specific for a specific subject, such as a human being, the term “patient” may be interchangeably used. In some instances in the description of the present invention, the term “patient” will refer to human patients suffering from a particular disease or disorder. In some embodiments, the subject may be a human suspected of having or being identified as at risk to develop a type of cancer more severe or invasive than initially diagnosed. In some embodiments, the subject may be diagnosed as having at resistance to one or a plurality of treatments to treat a disease or disorder afflicting the subject. In some embodiments, the subject is suspected of having or has been diagnosed with stage I, II, III or greater stage of cancer. In some embodiments, the subject may be a human suspected of having or being identified as at risk to a terminal condition or disorder. In some embodiments, the subject may be a mammal which functions as a source of the isolated sample of biopsy or bodily fluid. In some embodiments, the subject may be a non-human animal from which a sample of biopsy or bodily fluid is isolated or provided. The term “mammal” encompasses both humans and non-humans and includes but is not limited to humans, non-human primates, canines, felines, murines, bovines, equines, and porcines.

A “therapeutically effective amount” or “effective amount” of a composition (e.g, any therapy or combination of therapies) is a predetermined amount calculated to achieve the desired effect, i.e., to improve and/or to decrease one or more symptoms of a disease or disorder. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. The compounds are effective over a wide dosage range and, for example, dosages per day will normally fall within the range of from 0.001 to 10 mg/kg, more usually in the range of from 0.01 to 1 mg/kg. However, it will be understood that the effective amount administered will be determined by the physician in the light of the relevant circumstances including the condition to be treated, the choice of compound to be administered, and the chosen route of administration, and therefore the above dosage ranges are not intended to limit the scope of the disclosure in any way. A therapeutically effective amount of compound of embodiments of this disclosure is typically an amount such that when it is administered in a physiologically tolerable excipient composition, it is sufficient to achieve an effective systemic concentration or local concentration in the tissue.

The terms “threshold value” as used herein refer to the quantitative value above which or below which a probability value is considered statistically significant as compared to a control set of data. For example, in the case of the disclosed method of determining the whether a nucleic acid pair corresponds to a likelihood of a subject or population of subjects to develop resistance to a therapy (such as therapy for breast cancer subjects), the threshold value is the quantitative value that is about 20%, 15%, 10%, 5%, 4%, 3%, 2%, or 1% below the greatest probability score assigned to a nucleic acid pair after the probability score is calculated by input of information associated with a disease or disorder into one or more of the statistical tests provided herein.

“Treatment” or “treating,” as used herein can mean protecting of an animal from a disease or disorder through means of preventing, suppressing, repressing, or completely eliminating the disease or symptom of a disease or disorder. Preventing the disease involves administering a therapy (such as a vaccine, antibody, biologic, gene therapy with or without viral vectors, small chemical compound, etc.) to a subject or population of subjects prior to onset of the disease or disorder. Suppressing the disease involves administering a therapy to a subject or population of subjects after induction of the disease but before its clinical appearance. Repressing the disease involves administering a therapy of to a subject or population of subjects after clinical appearance of the disease.

As used herein the term “web browser” means any software used by a user device to access the internet. In some embodiments, the web browser is selected from: Internet Explorer®, Firefox®, Safari®, Chrome®, SeaMonkey®, K-Meleon, Camino, OmniWeb®, iCab, Konqueror, Epiphany, Opera™, and WebKit®.

The disclosure further relates to a computer program product encoded on a computer-readable storage medium that comprises instructions for performing any of the methods described herein. In some embodiments, the disclosure relates to any of the disclosed methods on a system or software that accesses the internet.

One application of such computers, computer program products, systems and methods is the identification of specific diseases/conditions for which a given chemical agent or pharmaceutical drug would provide effective therapeutic treatment. For example, the present invention provides systems and methods for identifying genetic profiles of specific cancers for which currently available chemical agents, pharmaceutical drugs, or other therapies of interest would provide either effective to treatment or ineffective due to resistance of treatment. The present invention also provides systems and methods for identifying genetic profiles of specific cancers for which currently available chemical agents, pharmaceutical drugs, or other therapies of interest would provide a therapeutically effective amount of a treatment or an adjuvant treatment.

In one embodiment, the subject invention provides systems and methods for defining and analyzing genetic profiles for at least one or two specific disease states (e.g., cancers); (2) identifying a therapy of interest (e.g., one or more chemical agents or one or more pharmaceutical drugs) known to be therapeutically effective in treating a specific disease state whose expression signature is defined by accessing and inputting information associated with the disease state or disorder from a database, (3) defining a discrimination set of genetic interactions that are representative of changes in expression signatures or “response signature” for the genetic profile of the specific disease or disorder before, after administration of a therapy of interest induces a therapeutic effect; and (4) analyzing the screenable database to identify any other disease states that include a similar response signature for which the therapy of interest may be therapeutically effective in treating.

In one embodiment, genetic interaction profiles for specific diseases (e.g., cancers) are identified and stored in a screenable database in accordance with the subject invention. A therapy of interest that is known to be therapeutically effective for a specific disease is selected. A biological sample for which the therapy of interest is known to therapeutically affect is then exposed to the therapy of interest and its molecular profile is obtained. This molecular profile may be measurements of cellular constituents in the biological sample prior to exposure. Alternatively, this molecular profile may be differential measurements of cellular constituents in the biological sample before and after exposure to the therapy of interest, where a change in the expression of specific cellular constituents serves as a “response signature” for the change in cellular response to the therapy of interest. The use of response signatures in screening the database expands the number of disease states that can be searched or identified for which the therapy of interest would be therapeutically effective in treating.

In some embodiments, a genetic interaction discriminates between the responder set of biological samples (“responders”) and the nonresponder set of biological samples (“nonresponders”) because it contains one or more nucleic acid sequence pairs that are differentially present or differentially expressed in the responders versus the nonrepsonders. In some embodiments, a genetic interaction is, in fact, a site on a genome that is characterized by one or more genetic markers. Such genetic markers include, but are not limited to, single nucleotide polymorphisms (SNPs), SNP haplotypes, microsatellite markers, restriction fragment length polymorphisms (RFLPs), short tandem repeats, sequence length polymorphisms, DNA methylation, random amplified polymorphic DNA (RAPD), amplified fragment length polymorphisms (AFLP), expressible genes and “simple sequence repeats.” For more information on molecular marker methods, see generally, The DNA Revolution by Andrew H. Paterson 1996 (Chapter 2) in: Genome Mapping in Plants (ed. Andrew H. Paterson) by Academic Press/R. G. Landis Company, Austin, Tex., 7-21, which is hereby incorporated by reference herein in its entirety. For example, a particular cellular constituent may contain one or more nucleic acid sequence pairs that are more often present in the responders versus the nonresponders. The statistical tests described herein can be used to determine whether such a differential presence of genetic markers exists. For example, a t-test can be used to determine whether the prevalence of one or more nucleic acid sequence pairs in a genetic interaction discriminates between the responders and the nonresponders. A particular p value for the t-test can be chosen as the threshold for determining whether the cellular constituent discriminates between responders and nonresponders. For instance, of the p value for the t-test (or other form of statistical test such as the ones described above) is 0.05 or less, the genetic interaction is deemed to discriminate between responders and nonresponders in some embodiments of the present invention based on differential presence or absence of one or more nucleic acid sequences within the genetic interaction.

According to some embodiments, the invention provides a software component or other non-transitory computer program product that is encoded on a computer-readable storage medium, and which optionally includes instructions (such as a programmed script or the like) that, when executed, cause operations related to the identification of rescue mutants and/or nucleic acid pairs and/or the probability of a subject or population of subjects having a prognosis or disease state caused by expression of one or a plurality of rescue mutations. In some embodiments, the computer program product is encoded on a computer-readable storage medium that, when executed: identifies or quantifies one or more rescue mutants; normalizes the one or more values corresponding to expression of one or more rescue mutants over a control set of data; creates a rescue mutant profile or signature of a subject; and displays the profile or signature to a user of the computer program product. In some embodiments, the computer program product is encoded on a computer-readable storage medium that, when executed: identifies or quantifies one or more rescue mutants; normalizes the one or more values corresponding to expression of one or more rescue mutants over a control set of data; creates a rescue mutant profile or signature of a subject, wherein the computer program product optionally displays the rescue mutant signature and/or profile or values on a display operated by a user. In some embodiments, the invention relates to a non-transitory computer program product encoded on a computer-readable storage medium comprising instructions for: identifies or quantifies one or more rescue mutants; normalizes the one or more values corresponding to expression of one or more rescue mutants over a control set of data; creates a rescue mutant profile or signature (also known as a genetic interaction profile) of a subject; and displaying the one or more rescue mutant profiles or signatures to a user of the computer program product.

In some embodiments, the step of identifying one or more pairs of nucleic acid sequences as a genetic interaction comprises quantifying an average and standard deviation of counts on replicate trials of applying any one or more datasets (information) associated with a disease or disorder in a subject or population of subjects through one, two, three or four or more algorithms disclosed herein. Some operations or sets of operations may be repeated, for example, substantially continuously, for a pre-defined number of iterations, or until one or more conditions are met. In some embodiments, some operations may be performed in parallel, in sequence, or in other suitable orders of execution. Quantification of the output of an algorithm or algorithms is defined as a probability score. One or a plurality of probability scores may be used to compare a threshold value (in some embodiments, predetermined for a given control population) with the score to identify whether there is a statistically significant change in the experimental dataset as compared to the control.

In some embodiments, the step of identifying one or more pairs of nucleic acid sequences as a genetic interaction comprises quantifying an average and standard deviation of counts on replicate trials of applying any one or more datasets (information) associated with a disease or disorder in a subject or population of subjects through one, two, three or four or more algorithms disclosed herein. Some operations or sets of operations may be repeated, for example, substantially continuously in parallel or sequentially, for a pre-defined number of iterations, or until one or more conditions are met. In some embodiments, some operations may be performed in parallel, in sequence, or in other suitable orders of execution. Quantification of the output of an algorithm or algorithms is defined as a probability score. One or a plurality of probability scores may be used to compare a threshold value (in some embodiments, predetermined for a given control population) with the score to identify whether there is a statistically significant change in the experimental dataset as compared to the control. In some embodiments, the use of the terms “probability score” actually includes consideration of individual probability scores for each step of the method, which, when taken together, create one combined probability score. Nevertheless, one of skill in the art would recognize that in some embodiments, the recitation of calculating a probability score may comprise calculation of distinct probability scores for one or more, or each step of the methods disclosed herein such that one recited step actually includes a normalized and weighed consideration of a threshold value corresponding to each such step.

In some embodiments comprising one or a plurality of steps of identifying SR interactions, any of the disclosed methods comprise single statistical tests for each step, but alternative tests may be performed to obtain the comparable results, for instance, as is the case for running the method steps in duplicate, triplicate or more to increase the statistiscal significance of the result(s). In some embodiments comprising a step of molecular screening (or SOF as set forth in the Examples), the methods comprise a step of evaluating candidate nucleic acid pairs that have a molecular expression pattern that is consistent with SR. We made a specific choice of using binomial test because it was most adequate test for the given problem. However, such pairs can be also identified using Wilcoxon ranksum test, t-test or any statistical tests that compares the level of gene A conditioned on the level of gene B, or vice versa.

The present disclosure also relates to clinical screening of data or information associated with human or non-human patients. In some embodiments, the methods disclosed herein comprise obtaining information associated with a disease or disorder from a subject or population of subjects and analyzing the information for correlation between expression of any pair of nucleic acids with patient survival using Cox multivariate regression analysis because it is the most standardized approach in the field for this type of problems. However, this can be achieved by other statistical methods that find association between patient survival or any other clinical variables such as, but not limited to, tumor size, tumor grade, tumor stage that are associated with patient prognosis. Such statistical analyses include parametric and non-parametric models and Kaplan-Meier analysis (which leads to logrank test statistic) is one of the most representative examples among non-parametric approaches.

The present disclosure also relates to methods that comprise a step of analyzing information associated with a subject or population of subjects and a step of phylogenetic analysis. In some embodiments, the methods or systems herein perform a step of phenotypic screening, in which we calculate essentiality of gene A conditioned on the activity of gene B and vice versa. In some embodiments, the methods comprise essentiality screenings of cancer cell lines based on shRNA. However, any data can be used that quantifies cancer cell's fitness in response to genetic perturbations (knockout, knock-down, over-expression, etc). Fitness measure could be proliferation (as in the dataset we used), migration, invasion, immune response, etc. Gene perturbation can be performed by different ways including, but not limited to, shRNA functional analysis, siRNA functional analysis, functional analysis performed in the presence of small molecule inhibitors, and/or nucleic acids expressing CRISPR complex (CRSIPR enzyme with or without trcrRNA or sgRNA directed specifically to genes to modify). In some embodiments, this step may be performed using a Wilconxon rank-sum test, one of the standard tests for non-parametric comparison. This can be also achieved any other statistical tests that compares the essentiality of one gene under the condition of activity of another gene including t-test, KS test, hypergeometric test, etc.

The methods and kits described herein may contain any combination or permutation or individual shRNAs disclosed herein or homologues thereof with at least 70, 80, 85, 90, 91, 92, 93, 94, 95, 96, 97, 98, or 99% homology to the sequences of Table 6.

The present disclosure also relates to methods of detecting or analyzing any amino acids or nucleic acids disclosed herin or varints of those amino acids or nucleic acids that are with at least 70, 80, 85, 90, 91, 92, 93, 94, 95, 96, 97, 98, or 99% homology to the representative sequences.

In phylogenetic screening, we incorporate the evolutionary evidence that supports the genetic interactions. In some embodiments, any of the disclosed methods may comprise a step of calculating the phylogenetic distance between a pair of genes in three steps: (i) the mapping between homologs in different organisms, (ii) matrix transformation to account for the fact that the species belong to different positions in the tree of life, and (iii) measuring distances of the pair of genes based on the phylogeny in Euclieadian metric. This can be achieved by potentially different alternative ways to identify phylogeny, how to account for the tree of life, and measuring the distance.

In all the above screenings, we determined a gene's activity based on molecular data. Such molecular data include different types measurements such as, but not limited to, DNA sequencing (mutation presence or frequency), RNA sequencing (gene expression; transcriptomics), SCNA, methylation quantification, miRNA expression, IcRNA presence or frequency, proteomic pattern expression, and fluxomics. In some embodiments, any of the methods disclosed herein comprise performing analysis to identify the pairs that are common across many cancer types in all cancer patient population. The same methods can be modified to identify the interaction in particular sub-populations of subjects with conditions or parameters designed to correlate specific cancer type, sub-types, genetic background (eg. cancer driven by specific driver mutations), specific gender, ethnic group, race, stage, grade, and age-group. The type of interaction one can identify is not limited to SR. As an example, methods of the present disclosure relate to identifying the nucleic acid sequence pairs that contribute to synthetic lethality (where single deletion of either a first or second nucleic acid sequences is not lethal while deletion of both the first or second nucleic acid sequences are lethal) and synthetic dosage lethality (where overactivation of one nucleic acid sequence in the pair renders expression or frequency of the other nucleic acid sequence lethal).

In some embodiments, any of the methods disclosed herein can be adapted or replaced with steps to select for or identify a genetic interaction among three, four, five, six or higher order of nucleic acid sequences. In some embodiments, any of the methods disclosed herein can be adapted, supplemented or replaced with steps to select for or identify a genetic interaction determined by analysis of any one or plurality of: protein expression, RNA expression, epigenetic modifications, and/or environmental perturbations.

In some embodiments, the probability score is calculated by normalizing an experimental set of data against a control set of data. Data can be provided in a database or generated through use of normalization of data on a device, such as a microarray. Normalization of data on microarrays can be performed in several ways. A number of different normalization protocols can be used to normalize cellular constituent abundance data. Some such normalization protocols are described in this section. Typically, the normalization comprises normalizing the expression level measurement of each gene in a plurality of genes that is expressed by a subject. Many of the normalization protocols described in this section are used to normalize microarray data. It will be appreciated that there are many other suitable normalization protocols that may be used in accordance with the present invention. All such protocols are within the scope of the present invention. Many of the normalization protocols found in this section are found in publicly available software, such as Microarray Explorer (Image Processing Section, Laboratory of Experimental and Computational Biology, National Cancer Institute, Frederick, Md. 21702, USA).

One normalization protocol is Z-score of intensity. In this protocol, raw expression intensities are normalized by the (mean intensity)/(standard deviation) of raw intensities for all spots in a sample. For microarray data, the Z-score of intensity method normalizes each hybridized sample by the mean and standard deviation of the raw intensities for all of the spots in that sample. The mean intensity mnIᵢ and the standard deviation sdIᵢ are computed for the raw intensity of control genes. It is useful for standardizing the mean (to 0.0) and the range of data between hybridized samples to about −3.0 to +3.0. When using the Z-score, the Z differences (Z_{diff}) are computed rather than ratios, The Z-score intensity (Z-scoreᵢⱼ) for intensity Iᵢⱼ for probe i (hybridization probe, protein, or other binding entity) and spot j is computed as: Z-scoreᵢⱼ=(Iᵢⱼ−mnIᵢ)/sdIᵢ, and Zdiffⱼ(x,y)=Z-scoreₓᵢ−Z-score_{yj} where x represents the x channel and y represents the y channel.

Another normalization protocol is the median intensity normalization protocol in which the raw intensities for all spots in each sample are normalized by the median of the raw intensities. For microarray data, the median intensity normalization method normalizes each hybridized sample by the median of the raw intensities of control genes (medianIᵢ) for all of the spots in that sample. Thus, upon normalization by the median intensity normalization method, the raw intensity Iᵢⱼ for probe i and spot j, has the value Imᵢⱼ where, Imᵢⱼ=(Iᵢⱼ/medianIᵢ).

Another normalization protocol is the log median intensity protocol. In this protocol, raw expression intensities are normalized by the log of the median scaled raw intensities of representative spots for all spots in the sample. For microarray data, the log median intensity method normalizes each hybridized sample by the log of median scaled raw intensities of control genes (medianIᵢ) for all of the spots in that sample. As used herein, control genes are a set of genes that have reproducible accurately measured expression values. The value 1.0 is added to the intensity value to avoid taking the log(0.0) when intensity has zero value. Upon normalization by the median intensity normalization method, the raw intensity Iᵢⱼ for probe i and spot j, has the value Imᵢⱼ where, Im.sub.ij=log(1.0+(Iᵢⱼ/medianIᵢ)).

Yet another normalization protocol is the Z-score standard deviation log of intensity protocol. In this protocol, raw expression intensities are normalized by the mean log intensity (mnLIᵢ) and standard deviation log intensity (sdLIᵢ). For microarray data, the mean log intensity and the standard deviation log intensity is computed for the log of raw intensity of control genes. Then, the Z-score intensity Z log S.sub.ij for probe i and spot j is: Z log Sᵢⱼ=(log(Iᵢⱼ)−mnLIᵢ)/sdLIᵢ.

Still another normalization protocol is the Z-score mean absolute deviation of log intensity protocol. In this protocol, raw expression intensities are normalized by the Z-score of the log intensity using the equation (log(intensity)−mean logarithm)/standard deviation logarithm. For microarray data, the Z-score mean absolute deviation of log intensity protocol normalizes each bound sample by the mean and mean absolute deviation of the logs of the raw intensities for all of the spots in the sample. The mean log intensity mnLIᵢ and the mean absolute deviation log intensity madLIᵢ are computed for the log of raw intensity of control genes. Then, the Z-score intensity Z log Aᵢⱼ for probe i and spot j is: Z log Aᵢⱼ=(log(Iᵢⱼ)−mnLIᵢ)/madLIᵢ.

Another normalization protocol is the user normalization gene set protocol. In this protocol, raw expression intensities are normalized by the sum of the genes in a user defined gene set in each sample. This method is useful if a subset of genes has been determined to have relatively constant expression across a set of samples. Yet another normalization protocol is the calibration DNA gene set protocol in which each sample is normalized by the sum of calibration DNA genes. As used herein, calibration DNA genes are genes that produce reproducible expression values that are accurately measured. Such genes tend to have the same expression values on each of several different microarrays. The algorithm is the same as user normalization gene set protocol described above, but the set is predefined as the genes flagged as calibration DNA.

Yet another normalization protocol is the ratio median intensity correction protocol. This protocol is useful in embodiments in which a two-color fluorescence labeling and detection scheme is used. In the case where the two fluors in a two-color fluorescence labeling and detection scheme are Cy3 and Cy5, measurements are normalized by multiplying the ratio (Cy3/Cy5) by medianCy5/medianCy3 intensities. If background correction is enabled, measurements are normalized by multiplying the ratio (Cy3/Cy5) by (medianCy5−medianBkgdCy5)/(medianCy3−medianBkgdCy3) where medianBkgd means median background levels.

In some embodiments, intensity background correction is used to normalize measurements. The background intensity data from quantification programs may be used to correct spot intensity from fluorescence measurements made to complete a dataset. Background may be specified as either a global value or on a per-spot basis. If the array images have low background, then intensity background correction may not be necessary.

The disclosure relates to methods of identifying a genetic interaction between at least two nucleic acid sequences. In some embodiments, the genetic interaction between the nucleic acid sequence is based upon their protein expression of the first and second nucleic acid seqeunces. In some embodiments, the first and/or second nucleic acid sequences are based upon the expressible portion of genes identified In some embodiments, components and/or units of the devices described herein may be able to interact through one or more communication channels or mediums or links, for example, a shared access medium, a global communication network, the Internet, the World Wide Web, a wired network, a wireless network, a combination of one or more wired networks and/or one or more wireless networks, one or more communication networks, an a-synchronic or asynchronous wireless network, a synchronic wireless network, a managed wireless network, a non-managed wireless network, a burstable wireless network, a non-burstable wireless network, a scheduled wireless network, a non-scheduled wireless network, or the like.

Discussions herein utilizing terms such as, for example, “processing,” “computing,” “calculating,” “determining,” or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

Some embodiments may take the form of an entirely hardware embodiment, an entirely software embodiment, or an embodiment including both hardware and software elements. Some embodiments may be implemented in software, which includes but is not limited to firmware, resident software, microcode, or the like.

Furthermore, some embodiments may take the form of a computer program product accessible from a computer-usable or computer-readable medium providing program code for use by or in connection with a computer or any instruction execution system. For example, a computer-usable or computer-readable medium may be or may include any apparatus that can contain, store, communicate, propagate, or transport the program for use by or in connection with the instruction execution system, apparatus, or device.

In some embodiments, the medium may be or may include an electronic, magnetic, optical, electromagnetic, InfraRed (IR), or semiconductor system (or apparatus or device) or a propagation medium. Some demonstrative examples of a computer-readable medium may include a semiconductor or solid state memory, magnetic tape, a removable computer diskette, a Random Access Memory (RAM), a Read-Only Memory (ROM), a rigid magnetic disk, an optical disk, or the like. Some demonstrative examples of optical disks include Compact Disk-Read-Only Memory (CD-ROM), Compact Disk-Read/Write (CD-R/W), DVD, or the like.

In some embodiments, a data processing system suitable for storing and/or executing program code may include at least one processor coupled directly or indirectly to memory elements, for example, through a system bus. The memory elements may include, for example, local memory employed during actual execution of the program code, bulk storage, and cache memories which may provide temporary storage of at least some program code in order to reduce the number of times code must be retrieved from bulk storage during execution.

In some embodiments, input/output or I/O devices (including but not limited to keyboards, displays, pointing devices, etc.) may be coupled to the system either directly or through intervening I/O controllers. In some embodiments, network adapters may be coupled to the system to enable the data processing system to become coupled to other data processing systems or remote printers or storage devices, for example, through intervening private or public networks. In some embodiments, modems, cable modems and Ethernet cards are demonstrative examples of types of network adapters. Other suitable components may be used.

Some embodiments may be implemented by software, by hardware, or by any combination of software and/or hardware as may be suitable for specific applications or in accordance with specific design requirements. Some embodiments may include units and/or sub-units, which may be separate of each other or combined together, in whole or in part, and may be implemented using specific, multi-purpose or general processors or controllers. Some embodiments may include buffers, registers, stacks, storage units and/or memory units, for temporary or long-term storage of data or in order to facilitate the operation of particular implementations.

Some embodiments may be implemented, for example, using a machine-readable medium or article which may store an instruction or a set of instructions that, if executed by a machine, cause the machine to perform a method and/or operations described herein. Such machine may include, for example, any suitable processing platform, computing platform, computing device, processing device, electronic device, electronic system, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit; for example, memory, removable or non-removable media, erasable or non-erasable media, writeable or re-writeable media, digital or analog media, hard disk drive, floppy disk, Compact Disk Read Only Memory (CD-ROM), Compact Disk Recordable (CD-R), Compact Disk Re-Writeable (CD-RW), optical disk, magnetic media, various types of Digital Versatile Disks (DVDs), a tape, a cassette, or the like. The instructions may include any suitable type of code, for example, source code, compiled code, interpreted code, executable code, static code, dynamic code, or the like, and may be implemented using any suitable high-level, low-level, object-oriented, visual, compiled and/or interpreted programming language, e.g., C, C++, Java, BASIC, Pascal, Fortran, Cobol, assembly language, machine code, or the like.

Functions, operations, components and/or features described herein with reference to one or more embodiments, may be combined with, or may be utilized in combination with, one or more other functions, operations, components and/or features described herein with reference to one or more other embodiments, or vice versa.

In one embodiment, the methods of this invention can be implemented by use of kits. Such kits contain software and/or software systems, such as those described herein. In some embodiments, the kits may comprise microarrays comprising a solid phase, e.g., a surface, to which probes are hybridized or bound at a known location of the solid phase. Preferably, these probes consist of nucleic acids of known, different sequence, with each nucleic acid being capable of hybridizing to an RNA species or to a cDNA species derived therefrom. In a particular embodiment, the probes contained in the kits of this invention are nucleic acids capable of hybridizing specifically to nucleic acid sequences derived from RNA species in cells collected from subject of interest. In some embodiments, any of the disclosed methods comprise a step of obtaining or providing information associated with a disease or disorder. In some embodiments, the step of obtaining or providing comprises isolating a sample from a subject or population of subjects and, optionally performing a genetic screen to obtain expression data or nucleic acid sequence activity data which can then be analyzed with other disclosed steps as compared to a control subject or control population of subjects.

In some embodiments, data or information associated with a subject or population of subjects may be obtained by an individual patient and scored across any or all of the steps disclosed herein by comparing the analysis to information associated with a disease or disorder from a control subject or control population of subjects. In some embodiments, the disease is cancer. In some embodiments, the data or information associated with a disease is taken from any of the data provided in https://gdc-portal.nci.nih.gov, an NIH database of clinical data, which is hereby incorporated by reference in its entirety. Any of the data from the website may be analyzed across one or a plurality of conditions including cancer types disclosed on within the NIH database.

In some embodiments, a kit of the invention also contains one or more databases described above, encoded on computer readable medium, and/or an access authorization to use the databases described above from a remote networked computer.

In another embodiment, a kit of the invention further contains software capable of being loaded into the memory of a computer system such as the one described above. The software contained in the kit of this invention, is essentially identical to the software described above.

Alternative kits for implementing the analytic methods of this invention will be apparent to one of skill in the art and are intended to be comprehended within the accompanying claims.

Although the disclosure has been described with reference to exemplary embodiments, it is not limited thereto. Those skilled in the art will appreciate that numerous changes and modifications may be made to the preferred embodiments of the disclosure and that such changes and modifications may be made without departing from the true spirit of the disclosure. It is therefore intended that the appended claims be construed to cover all such equivalent variations as fall within the true spirit and scope of the disclosure.

Any and all journal articles, patent applications, geneID references, websites or other GenBank or Accession Numbers are hereby incorporated by reference in their entireties.

TABLE 6
Experimental data of the genes screened in the mTOR shRNA experimental analysis The table
lists the sequence for shRNA knockout for each gene, and the measured cell counts of the
genes in the mTOR experimental analysis

| SEQ | | | | | |
|---|---|---|---|---|---|
| ID | | | Gene_ | Gene_ | |
| NO: | 22.mer_sequence | refSeq_Acc | ID | symbol | Gene_description |
| 1 | TTATTGGAAGATCATTGCTGTT | NM_007065 | 11140 | CDC37 | Homo sapiens cell division cycle 37 homolog |
| | | | | | (S. cerevisiae)(CDC37), mRNA. |
| 2 | TACAGATACAGGTGAACTGGCC | NM_000435 | 4854 | NOTCH3 | Homo sapiens notch 3 (NOTCH3), mRNA. |
| 3 | ATACAGATACAGGTGAACTGGC | NM_000435 | 4854 | NOTCH3 | Homo sapiens notch 3 (NOTCH3), mRNA. |
| 4 | TATACTCTGCCTCCAGGGACGT | NM_181710 | 148066 | ZNRF4 | zinc and ring finger 4 |
| 5 | TTATAAATAGGTCTTGCCGTCC | NM_012398, | 23396 | PIP5K1C | phosphatidylinositol-4-phosphate 5-kinase, |
| | | NM_001195733 | | | type I, gamma |
| 6 | TATTATAAATAGGTCTTGCCGT | NM_012398, | 23396 | PIP5K1C | phosphatidylinositol-4-phosphate 5-kinase, |
| | | NM_001195733 | | | type I, gamma |
| 7 | AACTCGGCAAGTTTATTCTGGT | NM_004359 | 997 | CDC34 | Homo sapiens cell division cycle 34 homolog |
| | | | | | (S. cerevisiae)(CDC34), mRNA. |
| 8 | ATCACACTCAGGAGAATGGTCC | NM_004359 | 997 | CDC34 | Homo sapiens cell division cycle 34 homolog |
| | | | | | (S. cerevisiae)(CDC34), mRNA. |
| 9 | ATGAGGTTGCAGAAGAACACGG | NM_139355 | 4145 | MATK | Homo sapiens megakaryocyte-associated |
| | | | | | tyrosine kinase (MATK), transcript variant 1, |
| | | | | | mRNA. |
| 10 | ATATAGATATCTATGCTTCCCA | NM_015675 | 4616 | GADD45B | Homo sapiens growth arrest and DNA- |
| | | | | | damage-inducible, beta (GADD45B), mRNA. |
| 11 | AATATAGATATCTATGCTTCCC | NM_015675 | 4616 | GADD45B | Homo sapiens growth arrest and DNA- |
| | | | | | damage-inducible, beta (GADD45B), mRNA. |
| 12 | ATATCTATGCTTCCCATCTCGC | NM_015675 | 4616 | GADD45B | Homo sapiens growth arrest and DNA- |
| | | | | | damage-inducible, beta (GADD45B), mRNA. |
| 13 | TTAGTAAGGCAGTCTTTGACGA | NM_145185 | 5609 | MAP2K7 | mitogen-activated protein kinase kinase 7 |
| 14 | GTTCTTGTAGGGAAACTGTCCT | NM_145185 | 5609 | MAP2K7 | mitogen-activated protein kinase kinase 7 |
| 15 | TTGACGAAGGACTGGAAGTCCC | NM_145185 | 5609 | MAP2K7 | mitogen-activated protein kinase kinase 7 |
| 16 | TATTCCATGACCATACATAGGT | NM_015016 | 23031 | MAST3 | microtubule associated serine/threonine kinase |
| | | | | | 3 |
| 17 | AATTCCGAGGACTATCCAAGGG | NM_015016 | 23031 | MAST3 | microtubule associated serine/threonine kinase |
| | | | | | 3 |
| 18 | TATTCAGGAGAGATGGGCTGGG | NM_015016 | 23031 | MAST3 | microtubule associated serine/threonine kinase |
| | | | | | 3 |
| 19 | TTACAGATATCCATCATATCCA | NM_001199125, | 8533 | COPS3 | COP9 signalosome subunit 3 |
| | | NM_003653 | | | |
| 20 | TAATGCAGTAACAATAATCTGA | NM_003653 | 8533 | COPS3 | Homo sapiens COP9 constitutive |
| | | | | | photomorphogenic homolog subunit 3 |
| | | | | | (Arabidopsis)(COPS3), transcript variant 1, |
| | | | | | mRNA. |
| 21 | TTACAAGTGCTGATGAAGAGCT | NM_003653 | 8533 | COPS3 | Homo sapiens COP9 constitutive |
| | | | | | photomorphogenic homolog subunit 3 |
| | | | | | (Arabidopsis)(COPS3), transcript variant 1, |
| | | | | | mRNA. |
| 22 | TAAATAAATCCACGACAGACTT | NM_003653 | 8533 | COPS3 | Homo sapiens COP9 constitutive |
| | | | | | photomorphogenic homolog subunit 3 |
| | | | | | (Arabidopsis)(COPS3), transcript variant 1, |
| | | | | | mRNA. |
| 23 | TGATTCCAACATGATATGACTG | NM_003653 | 8533 | COPS3 | Homo sapiens COP9 constitutive |
| | | | | | photomorphogenic homolog subunit 3 |
| | | | | | (Arabidopsis)(COPS3), transcript variant 1, |
| | | | | | mRNA. |
| 24 | TAAAGAGATGACAAGCATTGCT | NM_003653 | 8533 | COPS3 | Homo sapiens COP9 constitutive |
| | | | | | photomorphogenic homolog subunit 3 |
| | | | | | (Arabidopsis)(COPS3), transcript variant 1, |
| | | | | | mRNA. |
| 25 | TATACCTAAGGGCAGAGTTGGT | NM_004656 | 8314 | BAP1 | Homo sapiens BRCA1 associated protein-1 |
| | | | | | (ubiquitin carboxy-terminal hydrolase) |
| | | | | | (BAP1), mRNA. |
| 26 | ATAAAGGTGCAGATGAACTCAT | NM_004656 | 8314 | BAP1 | Homo sapiens BRCA1 associated protein-1 |
| | | | | | (ubiquitin carboxy-terminal hydrolase) |
| | | | | | (BAP1), mRNA. |
| 27 | ATACTTGATCCTGCGGTCGGGC | NM_004656 | 8314 | BAP1 | Homo sapiens BRCA1 associated protein-1 |
| | | | | | (ubiquitin carboxy-terminal hydrolase) |
| | | | | | (BAP1), mRNA. |
| 28 | ATAAATCCATATACAGGGCCCT | NM_004656 | 8314 | BAP1 | Homo sapiens BRCA1 associated protein-1 |
| | | | | | (ubiquitin carboxy-terminal hydrolase) |
| | | | | | (BAP1), mRNA. |
| 29 | TTCGGGCCCATGATGGTGGCCT | NM_015983 | 51619 | UBE2D4 | Homo sapiens ubiquitin-conjugating enzyme |
| | | | | | E2D 4 (putative)(UBE2D4), mRNA. |
| 30 | TACGTTTAAGAGTCTCTCTCCC | NM_015983 | 51619 | UBE2D4 | Homo sapiens ubiquitin-conjugating enzyme |
| | | | | | E2D 4 (putative)(UBE2D4), mRNA. |
| 31 | ATTTGGCATCAAAGAGGTGGCA | NM_015983 | 51619 | UBE2D4 | Homo sapiens ubiquitin-conjugating enzyme |
| | | | | | E2D 4 (putative)(UBE2D4), mRNA. |
| 32 | ATTCCAATTGGAATGTCGTGGT | NM_001145777, | 2289 | FKBP5 | FK506 binding protein 5 |
| | | NM_001145776, | | | |
| | | NM_001145775, | | | |
| | | NM_004117 | | | |
| 33 | ATATATAAGCTCAGCATTAGGT | NM_004117 | 2289 | FKBP5 | Homo sapiens FK506 binding protein 5 |
| | | | | | (FKBP5), transcript variant 1, mRNA. |
| 34 | TTTCCAGATTTGAAAGTGACCA | NM_020903 | 57663 | USP29 | Homo sapiens ubiquitin specific peptidase 29 |
| | | | | | (USP29), mRNA. |
| 35 | TTATCTTCCTTCAGAATGTCCT | NM_020903 | 57663 | USP29 | Homo sapiens ubiquitin specific peptidase 29 |
| | | | | | (USP29), mRNA. |
| 36 | ATATTTCTTGTTTGGTACAGGG | NM_020903 | 57663 | USP29 | Homo sapiens ubiquitin specific peptidase 29 |
| | | | | | (USP29), mRNA. |
| 37 | AATTCTGTAGACTGATTGAGGG | NM_020903 | 57663 | USP29 | Homo sapiens ubiquitin specific peptidase 29 |
| | | | | | (USP29), mRNA. |
| 38 | AATTCATCTATGATGCTCTCCT | NM_020903 | 57663 | USP29 | Homo sapiens ubiquitin specific peptidase 29 |
| | | | | | (USP29), mRNA. |
| 39 | TTGATCTCAGAAATCATCTCCT | NM_020903 | 57663 | USP29 | Homo sapiens ubiquitin specific peptidase 29 |
| | | | | | (USP29), mRNA. |
| 40 | TTGTATAAGTAGGTGGAGACCC | NM_014323 | 23598 | PATZ1 | Homo sapiens POZ (BTB) and AT hook |
| | | | | | containing zinc finger 1 (PATZ1), transcript |
| | | | | | variant 1, mRNA. |
| 41 | ATACTGCAGAAGTTGCTGGGCC | NM_014323 | 23598 | PATZ1 | Homo sapiens POZ (BTB) and AT hook |
| | | | | | containing zinc finger 1 (PATZ1), transcript |
| | | | | | variant 1, mRNA. |
| 42 | TTCACCAATAGGTTGGAGGGCT | NM_139034 | 5598 | MAPK7 | Homo sapiens mitogen-activated protein |
| | | | | | kinase 7 (MAPK7), transcript variant 4, |
| | | | | | mRNA. |
| 43 | TGAAGTACTGATGTTCAGCGGG | NM_139033 | 5598 | MAPK7 | Homo sapiens mitogen-activated protein |
| | | | | | kinase 7 (MAPK7), transcript variant 1, |
| | | | | | mRNA. |
| 44 | TAGTTCAGTCGCCCAAAGGGCA | NM_006712 | 10922 | FASTK | Homo sapiens Fas-activated serine/threonine |
| | | | | | kinase (FASTK), transcript variant 1, mRNA. |
| 45 | TAATTCAATCCAATTTACAGCA | NM_002490 | 4700 | NDUFA6 | Homo sapiens NADH dehydrogenase |
| | | | | | (ubiquinone) 1 alpha subcomplex, 6, 14 kDa |
| | | | | | (NDUFA6), nuclear gene encoding |
| | | | | | mitochondrial protein, mRNA. |
| 46 | TTCTTCATAAACATTTCTCGGA | NM_002490 | 4700 | NDUFA6 | Homo sapiens NADH dehydrogenase |
| | | | | | (ubiquinone) 1 alpha subcomplex, 6, 14 kDa |
| | | | | | (NDUFA6), nuclear gene encoding |
| | | | | | mitochondrial protein, mRNA. |
| 47 | TTTAAGAGAGAATAGTAGTGCT | NM_002711 | 5506 | PPP1R3A | Homo sapiens protein phosphatase 1, |
| | | | | | regulatory subunit 3A (PPP1R3A), mRNA. |
| 48 | TTTGATAATTCTTGAACCTGCC | NM_002711 | 5506 | PPP1R3A | Homo sapiens protein phosphatase 1, |
| | | | | | regulatory subunit 3A (PPP1R3A), mRNA. |
| 49 | AATTATATAGGCTGTACCAGCT | NM_002711 | 5506 | PPP1R3A | Homo sapiens protein phosphatase 1, |
| | | | | | regulatory subunit 3A (PPP1R3A), mRNA. |
| 50 | AGGTAAGATGATGTAGAGGGTG | NM_006833 | 10980 | COPS6 | Homo sapiens COP9 constitutive |
| | | | | | photomorphogenic homolog subunit 6 |
| | | | | | (Arabidopsis)(COPS6), mRNA. |
| 51 | TTATCATGTTTATAAGGTTGGG | NM_006833 | 10980 | COPS6 | Homo sapiens COP9 constitutive |
| | | | | | photomorphogenic homolog subunit 6 |
| | | | | | (Arabidopsis)(COPS6), mRNA. |
| 52 | TTGACCAACCAGTGTGGTGCCT | NM_006833 | 10980 | COPS6 | Homo sapiens COP9 constitutive |
| | | | | | photomorphogenic homolog subunit 6 |
| | | | | | (Arabidopsis)(COPS6), mRNA. |
| 53 | TTAAAGTGTAGAACAGAGACCA | NM_006833 | 10980 | COPS6 | Homo sapiens COP9 constitutive |
| | | | | | photomorphogenic homolog subunit 6 |
| | | | | | (Arabidopsis)(COPS6), mRNA. |
| 54 | TTGGACTGGTACAGGGTGAGGT | NM_000852 | 2950 | GSTP1 | Homo sapiens glutathione S-transferase pi 1 |
| | | | | | (GSTP1), mRNA. |
| 55 | AATTACTCTTCATATTACACCA | NM_002407 | 4246 | SCGB2A1 | Homo sapiens secretoglobin, family 2A, |
| | | | | | member 1 (SCGB2A1), mRNA. |
| 56 | TTCAGAGTTCTATGTGACTGGT | NM_002407 | 4246 | SCGB2A1 | Homo sapiens secretoglobin, family 2A, |
| | | | | | member 1 (SCGB2A1), mRNA. |
| 57 | TTATGTTCAATCATGGTCTGGG | NM_006281 | 6788 | STK3 | Homo sapiens serine/threonine kinase 3 |
| | | | | | (STK3), transcript variant 1, mRNA. |
| 58 | TTTAATTGCGACAACTTGACCG | NM_006281 | 6788 | STK3 | Homo sapiens serine/threonine kinase 3 |
| | | | | | (STK3), transcript variant 1, mRNA. |
| 59 | TATACACATTTGTTTCCTTCCC | NM_002634 | 5245 | PHB | Homo sapiens prohibitin (PHB), mRNA. |
| 60 | TTATATAAGGCAGAGTTCACCA | NM_002634 | 5245 | PHB | Homo sapiens prohibitin (PHB), mRNA. |
| 61 | TTTAGGATGAAGAATACGGTCT | NM_004591 | 6364 | CCL20 | Homo sapiens chemokine (C-C motif) ligand |
| | | | | | 20 (CCL20), transcript variant 1, mRNA. |
| 62 | AATTTAGGATGAAGAATACGGT | NM_004591 | 6364 | CCL20 | Homo sapiens chemokine (C-C motif) ligand |
| | | | | | 20 (CCL20), transcript variant 1, mRNA. |
| 63 | TAACATTCCTGGTGACTCAGGG | NM_000376 | 7421 | VDR | Homo sapiens vitamin D (1,25- |
| | | | | | dihydroxyvitamin D3) receptor (VDR), |
| | | | | | transcript variant 1, mRNA. |
| 64 | ATTTATCGTGAGTAAGGCAGGA | NM_000376 | 7421 | VDR | Homo sapiens vitamin D (1,25- |
| | | | | | dihydroxyvitamin D3) receptor (VDR), |
| | | | | | transcript variant 1, mRNA. |
| 65 | AAGATTAAGCGATATATATGCT | NM_001017535, | 7421 | VDR | vitamin D (1,25- dihydroxyvitamin D3) |
| | | NM_000376, | | | receptor |
| | | NM_001017536 | | | |
| 66 | TTTGGAAATCATTCAGCAGGCA | NM_001017535, | 7421 | VDR | vitamin D (1,25- dihydroxyvitamin D3) |
| | | NM_000376, | | | receptor |
| | | NM_001017536 | | | |
| 67 | ATTCTGCAGTAAGGAACGTGGC | NM_001017535, | 7421 | VDR | vitamin D (1,25- dihydroxyvitamin D3) |
| | | NM_000376, | | | receptor |
| | | NM_001017536 | | | |
| 68 | AAGTGCTATATAAGTATGAGCC | NM_001017535, | 7421 | VDR | vitamin D (1,25- dihydroxyvitamin D3) |
| | | NM_000376, | | | receptor |
| | | NM_001017536 | | | |
| 69 | ATCTTAGCAAAGCCAATGACCT | NM_001017535, | 7421 | VDR | vitamin D (1,25- dihydroxyvitamin D3) |
| | | NM_000376, | | | receptor |
| | | NM_001017536 | | | |
| 70 | TTATTACAGGATCCACATAGGA | NM_000245 | 4233 | MET | Homo sapiens met proto-oncogene (hepatocyte |
| | | | | | growth factor receptor)(MET), transcript |
| | | | | | variant 2, mRNA. |
| 71 | ATAGACAATGGGATCTTCACGG | NM_000245 | 4233 | MET | Homo sapiens met proto-oncogene (hepatocyte |
| | | | | | growth factor receptor)(MET), transcript |
| | | | | | variant 2, mRNA. |
| 72 | TTTACGTTCACATAAGTAGCGT | NM_000245 | 4233 | MET | Homo sapiens met proto-oncogene (hepatocyte |
| | | | | | growth factor receptor)(MET), transcript |
| | | | | | variant 2, mRNA. |
| 73 | TATATTCTACCCAAGGACAGCA | NM_000784 | 1593 | CYP27A1 | Homo sapiens cytochrome P450, family 27, |
| | | | | | subfamily A, polypeptide 1 (CYP27A1), |
| | | | | | nuclear gene encoding mitochondrial protein, |
| | | | | | mRNA. |
| 74 | TTCTGGATCAGCCTTGCGAGGA | NM_000784 | 1593 | CYP27A1 | Homo sapiens cytochrome P450, family 27, |
| | | | | | subfamily A, polypeptide 1 (CYP27A1), |
| | | | | | nuclear gene encoding mitochondrial protein, |
| | | | | | mRNA. |
| 75 | TAACTGGTGCAGTTGCAGGGCA | NM_000784 | 1593 | CYP27A1 | Homo sapiens cytochrome P450, family 27, |
| | | | | | subfamily A, polypeptide 1 (CYP27A1), |
| | | | | | nuclear gene encoding mitochondrial protein, |
| | | | | | mRNA. |
| 76 | TAGAGGAAGAAGTGGTAGCGGG | NM_007284 | 11344 | TWF2 | Homo sapiens twinfilin, actin-binding protein, |
| | | | | | homolog 2 (Drosophila)(TWF2), mRNA. |
| 77 | CATAGTCCTGATCCCAGCGGCC | NM_007284 | 11344 | TWF2 | Homo sapiens twinfilin, actin-binding protein, |
| | | | | | homolog 2 (Drosophila)(TWF2), mRNA. |
| 78 | ATTCCTTCAGCTCTTCCGTGGC | NM_007284 | 11344 | TWF2 | Homo sapiens twinfilin, actin-binding protein, |
| | | | | | homolog 2 (Drosophila)(TWF2), mRNA. |
| 79 | ATTCTCCAGGACCCTGTCTGGG | NM_015695 | 27154 | BRPF3 | bromodomain and PHD finger containing, 3 |
| 80 | ATTGTCAATGCCCTCAATAGGT | NM_015695 | 27154 | BRPF3 | bromodomain and PHD finger containing, 3 |
| 81 | TTTAATATGGCAATAAATGCCT | NM_003391 | 7472 | WNT2 | Homo sapiens wingless-type MMTV |
| | | | | | integration site family member 2 (WNT2), |
| | | | | | transcript variant 1, mRNA. |
| 82 | TATACTTCTGATATTCCATCCA | NM_003391 | 7472 | WNT2 | Homo sapiens wingless-type MMTV |
| | | | | | integration site family member 2 (WNT2), |
| | | | | | transcript variant 1, mRNA. |
| 83 | AGAATAAATCACATGGTGACAG | NM_012289 | 9817 | KEAP1 | Homo sapiens kelch-like ECH-associated |
| | | | | | protein 1 (KEAP1), transcript variant 2, |
| | | | | | mRNA. |
| 84 | AATAAATCACATGGTGACAGCT | NM_203500 | 9817 | KEAP1 | Homo sapiens kelch-like ECH-associated |
| | | | | | protein 1 (KEAP1), transcript variant 1, |
| | | | | | mRNA. |
| 85 | AACACTCAGCTGAATTAAGGCG | NM_203500 | 9817 | KEAP1 | Homo sapiens kelch-like ECH-associated |
| | | | | | protein 1 (KEAP1), transcript variant 1, |
| | | | | | mRNA. |
| 86 | GAATTAAGGCGGTTTGTCCCGT | NM_012289 | 9817 | KEAP1 | Homo sapiens kelch-like ECH-associated |
| | | | | | protein 1 (KEAP1), transcript variant 2, |
| | | | | | mRNA. |
| 87 | TTTAACACTGAGGCATCCTGGC | NM_012289 | 9817 | KEAP1 | Homo sapiens kelch-like ECH-associated |
| | | | | | protein 1 (KEAP1), transcript variant 2, |
| | | | | | mRNA. |
| 88 | ATGCATGTAGATGTACTCCCGG | NM_203500 | 9817 | KEAP1 | Homo sapiens kelch-like ECH-associated |
| | | | | | protein 1 (KEAP1), transcript variant 1, |
| | | | | | mRNA. |
| 89 | TTAAATTCTGGCAGACTTGGCA | NM_017662 | 140803 | TRPM6 | Homo sapiens transient receptor potential |
| | | | | | cation channel, subfamily M, member 6 |
| | | | | | (TRPM6), transcript variant a, mRNA. |
| 90 | TTTCCTGAGGAGTGTCTCTGGT | NM_017662 | 140803 | TRPM6 | Homo sapiens transient receptor potential |
| | | | | | cation channel, subfamily M, member 6 |
| | | | | | (TRPM6), transcript variant a, mRNA. |
| 91 | TAATCTCATTCCATTCCACGGG | NM_003766 | 8678 | BECN1 | Homo sapiens beclin 1, autophagy related |
| | | | | | (BECN1), mRNA. |
| 92 | GTATTCTCTCTGATACTGAGCT | NM_003766 | 8678 | BECN1 | Homo sapiens beclin 1, autophagy related |
| | | | | | (BECN1), mRNA. |
| 93 | TTTCAGACCCATCTTATTGGCC | NM_003766 | 8678 | BECN1 | Homo sapiens beclin 1, autophagy related |
| | | | | | (BECN1), mRNA. |
| 94 | AATCTCCACTGGAGAGAAAGGT | NM_024083 | 79058 | ASPSCR1 | Homo sapiens alveolar soft part sarcoma |
| | | | | | chromosome region, candidate 1 (ASPSCR1), |
| | | | | | transcript variant 1, mRNA. |
| 95 | TTATCTGCGCCTCCCTGAAGGC | NM_024083 | 79058 | ASPSCR1 | Homo sapiens alveolar soft part sarcoma |
| | | | | | chromosome region, candidate 1 (ASPSCR1), |
| | | | | | transcript variant 1, mRNA. |
| 96 | ATTCCAAGGGAAGTGGAGCGCT | NM_024083 | 79058 | ASPSCR1 | Homo sapiens alveolar soft part sarcoma |
| | | | | | chromosome region, candidate 1 (ASPSCR1), |
| | | | | | transcript variant 1, mRNA. |
| 97 | TATTCCTGCTGGCAGAGGAGGT | NM_024083 | 79058 | ASPSCR1 | Homo sapiens alveolar soft part sarcoma |
| | | | | | chromosome region, candidate 1 (ASPSCR1), |
| | | | | | transcript variant 1, mRNA. |
| 98 | TTGAGGGTGGAAATGATGAGGT | NM_017859 | 54963 | UCKL1 | Homo sapiens uridine-cytidine kinase 1-like 1 |
| | | | | | (UCKL1), transcript variant 1, mRNA. |
| 99 | TTGGAGTAGAAGATGAACTCGT | NM_017859 | 54963 | UCKL1 | Homo sapiens uridine-cytidine kinase 1-like 1 |
| | | | | | (UCKL1), transcript variant 1, mRNA. |
| 100 | AATGCATAGGCCACTGAGTGCA | NM_017859 | 54963 | UCKL1 | Homo sapiens uridine-cytidine kinase 1-like 1 |
| | | | | | (UCKL1), transcript variant 1, mRNA. |
| 101 | TTTCTCAGCCTGCCGGCCTGGT | NM_017859 | 54963 | UCKL1 | Homo sapiens uridine-cytidine kinase 1-like 1 |
| | | | | | (UCKL1), transcript variant 1, mRNA. |
| 102 | ATGGACACACCGGTGATCTGCT | NM_017859 | 54963 | UCKL1 | Homo sapiens uridine-cytidine kinase 1-like 1 |
| | | | | | (UCKL1), transcript variant 1, mRNA. |
| 103 | ATATGTGTACATATGTATACGG | NM_014975 | 22983 | MAST1 | microtubule associated serine/threonine kinase |
| | | | | | 1 |
| 104 | TTAGCCTTGTAGCTGCTGCGCC | NM_014975 | 22983 | MAST1 | microtubule associated serine/threonine kinase |
| | | | | | 1 |
| 105 | TTGTCCAGGAACTCTCGGGCGT | NM_014975 | 22983 | MAST1 | microtubule associated serine/threonine kinase |
| | | | | | 1 |
| 106 | TTCAGCGACGACAGCGAGCGGC | NM_014975 | 22983 | MAST1 | microtubule associated serine/threonine kinase |
| | | | | | 1 |
| 107 | ATTAGATGCAAGGAACTCTGGG | NM_002730 | 5566 | PRKACA | Homo sapiens protein kinase, cAMP- |
| | | | | | dependent, catalytic, alpha (PRKACA), |
| | | | | | transcript variant 1, mRNA. |
| 108 | TACTCCGAAAGGAAGGTTGGCG | NM_002730 | 5566 | PRKACA | Homo sapiens protein kinase, cAMP- |
| | | | | | dependent, catalytic, alpha (PRKACA), |
| | | | | | transcript variant 1, mRNA. |
| 109 | TTTGCTCAGGATAATCTCAGGG | NM_002730 | 5566 | PRKACA | Homo sapiens protein kinase, cAMP- |
| | | | | | dependent, catalytic, alpha (PRKACA), |
| | | | | | transcript variant 1, mRNA. |
| 110 | TTTGTTCTTAGGAAGCTTGGCC | NM_002827 | 5770 | PTPN1 | Homo sapiens protein tyrosine phosphatase, |
| | | | | | non-receptor type 1 (PTPN1), mRNA. |
| 111 | AAGAAAGTTCAAGAATGAGGCT | NM_002827 | 5770 | PTPN1 | Homo sapiens protein tyrosine phosphatase, |
| | | | | | non-receptor type 1 (PTPN1), mRNA. |
| 112 | ATAAACGATTTCTCAATTGCAT | NM_005370 | 4218 | RAB8A | Homo sapiens RAB8A, member RAS |
| | | | | | oncogene family (RAB8A), mRNA. |
| 113 | TTTCTCAATTGCATTCTGGTGG | NM_005370 | 4218 | RAB8A | Homo sapiens RAB8A, member RAS |
| | | | | | oncogene family (RAB8A), mRNA. |
| 114 | TAGAAGTCTGAGGAGAGAAGCC | NM_005234 | 2063 | NR2F6 | Homo sapiens nuclear receptor subfamily 2, |
| | | | | | group F, member 6 (NR2F6), mRNA. |
| 115 | TTCTTGAGACGGCAGTACTGGC | NM_005234 | 2063 | NR2F6 | Homo sapiens nuclear receptor subfamily 2, |
| | | | | | group F, member 6 (NR2F6), mRNA. |
| 116 | TTCTGCAACCAGAGATAACTCC | NM_007181 | 11184 | MAP4K1 | Homo sapiens mitogen-activated protein |
| | | | | | kinase kinase kinase kinase 1 (MAP4K1), |
| | | | | | transcript variant 2, mRNA. |
| 117 | ATTGATGAGGATGTTAGCTCCC | NM_007181 | 11184 | MAP4K1 | Homo sapiens mitogen-activated protein |
| | | | | | kinase kinase kinase kinase 1 (MAP4K1), |
| | | | | | transcript variant 2, mRNA. |
| 118 | AAGTATGGAAATGAAGTTGGGC | NM_003290 | 7171 | TPM4 | Homo sapiens tropomyosin 4 (TPM4), |
| | | | | | transcript variant 2, mRNA. |
| 119 | TTTAGAATGAAGGAAATATGCA | NM_003290 | 7171 | TPM4 | Homo sapiens tropomyosin 4 (TPM4), |
| | | | | | transcript variant 2, mRNA. |
| 120 | TTTCACACGCGAAATAGGCCTG | NM_005053 | 5886 | RAD23A | Homo sapiens RAD23 homolog A (S. |
| | | | | | cerevisiae)(RAD23A), mRNA. |

| SEQ | | | | | | | |
|---|---|---|---|---|---|---|---|
| ID | | | | | | | |
| NO: | raw_RBI.01 | raw_RBI.02 | raw_RBI.03 | raw_RBI.10 | raw_RBI.11 | raw_RBI.11 | raw_RBI.12 |
| 1 | 777 | 113 | 480 | 864 | 720 | 720 | 967 |
| 2 | [symbol] | [symbol] | [symbol] | 581 | 401 | 401 | 454 |
| 3 | 710 | 140 | 644 | 583 | 404 | 404 | 459 |
| 4 | 97 | 12 | 10 | 48 | 43 | 43 | 53 |
| 5 | 68 | 6 | 117 | 77 | 103 | 103 | 80 |
| 6 | 68 | 6 | 117 | 77 | 103 | 103 | 81 |
| 7 | 107 | 139 | 9 | 56 | 53 | 53 | 66 |
| 8 | 40 | 0 | 7 | 29 | 34 | 34 | 12 |
| 9 | 33 | 0 | 34 | 38 | 6 | 6 | 35 |
| 10 | 2810 | 622 | 3263 | 4504 | 3857 | 3857 | 3886 |
| 11 | 2810 | 623 | 3259 | 4501 | 3855 | 3855 | 3883 |
| 12 | 112 | 35 | 108 | 51 | 38 | 38 | 40 |
| 13 | 261 | 1157 | 24 | 435 | 448 | 448 | 311 |
| 14 | 44 | 0 | 0 | 11 | 16 | 16 | 10 |
| 15 | 25 | 0 | 0 | 8 | 1 | 1 | 14 |
| 16 | 490 | 557 | 619 | 494 | 523 | 523 | 489 |
| 17 | 14 | 73 | 0 | 7 | 14 | 14 | 53 |
| 18 | 9 | 0 | 61 | 10 | 14 | 14 | 5 |
| 19 | 94 | 0 | 119 | 85 | 82 | 82 | 71 |
| 20 | 915 | 2833 | 6876 | 1940 | 1124 | 1124 | 1450 |
| 21 | 65 | 22 | 337 | 43 | 20 | 20 | 26 |
| 22 | 593 | 1130 | 301 | 1002 | 832 | 832 | 861 |
| 23 | 89 | 0 | 25 | 55 | 67 | 67 | 110 |
| 24 | 31 | 0 | 0 | 6 | 19 | 19 | 6 |
| 25 | 319 | 645 | 538 | 284 | 443 | 443 | 343 |
| 26 | 98 | 25 | 3 | 22 | 41 | 41 | 30 |
| 27 | 29 | 0 | 17 | 1 | 9 | 9 | 2 |
| 28 | 19 | 6 | 0 | 10 | 4 | 4 | 5 |
| 29 | 112 | 1 | 61 | 114 | 384 | 384 | 295 |
| 30 | 47 | 38 | 0 | 41 | 35 | 35 | 51 |
| 31 | 32 | 5 | 53 | 46 | 12 | 12 | 12 |
| 32 | 92 | 31 | 48 | 75 | 64 | 64 | 68 |
| 33 | 1050 | 225 | 1471 | 1266 | 1381 | 1381 | 1300 |
| 34 | 167 | 54 | 6 | 193 | 177 | 177 | 151 |
| 35 | 256 | 351 | 120 | 217 | 273 | 273 | 316 |
| 36 | 102 | 0 | 1 | 132 | 94 | 94 | 127 |
| 37 | 348 | 385 | 47 | 437 | 388 | 388 | 374 |
| 38 | 27 | 1 | 0 | 12 | 7 | 7 | 5 |
| 39 | 22 | 0 | 5 | 40 | 13 | 13 | 30 |
| 40 | 3 | 0 | 0 | 6 | 3 | 3 | 5 |
| 41 | 1 | 0 | 20 | 0 | 2 | 2 | 4 |
| 42 | 105 | 161 | 51 | 52 | 24 | 24 | 124 |
| 43 | 152 | 102 | 5 | 44 | 433 | 433 | 216 |
| 44 | 54 | 38 | 25 | 103 | 100 | 100 | 45 |
| 45 | 86 | 0 | 88 | 201 | 228 | 228 | 167 |
| 46 | 71 | 1672 | 0 | 220 | 137 | 137 | 113 |
| 47 | 719 | 645 | 483 | 1423 | 1042 | 1042 | 1476 |
| 48 | 329 | 403 | 1916 | 562 | 522 | 522 | 523 |
| 49 | 22 | 3 | 12 | 15 | 3 | 3 | 38 |
| 50 | 203 | 1 | 235 | 157 | 148 | 148 | 186 |
| 51 | 22 | 0 | 0 | 35 | 29 | 29 | 57 |
| 52 | 18 | 4 | 0 | 3 | 30 | 30 | 4 |
| 53 | 14 | 0 | 0 | 14 | 17 | 17 | 8 |
| 54 | 55 | 0 | 52 | 9 | 84 | 84 | 2 |
| 55 | 1942 | 1467 | 106 | 2645 | 2610 | 2610 | 2648 |
| 56 | 468 | 121 | 932 | 684 | 534 | 534 | 564 |
| 57 | 91 | 57 | 417 | 125 | 74 | 74 | 156 |
| 58 | 62 | 1 | 26 | 43 | 53 | 53 | 38 |
| 59 | 375 | 281 | 69 | 490 | 822 | 822 | 478 |
| 60 | 228 | 146 | 5 | 350 | 338 | 338 | 352 |
| 61 | 434 | 80 | 208 | 363 | 265 | 265 | 222 |
| 62 | 434 | 79 | 206 | 359 | 264 | 264 | 219 |
| 63 | 60 | 18 | 4 | 50 | 60 | 60 | 123 |
| 64 | 145 | 2015 | 48 | 100 | 229 | 229 | 101 |
| 65 | 801 | 166 | 1663 | 753 | 657 | 657 | 839 |
| 66 | 175 | 0 | 4 | 128 | 267 | 267 | 77 |
| 67 | 86 | 4 | 81 | 48 | 98 | 98 | 99 |
| 68 | 44190 | 31126 | 20847 | 60575 | 44395 | 44395 | 48464 |
| 69 | 5 | 0 | 41 | 17 | 2 | 2 | 5 |
| 70 | 105 | 8 | 0 | 111 | 85 | 85 | 73 |
| 71 | 13 | 0 | 0 | 5 | 6 | 6 | 8 |
| 72 | 8 | 8 | 0 | 18 | 16 | 16 | 20 |
| 73 | 185 | 284 | 242 | 229 | 168 | 168 | 195 |
| 74 | 95 | 13 | 42 | 132 | 13 | 13 | 56 |
| 75 | 0 | 0 | 0 | 4 | 0 | 0 | 0 |
| 76 | 56 | 5 | 58 | 70 | 38 | 38 | 29 |
| 77 | 247 | 20 | 29 | 503 | 311 | 311 | 49 |
| 78 | 20 | 6 | 24 | 12 | 14 | 14 | 44 |
| 79 | 89 | 0 | 11 | 16 | 26 | 26 | 16 |
| 80 | 6 | 0 | 0 | 5 | 2 | 2 | 0 |
| 81 | 302 | 284 | 1087 | 325 | 262 | 262 | 329 |
| 82 | 179 | 0 | 0 | 436 | 502 | 502 | 256 |
| 83 | 87 | 0 | 115 | 65 | 81 | 81 | 48 |
| 84 | 87 | 0 | 114 | 65 | 81 | 81 | 48 |
| 85 | 101 | 42 | 40 | 48 | 70 | 70 | 93 |
| 86 | 31 | 0 | 0 | 13 | 0 | 0 | 0 |
| 87 | 22 | 37 | 0 | 4 | 4 | 4 | 5 |
| 88 | 2 | 0 | 0 | 1 | 1 | 1 | 0 |
| 89 | 64 | 0 | 12 | 36 | 140 | 140 | 367 |
| 90 | 45 | 7 | 10 | 85 | 45 | 45 | 62 |
| 91 | 120 | 38 | 13 | 110 | 100 | 100 | 91 |
| 92 | 85 | 0 | 3 | 328 | 215 | 215 | 24 |
| 93 | 0 | 0 | 0 | 5 | 2 | 2 | 1 |
| 94 | 286 | 71 | 204 | 110 | 63 | 63 | 169 |
| 95 | 98 | 157 | 1 | 43 | 36 | 36 | 34 |
| 96 | 16 | 0 | 51 | 1 | 0 | 0 | 1 |
| 97 | 0 | 0 | 0 | 2 | 0 | 0 | 0 |
| 98 | 246 | 21 | 44 | 107 | 169 | 169 | 198 |
| 99 | 77 | 0 | 15 | 40 | 58 | 58 | 37 |
| 100 | 47 | 19 | 0 | 12 | 7 | 7 | 14 |
| 101 | 34 | 3599 | 17 | 11 | 0 | 0 | 1 |
| 102 | 0 | 0 | 0 | 0 | 0 | 0 | 1 |
| 103 | 1402 | 0 | 103 | 1815 | 1546 | 1546 | 1479 |
| 104 | 26 | 0 | 0 | 0 | 0 | 0 | 0 |
| 105 | 8 | 0 | 1 | 11 | 4 | 4 | 8 |
| 106 | 0 | 0 | 0 | 0 | 1 | 1 | 6 |
| 107 | 227 | 47 | 116 | 272 | 219 | 219 | 219 |
| 108 | 441 | 0 | 0 | 434 | 253 | 253 | 176 |
| 109 | 224 | 23 | 143 | 161 | 324 | 324 | 159 |
| 110 | 130 | 81 | 8441 | 190 | 142 | 142 | 167 |
| 111 | 47 | 90 | 5 | 91 | 66 | 66 | 51 |
| 112 | 166 | 0 | 27 | 177 | 206 | 206 | 184 |
| 113 | 116 | 19 | 1 | 207 | 149 | 149 | 57 |
| 114 | 85 | 0 | 0 | 62 | 39 | 39 | 68 |
| 115 | 3 | 0 | 1 | 7 | 7 | 7 | 15 |
| 116 | 138 | 562 | 17 | 131 | 107 | 107 | 86 |
| 117 | 7 | 0 | 0 | 6 | 0 | 0 | 10 |
| 118 | 280 | 0 | 220 | 195 | 263 | 263 | 153 |
| 119 | 9410 | 11167 | 14166 | 14241 | 12800 | 12800 | 12113 |
| 120 | 73 | 9 | 115 | 37 | 19 | 19 | 13 |

| SEQ | | | | | |
|---|---|---|---|---|---|
| ID | | | | | |
| NO: | raw_RBI.13 | raw_RBI.14 | raw_RBI.15 | n_RBI.01 | n_RBI.02 |
| 1 | 1774 | 3214 | 2867 | 674.5719203 | 98.95572808 |
| 2 | 1796 | 1003 | 100 [symbol] | [symbol] | [symbol] 000171 |
| 3 | 1799 | 1005 | 1009 | 616.4042 | 122.6000171 |
| 4 | 96 | 137 | 49 | 84.21296817 | 10.50857289 |
| 5 | 174 | 106 | 75 | 59.03589521 | 5.254286447 |
| 6 | 175 | 107 | 75 | 59.03589521 | 5.254286447 |
| 7 | 10 | 81 | 73 | 92.89471747 | 121.7243027 |
| 8 | 4 | 20 | 17 | 34.72699718 | 0 |
| 9 | 10 | 39 | 12 | 28.64977268 | 0 |
| 10 | 1964 | 2458 | 3278 | 2439.571552 | 544.6943617 |
| 11 | 1962 | 2460 | 3280 | 2439.571552 | 545.5700761 |
| 12 | 25 | 480 | 158 | 97.23559212 | 30.65000427 |
| 13 | 1771 | 196 | 192 | 226.5936566 | 1013.20157 |
| 14 | 31 | 22 | 105 | 38.1996969 | 0 |
| 15 | 0 | 2 | 0 | 21.70437324 | 0 |
| 16 | 218 | 378 | 120 | 425.4057155 | 487.7729252 |
| 17 | 0 | 4 | 0 | 12.15444901 | 63.92715177 |
| 18 | 0 | 0 | 0 | 7.813574366 | 0 |
| 19 | 576 | 548 | 1013 | 81.60844338 | 0 |
| 20 | 8871 | 8212 | 4981 | 794.3800606 | 2480.898917 |
| 21 | 316 | 30 | 21 | 56.43137042 | 19.26571697 |
| 22 | 504 | 1240 | 776 | 514.8277333 | 989.5572808 |
| 23 | 85 | 191 | 7 | 77.26756874 | 0 |
| 24 | 33 | 20 | 44 | 26.91342282 | 0 |
| 25 | 476 | 174 | 259 | 276.9478025 | 564.835793 |
| 26 | 31 | 30 | 21 | 85.0811431 | 21.8928602 |
| 27 | 12 | 40 | 11 | 25.17707296 | 0 |
| 28 | 29 | 14 | 5 | 16.49532366 | 5.254286447 |
| 29 | 148 | 80 | 155 | 97.23559212 | 0.875714408 |
| 30 | 140 | 121 | 80 | 40.80422169 | 33.2771475 |
| 31 | 27 | 24 | 99 | 27.78159775 | 4.378572039 |
| 32 | 134 | 172 | 123 | 79.87209352 | 27.14714664 |
| 33 | 993 | 911 | 1021 | 911.5836761 | 197.0357418 |
| 34 | 133 | 85 | 106 | 144.9852132 | 47.28857802 |
| 35 | 177 | 144 | 200 | 222.252782 | 307.3757571 |
| 36 | 4 | 28 | 47 | 88.55384282 | 0 |
| 37 | 340 | 305 | 584 | 302.1248755 | 337.150047 |
| 38 | 45 | 40 | 12 | 23.4407231 | 0.875714408 |
| 39 | 3 | 63 | 25 | 19.09984845 | 0 |
| 40 | 0 | 0 | 2 | 2.604524789 | 0 |
| 41 | 0 | 4 | 0 | 0.86817493 | 0 |
| 42 | 209 | 188 | 63 | 91.15836761 | 140.9900197 |
| 43 | 16 | 348 | 37 | 131.9625893 | 89.3228696 |
| 44 | 146 | 67 | 44 | 46.8814462 | 33.2771475 |
| 45 | 27 | 70 | 136 | 74.66304395 | 0 |
| 46 | 191 | 543 | 146 | 61.64042 | 1464.19449 |
| 47 | 286 | 633 | 856 | 624.2177744 | 564.835793 |
| 48 | 220 | 491 | 480 | 285.6295518 | 352.9129063 |
| 49 | 0 | 2 | 8 | 19.09984845 | 2.627143223 |
| 50 | 126 | 67 | 84 | 176.2395107 | 0.875714408 |
| 51 | 2 | 2 | 0 | 19.09984845 | 0 |
| 52 | 20 | 1 | 11 | 15.62714873 | 3.502857631 |
| 53 | 24 | 26 | 132 | 12.15444901 | 0 |
| 54 | 57 | 4 | 11 | 47.74962113 | 0 |
| 55 | 1288 | 2149 | 2340 | 1685.995713 | 1284.673036 |
| 56 | 111 | 783 | 256 | 406.3058671 | 105.9614433 |
| 57 | 348 | 420 | 312 | 79.00391859 | 49.91572125 |
| 58 | 29 | 40 | 5 | 53.82684564 | 0.875714408 |
| 59 | 297 | 302 | 398 | 325.5655986 | 246.0757486 |
| 60 | 85 | 288 | 180 | 197.943884 | 127.8543035 |
| 61 | 124 | 182 | 194 | 376.7879195 | 70.05715263 |
| 62 | 124 | 182 | 194 | 376.7879195 | 69.18143822 |
| 63 | 14 | 4 | 8 | 52.09049578 | 15.76285934 |
| 64 | 89 | 99 | 144 | 125.8853648 | 1764.564532 |
| 65 | 345 | 572 | 380 | 695.4081186 | 145.3685917 |
| 66 | 119 | 44 | 9 | 151.9306127 | 0 |
| 67 | 5 | 95 | 35 | 74.66304395 | 3.502857631 |
| 68 | 84893 | 41873 | 34926 | 38364.65014 | 27257.48666 |
| 69 | 0 | 4 | 6 | 4.340874648 | 0 |
| 70 | 10 | 285 | 164 | 91.15836761 | 7.005715263 |
| 71 | 0 | 10 | 5 | 11.28627408 | 0 |
| 72 | 0 | 23 | 2 | 6.945399437 | 7.005715263 |
| 73 | 20 | 39 | 139 | 160.612362 | 248.7028918 |
| 74 | 58 | 27 | 29 | 82.47661831 | 11.3842873 |
| 75 | 0 | 0 | 0 | 0 | 0 |
| 76 | 32 | 16 | 33 | 48.61779606 | 4.378572039 |
| 77 | 261 | 56 | 35 | 214.4392076 | 17.51428816 |
| 78 | 0 | 13 | 24 | 17.36349859 | 5.254286447 |
| 79 | 33 | 38 | 33 | 77.26756874 | 0 |
| 80 | 0 | 0 | 0 | 5.209049578 | 0 |
| 81 | 76 | 143 | 184 | 262.1888287 | 248.7028918 |
| 82 | 8 | 86 | 71 | 155.4033124 | 0 |
| 83 | 40 | 10 | 21 | 75.53121888 | 0 |
| 84 | 40 | 10 | 21 | 75.53121888 | 0 |
| 85 | 57 | 35 | 38 | 87.68566789 | 36.78000513 |
| 86 | 0 | 0 | 0 | 26.91342282 | 0 |
| 87 | 0 | 17 | 6 | 19.09984845 | 32.40143309 |
| 88 | 0 | 0 | 0 | 1.736349859 | 0 |
| 89 | 12 | 54 | 8 | 55.56319549 | 0 |
| 90 | 2 | 40 | 31 | 39.06787183 | 6.130000855 |
| 91 | 50 | 15 | 16 | 104.1809916 | 33.2771475 |
| 92 | 8 | 34 | 9 | 73.79486902 | 0 |
| 93 | 0 | 0 | 0 | 0 | 0 |
| 94 | 475 | 109 | 132 | 248.2980299 | 62.17572295 |
| 95 | 50 | 6 | 23 | 85.0811431 | 137.487162 |
| 96 | 5 | 0 | 4 | 13.89079887 | 0 |
| 97 | 0 | 0 | 0 | 0 | 0 |
| 98 | 97 | 62 | 51 | 213.5710327 | 18.39000256 |
| 99 | 8 | 27 | 38 | 66.84946958 | 0 |
| 100 | 39 | 14 | 0 | 40.80422169 | 16.63857375 |
| 101 | 0 | 56 | 0 | 29.51794761 | 3151.696154 |
| 102 | 0 | 0 | 0 | 0 | 0 |
| 103 | 900 | 1214 | 530 | 1217.181251 | 0 |
| 104 | 0 | 0 | 0 | 22.57254817 | 0 |
| 105 | 0 | 1 | 49 | 6.945399437 | 0 |
| 106 | 0 | 1 | 0 | 0 | 0 |
| 107 | 66 | 104 | 69 | 197.075709 | 41.15857717 |
| 108 | 216 | 87 | 16 | 382.865144 | 0 |
| 109 | 146 | 197 | 233 | 194.4711842 | 20.14143138 |
| 110 | 48 | 39 | 24 | 112.8627408 | 70.93286703 |
| 111 | 23 | 369 | 7 | 40.80422169 | 78.8142967 |
| 112 | 48 | 253 | 37 | 144.1170383 | 0 |
| 113 | 118 | 46 | 72 | 100.7082918 | 16.63857375 |
| 114 | 14 | 12 | 3 | 73.79486902 | 0 |
| 115 | 0 | 4 | 0 | 2.604524789 | 0 |
| 116 | 2 | 112 | 118 | 119.8081403 | 492.1514972 |
| 117 | 0 | 2 | 1 | 6.077224507 | 0 |
| 118 | 17 | 169 | 36 | 243.0889803 | 0 |
| 119 | 21311 | 11112 | 14490 | 8169.526088 | 9779.102792 |
| 120 | 2 | 52 | 27 | 63.37676986 | 7.88142967 |

[symbol]

indicates data missing or illegible when filed

| SEQ | | | | | |
|---|---|---|---|---|---|
| ID | | | | | |
| NO: | n_RBI.03 | n_RBI.10 | n_RBI.11 | n_RBI.12 | n_RBI.13 |
| 1 | 464.9764257 | 513.7757307 | 469.8272993 | 649.6282675 | 1108.69706 |
| 2 | 622.8746703 | 345.490393 | 261.6677042 | 304.9961049 | 1122.446403 |
| 3 | 623.8433711 | 346.6796887 | 263.6253179 | 308.3550929 | 1124.321314 |
| 4 | 9.687008869 | 28.54309615 | 28.05913038 | 35.60527216 | 59.99713514 |
| 5 | 113.3380038 | 45.78788341 | 67.21140532 | 53.74380703 | 108.7448074 |
| 6 | 113.3380038 | 45.78788341 | 67.21140532 | 54.41560462 | 109.3697776 |
| 7 | 8.718307982 | 33.30027884 | 34.58450953 | 44.3386408 | 6.249701577 |
| 8 | 6.780906208 | 17.24478726 | 22.18628913 | 8.061571055 | 2.499880631 |
| 9 | 32.93583015 | 22.59661779 | 3.915227494 | 23.51291558 | 6.249701577 |
| 10 | 3160.870994 | 2678.293855 | 2516.838741 | 2610.605427 | 1227.44139 |
| 11 | 3156.99619 | 2676.509912 | 2515.533665 | 2608.590034 | 1226.191449 |
| 12 | 104.6196958 | 30.32703966 | 24.7964408 | 26.87190352 | 15.62425394 |
| 13 | 23.24882128 | 258.6718089 | 292.3369862 | 208.9290498 | 1106.822149 |
| 14 | 0 | 6.541126201 | 10.44060665 | 6.717975879 | 19.37407489 |
| 15 | 0 | 4.757182692 | 0.652537916 | 9.405166231 | 0 |
| 16 | 599.625849 | 293.7560312 | 341.2773299 | 328.5090205 | 136.2434944 |
| 17 | 0 | 4.162534855 | 9.13553082 | 35.60527216 | 0 |
| 18 | 59.0907541 | 5.946478365 | 9.13553082 | 3.35898794 | 0 |
| 19 | 115.2754055 | 50.5450661 | 53.50810909 | 47.69762874 | 359.9828108 |
| 20 | 6660.787298 | 1153.616803 | 733.4526173 | 974.1065025 | 5544.110269 |
| 21 | 326.4521989 | 25.56985697 | 13.05075831 | 17.46673729 | 197.4905698 |
| 22 | 291.5789669 | 595.8371321 | 542.9115459 | 578.4177232 | 314.9849595 |
| 23 | 24.21752217 | 32.70563101 | 43.72004035 | 73.89773467 | 53.1224634 |
| 24 | 0 | 3.567887019 | 12.3982204 | 4.030785528 | 20.6240152 |
| 25 | 521.1610771 | 168.8799856 | 289.0742967 | 230.4265727 | 297.4857951 |
| 26 | 2.906102661 | 13.0822524 | 26.75405454 | 20.15392764 | 19.37407489 |
| 27 | 16.46791508 | 0.594647836 | 5.872841241 | 1.343595176 | 7.499641892 |
| 28 | 0 | 5.946478365 | 2.610151663 | 3.35898794 | 18.12413457 |
| 29 | 59.0907541 | 67.78985336 | 250.5745596 | 198.1802884 | 92.49558334 |
| 30 | 0 | 24.3805613 | 22.83882705 | 34.26167698 | 87.49582207 |
| 31 | 51.341147 | 27.35380048 | 7.830454989 | 8.061571055 | 16.87419426 |
| 32 | 46.49764257 | 44.59858774 | 41.76242661 | 45.68223598 | 83.74600113 |
| 33 | 1424.959005 | 752.824161 | 901.1548616 | 873.3368643 | 620.5953666 |
| 34 | 5.812205321 | 114.7670324 | 115.4992111 | 101.4414358 | 83.12103097 |
| 35 | 116.2441064 | 129.0385805 | 178.142851 | 212.2880378 | 110.6197179 |
| 36 | 0.968700887 | 78.49351441 | 61.33856408 | 85.31829367 | 2.499880631 |
| 37 | 45.52894168 | 259.8611045 | 253.1847113 | 251.2522979 | 212.4898536 |
| 38 | 0 | 7.135774038 | 4.56776541 | 3.35898794 | 28.1236571 |
| 39 | 4.843504434 | 23.78591346 | 8.482992904 | 20.15392764 | 1.874910473 |
| 40 | 0 | 3.567887019 | 1.957613747 | 3.35898794 | 0 |
| 41 | 19.37401774 | 0 | 1.305075831 | 2.687190352 | 0 |
| 42 | 49.40374523 | 30.9216875 | 15.66090998 | 83.3029009 | 130.618763 |
| 43 | 4.843504434 | 26.1645048 | 282.5489175 | 145.108279 | 9.999522523 |
| 44 | 24.21752217 | 61.24872716 | 65.25379157 | 30.23089146 | 91.24564302 |
| 45 | 85.24567804 | 119.5242151 | 148.7786448 | 112.1901972 | 16.87419426 |
| 46 | 0 | 130.822524 | 89.39769445 | 75.91312744 | 119.3693001 |
| 47 | 467.8825284 | 846.1838713 | 679.9445082 | 991.5732398 | 178.7414651 |
| 48 | 1856.030899 | 334.1920841 | 340.624792 | 351.3501385 | 137.4934347 |
| 49 | 11.62441064 | 8.919717547 | 1.957613747 | 25.52830834 | 0 |
| 50 | 227.6447084 | 93.35971033 | 96.57561153 | 124.9543514 | 78.74623987 |
| 51 | 0 | 20.81267428 | 18.92359956 | 38.29246251 | 1.249940315 |
| 52 | 0 | 1.783943509 | 19.57613747 | 2.687190352 | 12.49940315 |
| 53 | 0 | 8.325069711 | 11.09314457 | 5.374380703 | 14.99928378 |
| 54 | 50.37244612 | 5.351830528 | 54.81318492 | 1.343595176 | 35.62329899 |
| 55 | 102.682294 | 1572.843527 | 1703.12396 | 1778.920013 | 804.9615631 |
| 56 | 902.8292266 | 406.7391201 | 348.455247 | 378.8938396 | 69.3716875 |
| 57 | 403.9482698 | 74.33097956 | 48.28780576 | 104.8004237 | 217.4896149 |
| 58 | 25.18622306 | 25.56985697 | 34.58450953 | 25.52830834 | 18.12413457 |
| 59 | 66.84036119 | 291.3774399 | 536.3861667 | 321.119247 | 185.6161368 |
| 60 | 4.843504434 | 208.1267428 | 220.5578155 | 236.4727509 | 53.1224634 |
| 61 | 201.4897845 | 215.8571646 | 172.9225477 | 149.1390645 | 77.49629955 |
| 62 | 199.5523827 | 213.4785733 | 172.2700097 | 147.1236718 | 77.49629955 |
| 63 | 3.874803547 | 29.73239182 | 39.15227494 | 82.63110331 | 8.749582207 |
| 64 | 46.49764257 | 59.46478365 | 149.4311827 | 67.85155638 | 55.62234403 |
| 65 | 1610.949575 | 447.7698209 | 428.7174106 | 563.6381763 | 215.6147044 |
| 66 | 3.874803547 | 76.11492307 | 174.2276235 | 51.72841427 | 74.37144876 |
| 67 | 78.46477184 | 28.54309615 | 63.94871574 | 66.5079612 | 3.124850788 |
| 68 | 20194.50739 | 36020.79269 | 28969.42077 | 32557.9983 | 53055.5916 |
| 69 | 39.71673636 | 10.10901322 | 1.305075831 | 3.35898794 | 0 |
| 70 | 0 | 66.00590985 | 55.46572284 | 49.04122392 | 6.249701577 |
| 71 | 0 | 2.973239182 | 3.915227494 | 5.374380703 | 0 |
| 72 | 0 | 10.70366106 | 10.44060665 | 13.43595176 | 0 |
| 73 | 234.4256146 | 136.1743546 | 109.6263698 | 131.0005296 | 12.49940315 |
| 74 | 40.68543725 | 78.49351441 | 8.482992904 | 37.62066492 | 36.24826915 |
| 75 | 0 | 2.378591346 | 0 | 0 | 0 |
| 76 | 56.18465144 | 41.62534855 | 24.7964408 | 19.48213005 | 19.99904505 |
| 77 | 28.09232572 | 299.1078617 | 202.9392918 | 32.91808181 | 163.1172112 |
| 78 | 23.24882128 | 7.135774038 | 9.13553082 | 29.55909387 | 0 |
| 79 | 10.65570976 | 9.514365384 | 16.96598581 | 10.74876141 | 20.6240152 |
| 80 | 0 | 2.973239182 | 1.305075831 | 0 | 0 |
| 81 | 1052.977864 | 193.2605469 | 170.9649339 | 221.0214064 | 47.49773198 |
| 82 | 0 | 259.2664567 | 327.5740337 | 171.9801825 | 4.999761261 |
| 83 | 111.400602 | 38.65210937 | 52.85557117 | 32.24628422 | 24.99880631 |
| 84 | 110.4319011 | 38.65210937 | 52.85557117 | 32.24628422 | 24.99880631 |
| 85 | 38.74803547 | 28.54309615 | 45.6776541 | 62.47717568 | 35.62329899 |
| 86 | 0 | 7.730421874 | 0 | 0 | 0 |
| 87 | 0 | 2.378591346 | 2.610151663 | 3.35898794 | 0 |
| 88 | 0 | 0.594647836 | 0.652537916 | 0 | 0 |
| 89 | 11.62441064 | 21.40732211 | 91.3553082 | 246.5497148 | 7.499641892 |
| 90 | 9.687008869 | 50.5450661 | 29.36420621 | 41.65145045 | 1.249940315 |
| 91 | 12.59311153 | 65.41126201 | 65.25379157 | 61.1335805 | 31.24850788 |
| 92 | 2.906102661 | 195.0444904 | 140.2956519 | 16.12314211 | 4.999761261 |
| 93 | 0 | 2.973239182 | 1.305075831 | 0.671797588 | 0 |
| 94 | 197.6149809 | 65.41126201 | 41.10988869 | 113.5337924 | 296.8608249 |
| 95 | 0.968700887 | 25.56985697 | 23.49136497 | 22.84111799 | 31.24850788 |
| 96 | 49.40374523 | 0.594647836 | 0 | 0.671797588 | 3.124850788 |
| 97 | 0 | 1.189295673 | 0 | 0 | 0 |
| 98 | 42.62283902 | 63.6273185 | 110.2789078 | 133.0159224 | 60.62210529 |
| 99 | 14.5305133 | 23.78591346 | 37.84719911 | 24.85651075 | 4.999761261 |
| 100 | 0 | 7.135774038 | 4.56776541 | 9.405166231 | 24.37383615 |
| 101 | 16.46791508 | 6.541126201 | 0 | 0.671797588 | 0 |
| 102 | 0 | 0 | 0 | 0.671797588 | 0 |
| 103 | 99.77619135 | 1079.285823 | 1008.823618 | 993.5886325 | 562.4731419 |
| 104 | 0 | 0 | 0 | 0 | 0 |
| 105 | 0.968700887 | 6.541126201 | 2.610151663 | 5.374380703 | 0 |
| 106 | 0 | 0 | 0.652537916 | 4.030785528 | 0 |
| 107 | 112.3693029 | 161.7442115 | 142.9058035 | 147.1236718 | 41.24803041 |
| 108 | 0 | 258.077161 | 165.0920927 | 118.2363755 | 134.9935541 |
| 109 | 138.5242268 | 95.73830167 | 211.4222847 | 106.8158165 | 91.24564302 |
| 110 | 8176.804186 | 112.9830889 | 92.66038403 | 112.1901972 | 29.99856757 |
| 111 | 4.843504434 | 54.11295312 | 43.06750244 | 34.26167698 | 14.37431363 |
| 112 | 26.15492395 | 105.2526671 | 134.4228106 | 123.6107562 | 29.99856757 |
| 113 | 0.968700887 | 123.0921021 | 97.22814944 | 38.29246251 | 73.74647861 |
| 114 | 0 | 36.86816586 | 25.44897871 | 45.68223598 | 8.749582207 |
| 115 | 0.968700887 | 4.162534855 | 4.56776541 | 10.07696382 | 0 |
| 116 | 16.46791508 | 77.89886658 | 69.82155698 | 57.77459256 | 1.249940315 |
| 117 | 0 | 3.567887019 | 0 | 6.717975879 | 0 |
| 118 | 213.1141951 | 115.9563281 | 171.6174718 | 102.785031 | 10.62449268 |
| 119 | 13722.61676 | 8468.379839 | 8352.485321 | 8137.484183 | 13318.73903 |
| 120 | 111.400602 | 22.00196995 | 12.3982204 | 8.733368643 | 1.249940315 |

| SEQ | | | | |
|---|---|---|---|---|
| ID | | | | |
| NO: | n_RBI.14 | n_RBI.15 | log2_RBI.01 | log2_RBI.02 |
| 1 | 1925.955577 | 1957.30067 | 0 | −2.769117016 |
| 2 | 601.0371636 | 685.4307195 | 0 | −2.32788402 |
| 3 | 602.2356425 | 688.8442191 | 0 | −2.329917418 |
| 4 | 82.09580401 | 33.45229607 | 0 | −3.002474454 |
| 5 | 63.5193812 | 51.20249398 | 0 | −3.490023154 |
| 6 | 64.11862065 | 51.20249398 | 0 | −3.490023154 |
| 7 | 48.53839507 | 49.83709415 | 0 | 0.389948735 |
| 8 | 11.98478891 | 11.60589864 | 0 | −21.72762665 |
| 9 | 23.37033837 | 8.192399038 | 0 | −21.45009276 |
| 10 | 1472.930557 | 2237.890337 | 0 | −2.163108939 |
| 11 | 1474.129035 | 2239.255737 | 0 | −2.160791356 |
| 12 | 287.6349337 | 107.8665873 | 0 | −1.665596897 |
| 13 | 117.4509313 | 131.0783846 | 0 | 2.160741789 |
| 14 | 13.1832678 | 71.68349158 | 0 | −21.86513014 |
| 15 | 1.198478891 | 0 | 0 | −21.049555 |
| 16 | 226.5125103 | 81.92399038 | 0 | 0.19737026 |
| 17 | 2.396957781 | 0 | 0 | 2.394943361 |
| 18 | 0 | 0 | 0 | −19.57562499 |
| 19 | 328.383216 | 691.5750188 | 0 | −22.96028717 |
| 20 | 4920.954325 | 3400.528301 | 0 | 1.642961626 |
| 21 | 17.97718336 | 14.33669832 | 0 | −1.550461016 |
| 22 | 743.0569122 | 529.7751378 | 0 | 0.942693435 |
| 23 | 114.4547341 | 4.778899439 | 0 | −22.88143176 |
| 24 | 11.98478891 | 30.03879647 | 0 | −21.35989499 |
| 25 | 104.2676635 | 176.8192792 | 0 | 1.028217396 |
| 26 | 17.97718336 | 14.33669832 | 0 | −1.958378479 |
| 27 | 23.96957781 | 7.509699118 | 0 | −21.26367971 |
| 28 | 8.389352234 | 3.413499599 | 0 | −1.650488456 |
| 29 | 47.93915562 | 105.8184876 | 0 | −6.79486391 |
| 30 | 72.50797288 | 54.61599358 | 0 | −0.294186573 |
| 31 | 14.38174669 | 67.58729206 | 0 | −2.665594444 |
| 32 | 103.0691846 | 83.97209013 | 0 | −1.556890609 |
| 33 | 545.9071347 | 697.0366181 | 0 | −2.209917678 |
| 34 | 50.93535285 | 72.3661915 | 0 | −1.616341899 |
| 35 | 86.29048012 | 136.539984 | 0 | 0.467801888 |
| 36 | 16.77870447 | 32.08689623 | 0 | −23.07812365 |
| 37 | 182.7680308 | 398.6967532 | 0 | 0.15824582 |
| 38 | 23.96957781 | 8.192399038 | 0 | −4.742396958 |
| 39 | 37.75208505 | 17.06749799 | 0 | −20.86513052 |
| 40 | 0 | 1.36539984 | 0 | −17.99066618 |
| 41 | 2.396957781 | 0 | 0 | −16.40571476 |
| 42 | 112.6570157 | 43.01009495 | 0 | 0.62914599 |
| 43 | 208.535327 | 25.25989703 | 0 | −0.563027434 |
| 44 | 40.14904283 | 30.03879647 | 0 | −0.494485177 |
| 45 | 41.94676117 | 92.84718909 | 0 | −22.83196309 |
| 46 | 325.3870188 | 99.67418829 | 0 | 4.570086474 |
| 47 | 379.3185689 | 584.3911313 | 0 | −0.144217922 |
| 48 | 294.2265676 | 327.6959615 | 0 | 0.305166931 |
| 49 | 1.198478891 | 5.461599358 | 0 | −2.861989696 |
| 50 | 40.14904283 | 57.34679326 | 0 | −7.652844839 |
| 51 | 1.198478891 | 0 | 0 | −20.86513052 |
| 52 | 0.599239445 | 7.509699118 | 0 | −2.15744712 |
| 53 | 15.58022558 | 90.11638941 | 0 | −20.21305425 |
| 54 | 2.396957781 | 7.509699118 | 0 | −22.18705816 |
| 55 | 1287.765568 | 1597.517812 | 0 | −0.392199641 |
| 56 | 469.2044857 | 174.7711795 | 0 | −1.939026696 |
| 57 | 251.680567 | 213.002375 | 0 | −0.662429834 |
| 58 | 23.96957781 | 3.413499599 | 0 | −5.941705418 |
| 59 | 180.9703125 | 271.7145681 | 0 | −0.403845765 |
| 60 | 172.5809602 | 122.8859856 | 0 | −0.63059073 |
| 61 | 109.061579 | 132.4437844 | 0 | −2.427148284 |
| 62 | 109.061579 | 132.4437844 | 0 | −2.445295628 |
| 63 | 2.396957781 | 5.461599358 | 0 | −1.72449027 |
| 64 | 59.32470508 | 98.30878845 | 0 | 3.809129613 |
| 65 | 342.7649627 | 259.4259695 | 0 | −2.258144237 |
| 66 | 26.36653559 | 6.144299278 | 0 | −23.85690935 |
| 67 | 56.9277473 | 23.89449719 | 0 | −4.413786144 |
| 68 | 25091.95329 | 23843.9774 | 0 | −0.493125057 |
| 69 | 2.396957781 | 4.096199519 | 0 | −18.72762956 |
| 70 | 170.7832419 | 111.9627868 | 0 | −3.701768931 |
| 71 | 5.992394453 | 3.413499599 | 0 | −20.10613914 |
| 72 | 13.78250724 | 1.36539984 | 0 | 0.012474668 |
| 73 | 23.37033837 | 94.89528885 | 0 | 0.630840313 |
| 74 | 16.17946502 | 19.79829767 | 0 | −2.856940112 |
| 75 | 0 | 0 | 0 | 0 |
| 76 | 9.587831125 | 22.52909735 | 0 | −3.472949143 |
| 77 | 33.55740894 | 23.89449719 | 0 | −3.613963695 |
| 78 | 7.790112789 | 16.38479808 | 0 | −1.724488994 |
| 79 | 22.77109892 | 22.52909735 | 0 | −22.88143176 |
| 80 | 0 | 0 | 0 | −18.99066341 |
| 81 | 85.69124068 | 125.6167852 | 0 | −0.076182931 |
| 82 | 51.5345923 | 48.47169431 | 0 | −23.88951401 |
| 83 | 5.992394453 | 14.33669832 | 0 | −22.84864183 |
| 84 | 5.992394453 | 14.33669832 | 0 | −22.84864183 |
| 85 | 20.97338059 | 25.94259695 | 0 | −1.253419147 |
| 86 | 0 | 0 | 0 | −21.35989499 |
| 87 | 10.18707057 | 4.096199519 | 0 | 0.762496122 |
| 88 | 0 | 0 | 0 | −17.40570645 |
| 89 | 32.35893005 | 5.461599358 | 0 | −22.4056984 |
| 90 | 23.96957781 | 21.16369751 | 0 | −2.672021504 |
| 91 | 8.988591679 | 10.92319872 | 0 | −1.646488102 |
| 92 | 20.37414114 | 6.144299278 | 0 | −22.81508927 |
| 93 | 0 | 0 | 0 | 0 |
| 94 | 65.31709954 | 90.11638941 | 0 | −1.997649358 |
| 95 | 3.595436672 | 15.70209816 | 0 | 0.692385526 |
| 96 | 0 | 2.730799679 | 0 | −20.40569918 |
| 97 | 0 | 0 | 0 | 0 |
| 98 | 37.15284561 | 34.81769591 | 0 | −3.53772168 |
| 99 | 16.17946502 | 25.94259695 | 0 | −22.6724849 |
| 100 | 8.389352234 | 0 | 0 | −1.294186139 |
| 101 | 33.55740894 | 0 | 0 | 6.738391747 |
| 102 | 0 | 0 | 0 | 0 |
| 103 | 727.4766866 | 361.8309575 | 0 | −26.85896879 |
| 104 | 0 | 0 | 0 | −21.1061385 |
| 105 | 0.599239445 | 33.45229607 | 0 | −19.40570022 |
| 106 | 0.599239445 | 0 | 0 | 0 |
| 107 | 62.32090231 | 47.10629447 | 0 | −2.259484673 |
| 108 | 52.13383174 | 10.92319872 | 0 | −25.19033303 |
| 109 | 118.0501707 | 159.0690813 | 0 | −3.271317638 |
| 110 | 23.37033837 | 16.38479808 | 0 | −0.670043049 |
| 111 | 221.1193553 | 4.778899439 | 0 | 0.94973876 |
| 112 | 151.6075797 | 25.25989703 | 0 | −23.78073767 |
| 113 | 27.56501448 | 49.15439423 | 0 | −2.597578072 |
| 114 | 7.190873344 | 2.048099759 | 0 | −22.81508927 |
| 115 | 2.396957781 | 0 | 0 | −17.99066618 |
| 116 | 67.11481787 | 80.55859054 | 0 | 2.038376458 |
| 117 | 1.198478891 | 0.68269992 | 0 | −19.21305544 |
| 118 | 101.2714663 | 24.57719711 | 0 | −24.53498122 |
| 119 | 6658.748716 | 9892.321838 | 0 | 0.259449717 |
| 120 | 31.16045116 | 18.43289783 | 0 | −3.007423269 |

| SEQ | | | | | | | | In vitro |
|---|---|---|---|---|---|---|---|---|
| ID | | | | | | | | Ctrl log2 |
| NO: | log2_RBI.03 | log2_RBI.10 | log2_RBI.11 | log2_RBI.12 | log2_RBI.13 | log2_RBI.14 | log2_RBI.15 | mean |
| 1 | −0.536814683 | −0.392833516 | −0.521841713 | −0.054357855 | 0.716821038 | 1.513530242 | 1.536821207 | −0.3 |
| 2 | 0.01709861 | −0.833197682 | −1.234107389 | −1.013052453 | 0.866731347 | −0.034389095 | 0.155167554 | −1 |
| 3 | 0.017307164 | −0.83027336 | −1.225387731 | −0.999283994 | 0.867105787 | −0.033548597 | 0.160301062 | −1 |
| 4 | −3.119917929 | −1.560900244 | −1.585571775 | −1.2419513 | −0.489148732 | −0.036733924 | −1.331936918 | −1.5 |
| 5 | 0.940967261 | −0.366626467 | 0.187113626 | −0.135493869 | 0.881282083 | 0.105604427 | −0.205378293 | −0.1 |
| 6 | 0.940967261 | −0.366626467 | 0.187113626 | −0.117571964 | 0.889549699 | 0.119150957 | −0.205378293 | −0.1 |
| 7 | −3.413474985 | −1.480062023 | −1.4254703 | −1.067031844 | −3.893735198 | −0.936470011 | −0.898376473 | −1.3 |
| 8 | −2.356505961 | −1.009896915 | −0.646389049 | −2.106923367 | −3.796121199 | −1.534852381 | −1.581198606 | −1.3 |
| 9 | 0.201134157 | −0.342416708 | −2.871352468 | −0.285070139 | −2.196662679 | −0.293844956 | −1.806164541 | −1.2 |
| 10 | 0.373694356 | 0.13468646 | 0.044984985 | 0.097756623 | −0.990973655 | −0.72793838 | −0.124488455 | 0.1 |
| 11 | 0.37192472 | 0.133725197 | 0.044236699 | 0.09664243 | −0.992443543 | −0.72676498 | −0.123608495 | 0.1 |
| 12 | 0.10559807 | −1.680879489 | −1.971351006 | −1.855385585 | −2.637696417 | 1.564682406 | 0.149691632 | −1.8 |
| 13 | −3.284877439 | 0.19101535 | 0.367524881 | −0.117094368 | 2.28824399 | −0.948049262 | −0.789677629 | 0.1 |
| 14 | −21.86513014 | −2.545948409 | −1.871354645 | −2.507460901 | −0.979433401 | −1.534852452 | 0.908079543 | −2.3 |
| 15 | −21.049555 | −2.189804059 | −5.055758776 | −1.206459545 | −21.049555 | −4.178697986 | −21.049555 | −2.8 |
| 16 | 0.495223151 | −0.534220929 | −0.317894825 | −0.372906421 | −1.642652002 | −0.909248654 | −2.376481381 | −0.4 |
| 17 | −20.21305425 | −1.545947958 | −0.411923638 | 1.550605604 | −20.21305425 | −2.342203259 | −20.21305425 | −0.1 |
| 18 | 2.918876234 | −0.393946564 | 0.225505623 | −1.217953605 | −19.57562499 | −19.57562499 | −19.57562499 | −0.5 |
| 19 | 0.49829436 | −0.691148044 | −0.608960785 | −0.774800753 | 2.141137553 | 2.008589929 | 3.083095271 | −0.7 |
| 20 | 3.06779138 | 0.538262762 | −0.115125642 | 0.294250102 | 2.803054621 | 2.631036795 | 2.097857569 | 0.2 |
| 21 | 2.532302257 | −1.142052987 | −2.112362899 | −1.691886671 | 1.807214293 | −1.65032984 | −1.97678382 | −1.6 |
| 22 | −0.820203096 | 0.210828258 | 0.076627392 | 0.168021983 | −0.708806813 | 0.529382933 | 0.041290367 | 0.2 |
| 23 | −1.673811332 | −1.2403237 | −0.821568128 | −0.064332856 | −0.54054087 | 0.566842167 | −4.015109857 | −0.7 |
| 24 | −21.35989499 | −2.915180539 | −1.1181922 | −2.739189913 | −0.384000487 | −1.167120717 | 0.158501073 | −2.3 |
| 25 | 0.912115225 | −0.713615698 | 0.061826242 | −0.265306984 | 0.103206686 | −1.409322201 | −0.647338477 | −0.3 |
| 26 | −4.871677048 | −2.701227529 | −1.6690815 | −2.077777849 | −2.134711418 | −2.242671785 | −2.569125765 | −2.1 |
| 27 | −0.612452351 | −5.403907544 | −2.099978141 | −4.227929977 | −1.747215603 | −0.07090604 | −1.745282208 | −3.9 |
| 28 | −20.65362653 | −1.471948104 | −2.659846891 | −2.295955145 | 0.135854943 | −0.975424915 | −2.272730247 | −2.1 |
| 29 | −0.718551989 | −0.52041508 | 1.365683457 | 1.027257 | −0.072100008 | −1.020279844 | 0.122035291 | 0.6 |
| 30 | −21.96028735 | −0.742986846 | −0.837229587 | −0.252122589 | 1.100495517 | 0.829421062 | 0.420604974 | −0.6 |
| 31 | 0.885985716 | −0.022388273 | −1.826960207 | −1.784995376 | −0.719310622 | −0.949890185 | 1.282622134 | −1.2 |
| 32 | −0.780533826 | −0.840693359 | −0.935485822 | −0.806058126 | 0.068328766 | 0.367849589 | 0.072218361 | −0.9 |
| 33 | 0.644473413 | −0.276062157 | −0.016600039 | −0.061836851 | −0.554722157 | −0.739719527 | −0.387140637 | −0.1 |
| 34 | −4.640673909 | −0.337197466 | −0.328022747 | −0.515258657 | −0.802620242 | −1.509166342 | −1.002517918 | −0.4 |
| 35 | −0.935043845 | −0.784398957 | −0.319166873 | −0.066178395 | −1.006592844 | −1.364928065 | −0.702877949 | −0.4 |
| 36 | −6.514345112 | −0.173981438 | −0.529760448 | −0.053699796 | −5.146618193 | −2.399922892 | −1.464570384 | −0.3 |
| 37 | −2.730288875 | −0.217404255 | −0.254954675 | −0.266008173 | −0.507750999 | −0.725131201 | 0.400146872 | −0.2 |
| 38 | −21.16058626 | −1.715873832 | −2.359454068 | −2.802914874 | 0.262767032 | 0.03218741 | −1.516658036 | −2.3 |
| 39 | −1.9794358 | 0.316546091 | −1.170914986 | 0.077499791 | −3.348660638 | 0.982994763 | −0.162309519 | −0.3 |
| 40 | −17.99066618 | 0.454048268 | −0.4119222 | 0.367005203 | −17.99066618 | −17.99066618 | −0.931691654 | 0.1 |
| 41 | 4.479977722 | −16.40571476 | 0.588070407 | 1.630029605 | −16.40571476 | 1.465136232 | −16.40571476 | #NUM! |
| 42 | −0.883754542 | −1.559755728 | −2.541206284 | −0.130008339 | 0.518915107 | 0.305490135 | −1.083699597 | −1.4 |
| 43 | −4.767931049 | −2.334445689 | 1.098371613 | 0.137000832 | −3.72212464 | 0.660162773 | −2.385207866 | −0.4 |
| 44 | −0.952965524 | 0.385662716 | 0.477044571 | −0.632993383 | 0.960738448 | −0.223651429 | −0.642189891 | 0.1 |
| 45 | 0.19123234 | 0.678836627 | 0.994701133 | 0.587480326 | −2.14557505 | −0.831834756 | 0.314463869 | 0.8 |
| 46 | −22.5554455 | 1.085662234 | 0.53636086 | 0.300472652 | 0.953483136 | 2.400207909 | 0.69334317 | 0.6 |
| 47 | −0.415903074 | 0.438921743 | 0.12336757 | 0.66766989 | −1.804175026 | −0.718639425 | −0.095115152 | 0.4 |
| 48 | 2.700003529 | 0.226532302 | 0.254038184 | 0.298764206 | −1.054782465 | 0.04278227 | 0.198212635 | 0.3 |
| 49 | −0.716403132 | −1.098490398 | −3.286386534 | 0.418536558 | −20.86513052 | −3.994273505 | −1.806163912 | −1.3 |
| 50 | 0.369246511 | −0.916665332 | −0.867806514 | −0.496136219 | −1.162254352 | −2.134099618 | −1.619752505 | −0.8 |
| 51 | −20.86513052 | 0.123901099 | −0.013374647 | 1.00349887 | −3.933619291 | −3.994273505 | −20.86513052 | 0.4 |
| 52 | −20.57562407 | −3.130905574 | 0.325041378 | −2.539879703 | −0.322195135 | −4.704755019 | −1.057226565 | −1.8 |
| 53 | −20.21305425 | −0.545949691 | −0.131815998 | −1.177312571 | 0.303408894 | 0.358231366 | 2.890303991 | −0.6 |
| 54 | 0.077145489 | −3.157382555 | 0.19903364 | −5.151308425 | −0.422668056 | −4.316207166 | −2.668660656 | −2.7 |
| 55 | −4.037341399 | −0.100225715 | 0.014582576 | 0.077400774 | −1.066609058 | −0.388730884 | −0.07776885 | 0 |
| 56 | 1.151886906 | 0.001537559 | −0.221592812 | −0.100772511 | −2.55014714 | 0.207650604 | −1.217098852 | −0.1 |
| 57 | 2.354174287 | −0.087960581 | −0.710265189 | 0.407648387 | 1.46095028 | 1.671597583 | 1.430873284 | −0.1 |
| 58 | −1.095690787 | −1.073881496 | −0.638199737 | −1.076227647 | −1.570413247 | −1.167121051 | −3.978998437 | −0.9 |
| 59 | −2.284156657 | −0.160059078 | 0.72032375 | −0.019839123 | −0.810626091 | −0.84719518 | −0.260856336 | 0.2 |
| 60 | −5.352893513 | 0.072370857 | 0.156065384 | 0.256582446 | −1.897697339 | −0.197818171 | −0.687771053 | 0.2 |
| 61 | −0.903045981 | −0.803675703 | −1.123626668 | −1.337094454 | −2.281553234 | −1.788609667 | −1.5083725 | −1.1 |
| 62 | −0.916985171 | −0.819661406 | −1.129081098 | −1.35672326 | −2.281553234 | −1.788609667 | −1.5083725 | −1.1 |
| 63 | −3.748821649 | −0.808984434 | −0.411923939 | 0.665664661 | −2.573732761 | −4.441738023 | −3.253622411 | −0.2 |
| 64 | −1.436880893 | −1.082003009 | 0.24737065 | −0.891656656 | −1.178373974 | −1.08540551 | −0.356718236 | −0.6 |
| 65 | 1.211979509 | −0.635102603 | −0.69783289 | −0.303090574 | −1.689404294 | −1.020640243 | −1.422536969 | −0.5 |
| 66 | −5.293141983 | −0.997161254 | 0.197560777 | −1.554383534 | −1.030591709 | −2.52663221 | −4.628018037 | −0.8 |
| 67 | 0.071650739 | −1.387252179 | −0.223478909 | −0.166867258 | −4.578530696 | −0.391262255 | −1.643715507 | −0.6 |
| 68 | −0.925814644 | −0.090947668 | −0.40524676 | −0.236765594 | 0.467727208 | −0.612552816 | −0.686152687 | −0.2 |
| 69 | 3.19368645 | 1.219582613 | −1.733844394 | −0.369958175 | −18.72762956 | −0.856778569 | −0.083699576 | −0.3 |
| 70 | −23.11994382 | −0.465779828 | −0.71677851 | −0.89437997 | −3.866513732 | 0.905719348 | 0.296572278 | −0.7 |
| 71 | −20.10613914 | −1.924458286 | −1.527398841 | −1.070397459 | −20.10613914 | −0.913363663 | −1.725242855 | −1.5 |
| 72 | −19.40570022 | 0.623974035 | 0.588075274 | 0.951967945 | −19.40570022 | 0.988707756 | −2.346725691 | 0.7 |
| 73 | 0.545547249 | −0.238127893 | −0.550988026 | −0.294010273 | −3.683650761 | −2.780831883 | −0.759174506 | −0.4 |
| 74 | −1.019472506 | −0.071411717 | −3.281338395 | −1.132459624 | −1.18607285 | −2.349820559 | −2.058608239 | −1.5 |
| 75 | 0 | 17.85975397 | 0 | 0 | 0 | 0 | 0 | #DIV/0! |
| 76 | 0.208691533 | −0.224022092 | −0.971351154 | −1.31933263 | −1.281552957 | −2.342206883 | −1.109694639 | −0.8 |
| 77 | −2.93232029 | 0.480097102 | −0.079520488 | −2.703616163 | −0.394659673 | −2.675865116 | −3.165817858 | −0.8 |
| 78 | 0.421099696 | −1.28291464 | −0.926496455 | 0.767544034 | −20.72762707 | −1.156340525 | −0.083699724 | −0.5 |
| 79 | −2.858235145 | −3.021682338 | −2.18721708 | −2.845691422 | −1.905537258 | −1.76265864 | −1.778073038 | −2.7 |
| 80 | −18.99066341 | −0.80898256 | −1.996878246 | −18.99066341 | −18.99066341 | −18.99066341 | −18.99066341 | #NUM! |
| 81 | 2.005796945 | −0.440059049 | −0.616905739 | −0.246420102 | −2.464675437 | −1.613386458 | −1.061576903 | −0.4 |
| 82 | −23.88951401 | 0.738418274 | 1.075803697 | 0.146225067 | −4.958011444 | −1.592404005 | −1.680802633 | 0.7 |
| 83 | 0.560611994 | −0.966525737 | −0.515017441 | −1.227939885 | −1.595213474 | −3.655866354 | −2.397359438 | −0.9 |
| 84 | 0.548011958 | −0.966525737 | −0.515017441 | −1.227939885 | −1.595213474 | −3.655866354 | −2.397359438 | −0.9 |
| 85 | −1.17821768 | −1.619198878 | −0.940852345 | −0.489011752 | −1.299519688 | −2.063781112 | −1.757017758 | −1 |
| 86 | −21.35989499 | −1.799705499 | −21.35989499 | −21.35989499 | −21.35989499 | −21.35989499 | −21.35989499 | #NUM! |
| 87 | −20.86513052 | −3.005376545 | −2.871350877 | −2.507459131 | −20.86513052 | −0.906821286 | −2.221200531 | −2.8 |
| 88 | −17.40570645 | −1.545934284 | −1.41191023 | −17.40570645 | −17.40570645 | −17.40570645 | −17.40570645 | #NUM! |
| 89 | −2.256971018 | −1.376024821 | 0.717358885 | 2.149676905 | −2.889234295 | −0.779965481 | −3.346731798 | 0.5 |
| 90 | −2.011858381 | 0.371587519 | −0.411923908 | 0.092384044 | −4.966040383 | −0.704777938 | −0.884390653 | 0 |
| 91 | −3.04838437 | −0.671481037 | −0.674958354 | −0.769055005 | −1.737232542 | −3.534851703 | −3.253623593 | −0.7 |
| 92 | −4.666358166 | 1.40221071 | 0.92687779 | −2.194386883 | −3.883586706 | −1.856780751 | −3.586197962 | 0 |
| 93 | 0 | 18.18168085 | 16.99378517 | 16.03576047 | 0 | 0 | 0 | #DIV/0! |
| 94 | −0.32938048 | −1.924461698 | −2.594515151 | −1.128950978 | 0.257713897 | −1.926540018 | −1.462211295 | −1.9 |
| 95 | −6.45662962 | −1.734394932 | −1.856708429 | −1.897205688 | −1.445051822 | −4.56459667 | −2.437881319 | −1.8 |
| 96 | 1.830490096 | −4.545927014 | −20.40569918 | −4.36993871 | −2.152266786 | −20.40569918 | −2.346729935 | #NUM! |
| 97 | 0 | 16.85976004 | 0 | 0 | 0 | 0 | 0 | #DIV/0! |
| 98 | −2.325017116 | −1.746997596 | −0.953559036 | −0.683116991 | −1.816799952 | −2.523171043 | −2.616822996 | −1.1 |
| 99 | −2.201829668 | −1.490808292 | −0.820729411 | −1.427291957 | −3.740982331 | −2.046751532 | −1.365592867 | −1.2 |
| 100 | −21.96028735 | −2.515574919 | −3.159155155 | −2.117191896 | −0.743384854 | −2.282085733 | −21.96028735 | −2.6 |
| 101 | −0.841934113 | −2.173979743 | −21.49316147 | −5.457401002 | −21.49316147 | 0.185038853 | −21.49316147 | #NUM! |
| 102 | 0 | 0 | 0 | 16.03576047 | 0 | 0 | 0 | #DIV/0! |
| 103 | −3.608704474 | −0.173467036 | −0.270870058 | −0.292823441 | −1.113687889 | −0.742571089 | −1.750156236 | −0.2 |
| 104 | −21.1061385 | −21.1061385 | −21.1061385 | −21.1061385 | −21.1061385 | −21.1061385 | −21.1061385 | #NUM! |
| 105 | −2.841921684 | −0.08651849 | −1.41192058 | −0.36995854 | −19.40570022 | −3.534831171 | 2.267974018 | −0.6 |
| 106 | 0 | 0 | 15.99379622 | 18.62070508 | 0 | 15.87086905 | 0 | #DIV/0! |
| 107 | −0.810501937 | −0.285035867 | −0.46368543 | −0.42172055 | −2.256352551 | −1.66096178 | −2.064757977 | −0.4 |
| 108 | −25.19033303 | −0.569033832 | −1.213565252 | −1.695162289 | −1.503945734 | −2.87654428 | −5.131367742 | −1.2 |
| 109 | −0.489418055 | −1.022388204 | 0.120571044 | −0.864431053 | −1.091728739 | −0.720156224 | −0.289902941 | −0.6 |
| 110 | 6.17889577 | 0.001537558 | −0.284544696 | −0.008622666 | −1.911603419 | −2.271818274 | −2.78413874 | −0.1 |
| 111 | −3.074592632 | 0.407255465 | 0.077881219 | −0.252122589 | −1.505224705 | 2.438034697 | −3.093965444 | 0.1 |
| 112 | −2.462085978 | −0.453384081 | −0.100462927 | −0.221436605 | −2.264275009 | 0.073100969 | −2.512319775 | −0.3 |
| 113 | −6.699900745 | 0.2895557 | −0.0507365 | −1.395049894 | −0.449536353 | −1.869271828 | −1.034790023 | −0.4 |
| 114 | −22.81508927 | −1.001144667 | −1.535912376 | −0.691887121 | −3.07623302 | −3.359279794 | −5.171155767 | −1.1 |
| 115 | −1.426887647 | 0.676440111 | 0.81046601 | 1.951964841 | −17.99066618 | −0.11981519 | −17.99066618 | 1.1 |
| 116 | −2.86299536 | −0.621051627 | −0.778981415 | −1.052218719 | −6.582711495 | −0.836022609 | −0.572615529 | −0.8 |
| 117 | −19.21305544 | −0.768340988 | −19.21305544 | 0.1446138 | −19.21305544 | −2.342198427 | −3.154070344 | #NUM! |
| 118 | −0.189857795 | −1.067902875 | −0.502288033 | −1.24185424 | −4.516017337 | −1.263256667 | −3.306091668 | −0.9 |
| 119 | 0.748231319 | 0.051833591 | 0.031953152 | −0.005669556 | 0.705133204 | −0.295001292 | 0.276056787 | 0 |
| 120 | 0.813730894 | −1.526321002 | −2.35382014 | −2.859342563 | −5.664011704 | −1.024237775 | −1.781670682 | −2.2 |

| | In vitro | | | In vitro hits | | | | |
|---|---|---|---|---|---|---|---|---|
| SEQ | Rapa | | | (>=50 reads, | In vitro | | | |
| ID | log2 | In vitro | In vitro | log2.1< >1, | multiple | | | |
| NO: | mean | log2 diff | t.test | p < 0.05) | shRNA hits | ctrl.mean | treat.mean | log2_diff.13 |
| 1 | 1.3 | 1.6 | 0.014 | Yes | FALSE | −0.323011028 | 1.255724162 | 1.039832066 |
| 2 | 0.3 | 1.4 | 0.025 | Yes | TRUE | −1.026785842 | 0.329169936 | 1.893517189 |
| 3 | 0.3 | 1.3 | 0.025 | Yes | TRUE | −1.018315028 | 0.331286084 | 1.885420815 |
| 4 | −0.6 | 0.8 | 0.148 | | NA | −1.462807773 | −0.619273191 | 0.973659041 |
| 5 | 0.3 | 0.4 | 0.387 | | NA | −0.105002237 | 0.260502739 | 0.98628432 |
| 6 | 0.3 | 0.4 | 0.387 | | NA | −0.099028268 | 0.267774121 | 0.988577967 |
| 7 | −1.9 | −0.6 | 0.616 | | NA | −1.324188056 | −1.909527227 | −2.569547142 |
| 8 | −2.3 | −1 | 0.306 | | NA | −1.25440311 | −2.304057395 | −2.541718088 |
| 9 | −1.4 | −0.3 | 0.811 | | NA | −1.166279772 | −1.432224059 | −1.030382908 |
| 10 | −0.6 | −0.7 | 0.109 | | NA | 0.092476023 | −0.61446683 | −1.083449677 |
| 11 | −0.6 | −0.7 | 0.11 | | NA | 0.091534775 | −0.614272339 | −1.083978318 |
| 12 | −0.3 | 1.5 | 0.341 | | NA | −1.835872027 | −0.307774126 | −0.80182439 |
| 13 | 0.2 | 0 | 0.976 | | NA | 0.147148621 | 0.183505699 | 2.141095369 |
| 14 | −0.5 | 1.8 | 0.129 | | NA | −2.308254651 | −0.535402104 | 1.328821251 |
| 15 | #NUM! | #NUM! | #NUM! | | NA | −2.817340793 | −15.42593599 | −18.2322142 |
| 16 | −1.6 | −1.2 | 0.097 | | NA | −0.408340725 | −1.642794012 | −1.234311277 |
| 17 | #NUM! | #NUM! | #NUM! | | NA | −0.135755331 | −14.25610392 | −20.07729892 |
| 18 | #NUM! | #NUM! | #NUM! | | NA | −0.462131515 | −19.57562499 | −19.11349347 |
| 19 | 2.4 | 3.1 | 0.011 | Yes | TRUE | −0.691636527 | 2.410940918 | 2.832774081 |
| 20 | 2.5 | 2.3 | 0.001 | Yes | TRUE | 0.239129074 | 2.510649661 | 2.563925547 |
| 21 | −0.6 | 1 | 0.482 | | NA | −1.648767519 | −0.606633122 | 3.455981812 |
| 22 | 0 | −0.2 | 0.639 | | NA | 0.151825878 | −0.046044504 | −0.86063269 |
| 23 | −1.3 | −0.6 | 0.701 | | NA | −0.708741561 | −1.329602853 | 0.168200691 |
| 24 | −0.5 | 1.8 | 0.069 | | NA | −2.257520884 | −0.46420671 | 1.873520397 |
| 25 | −0.7 | −0.3 | 0.533 | | NA | −0.305698813 | −0.65115133 | 0.408905499 |
| 26 | −2.3 | −0.2 | 0.65 | | NA | −2.149362293 | −2.315502989 | 0.014650874 |
| 27 | −1.2 | 2.7 | 0.087 | | NA | −3.910605221 | −1.187801284 | 2.163389617 |
| 28 | −1 | 1.1 | 0.253 | | NA | −2.14258338 | −1.037433406 | 2.278438323 |
| 29 | −0.3 | −0.9 | 0.25 | | NA | 0.624175126 | −0.323448187 | −0.696275134 |
| 30 | 0.8 | 1.4 | 0.007 | | NA | −0.610779674 | 0.783507184 | 1.711275191 |
| 31 | −0.1 | 1.1 | 0.309 | | NA | −1.211447952 | −0.128859557 | 0.49213733 |
| 32 | 0.2 | 1 | 0.004 | Yes | FALSE | −0.860745769 | 0.169465572 | 0.929074536 |
| 33 | −0.6 | −0.4 | 0.029 | | NA | −0.118166349 | −0.56052744 | −0.436555809 |
| 34 | −1.1 | −0.7 | 0.068 | | NA | −0.393492957 | −1.104768167 | −0.409127286 |
| 35 | −1 | −0.6 | 0.09 | | NA | −0.389914742 | −1.02479962 | −0.616678102 |
| 36 | −3 | −2.8 | 0.128 | | NA | −0.252480561 | −3.003703823 | −4.894137633 |
| 37 | −0.3 | 0 | 0.936 | | NA | −0.246122368 | −0.277578443 | −0.261628631 |
| 38 | −0.4 | 1.9 | 0.057 | | NA | −2.292747591 | −0.407234531 | 2.555514623 |
| 39 | −0.8 | −0.6 | 0.705 | | NA | −0.258956368 | −0.842658465 | −3.08970427 |
| 40 | #NUM! | #NUM! | #NUM! | | NA | 0.13637709 | −12.30434134 | −18.12704327 |
| 41 | #NUM! | #NUM! | #NUM! | | NA | −4.729204916 | −10.44876443 | −11.67650984 |
| 42 | −0.1 | 1.3 | 0.206 | | NA | −1.41032345 | −0.086431452 | 1.929238557 |
| 43 | −1.8 | −1.4 | 0.432 | | NA | −0.366357748 | −1.815723244 | −3.355766891 |
| 44 | 0 | 0 | 0.944 | | NA | 0.076571301 | 0.031632376 | 0.884167146 |
| 45 | −0.9 | −1.6 | 0.143 | | NA | 0.753672695 | −0.887648646 | −2.899247746 |
| 46 | 1.3 | 0.7 | 0.316 | | NA | 0.640831915 | 1.349011405 | 0.31265122 |
| 47 | −0.9 | −1.3 | 0.113 | | NA | 0.409986401 | −0.872643201 | −2.214161427 |
| 48 | −0.3 | −0.5 | 0.31 | | NA | 0.259778231 | −0.27126252 | −1.314560696 |
| 49 | #NUM! | #NUM! | #NUM! | | NA | −1.322113458 | −8.888522645 | −19.54301706 |
| 50 | −1.6 | −0.9 | 0.07 | | NA | −0.760202689 | −1.638702158 | −0.402051663 |
| 51 | #NUM! | #NUM! | #NUM! | | NA | 0.371341774 | −9.597674438 | −4.304961066 |
| 52 | −2 | −0.2 | 0.894 | | NA | −1.781914633 | −2.028058906 | 1.459719498 |
| 53 | 1.2 | 1.8 | 0.159 | | NA | −0.61835942 | 1.183981417 | 0.921768314 |
| 54 | −2.5 | 0.2 | 0.91 | | NA | −2.703219113 | −2.469178626 | 2.280551057 |
| 55 | −0.5 | −0.5 | 0.221 | | NA | −0.002747455 | −0.511036264 | −1.063861603 |
| 56 | −1.2 | −1.1 | 0.308 | | NA | −0.106942588 | −1.186531796 | −2.443204552 |
| 57 | 1.5 | 1.7 | 0.031 | Yes | FALSE | −0.130192461 | 1.521140382 | 1.591142741 |
| 58 | −2.2 | −1.3 | 0.273 | | NA | −0.929436293 | −2.238844245 | −0.640976954 |
| 59 | −0.6 | −0.8 | 0.077 | | NA | 0.18014185 | −0.639559202 | −0.99076794 |
| 60 | −0.9 | −1.1 | 0.162 | | NA | 0.161672896 | −0.927762188 | −2.059370235 |
| 61 | −1.9 | −0.8 | 0.055 | | NA | −1.088132275 | −1.8595118 | −1.193420959 |
| 62 | −1.9 | −0.8 | 0.058 | | NA | −1.101821921 | −1.8595118 | −1.179731312 |
| 63 | −3.4 | −3.2 | 0.011 | Yes | FALSE | −0.185081238 | −3.423031065 | −2.388651524 |
| 64 | −0.9 | −0.3 | 0.581 | | NA | −0.575429672 | −0.87349924 | −0.602944302 |
| 65 | −1.4 | −0.8 | 0.03 | | NA | −0.545342022 | −1.377527169 | −1.144062272 |
| 66 | −2.7 | −1.9 | 0.196 | | NA | −0.784661337 | −2.728413985 | −0.245930372 |
| 67 | −2.2 | −1.6 | 0.323 | | NA | −0.592532782 | −2.204502819 | −3.985997914 |
| 68 | −0.3 | 0 | 0.939 | | NA | −0.244320007 | −0.276992765 | 0.712047215 |
| 69 | #NUM! | #NUM! | #NUM! | | NA | −0.294739985 | −6.556035902 | −18.43288957 |
| 70 | −0.9 | −0.2 | 0.908 | | NA | −0.692312769 | −0.888074035 | −3.174200963 |
| 71 | #NUM! | #NUM! | #NUM! | | NA | −1.507418195 | −7.581581885 | −18.59872094 |
| 72 | #NUM! | #NUM! | #NUM! | | NA | 0.721339085 | −6.921239385 | −20.1270393 |
| 73 | −2.4 | −2 | 0.14 | | NA | −0.361042064 | −2.407885717 | −3.322608697 |
| 74 | −1.9 | −0.4 | 0.742 | | NA | −1.495069912 | −1.864833883 | 0.308997062 |
| 75 | #DIV/0! | #DIV/0! | #DIV/0! | | NA | 5.953251323 | 0 | −5.953251323 |
| 76 | −1.6 | −0.7 | 0.217 | | NA | −0.838235292 | −1.57781816 | −0.443317665 |
| 77 | −2.1 | −1.3 | 0.372 | | NA | −0.76767985 | −2.078780883 | 0.373020176 |
| 78 | #NUM! | #NUM! | #NUM! | | NA | −0.480622354 | −7.322555772 | −20.24700471 |
| 79 | −1.8 | 0.9 | 0.071 | | NA | −2.684863613 | −1.815422979 | 0.779326355 |
| 80 | #NUM! | #NUM! | #NUM! | | NA | −7.265508073 | −18.99066341 | −11.72515534 |
| 81 | −1.7 | −1.3 | 0.08 | | NA | −0.43446163 | −1.713212933 | −2.030213807 |
| 82 | −2.7 | −3.4 | 0.084 | | NA | 0.653482346 | −2.743739361 | −5.61149379 |
| 83 | −2.5 | −1.6 | 0.098 | | NA | −0.903161021 | −2.549479755 | −0.692052453 |
| 84 | −2.5 | −1.6 | 0.098 | | NA | −0.903161021 | −2.549479755 | −0.692052453 |
| 85 | −1.7 | −0.7 | 0.166 | | NA | −1.016354325 | −1.706772852 | −0.283165363 |
| 86 | #NUM! | #NUM! | #NUM! | | NA | −14.83983183 | −21.35989499 | −6.520063163 |
| 87 | #NUM! | #NUM! | #NUM! | | NA | −2.794728851 | −7.997717444 | −18.07040166 |
| 88 | #NUM! | #NUM! | #NUM! | | NA | −6.787850322 | −17.40570645 | −10.61785613 |
| 89 | −2.3 | −2.8 | 0.098 | | NA | 0.497003656 | −2.338643858 | −3.386237951 |
| 90 | −2.2 | −2.2 | 0.252 | | NA | 0.017349218 | −2.185069658 | −4.983389602 |
| 91 | −2.8 | −2.1 | 0.062 | | NA | −0.705164798 | −2.841902613 | −1.032067744 |
| 92 | −3.1 | −3.2 | 0.089 | | NA | 0.044900539 | −3.10885514 | −3.928487245 |
| 93 | #DIV/0! | #DIV/0! | #DIV/0! | | NA | 17.07040883 | 0 | −17.07040883 |
| 94 | −1 | 0.8 | 0.357 | | NA | −1.882642609 | −1.043679139 | 2.140356506 |
| 95 | −2.8 | −1 | 0.396 | | NA | −1.829436349 | −2.81584327 | 0.384384527 |
| 96 | #NUM! | #NUM! | #NUM! | | NA | −9.773854968 | −8.301565301 | 7.621588182 |
| 97 | #DIV/0! | #DIV/0! | #DIV/0! | | NA | 5.619920012 | 0 | −5.619920012 |
| 98 | −2.3 | −1.2 | 0.046 | Yes | FALSE | −1.127891208 | −2.318931331 | −0.688908744 |
| 99 | −2.4 | −1.1 | 0.244 | | NA | −1.246276553 | −2.384442243 | −2.494705777 |
| 100 | #NUM! | #NUM! | #NUM! | | NA | −2.597307324 | −8.328585978 | 1.853922469 |
| 101 | #NUM! | #NUM! | #NUM! | | NA | −9.708180739 | −14.2670947 | −11.78498073 |
| 102 | #DIV/0! | #DIV/0! | #DIV/0! | | NA | 5.34525349 | 0 | −5.34525349 |
| 103 | −1.2 | −1 | 0.081 | | NA | −0.245720178 | −1.202138405 | −0.86796771 |
| 104 | #NUM! | #NUM! | #NUM! | | NA | −21.1061385 | −21.1061385 | 0 |
| 105 | #NUM! | #NUM! | #NUM! | | NA | −0.622799203 | −6.890852457 | −18.78290102 |
| 106 | #DIV/0! | #DIV/0! | #DIV/0! | | NA | 11.5381671 | 5.290289683 | −11.5381671 |
| 107 | −2 | −1.6 | 0.007 | Yes | FALSE | −0.390147282 | −1.994024103 | −1.866205269 |
| 108 | −3.2 | −2 | 0.19 | | NA | −1.159253791 | −3.170619252 | −0.344691943 |
| 109 | −0.7 | −0.1 | 0.808 | | NA | −0.588749404 | −0.700595968 | −0.502979335 |
| 110 | −2.3 | −2.2 | 0.007 | Yes | FALSE | −0.097209935 | −2.322520144 | −1.814393484 |
| 111 | −0.7 | −0.8 | 0.676 | | NA | 0.077671365 | −0.720385151 | −1.58289607 |
| 112 | −1.6 | −1.3 | 0.252 | | NA | −0.258427871 | −1.567831272 | −2.005847138 |
| 113 | −1.1 | −0.7 | 0.331 | | NA | −0.385410232 | −1.117866068 | −0.064126121 |
| 114 | −3.9 | −2.8 | 0.038 | Yes | FALSE | −1.076314721 | −3.868889527 | −1.999918299 |
| 115 | #NUM! | #NUM! | #NUM! | | NA | 1.146290321 | −12.03371585 | −19.1369565 |
| 116 | −2.7 | −1.8 | 0.446 | | NA | −0.817417254 | −2.663783211 | −5.765294241 |
| 117 | #NUM! | #NUM! | #NUM! | | NA | −6.612260876 | −8.236441403 | −12.60079456 |
| 118 | −3 | −2.1 | 0.152 | | NA | −0.937348383 | −3.028455224 | −3.578668954 |
| 119 | 0.2 | 0.2 | 0.557 | | NA | 0.026039062 | 0.228729566 | 0.679094142 |
| 120 | −2.8 | −0.6 | 0.731 | | NA | −2.246494568 | −2.82330672 | −3.417517136 |

| SEQ ID | | | | | |
|---|---|---|---|---|---|
| NO: | log2_diff.14 | log2_diff.15 | rank_cnt.01 | rank_cnt.02 | rank_cnt.03 |
| 1 | 1.83654127 | 1.859832235 | 0.900570157 | 0.738005841 | 0.87818106 |
| 2 | 0.992396746 | 1.181953396 | 0.891948269 | 0.761159783 | 0.903977194 |
| 3 | 0.984766431 | 1.178616091 | 0.892156863 | 0.761159783 | 0.904185788 |
| 4 | 1.426073849 | 0.130870855 | 0.515575024 | 0.479627312 | 0.435196774 |
| 5 | 0.210606664 | −0.100376057 | 0.426574885 | 0.423585037 | 0.736337088 |
| 6 | 0.218179225 | −0.106350025 | 0.426574885 | 0.423585037 | 0.736337088 |
| 7 | 0.387718045 | 0.425811582 | 0.542205535 | 0.760186344 | 0.426088166 |
| 8 | −0.28044927 | −0.326795496 | 0.316854401 | 0.163398693 | 0.401543596 |
| 9 | 0.872434816 | −0.63988477 | 0.283687943 | 0.163398693 | 0.56549854 |
| 10 | −0.820414403 | −0.216964478 | 0.975316368 | 0.901126408 | 0.978167153 |
| 11 | −0.818299755 | −0.21514327 | 0.975316368 | 0.901265471 | 0.978028091 |
| 12 | 3.400554433 | 1.985563658 | 0.553678209 | 0.597413433 | 0.726950355 |
| 13 | −1.095197883 | −0.93682625 | 0.744333194 | 0.938881936 | 0.526700042 |
| 14 | 0.773402199 | 3.216334194 | 0.331942706 | 0.163398693 | 0.143860381 |
| 15 | −1.361357193 | −18.2322142 | 0.240856626 | 0.163398693 | 0.143860381 |
| 16 | −0.500907929 | −1.968140656 | 0.849116952 | 0.89459046 | 0.900431094 |
| 17 | −2.206447929 | −20.07729892 | 0.166040884 | 0.688916701 | 0.143860381 |
| 18 | −19.11349347 | −19.11349347 | 0.125086914 | 0.163398693 | 0.645459602 |
| 19 | 2.700226456 | 3.774731798 | 0.508969545 | 0.163398693 | 0.738214435 |
| 20 | 2.391907721 | 1.858728495 | 0.916214713 | 0.971631206 | 0.992212488 |
| 21 | −0.001562321 | −0.328016301 | 0.417883465 | 0.546377416 | 0.848838826 |
| 22 | 0.377557055 | −0.110535511 | 0.870741204 | 0.937838965 | 0.837922403 |
| 23 | 1.275583728 | −3.306368296 | 0.494785148 | 0.163398693 | 0.531497705 |
| 24 | 1.090400167 | 2.416021957 | 0.273258239 | 0.163398693 | 0.143860381 |
| 25 | −1.103623388 | −0.341639663 | 0.784313725 | 0.905020164 | 0.889792797 |
| 26 | −0.093309492 | −0.419763472 | 0.518773467 | 0.560492282 | 0.345084133 |
| 27 | 3.83969918 | 2.165323012 | 0.263315255 | 0.163398693 | 0.487623418 |
| 28 | 1.167158464 | −0.130146867 | 0.201501877 | 0.423585037 | 0.143860381 |
| 29 | −1.644454969 | −0.502139834 | 0.553678209 | 0.340981783 | 0.645459602 |
| 30 | 1.440200736 | 1.031384648 | 0.343832568 | 0.608121263 | 0.143860381 |
| 31 | 0.261557767 | 2.494070086 | 0.278055903 | 0.410791267 | 0.626268947 |
| 32 | 1.228595358 | 0.93296413 | 0.504032819 | 0.583715756 | 0.612362676 |
| 33 | −0.621553179 | −0.268974288 | 0.926644417 | 0.813377833 | 0.950771798 |
| 34 | −1.115673385 | −0.609024961 | 0.645598665 | 0.650326797 | 0.388749826 |
| 35 | −0.975013323 | −0.312963207 | 0.741343346 | 0.855235711 | 0.73897928 |
| 36 | −2.147442331 | −1.212089823 | 0.528994577 | 0.163398693 | 0.302600473 |
| 37 | −0.479008834 | 0.646269239 | 0.799193436 | 0.865178696 | 0.610068141 |
| 38 | 2.324935001 | 0.776089555 | 0.252955083 | 0.340981783 | 0.143860381 |
| 39 | 1.241951131 | 0.096646849 | 0.220970658 | 0.163398693 | 0.37463496 |
| 40 | −18.12704327 | −1.068068744 | 0.064107913 | 0.163398693 | 0.143860381 |
| 41 | 6.194341148 | −11.67650984 | 0.035947712 | 0.163398693 | 0.505214852 |
| 42 | 1.715813585 | 0.326623854 | 0.537199277 | 0.776456682 | 0.620497845 |
| 43 | 1.026520521 | −2.018850118 | 0.621679878 | 0.725559727 | 0.37463496 |
| 44 | −0.30022273 | −0.718761192 | 0.374704492 | 0.608121263 | 0.531497705 |
| 45 | −1.585507452 | −0.439208827 | 0.48616326 | 0.163398693 | 0.696565151 |
| 46 | 1.759375994 | 0.052511255 | 0.437491309 | 0.955082742 | 0.143860381 |
| 47 | −1.128625826 | −0.505101553 | 0.89354749 | 0.905020164 | 0.878876373 |
| 48 | −0.216995961 | −0.061565596 | 0.79015436 | 0.868446669 | 0.962453066 |
| 49 | −2.672160048 | −0.484050454 | 0.220970658 | 0.381379502 | 0.452718676 |
| 50 | −1.37389693 | −0.859549816 | 0.692601863 | 0.340981783 | 0.812891114 |
| 51 | −4.36561528 | −21.23647229 | 0.220970658 | 0.163398693 | 0.143860381 |
| 52 | −2.922840386 | 0.724688068 | 0.194131553 | 0.396537338 | 0.143860381 |
| 53 | 0.976590786 | 3.50866341 | 0.166040884 | 0.163398693 | 0.143860381 |
| 54 | −1.612988053 | 0.034558458 | 0.37818106 | 0.163398693 | 0.623070505 |
| 55 | −0.38598343 | −0.075021395 | 0.960784314 | 0.949172577 | 0.724794882 |
| 56 | 0.314593192 | −1.110156264 | 0.844527882 | 0.74461132 | 0.929842859 |
| 57 | 1.801790044 | 1.561065745 | 0.50152969 | 0.657001808 | 0.867681825 |
| 58 | −0.237684757 | −3.049562144 | 0.406063134 | 0.340981783 | 0.535669587 |
| 59 | −1.027337029 | −0.440998186 | 0.811987206 | 0.831177861 | 0.659435405 |
| 60 | −0.359491067 | −0.849443949 | 0.715894869 | 0.766722292 | 0.37463496 |
| 61 | −0.700477392 | −0.420240224 | 0.832081769 | 0.698929217 | 0.801070783 |
| 62 | −0.686787746 | −0.406550578 | 0.832081769 | 0.697399527 | 0.799819218 |
| 63 | −4.256656785 | −3.068541173 | 0.399735781 | 0.521554721 | 0.360589626 |
| 64 | −0.509975839 | 0.218711435 | 0.611180642 | 0.961479627 | 0.612362676 |
| 65 | −0.475298221 | −0.877194947 | 0.904324851 | 0.779029342 | 0.956542901 |
| 66 | −1.741970873 | −3.8433567 | 0.655750243 | 0.163398693 | 0.360589626 |
| 67 | 0.201270526 | −1.051182726 | 0.48616326 | 0.396537338 | 0.683423724 |
| 68 | −0.368232809 | −0.44183268 | 0.999860937 | 0.999026561 | 0.99847031 |
| 69 | −0.562038583 | 0.21104041 | 0.08851342 | 0.163398693 | 0.591294674 |
| 70 | 1.598032118 | 0.988885047 | 0.537199277 | 0.444096788 | 0.143860381 |
| 71 | 0.594054532 | −0.21782466 | 0.157697121 | 0.163398693 | 0.143860381 |
| 72 | 0.267368672 | −3.068064776 | 0.116534557 | 0.444096788 | 0.143860381 |
| 73 | −2.419789819 | −0.398132441 | 0.669517452 | 0.832498957 | 0.81525518 |
| 74 | −0.854750647 | −0.563538327 | 0.510707829 | 0.487832012 | 0.594075928 |
| 75 | −5.953251323 | −5.953251323 | 0.013350021 | 0.163398693 | 0.143860381 |
| 76 | −1.503971591 | −0.271459347 | 0.382631067 | 0.410791267 | 0.637880684 |
| 77 | −1.908185266 | −2.398138008 | 0.734181616 | 0.534487554 | 0.547281324 |
| 78 | −0.675718171 | 0.396922629 | 0.208663607 | 0.423585037 | 0.526700042 |
| 79 | 0.922204973 | 0.906790575 | 0.494785148 | 0.163398693 | 0.443818662 |
| 80 | −11.72515534 | −11.72515534 | 0.097900153 | 0.163398693 | 0.143860381 |
| 81 | −1.178924829 | −0.627115274 | 0.774092616 | 0.832498957 | 0.93756084 |
| 82 | −2.245886351 | −2.334284979 | 0.661034627 | 0.163398693 | 0.143860381 |
| 83 | −2.752705332 | −1.494198416 | 0.489083577 | 0.163398693 | 0.733416771 |
| 84 | −2.752705332 | −1.494198416 | 0.489083577 | 0.163398693 | 0.732234738 |
| 85 | −1.047426787 | −0.740663433 | 0.526839104 | 0.618620498 | 0.588165763 |
| 86 | −6.520063163 | −6.520063163 | 0.273258239 | 0.163398693 | 0.143860381 |
| 87 | 1.887907565 | 0.57352832 | 0.220970658 | 0.604992352 | 0.143860381 |
| 88 | −10.61785613 | −10.61785613 | 0.051522737 | 0.163398693 | 0.143860381 |
| 89 | −1.276969137 | −3.843735454 | 0.413850647 | 0.163398693 | 0.452718676 |
| 90 | −0.722127156 | −0.901739872 | 0.336114588 | 0.434292866 | 0.435196774 |
| 91 | −2.829686904 | −2.548458795 | 0.568279794 | 0.608121263 | 0.461271033 |
| 92 | −1.901681291 | −3.631098501 | 0.482825754 | 0.163398693 | 0.345084133 |
| 93 | −17.07040883 | −17.07040883 | 0.013350021 | 0.163398693 | 0.143860381 |
| 94 | −0.043897409 | 0.420431314 | 0.763384787 | 0.685718259 | 0.798567654 |
| 95 | −2.73516032 | −0.60844497 | 0.518773467 | 0.773953553 | 0.302600473 |
| 96 | −10.63184421 | 7.427125033 | 0.181824503 | 0.163398693 | 0.620497845 |
| 97 | −5.619920012 | −5.619920012 | 0.013350021 | 0.163398693 | 0.143860381 |
| 98 | −1.395279835 | −1.488931789 | 0.732790989 | 0.540606313 | 0.600194688 |
| 99 | −0.800474979 | −0.119316314 | 0.457168683 | 0.163398693 | 0.474690585 |
| 100 | 0.315221591 | −19.36298002 | 0.343832568 | 0.527812543 | 0.143860381 |
| 101 | 9.893219592 | −11.78498073 | 0.290502016 | 0.978584342 | 0.487623418 |
| 102 | −5.34525349 | −5.34525349 | 0.013350021 | 0.163398693 | 0.143860381 |
| 103 | −0.49685091 | −1.504436058 | 0.943679599 | 0.163398693 | 0.721248783 |
| 104 | 0 | 0 | 0.24718398 | 0.163398693 | 0.143860381 |
| 105 | −2.912031968 | 2.890773221 | 0.116534557 | 0.163398693 | 0.302600473 |
| 106 | 4.33270195 | −11.5381671 | 0.013350021 | 0.163398693 | 0.143860381 |
| 107 | −1.270814498 | −1.674610695 | 0.714712835 | 0.632665832 | 0.734876929 |
| 108 | −1.717290489 | −3.972113952 | 0.835349743 | 0.163398693 | 0.143860381 |
| 109 | −0.13140682 | 0.298846463 | 0.71137533 | 0.551314143 | 0.759630093 |
| 110 | −2.174608339 | −2.686928805 | 0.587887637 | 0.700389376 | 0.995132805 |
| 111 | 2.360363332 | −3.171636809 | 0.343832568 | 0.712209707 | 0.37463496 |
| 112 | 0.331528841 | −2.253891903 | 0.644347101 | 0.163398693 | 0.539354749 |
| 113 | −1.483861597 | −0.649379792 | 0.560770407 | 0.527812543 | 0.302600473 |
| 114 | −2.282965072 | −4.094841045 | 0.482825754 | 0.163398693 | 0.143860381 |
| 115 | −1.266105511 | −19.1369565 | 0.064107913 | 0.163398693 | 0.302600473 |
| 116 | −0.018605356 | 0.244801725 | 0.599360312 | 0.89507718 | 0.487623418 |
| 117 | 4.270062448 | 3.458190532 | 0.107008761 | 0.163398693 | 0.143860381 |
| 118 | −0.325908284 | −2.368743285 | 0.759351968 | 0.163398693 | 0.806772354 |
| 119 | −0.321040354 | 0.250017725 | 0.996245307 | 0.994993742 | 0.997496871 |
| 120 | 1.222256794 | 0.464823887 | 0.44472257 | 0.454526491 | 0.733416771 |

### EXAMPLES

### Example 1: Methods of Identifying Synthetic Rescue Interactions

### Overview

The emergence of resistance to cancer therapy remains a pressing challenge and has led to several major experimental and clinical efforts aiming to identify individual molecular events conferring resistance to specific cancer drugs¹⁻⁵. Here, by mining large-scale cancer genomic data, we demonstrate that these molecular events can be attributed to a class of genetic interactions termed synthetic rescues (SR). An SR denotes a functional interaction between two genes where a change in the activity of a vulnerable gene (which may be a target of a cancer drug) is lethal, but the subsequent altered activity of its partner (rescuer gene) restores cell viability. Our approach, INCISOR, mines a large collection of cancer patients' data (TCGA)⁶ to identify the first genome-wide SR networks, composed of SR interactions common to many cancer types. INCISOR accurately recapitulates known and experimentally verified SR interactions^{1-5,11,13,14}. Analyzing genome-wide shRNA and drug response datase^{10,15-18}, we demonstrate in vitro and in vivo emergence of synthetic rescue by shRNA or drug inhibition of INCISOR predicted rescuer genes, providing large-scale validations of the SR network. We then further test and validate a subset of these interactions involving key cancer genes in a set of new experiments. We show that SRs can be utilized to predict successfully patients' survival, response to the majority of current cancer drugs and an emergence of resistance. Finally, by in vitro and in vivo analyses, including our experiments, we show targeting particular rescuer gene of a drug re-sensitizes a resistant cell to the drug, revealing the therapeutic opportunities of SR network. Our analysis puts forward a new genome-wide approach for enhancing the effectiveness of existing cancer therapies by counteracting resistance pathways.

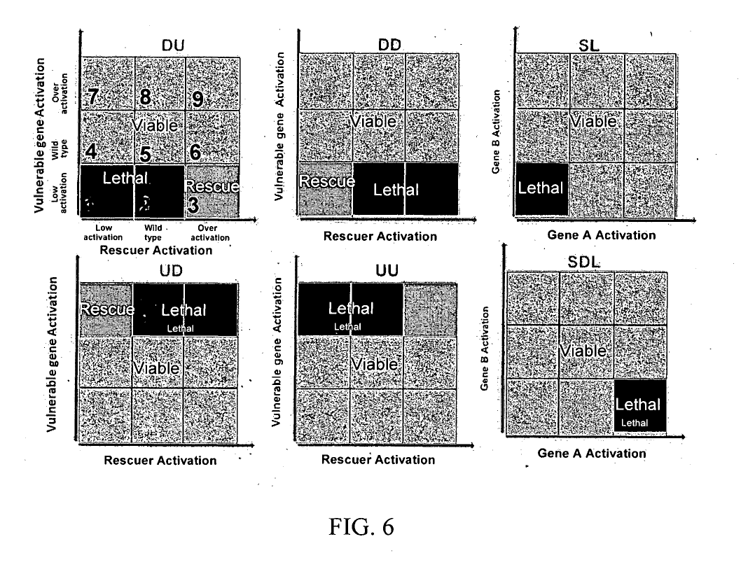

During the course of cancer progression fitness-reducing alterations in some genes may be compensated by cellular reprogramming that involves subsequent alterations in the activity of other genes. We term the former vulnerable genes and the latter rescuer genes and the functional relations between them synthetic rescues (SR). In an SR reprogramming, a change in the activity of one gene places the cell under stress and hinders its viability, but the cell retains its viability (is rescued) by an alteration of the activity of its SR partner. We define four possible different types of SR pairs using a conventional tri-state view of gene-activity in biology (under-activation, wild type and over-activation, see FIG. 6A). An SR pair may involve two inactive genes (DD), a downregulated (inactive) vulnerable gene and an upregulated (overactive) rescuer (DU), an overactive vulnerable gene and an inactive rescuer (UD), and two overactive genes (UU). Any of these SR reprogramming changes can lead to emerging resistance to treatment in cancer, as a drug targeting the vulnerable gene will lose its effectiveness if the tumor evolves an appropriately altered activation of any of its SR rescuer partners. Genetic interaction in SR are conceptually different from another class of genetic interactions termed synthetic lethality (SL)¹⁹⁻²¹, where the inactivation of either gene alone is viable but the inactivation of both genes is lethal. While the role of SL in cancer has been receiving tremendous attention in recent years²², SR reprogramming has received very little attention up to date, if any²³.

This example describes the INCISOR™ pipeline and the use of INCISOR™ to guide targeted therapies in cancer. It comprises of two main components: (a) A description of the INCISOR™ pipeline for identifying Synthetic Rescue (SR) interactions and ways tailoring INCISOR™ to identify other genetic interactions (GIs), specifically Synthetic Lethal (SL) interactions; and (b) an approach for harnessing the SR interactions (or other interactions including SLs) identified to predict drug response in a precision based manner and to identify new gene targets for precision based therapy. The document is organized into four sections: (I) the INCISOR™ pipeline for identify SRs, (II) Harnessing SRs to predict drug response and new targets for adjuvant cancer therapies, (III) auxiliary methods used for testing and validating the predictions made in (I) and (ii), and finally, (IV) a description of how the INCISOR™ pipeline could be modified for the identification of SLs.

### The INCISOR™ Pipeline to Identify SRs

INCISOR™ identifies candidate SR interactions employing four independent statistical screens, each tailored to test a distinct property of SR pairs. We describe here the identification process for the DU-type SR interactions (Down-Up interactions, where the up regulation of rescuer genes compensates for the down regulation of a vulnerable gene (e.g., by an inactivating drug). The methods to detect the other SR types (DD, UD and UU) are analogous to DU with appropriate modifications for the direction of gene activity. We identify pan-cancer SRs (those common across many cancer types) analyzing gene expression, SCNA, and patient survival data of TCGA from 7,995 patients in 28 different cancer types. The same approach can be used to identify cancer type specific SRs, in an analogous manner. INCISOR™ is composed of four sequential steps:
(1) Molecular survival of the fittest (SoF): We mine gene expression and SCNA of multiple tumor samples to identify vulnerable gene (V) and rescuer gene (R) pairs having the property that tumor samples in non-rescued state (that is samples with underactive gene V and non-overactive gene R) are significantly less frequent than expected (due to lethality), whereas samples in rescues state (that is samples with under-active gene V but over-active gene R) appear significantly more than expected (testifying to an explicit rescue from lethality). Specifically, we employ a simple binomial test to identify depletion or enrichment of samples in the different activity bins followed by standard false discovery correction.
(2) Patient Survival screening: The next steps utilize patient survival data to narrow down which of the SR candidate pairs from step 1 are the most promising candidates. This step aims to selects vulnerable gene (V) and rescuer gene (R) pair having the property that tumor samples in rescued state (that is samples with underactive gene V and overactive gene R) exhibits significantly worse patient's survival relative to non-rescued state tumors. Specifically, perform a stratified cox regression with an indicator variable indicating if a tumor is in rescued state for each patient. To infer an SR interaction, INCISOR™ checks association of the indicator variable with poor survival, controlling for individual gene effect on survival. The regression also controls for various confounding factors including, cancer types, sex, age, and race.
(3) shRNA screening: This screen is based on two concepts: (i) knockdown a vulnerable gene V is not essential in cell lines where its rescuer gene R is over-active, and (ii) knockdown of rescuer gene R is lethal in cell lines where V is inactive. Using genome-wide shRNA screens, INCISOR™ examines the samples where V and R show aforementioned conditional essentiality in cell lines depending on each other expression. Specifically, we perform two Wilcox rank sum test to check for the conditional essentiality of V and R.
(4) Phylogenetic distance screening: The final set of putative SRs is prioritized using an additional step of phylogenetic screening, which checks for phylogenetic similarity between the genes composing the candidate interacting pair. This allows to further prioritize SR interactions that are more likely to be true SRs.

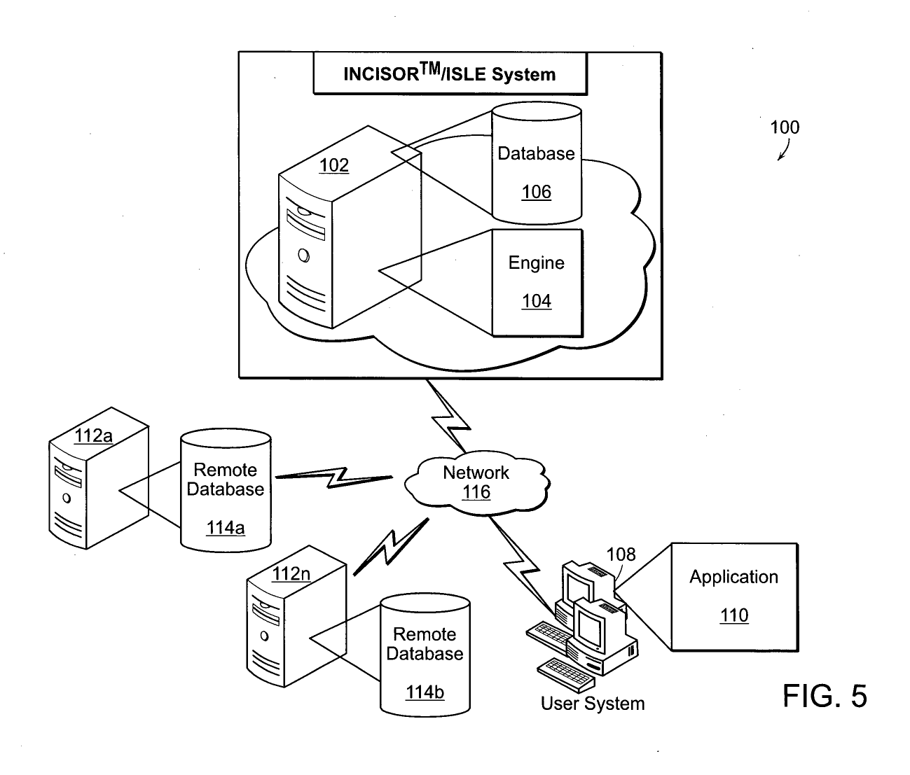

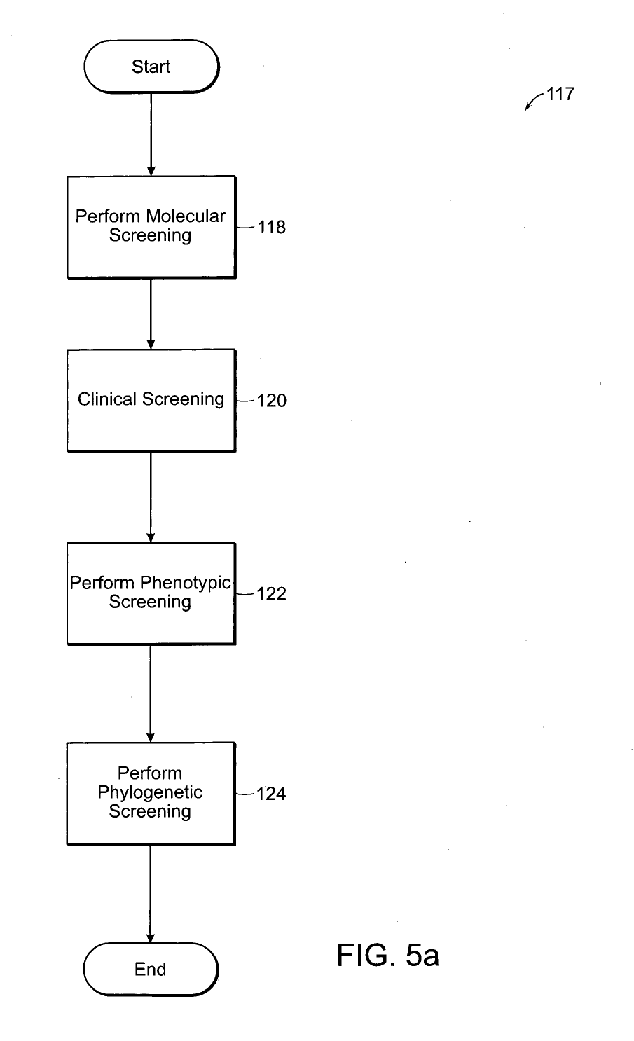

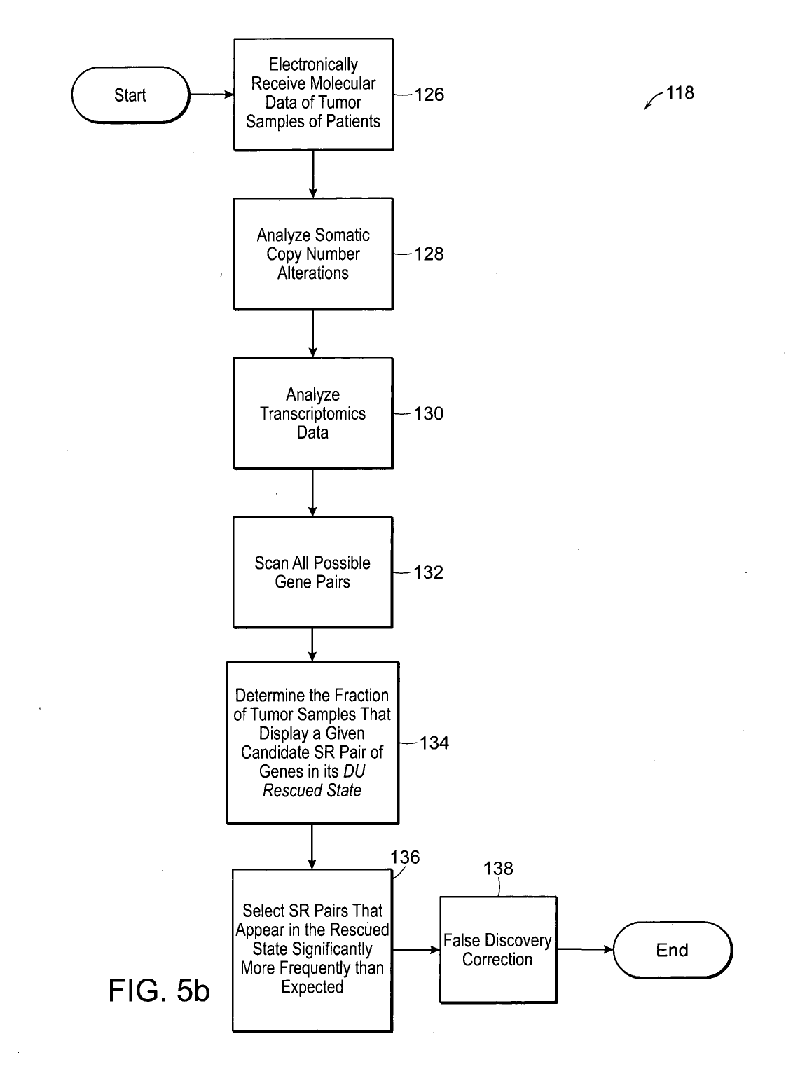

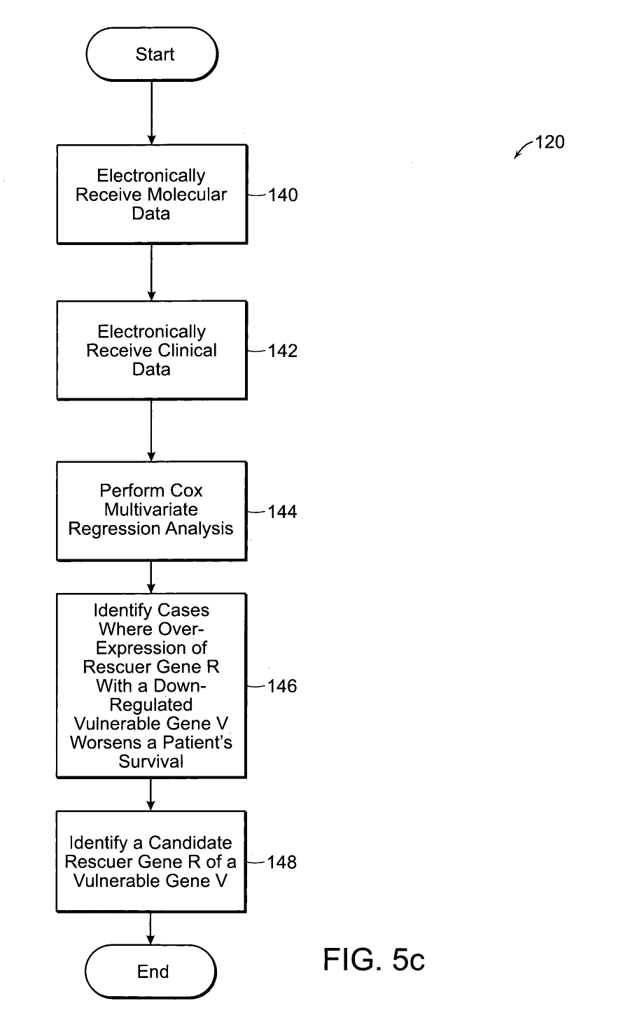

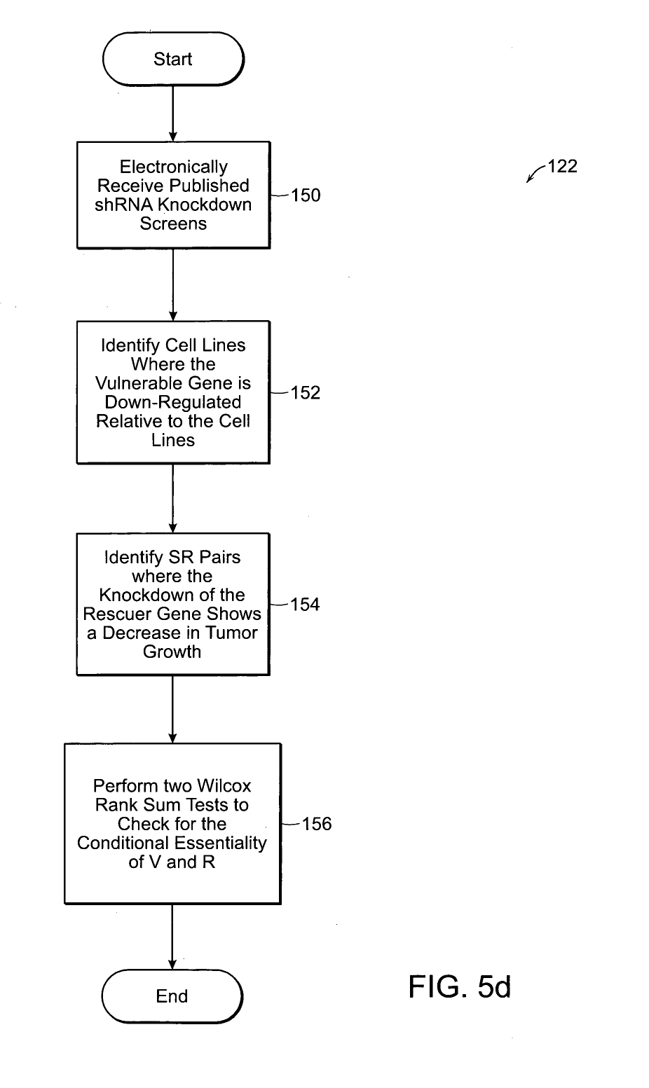

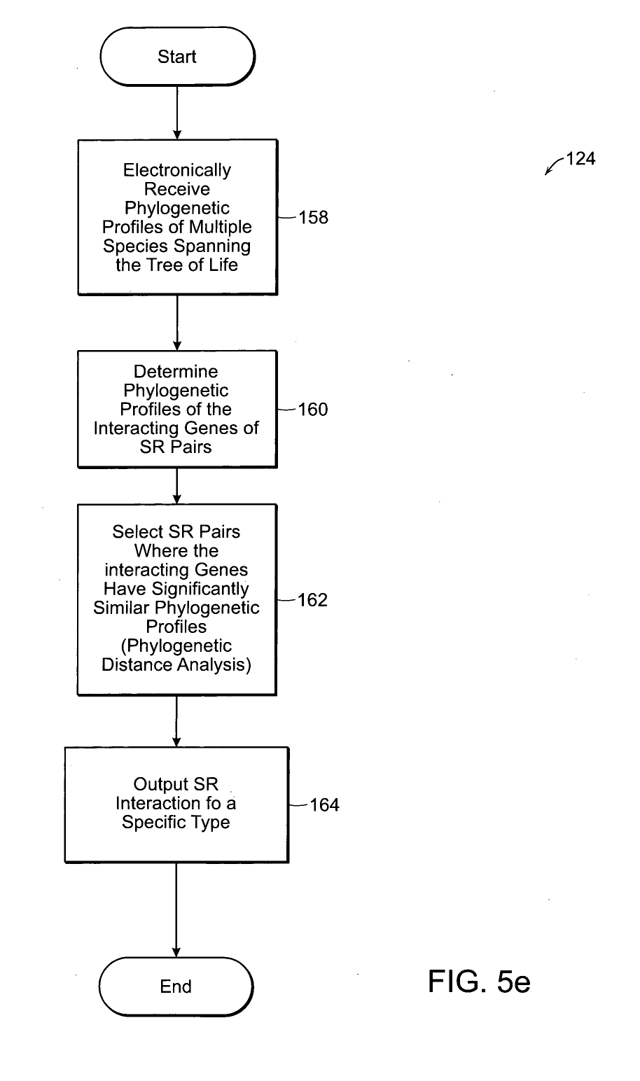

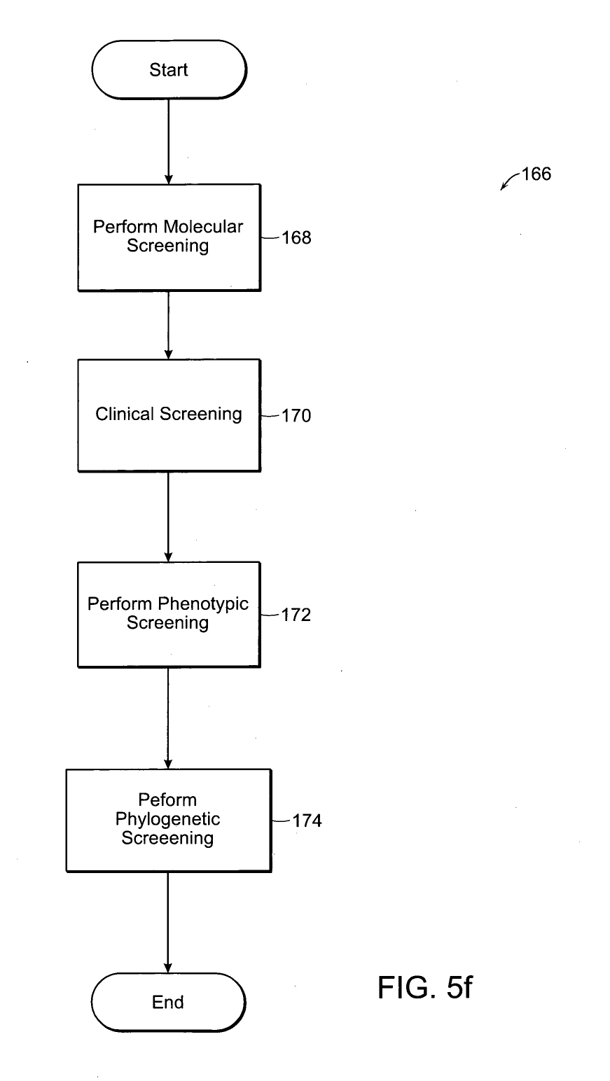

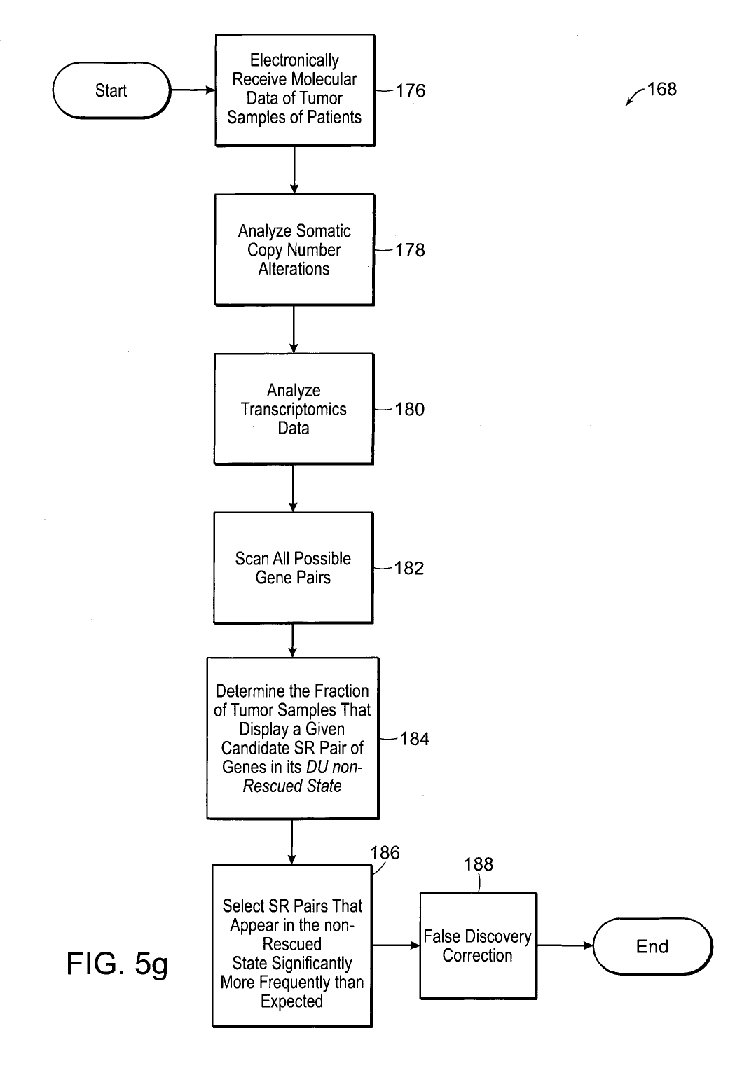

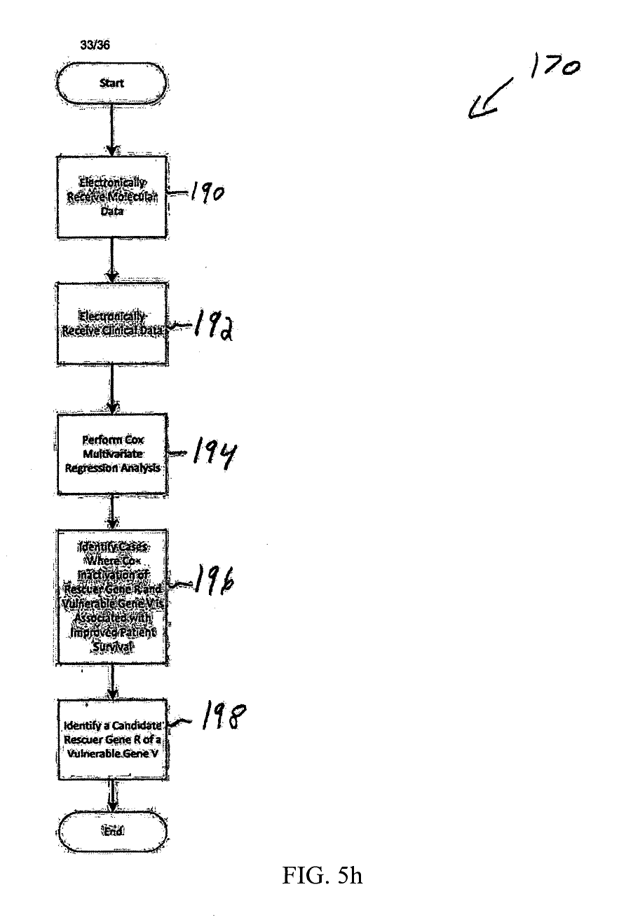

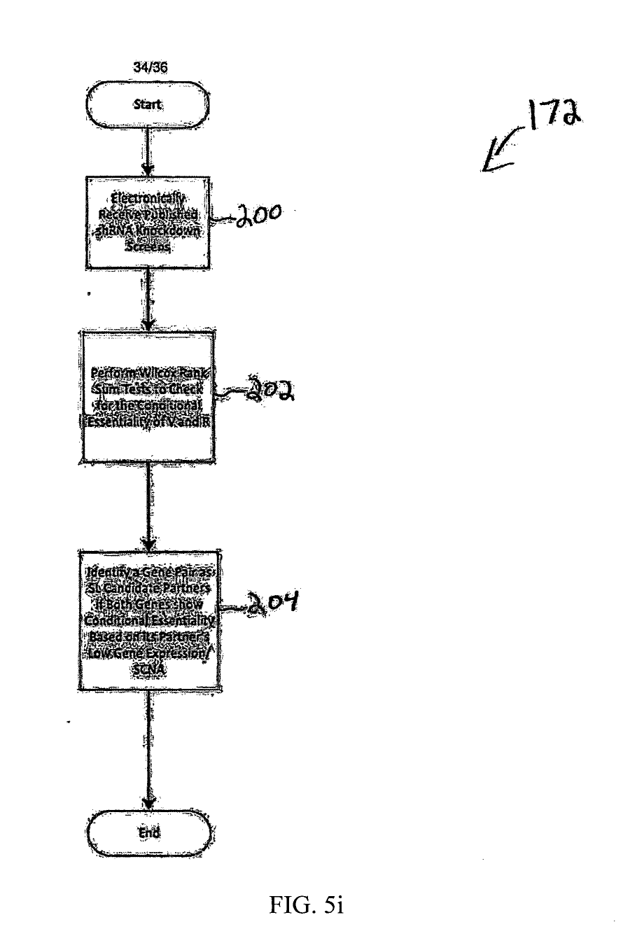

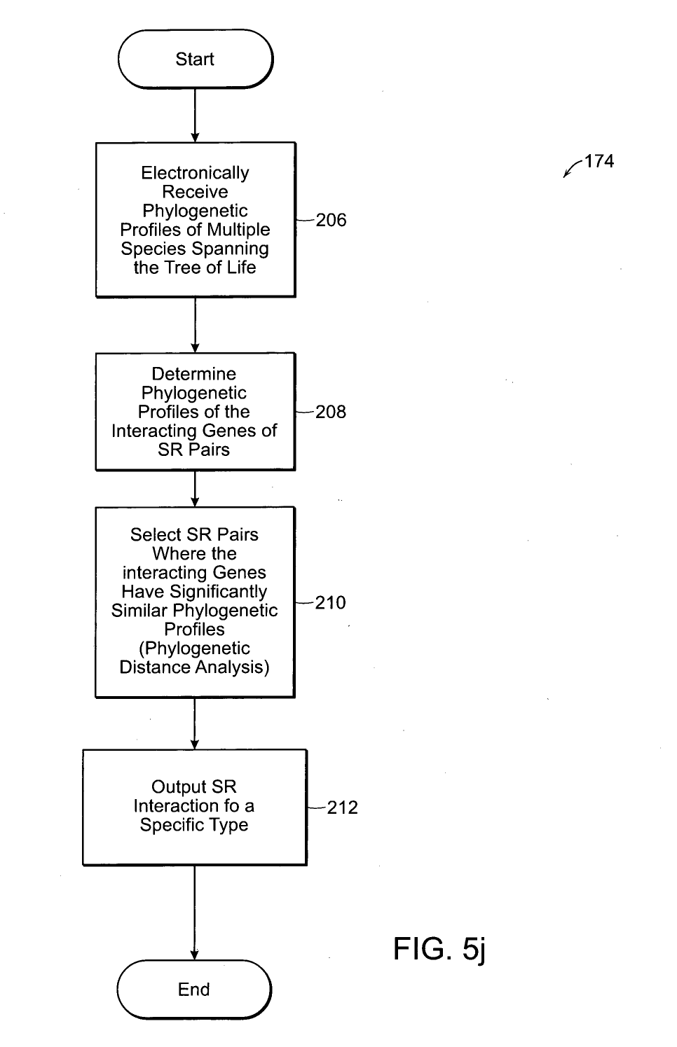

Referring to FIG. 5, a system 100 is shown which illustrates an example of an INCISOR™ system. More specifically, the system 100 could include a server 102 having an engine 104 and a database 106. The engine 104 can execute software code or instructions for carrying out the processing steps for increasing the efficiency of the system 100. The system 100 also includes a user system 108 having an application 110 stored thereon. The user system 108 can be a personal computer, laptop, table, phone, or any electronic device for executing the application 110 and interacting with the server 102. The system 100 further includes a plurality of remote servers 112a-112n having a plurality of remote databases 114a-114n stored thereon. The server 102, remote servers 112 and the user system 108 can communicate with one another over a network 116. As will be explained in greater detail below, the remote servers 112 can input information or data to the INCISOR™ software housed in server 102 via the network 116. It should be noted that the discussion of the system 100 can be adapted to be used for the ISLE software.

Referring now to FIG. 5A is a flowchart detailing the INCISOR™ algorithm 117 is illustrated in greater detail. In step 118, the algorithm 117 will perform molecular screening. In step 120, the algorithm 117 will perform clinical screening. In step 122, the algorithm 117 will perform phenotypic screening. In step 124, the algorithm 117 will perform phylogenetic screening.

In FIG. 5B, a flowchart is provided which illustrates process 118 for molecular screening in greater detail. In step 126, the process 118 electronically receives molecular data of tumor samples of patients. In step 128, the process 118 analyzes the somatic copy number alterations. In step 130, the process 118, analyzes transcriptomics data. In step 132, the process 118, scans all possible gene pairs. In step 134, the process 118 determines the fraction of tumor samples that display a given candidate SR pair of genes in its rescued state. In step 136, the process 118 can select pairs that appear in the rescued state significantly more frequently than expected. Finally, in step 138, the process 118 will apply standard false discovery correction to the results. It should be noted that the process 118 uses samples in different activity bins to improve efficiency and processing for the simple binomial test. The molecular screening process 118 can check if the candidate pairs have a molecular pattern that is consistent with SR. Although a binomial test can be used with the current process, such pairs can be also identified using Wilcoxon ranksum test, t-test or any statistical tests that compares the level of gene A conditioned on the level of gene B, or vice versa.

Reference will now be made to FIG. 5C which illustrates process 120 for clinical screening in greater detail. In step 140, the process 120 electronically receives molecular data. In step 142, the process 120 electronically receives clinical data, which can include various clinical factors including but not limited to patient survival data. In step 144, the process 120 performs a stratified cox multivariate regression analysis. However, this can be achieved by other statistical methods that find association between patient survival or any other clinical variables such as, but not limited to, tumor size, tumor grade, tumor stage that are associated with patient prognosis. Such statistical analyses include parametric and non-parametric models and Kaplan-Meier analysis (which leads to logrank test statistic). In step 146, the process 120 can identify cases where over-expression of rescuer gene R with a down-regulated vulnerable gene V worsens a patient's survival. In step 148, the process can identify a candidate rescuer gene R of a vulnerable gene V. An indicator variable can be used the regression analysis to determine if a tumor is in rescued state for each patient. Individual gene effect can impact the analysis so to make the algorithm more efficient, the process can check association of the indicator variable with poor survival. The process 120 can also control for various confounding factors including, cancer types, sex, age, and race.

Reference will now be made to FIG. 5D which illustrates the phenotypic screening process 122 in greater detail. This process is based on two concepts: (i) knockdown a vulnerable gene V is not essential in cell lines where its rescuer gene R is over-active, and (ii) knockdown of rescuer gene R is lethal in cell lines where V is inactive. In step 150, the process 122 electronically receives published shRNA knockdown screens. In step 152, the process 122 identifies cell lines where the vulnerable gene is down-regulated relative to the cell lines. In step 154, the process 122 identifies SR pairs where the knockdown of the rescuer gene shows a decrease in tumor growth. In step 156, the process 122 performs a wilcox rank sum test to check for the conditional essentiality of the R or V gene. This can be also achieved any other statistical tests that compares the essentiality of one gene under the condition of activity of another gene including t-test, KS test, hypergeometric test, etc. The order in which the aforementioned processing steps are carried out improves computational and processing efficiency. Although large-scale gene essentiality screenings of cancer cell lines based on shRNA are used, any other data can be used that quantifies cancer cell's fitness in response to genetic perturbations (knockout, knock-down, over-expression, etc). Fitness measure could be proliferation (as in the dataset we used), migration, invasion, immune response, etc. Gene perturbation can be performed by different ways including, not limited to, shRNA, siRNA, drug molecules, and CRISPR.

Reference will now be made to FIG. 5E which illustrates the phylogenetic screening process 124 in greater detail. The process 124 checks for phylogenetic similarity between the genes composing the candidate interacting pair. This allows to further prioritize SR interactions that are more likely to be true SRs, which improves computational and processing efficiency. In step 158, the process 124 electronically receives phylogenetic profiles of multiples species spanning the tree of life. In step 160, the process 124 determines phylogenetic profiles of the interacting genes of SR pairs. In step 162, the process 124 selects SR pairs where the interacting genes have significantly similar phylogenetic profiles. In step 164, the process 124 outputs SR interactions of a specific type. The phylogenetic distance between two genes can be calculated in three steps (i) the mapping between homologs in different organisms, (ii) matrix transformation to account for the fact that the species belong to different positions in the tree of life, and (iii) measuring distances of the pair of genes based on the phylogeny in Euclieadian metric. This can be achieved by potentially different alternative ways to identify phylogeny, how to account for the tree of life, and measuring the distance.

It should be noted that the above algorithm 117 improves the functioning of the computer system 100 and engine 104 by providing a framework for narrowing down the gene pairs in such a manner as to provide computational and processing efficiencies. In particular, the order of the process by first performing molecular screening, followed by clinical screening, followed by phenotypic screening and finally performing phylogenetic screening allows the system to run in a more efficient manner. Furthermore, the processing steps allow the system to utilize a growing body of publicly available data in a universal and unsupervised manner.

As noted above, the algorithm 117 can be adapted to run a ISLE process. The ISLE algorithm/process 166 is shown in FIG. 5F in greater detail. In step 168, the algorithm 166 will perform molecular screening. In step 170, the algorithm 117 will perform clinical screening. In step 172, the algorithm 117 will perform phenotypic screening. In step 174, the algorithm 117 will perform phylogenetic screening.

In FIG. 5G, a flowchart is provided which illustrates process 168 for molecular screening in greater detail. In step 176, the process 168 electronically receives molecular data of tumor samples of patients. In step 178, the process 168 analyzes the somatic copy number alterations. In step 180, the process 168, analyzes transcriptomics data. In step 182, the process 168, scans all possible gene pairs. In step 184, the process 168 determines the fraction of tumor samples that display a given candidate SR pair of genes in its non-rescued state. In step 186, the process 168 can select pairs that appear in the non-rescued state significantly less frequently than expected. Finally, in step 188, the process 168 will apply standard false discovery correction to the results. It should be noted that the process 168 uses samples in different activity bins to improve efficiency and processing for the simple binomial test. The molecular screening process 168 can check if the candidate pairs have a molecular pattern that is consistent with SR. Although a binomial test can be used with the current process, such pairs can be also identified using Wilcoxon ranksum test, t-test or any statistical tests that compares the level of gene A conditioned on the level of gene B, or vice versa.

Reference will now be made to FIG. 5H which illustrates process 170 for clinical screening in greater detail. In step 190, the process 170 electronically receives molecular data. In step 192, the process 170 electronically receives clinical data, which can include various clinical factors including but not limited to patient survival data. In step 194, the process 170 performs a stratified cox multivariate regression analysis. However, this can be achieved by other statistical methods that find association between patient survival or any other clinical variables such as, but not limited to, tumor size, tumor grade, tumor stage that are associated with patient prognosis. Such statistical analyses include parametric and non-parametric models and Kaplan-Meier analysis (which leads to logrank test statistic). In step 196, the process 170 can identify cases where co-inactivation of rescuer gene R and vulnerable gene V is associated with improved patient survival. In step 198, the process 170 can identify a candidate rescuer gene R of a vulnerable gene V. An indicator variable can be used the regression analysis to determine if a tumor is in rescued state for each patient. Individual gene effect can impact the analysis so to make the algorithm more efficient, the process can check association of the indicator variable with poor survival. The process 170 can also control for various confounding factors including, cancer types, sex, age, and race.

Reference will now be made to FIG. 5I which illustrates the phenotypic screening process 172 in greater detail. This process is based on two concepts: (i) knockdown a vulnerable gene V is not essential in cell lines where its rescuer gene R is over-active, and (ii) knockdown of rescuer gene R is lethal in cell lines where V is inactive. In step 200, the process 172 electronically receives published shRNA knockdown screens. In step 202, the process 172 performs a wilcox rank sum test to check for the conditional essentiality of the R or V gene. This can be also achieved any other statistical tests that compares the essentiality of one gene under the condition of activity of another gene including t-test, KS test, hypergeometric test, etc. In step 204, the process 172 identifies a gene pair as SL candidate partners if both genes show conditional essentiality based on its partner's low gene expression/SCNA. The order in which the aforementioned processing steps are carried out improves computational and processing efficiency. Although large-scale gene essentiality screenings of cancer cell lines based on shRNA are used, any other data can be used that quantifies cancer cell's fitness in response to genetic perturbations (knockout, knock-down, over-expression, etc). Fitness measure could be proliferation (as in the dataset we used), migration, invasion, immune response, etc. Gene perturbation can be performed by different ways including, not limited to, shRNA, siRNA, drug molecules, and CRISPR.

Reference will now be made to FIG. 5J which illustrates the phylogenetic screening process 174 in greater detail. The process 174 checks for phylogenetic similarity between the genes composing the candidate interacting pair. This allows to further prioritize SR interactions that are more likely to be true SRs, which improves computational and processing efficiency. In step 206, the process 174 electronically receives phylogenetic profiles of multiples species spanning the tree of life. In step 208, the process 174 determines phylogenetic profiles of the interacting genes of SR pairs. In step 210, the process 174 selects SR pairs where the interacting genes have significantly similar phylogenetic profiles. In step 212, the process 174 outputs SR interactions of a specific type. The phylogenetic distance between two genes can be calculated in three steps (i) the mapping between homologs in different organisms, (ii) matrix transformation to account for the fact that the species belong to different positions in the tree of life, and (iii) measuring distances of the pair of genes based on the phylogeny in Euclieadian metric. This can be achieved by potentially different alternative ways to identify phylogeny, how to account for the tree of life, and measuring the distance.

It should be noted that the above algorithm 166 improves the functioning of the computer system 100 and engine 104 by providing a framework for narrowing down the gene pairs in such a manner as to provide computational and processing efficiencies. In particular, the order of the process by first performing molecular screening, followed by clinical screening, followed by phenotypic screening and finally performing phylogenetic screening allows the system to run in a more efficient manner. Furthermore, the processing steps allow the system to utilize a growing body of publicly available data in a universal and unsupervised manner.

In all the above screening processes 118-124 and 168-174, a gene's activities can be based on molecular data. A gene's activities can also be based on different types measurements such as, but not limited to, DNA sequencing (mutation), RNA sequencing (gene expression; transcriptomics), SCNA, methylation, miRNA, lcRNA, proteomics, and fluxomics. The analysis can identify the pairs that are common across many cancer types in all cancer patient population. The same methods can be modified to identify the interaction in particular sub-populations of specific cancer type, sub-types, genetic background (eg. cancer driven by specific driver mutations), specific gender, ethnic group, race, stage, grade, and age-group. The type of interaction one can identify is not limited to SR. As an example, synthetic lethality (where single deletion of either gene is not lethal while deletion of both genes are lethal) and synthetic dosage lethality (where overactivation of one gene renders another gene lethality) can be used. The above processes can also focus on a pair of genes and this can be easily extended triple, quadruple and higher order of genetic interactions with multiple genes. Also, the biological entities are not limited to genes, and the above processes can also be applies to other entities of biological interest such as proteins, RNAs, epigenetic modifications, and environmental perturbations.

### Example 2: Using SR to Predict Drug Response and New Targets for Devising Adjuvant Cancer Therapies

### Constructing a Cancer-Drug DU SR Network

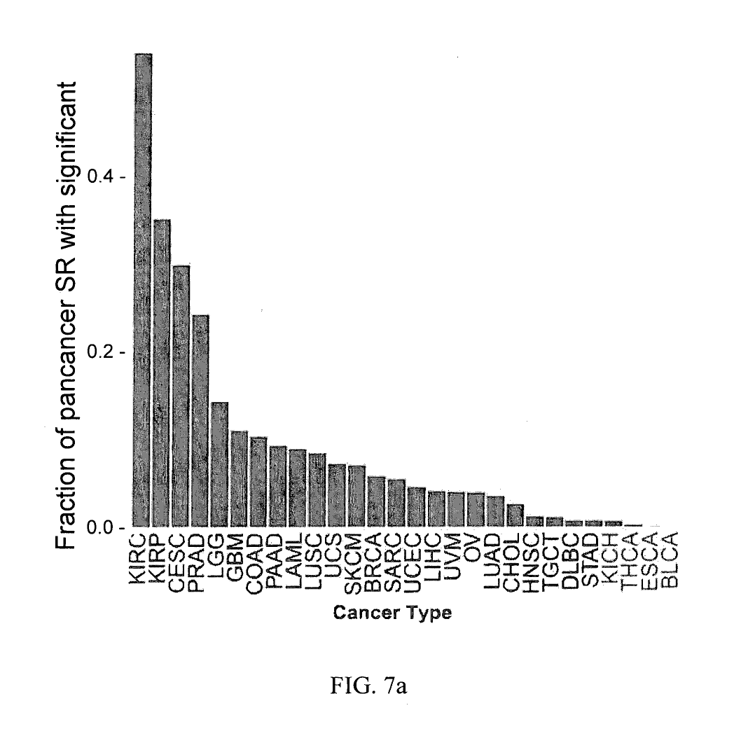

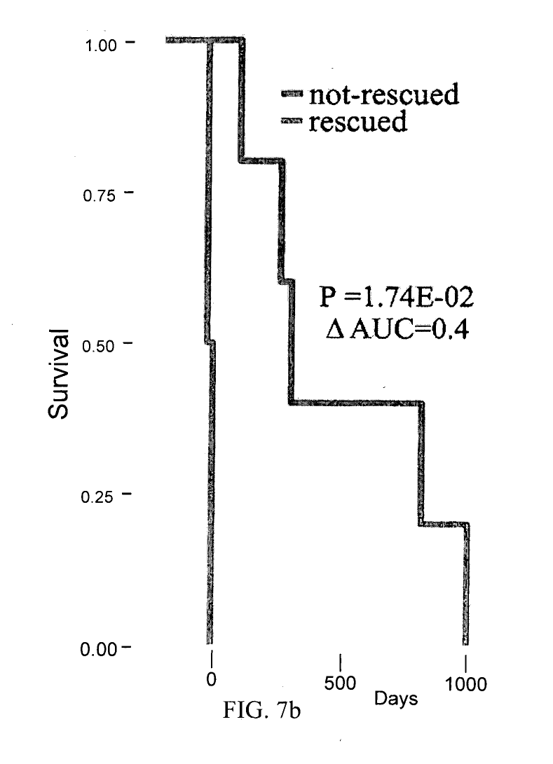

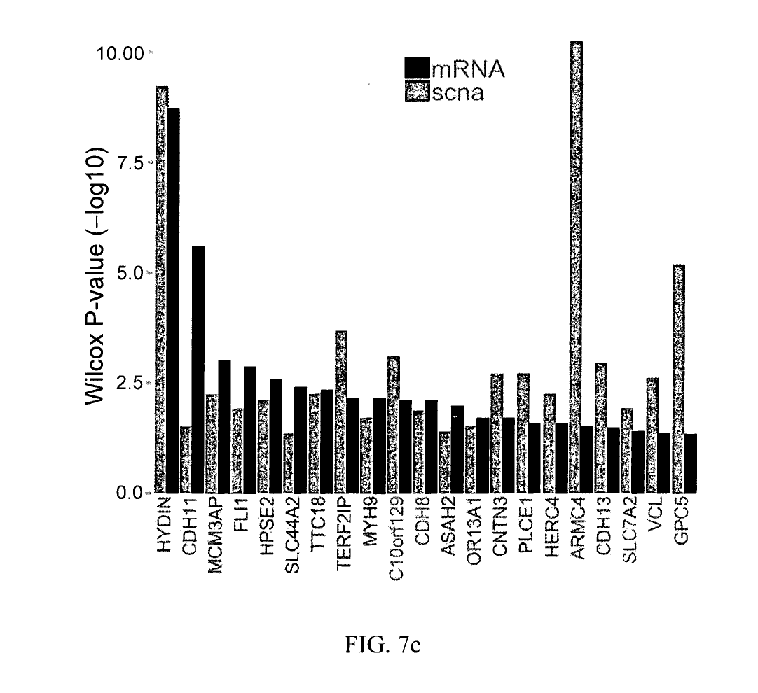

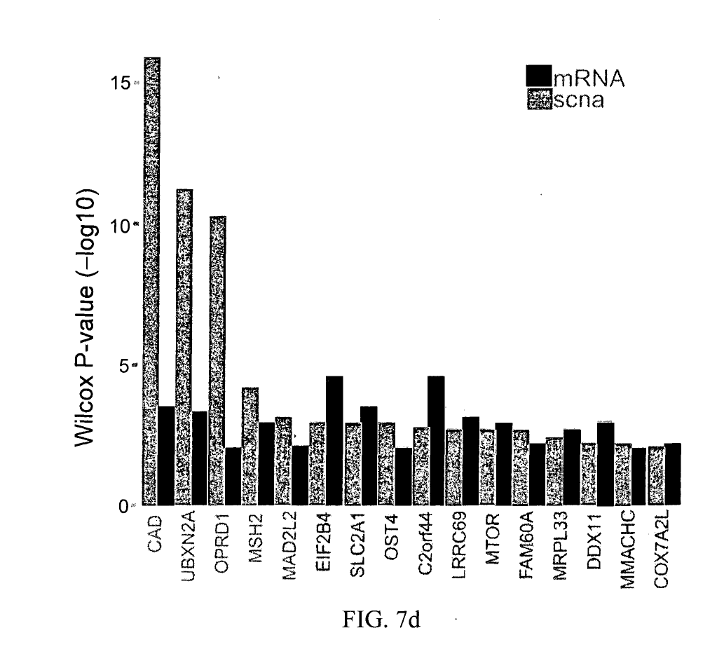

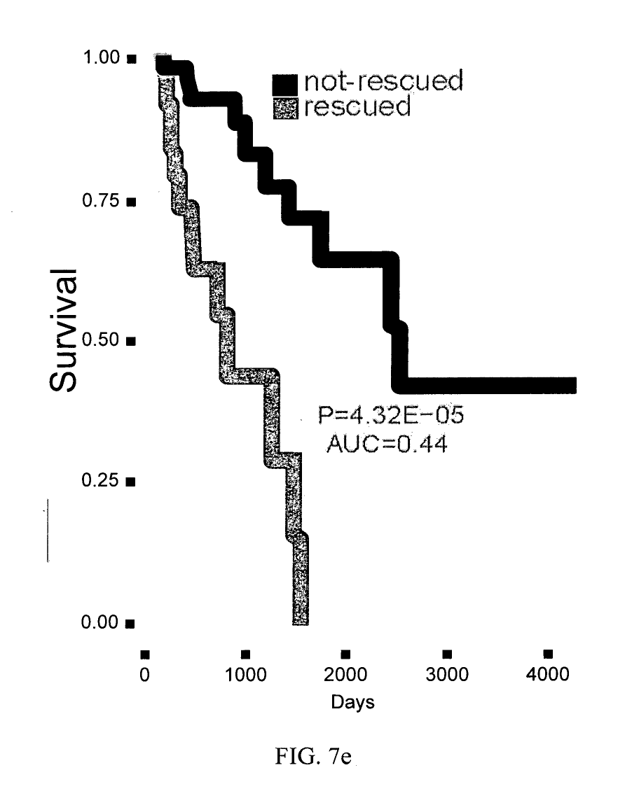

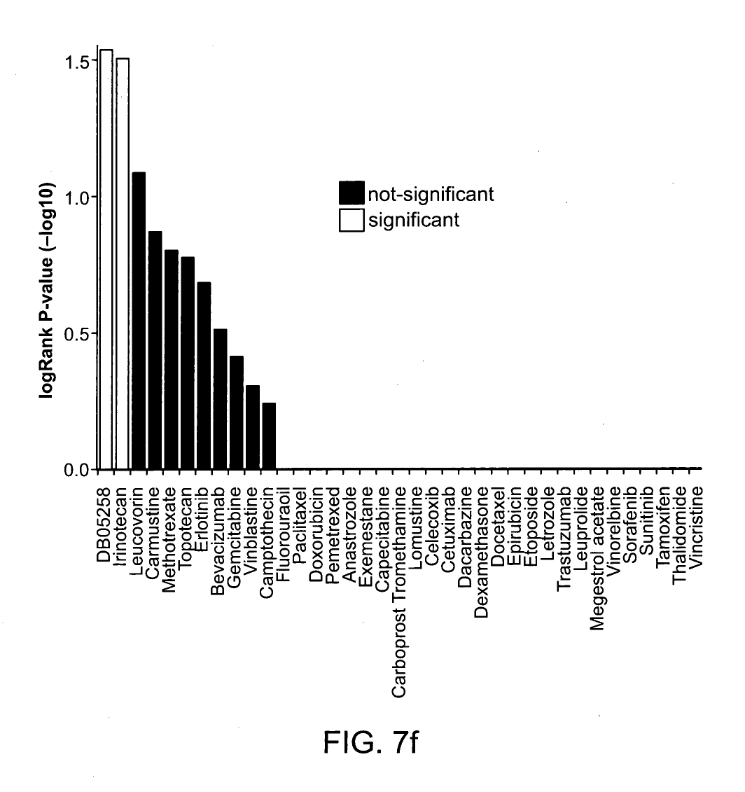

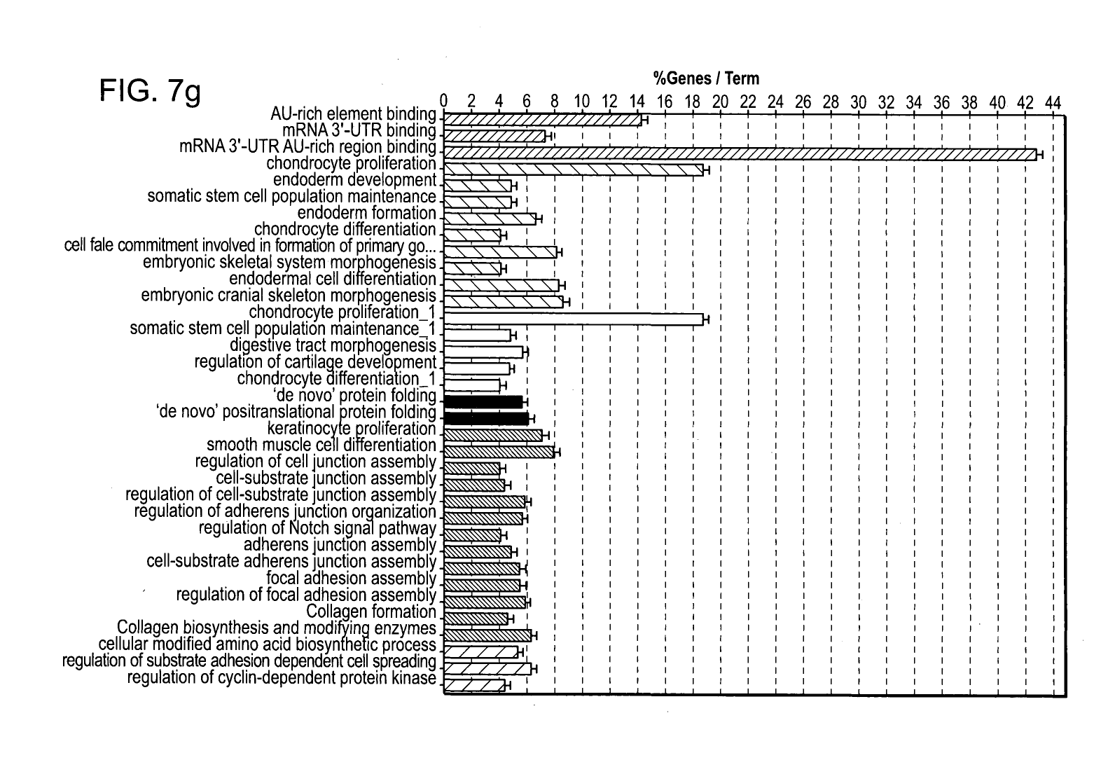

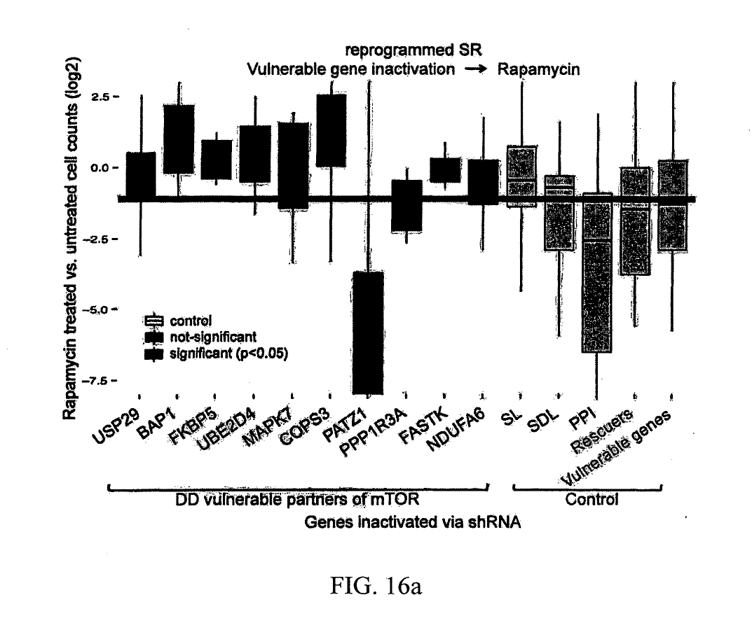

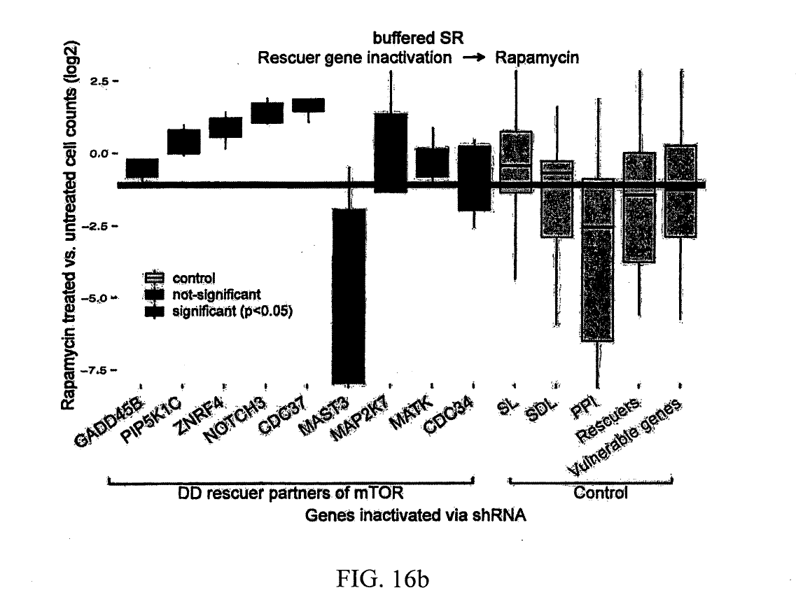

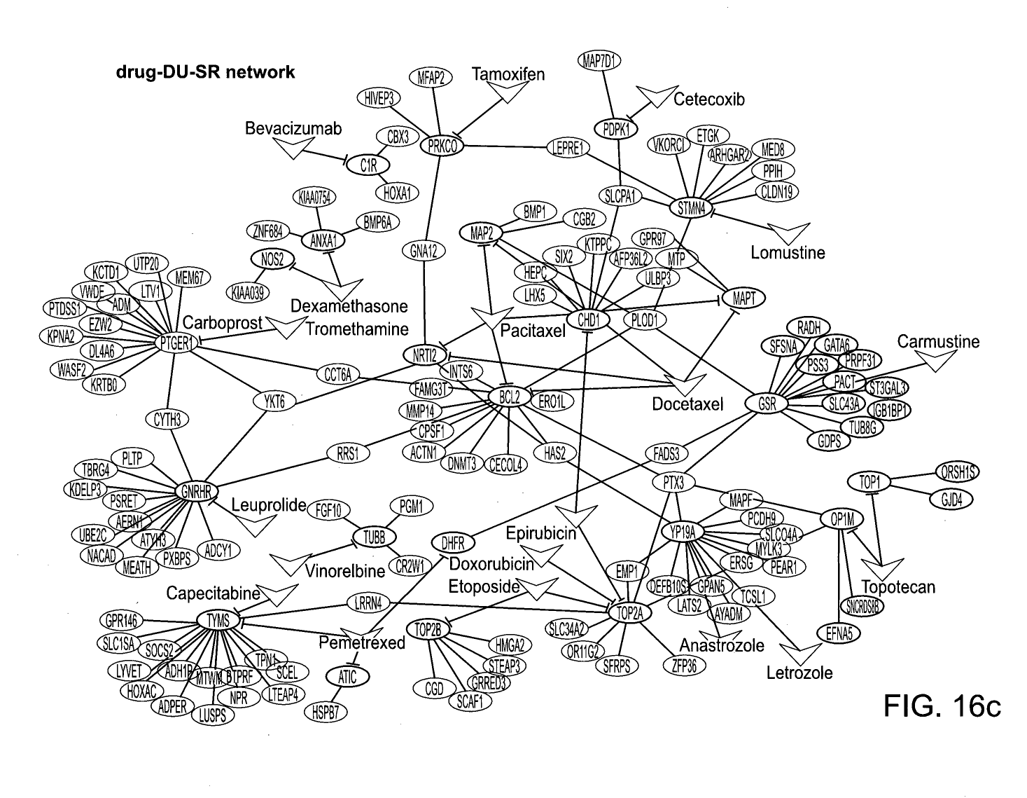

To show the utility of SR network in predicting drug resistance and response we constructed a cancer-drug DU SR network (drug-DU-SR) using pan-cancer TCGA data. Gene targets of 37 drugs that are included drug-DU-SR were identified using Drugbank database²⁴. In identifying the original genome-wide DU-SR network, we have applied very conservative criteria (FDR<0.01 wherever applicable) at each step of INCISOR™. As a result, the network contained only 2033 interactions (3.5×10⁻⁴% of all possible gene pairs), leaving out many potential rescuers of many drug targets. To capture DU-type rescuers of anti-cancer drug targets in a more comprehensive manner we modified INCISOR™ as follows: (i) An FDR correction was applied only at the last step, and (ii) The SR significance P-value threshold were relaxed to accommodate weaker SR interactions. The resultant network drug-DU-SR includes the targets of most of the 37 cancer drugs that were administered to TCGA patients, encompassing 170 interactions between 36 vulnerable genes (drug targets) and 103 rescuer nucleic acid sequences (FIG. 16c). A pathway enrichment analysis shows that the rescuers are highly enriched with lipid storage/transport, thioester/fatty acid metabolism, and drug efflux transporters (FIG. 7g).

### Predicting Pan-Cancer Drug Response I

Applying INCISOR to the pan-cancer TCGA data spanning 7,550 samples across 23 different cancer types⁶, we exerted the first genome-wide effort to systematically uncover SR reprogramming in cancer and study their translational value. Unless stated otherwise we focus the lion's share of the analysis on DU-SR reprogramming. The resulting SR network (DU-type) has 1,182 interactions involving 450 rescuer nucleic acid sequences and 589 vulnerable genes, and consists of two large disconnected subnetworks: Growth factor subnetwork and DNA-damage subnetwork. The vulnerable genes in the Growth factor subnetwork are enriched with processes associated with growth factor stimulus and nuclear chromatin, and are mainly rescued by genes related to vitamin metabolism and positive regulation of GTPase activity. In the DNA-damage subnetwork the vulnerable genes are broadly associated with DNA-damage, metal ion response and cell-junction, and are rescued by DNA mismatch, repair protein complex (MutS) and receptor signaling regulation genes. Notably, the deregulation of MutS has been previously reported to cause resistance to an array of cancer drugs, including etoposide, doxorubicin (hypergeometric p-value<0.06), as expected. SR pairs are not enriched with protein-protein interactions.

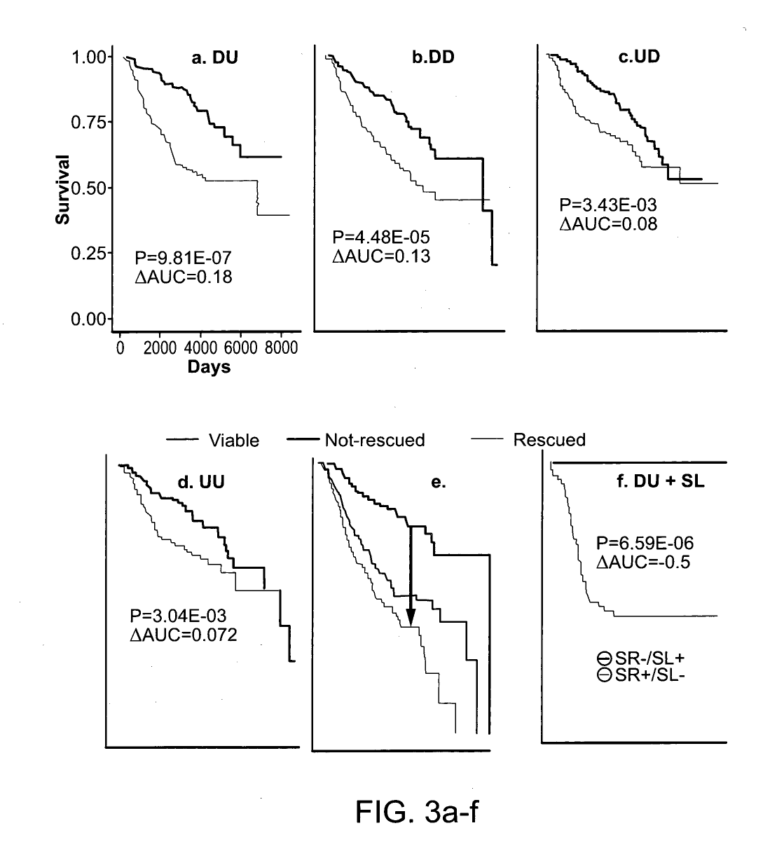

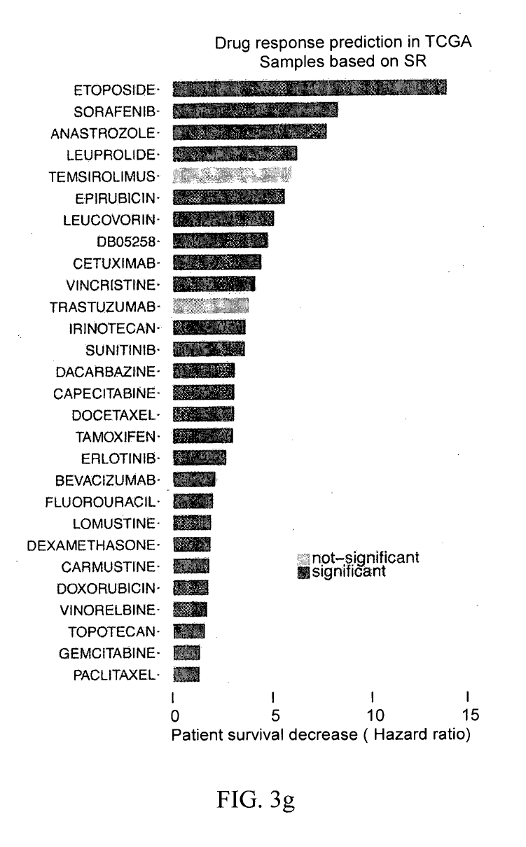

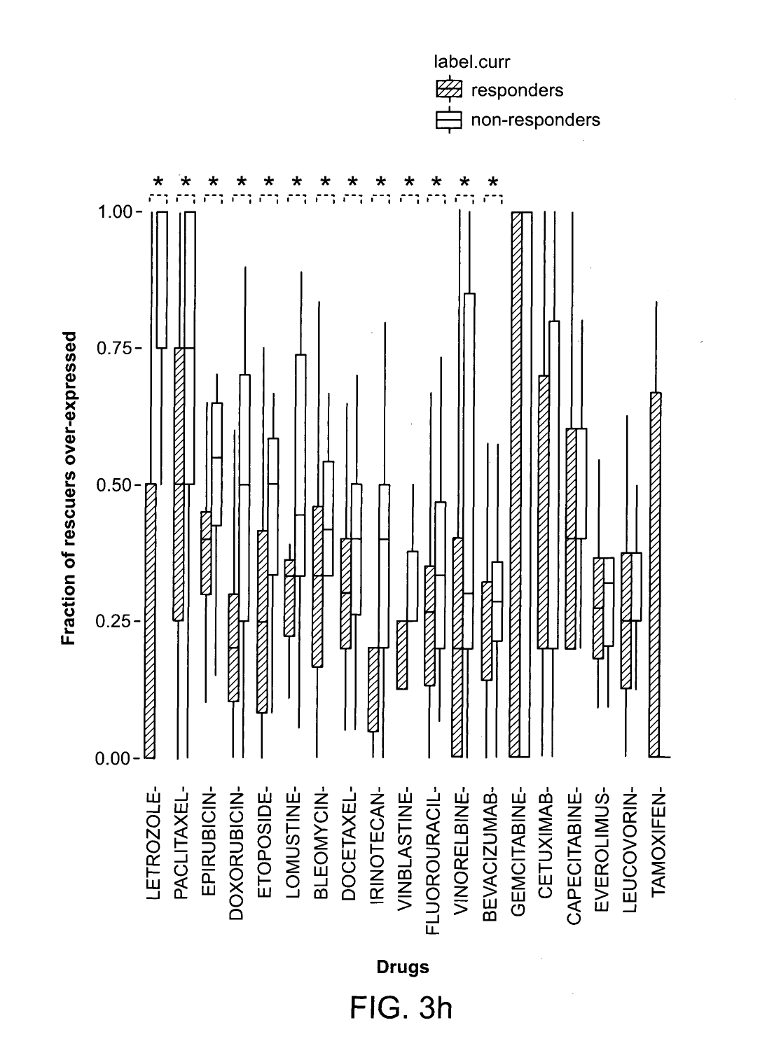

We first tested the clinical significance of the pan-cancer SRs inferred above in an independent METABRIC breast cancer (BC) dataset (Methods)²⁵. We quantified the number of functionally active SRs in each sample—that is, SR-DU pairs where a vulnerable gene is inactive and its rescuer partner is over-activated in the given sample. As expected, we find that breast cancer samples with a large number of functionally active pairs have significantly worse survival than samples with fewer active pairs, as the former are rescued (FIG. 3a). This finding is also true for the other three SR types, albeit to a lesser extent (FIG. 3 b,c,d). Notably, patients harboring tumors with extensive SR reprogramming (many functionally active SR pairs) have significantly worse survival than the rest (FIG. 3e). Combining SR with SL interactions only slightly improves the survival predictive power further (FIG. 3f). We further applied INCISOR to identify the four types of SRs in the TCGA BC data and then tested their clinical significance in a large independent BC cohort, and we confirmed that SR-DU shows the highest predictive survival signal. Interestingly, BC SR-DUs show a strong involvement of immune-related processes: while vulnerable SR-DU genes are enriched with tolerance against natural killer cells (the inactivation of which will lead the cancer cells susceptible to immune system), the rescuer genes are enriched with negative regulation of cytokines (which will prevent immune cells from being recruited by cytokines). Finally, we find that the copy number of DU rescuer genes is significantly higher in samples with mutated vulnerable genes than in samples without such mutations (Wilcoxon P<1.2e−100), and so is the rescuers' gene expression (Wilcoxon P<1.1E−17), testifying to the ongoing rescue reprogramming.

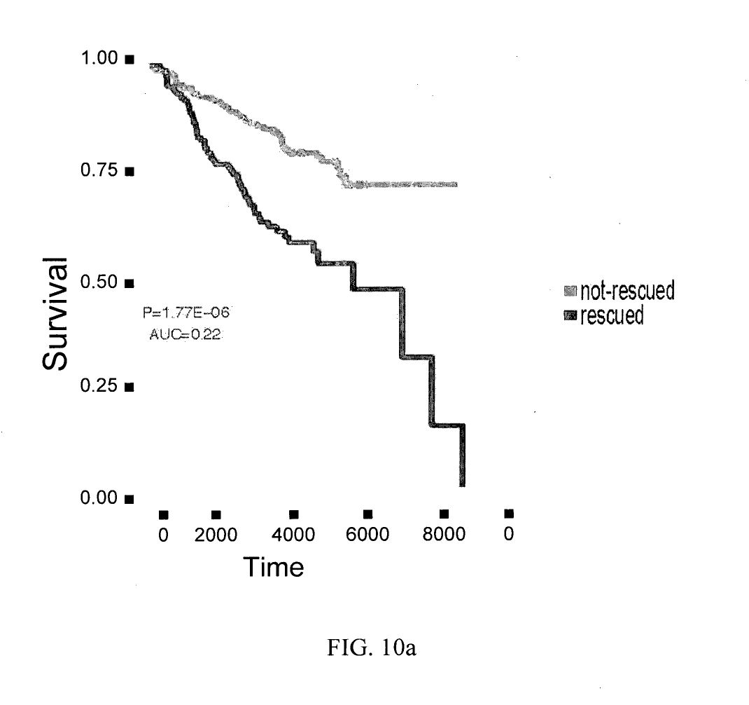

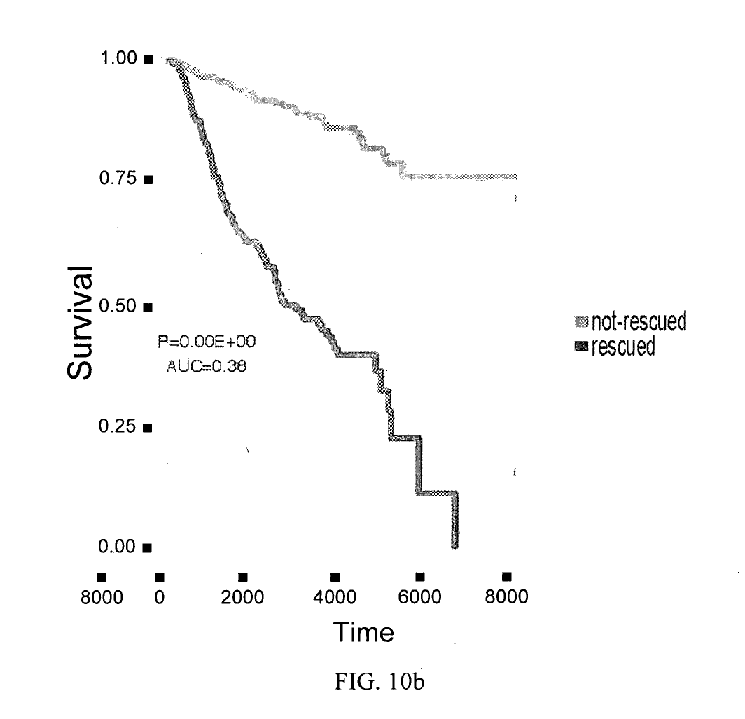

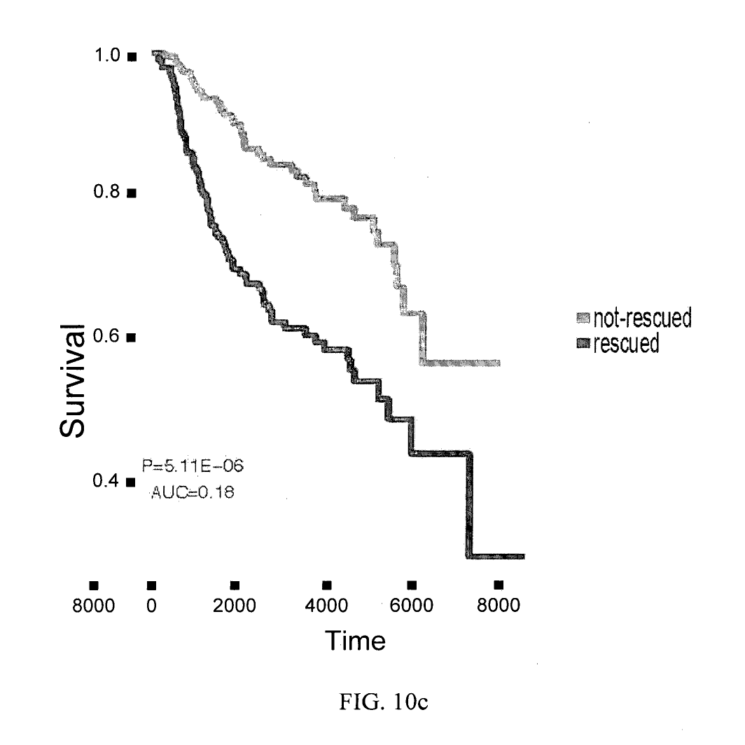

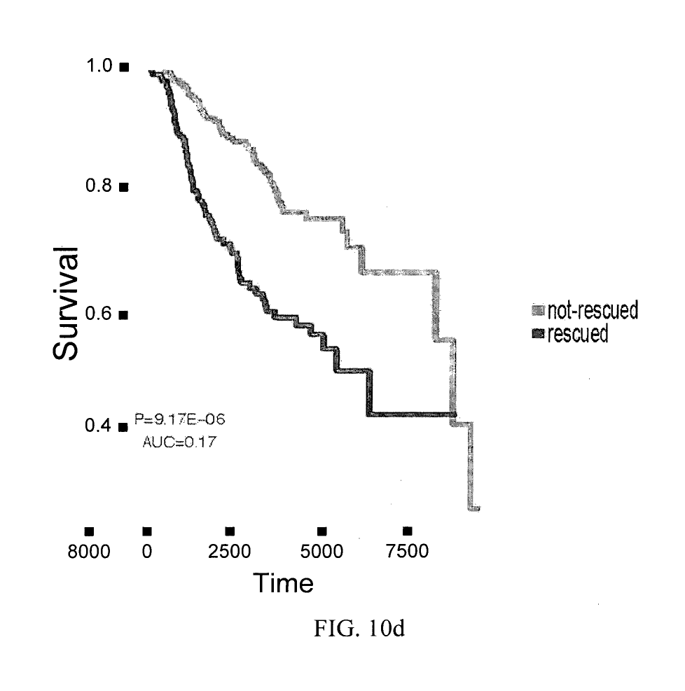

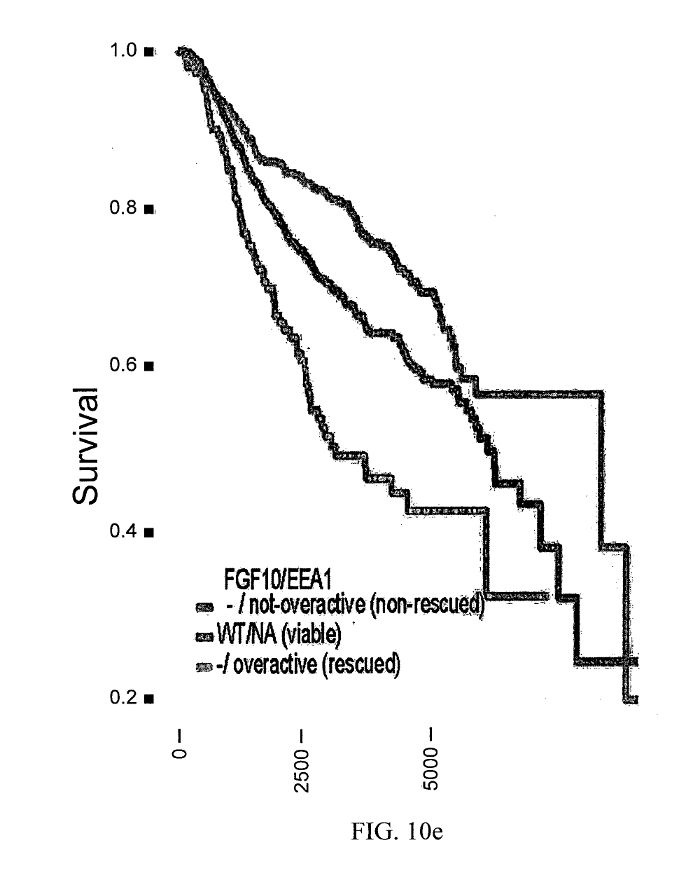

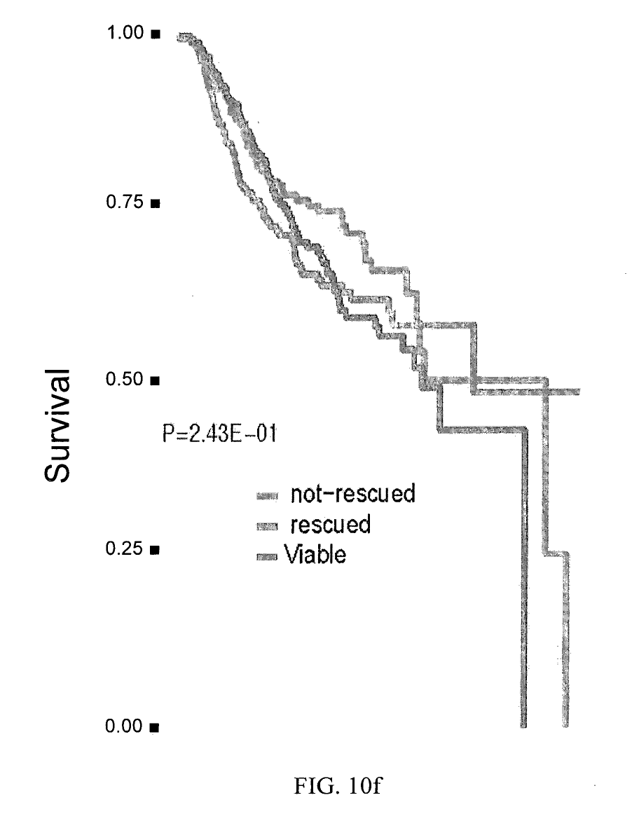

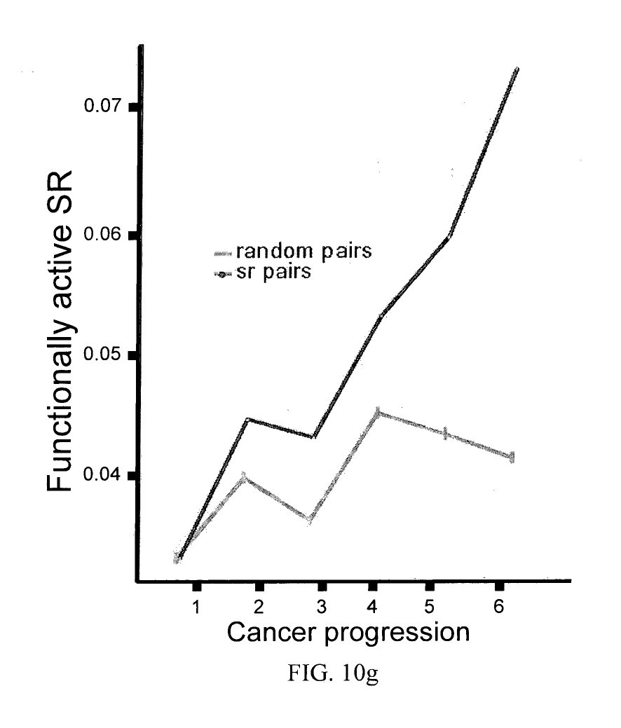

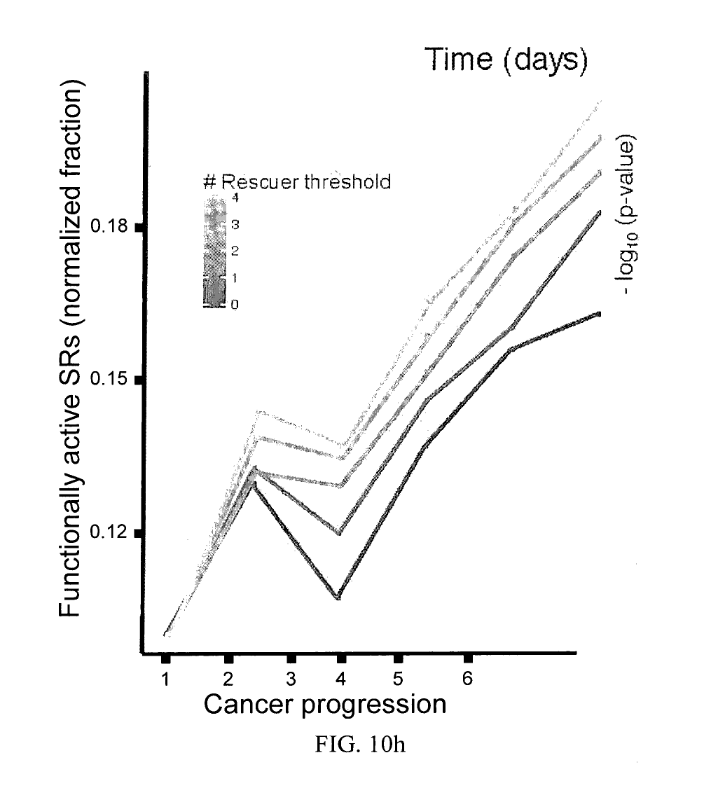

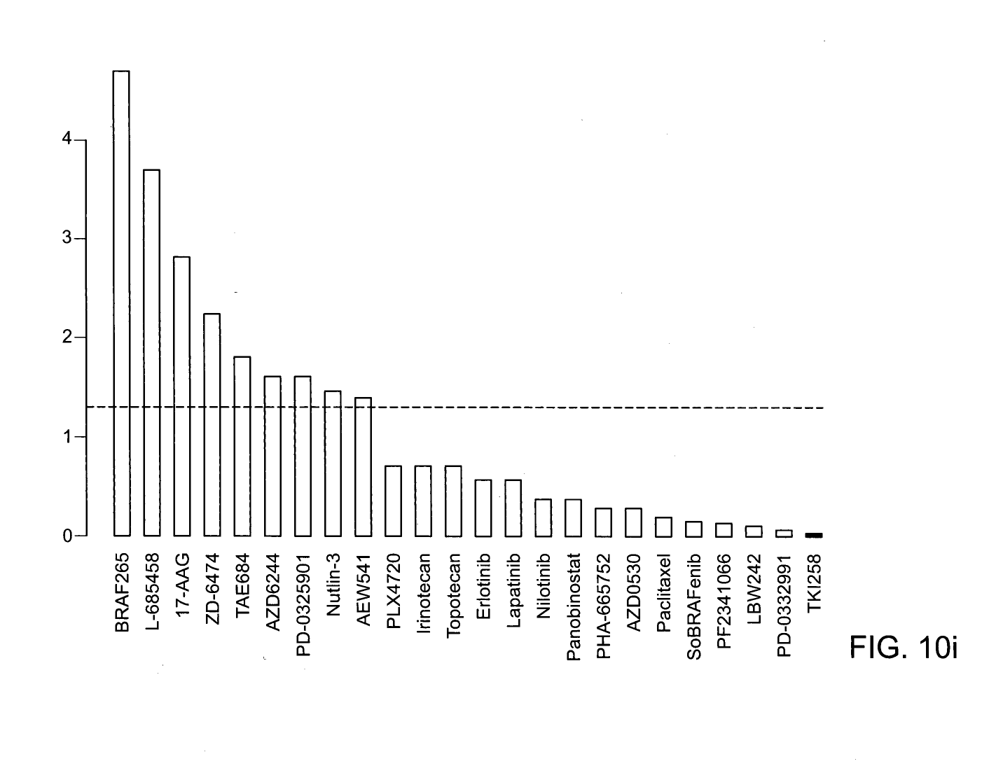

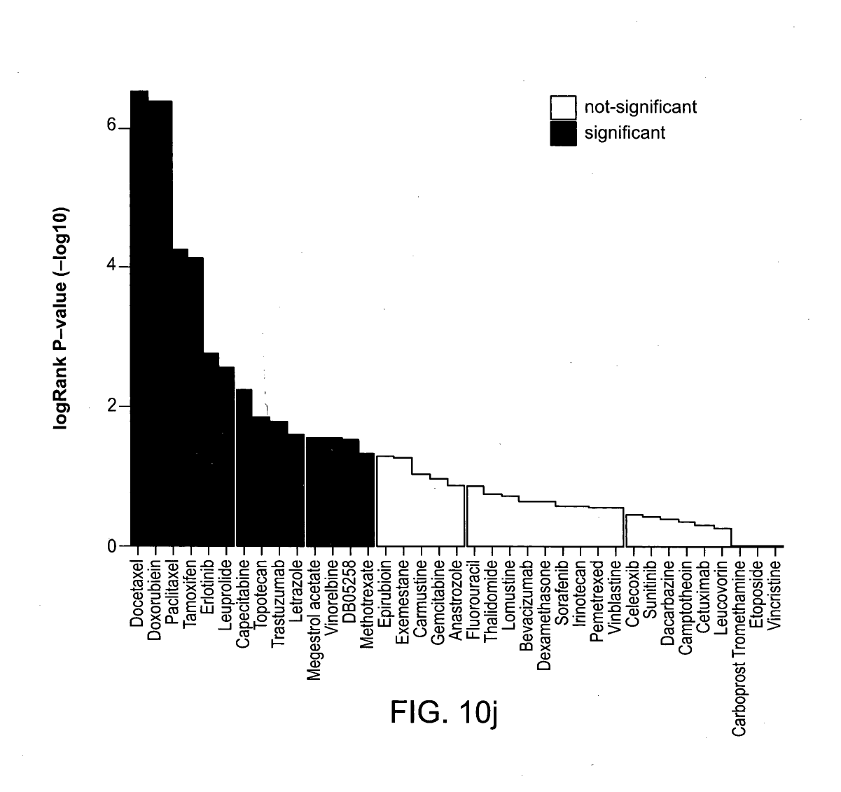

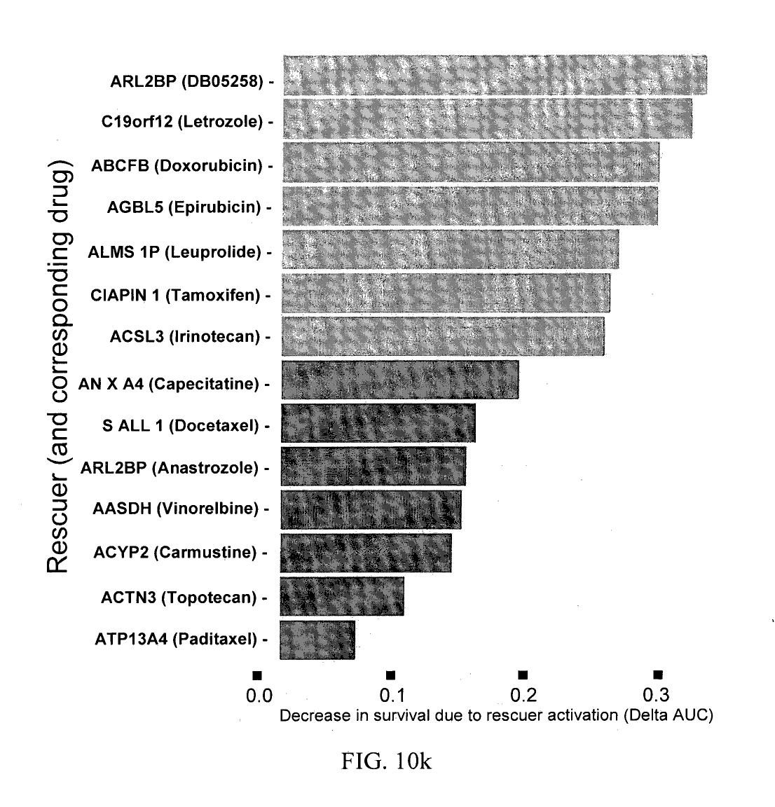

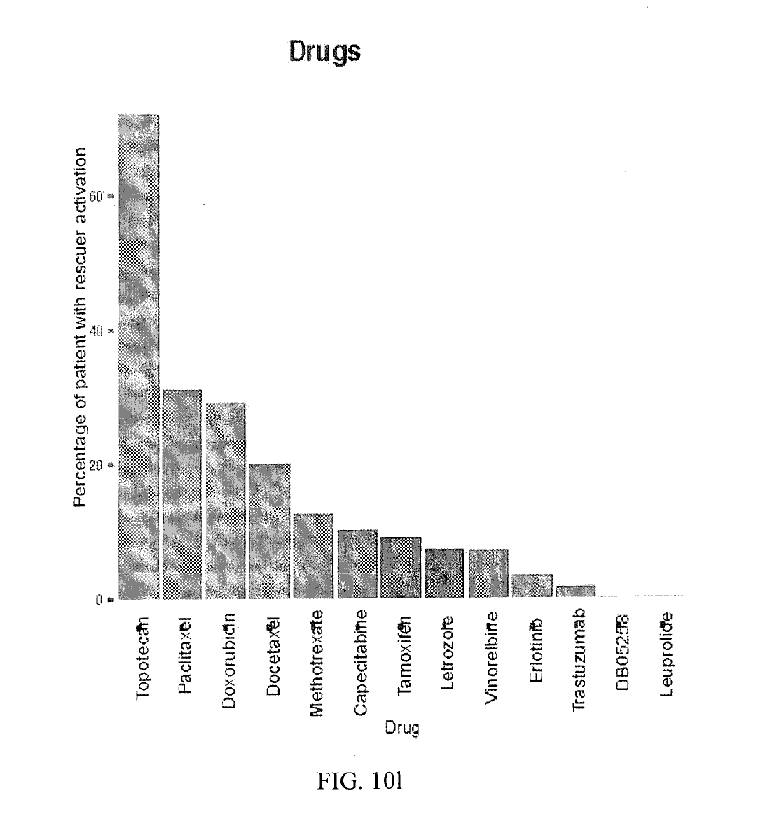

To study the dynamics of SR functional activity as cancer progresses, we stratified the BC patients in the METABRIC dataset into six different cancer progression bins by their survival times. As expected, cancer progression is accompanied by an increase in the number of functionally active SRs in the tumors (FIG. 10g) and by an increase in the number of inactive vulnerable genes that are rescued (FIG. 10f). We further distinguished between reprogrammed SRs (rSR), where the rescuer gene over-activation occurs after the inactivation of its paired vulnerable gene, to buffered SR (bSR), where the rescuer gene over-activation precedes the inactivation of the vulnerable gene. While in general SRs carry clinical significance irrespective of their order of occurrence, rSRs have a significantly stronger survival predictive signal than bSRs. This further emphasizes the active rescue role of SR events in cancer progression.

We next investigated the ability of the DU SR network to predict the clinical response to therapy with major anticancer drugs. This prediction is obtained in an unsupervised straightforward manner (no training) by quantifying how many of the rescuer partners of the targets of a given drug are over-activated in a given patient's tumor. As our original SR network does not include many of the cancer drug target genes, we applied INCISOR to build a specific cancer-drug DU SR network that includes drug targets by allowing for weaker interactions (Methods). Using the drug-DU SR network and molecular signatures of cancer patients we classified each patient to be a non-responder (responder) to a given drug if one or more of the rescuer partners of that drug are over-active (and as a responder if none), and compared the survival rates of predicted responders to those of non-responders. We analyzed drug response of 3873 patients in TCGA dataset, focusing on 36 common anticancer drugs that were administered for at least 30 patients. We correctly classify patients into responder and non-responders for 26 drugs (FIG. 3h). The prediction pipeline is generic and unsupervised and successfully predicts drug response in additional datasets as follows.

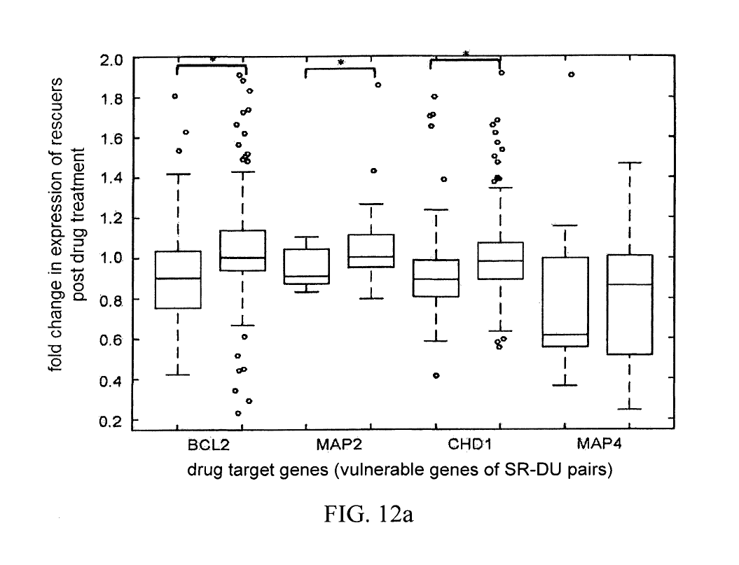

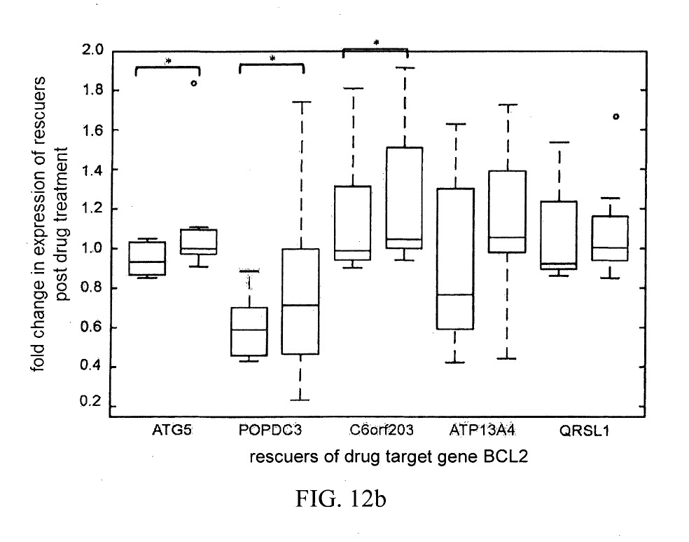

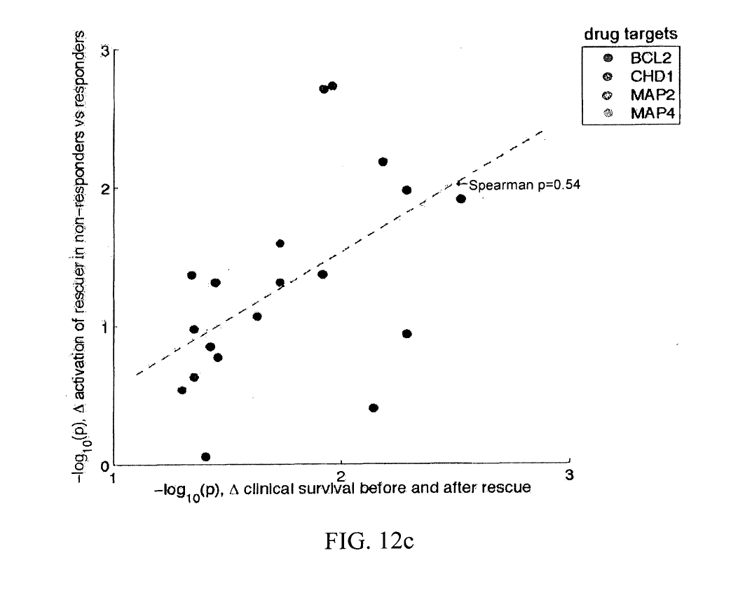

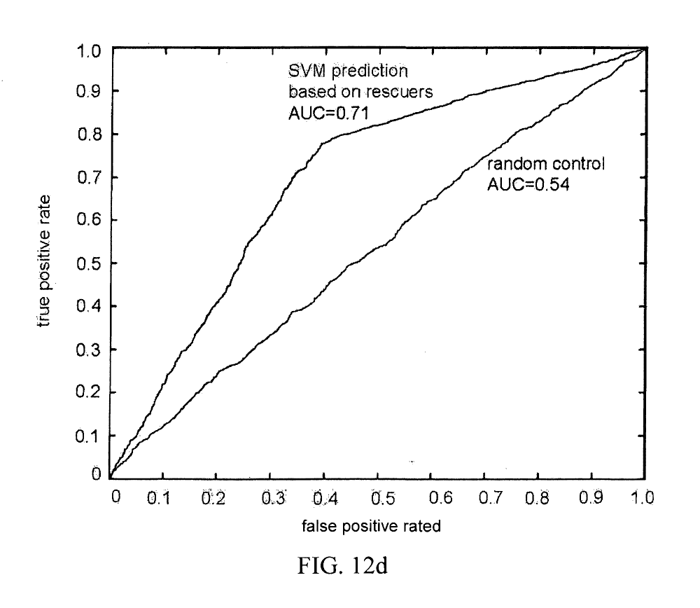

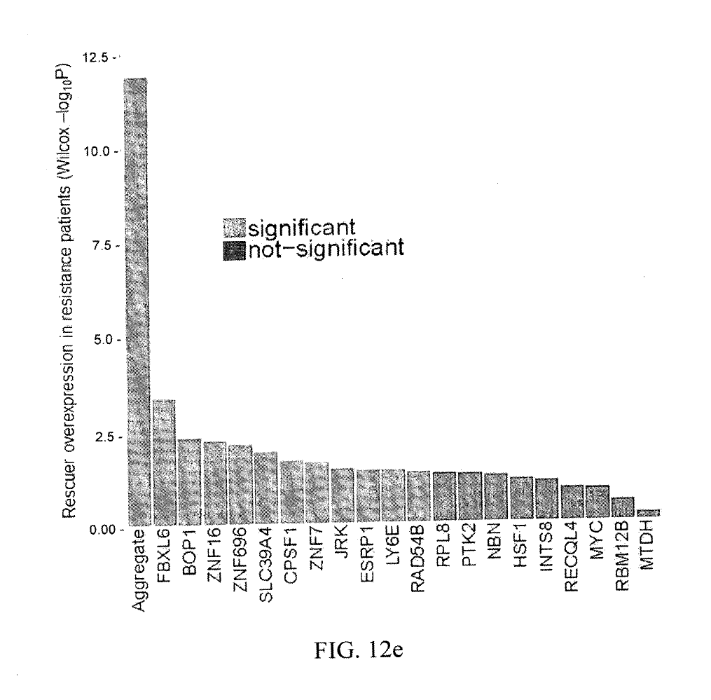

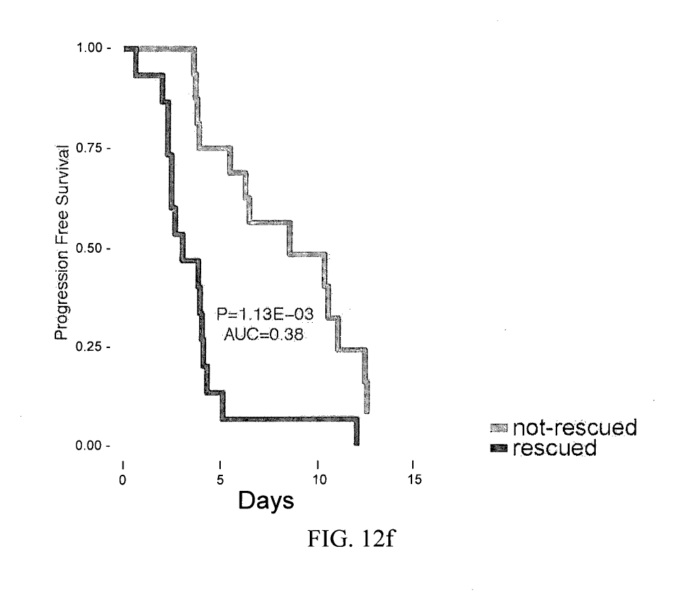

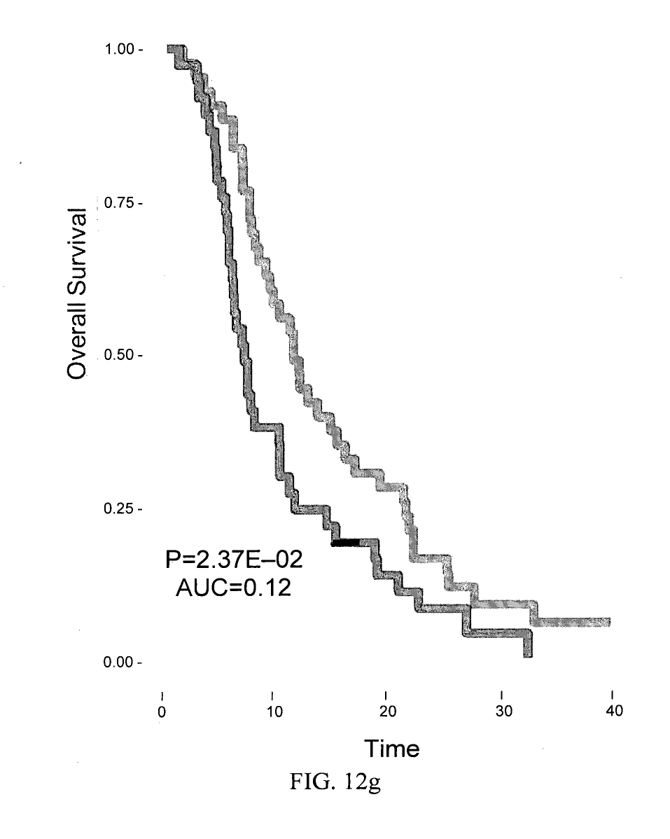

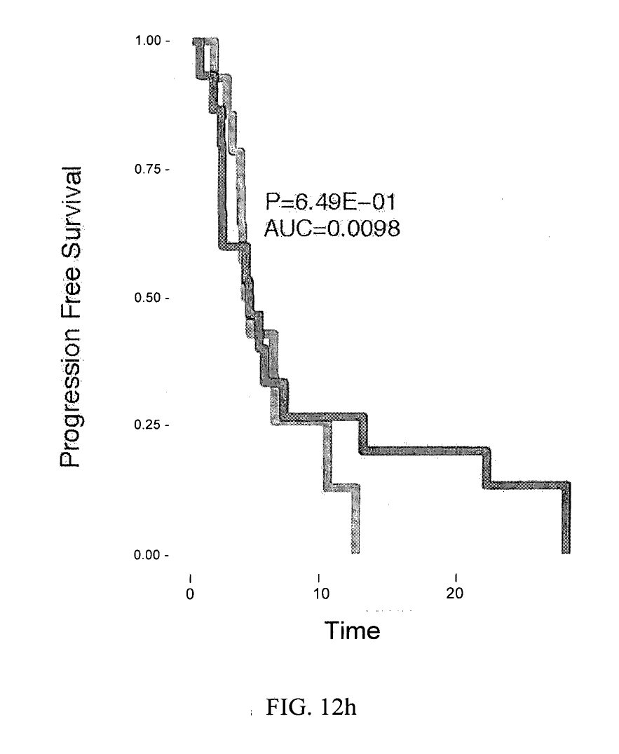

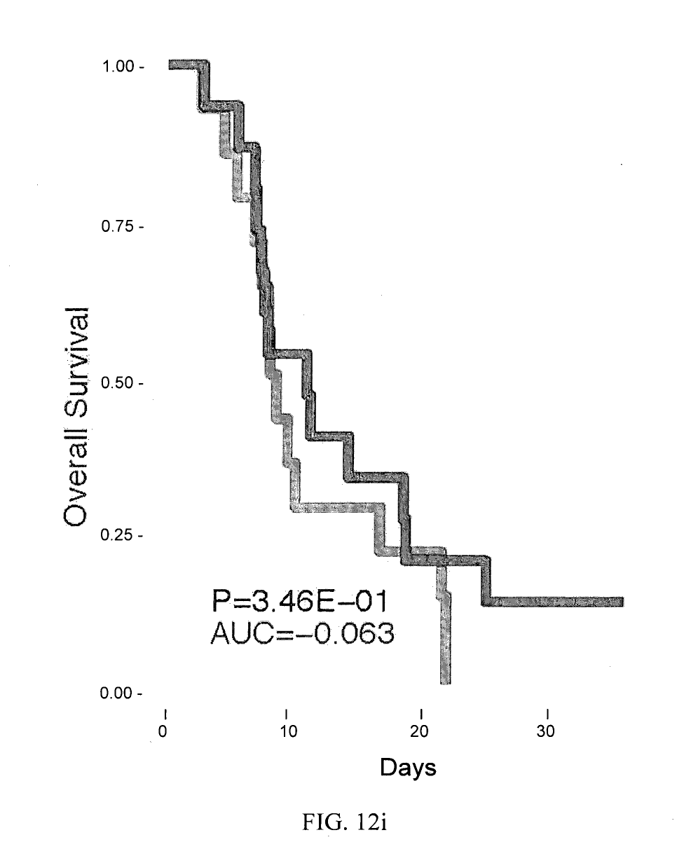

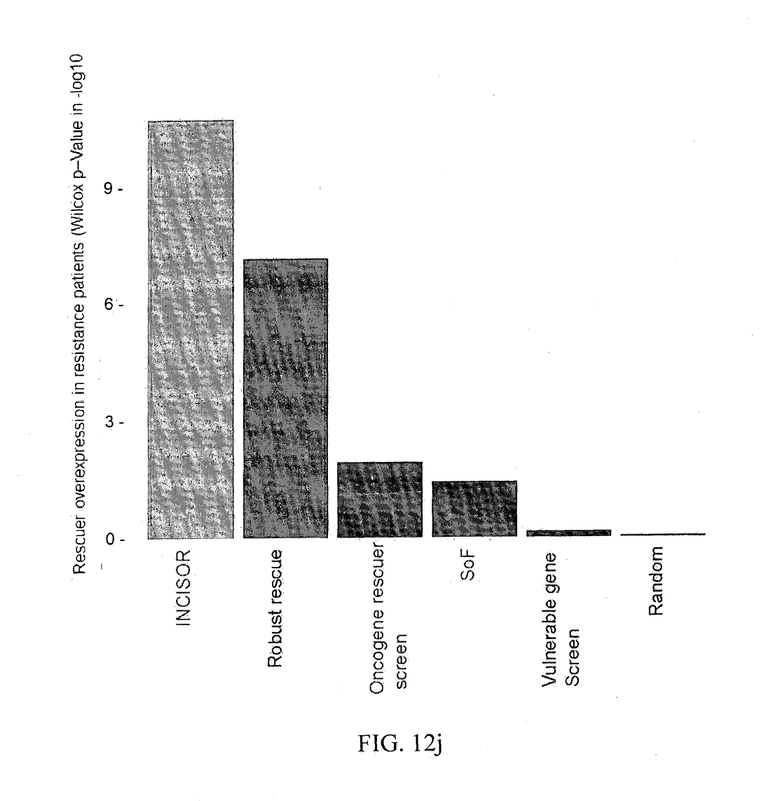

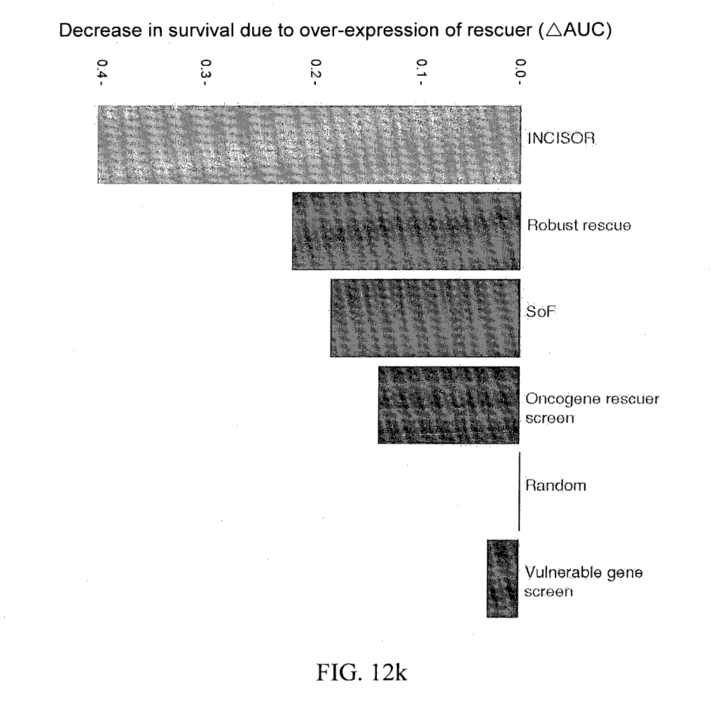

To study the ability of SR profiles of patients' tumors to identify specific molecular markers of the response to cancer therapy we analyzed a dataset of 25 breast cancer patients for which both pre- and post-treatment gene expression measurements are available²⁶. These patients, composed of 8 responders and 17 non-responders, were treated with a combination of epirubicine, cyclophosphamide, and docetaxel whose targets have 19 predicted rescuer genes encompassing 20 SR interactions. Remarkably, we found a significant increase in the post to pre expression levels of the predicted rescuer genes in non-responders vs responders (ranksum p-value<1E−7 (FIG. 12a,b). There is a notable correlation between the rescuers' increased expression level in the nonresponsive patients vs the survival predictive power (in pan-cancer TCGA) of the corresponding SR interactions (FIG. 12c). The treatment response could be predicted based on the pre-treatment expression of the 19 rescuer genes' signature (Methods, AUC of 0.71, FIG. 12d). Embedded feature selection reveals that the key rescuer genes determining the patients' response are ATAD2 and PBOV1. ATAD2 is required to induce the expression of a subset of target genes of estrogen receptor including MYC²⁷, and is also known to be associated with drug resistance to Tamoxifen and 5-Fluorouracil²⁸. A similar analysis applied to analyze the response of gastric cancer patients to Cisplatin and Fluorouracil treatment further demonstrates the generic ability of an SR based analysis to pinpoint network wide genomic alterations associated with resistance to these therapies²⁹.

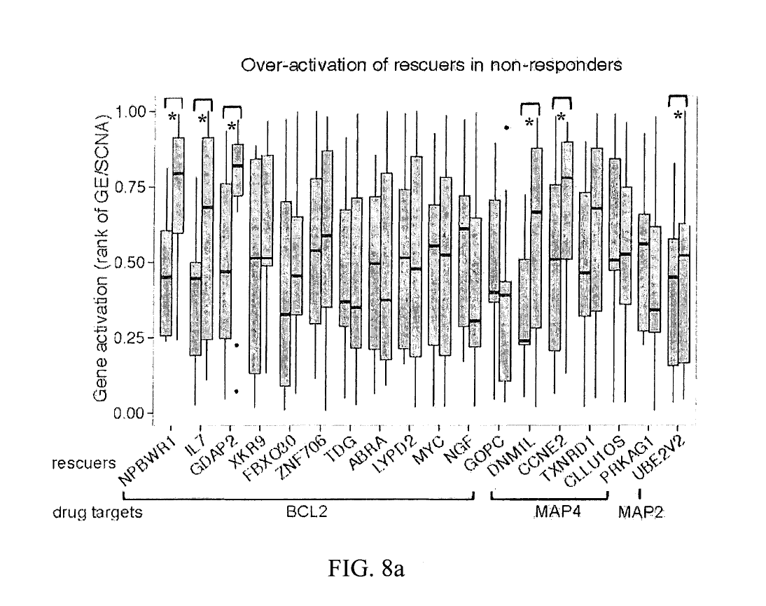

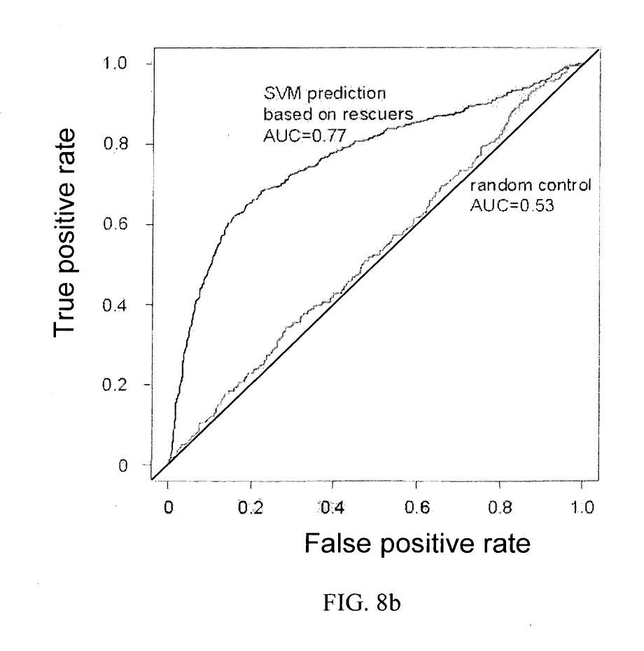

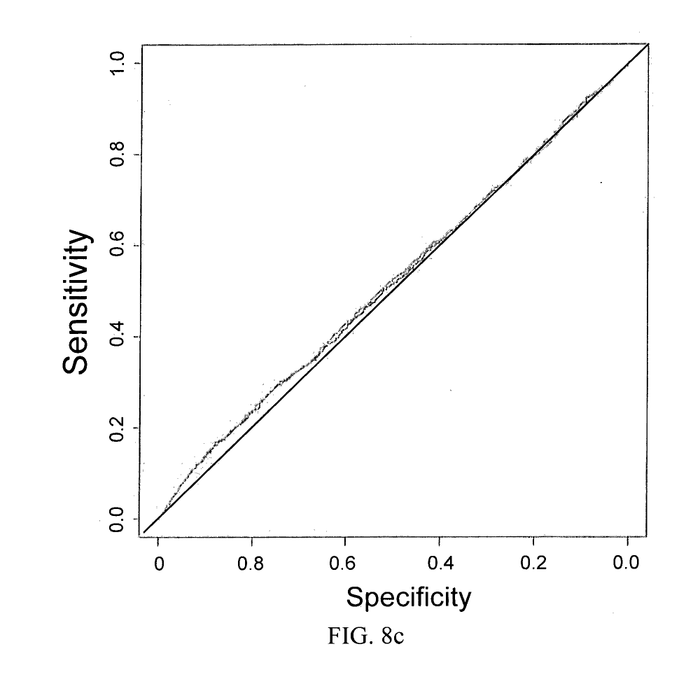

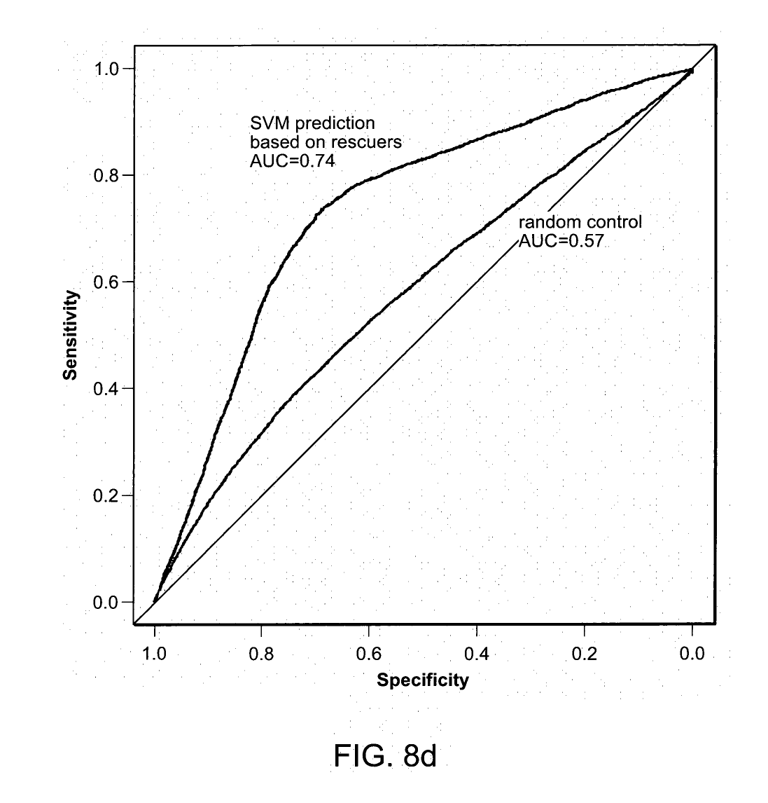

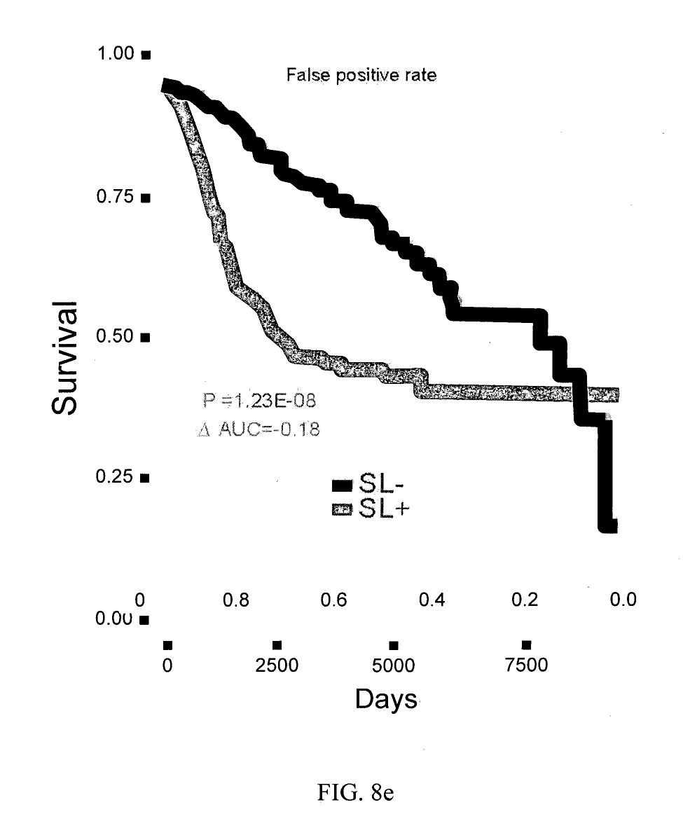

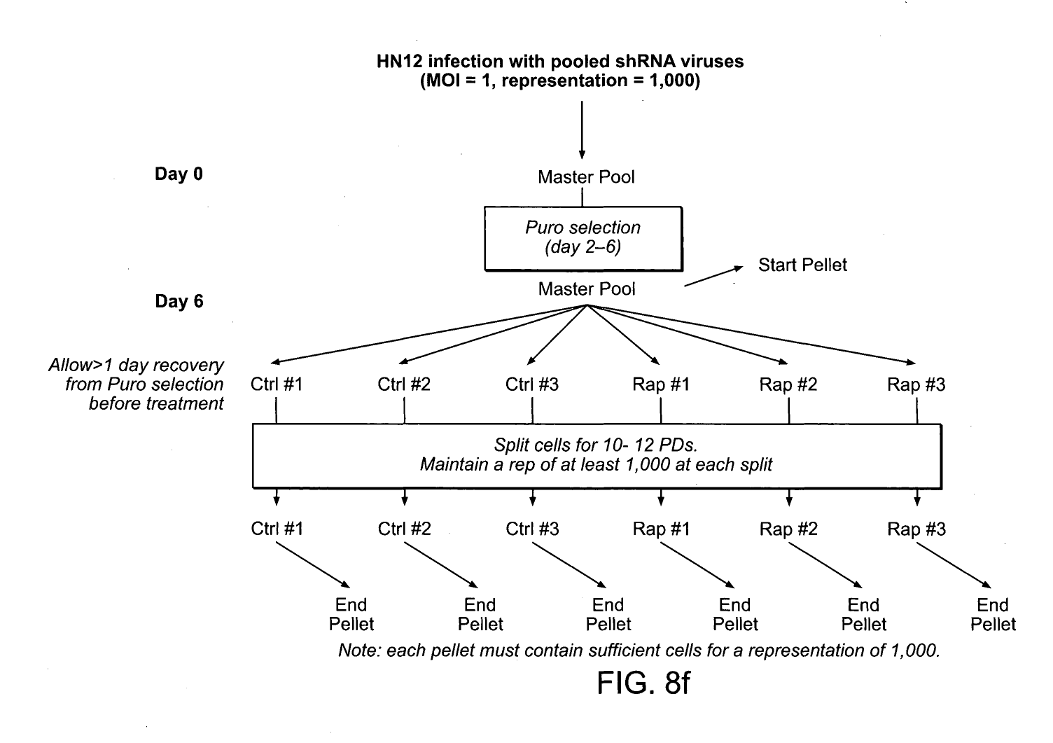

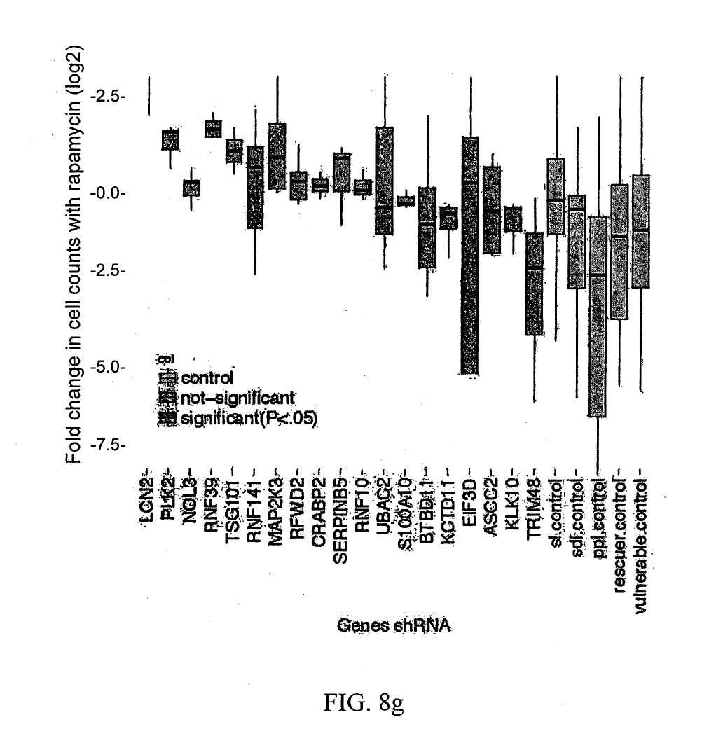

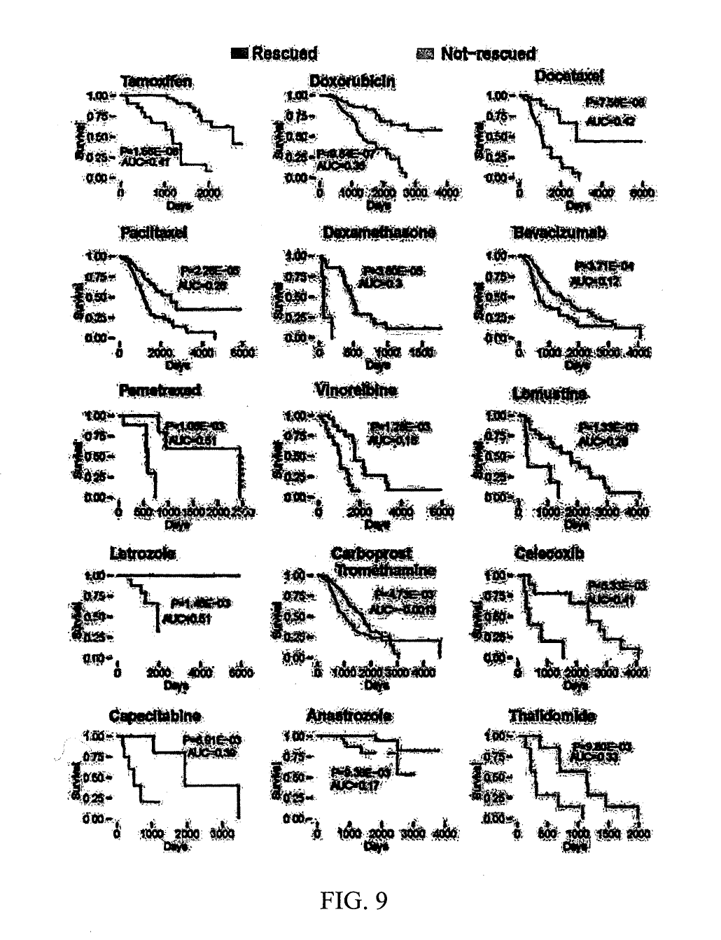

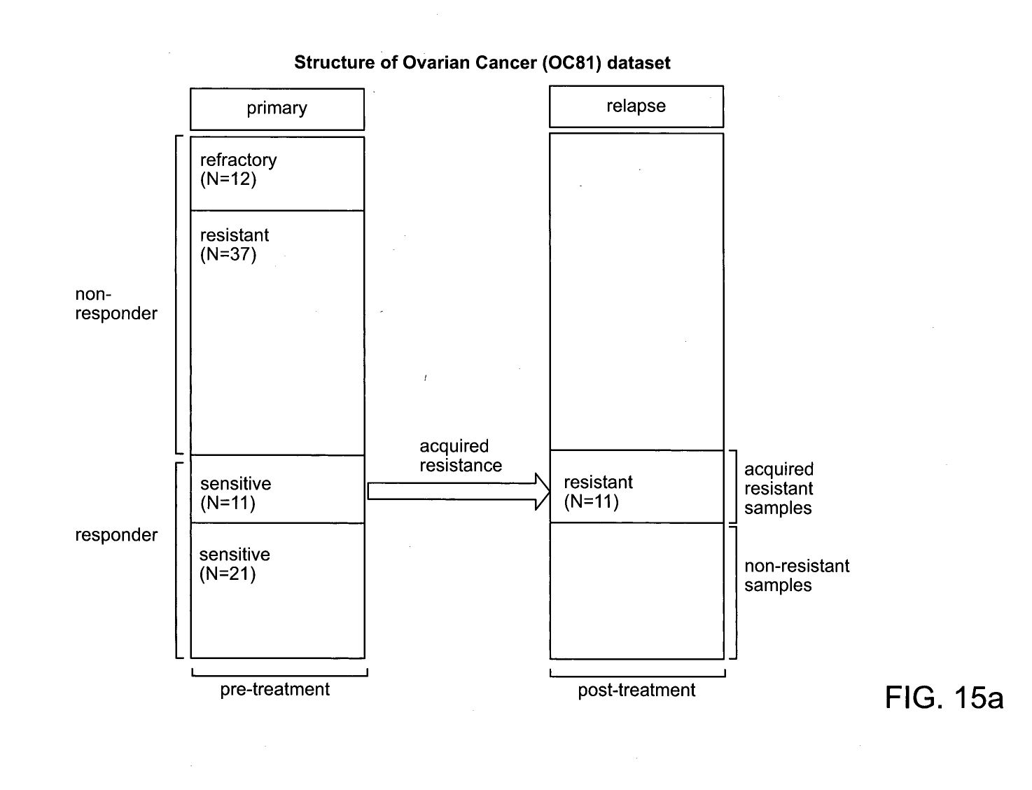

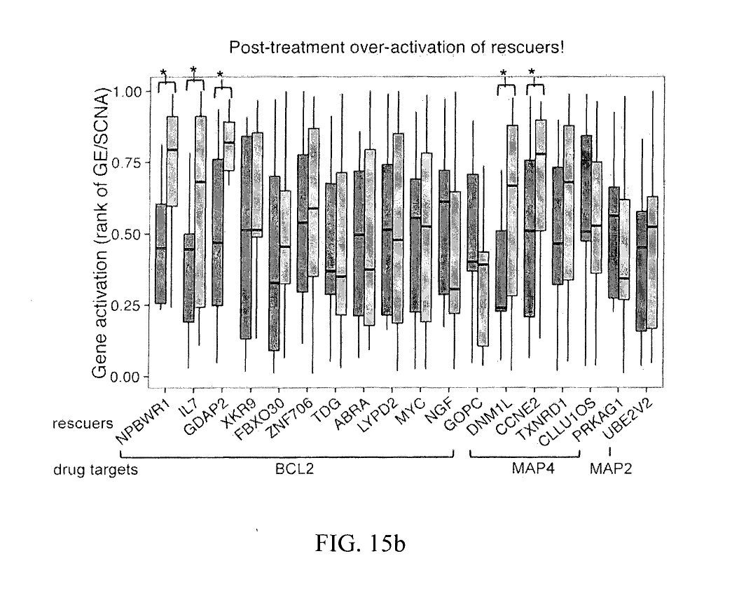

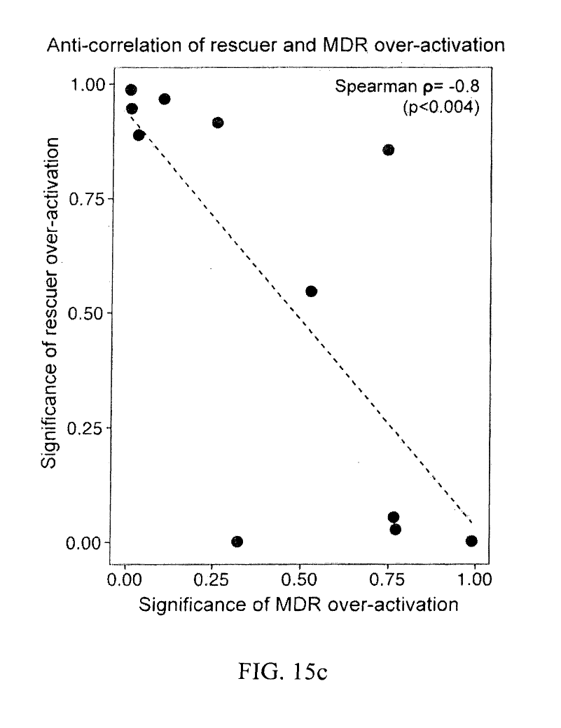

We turned to study the value of SR networks in predicting the molecular alterations associated with the emergence of resistance to cancer therapy, resulting in the relapse of tumors that were initially responsive to treatment. To this end we analyzed data longitudinal dataset of 81 ovarian cancer patients treated with Taxane (and Cisplatin), which includes tumor genomics data collected from patients after relapse (FIG. 15a)³⁰. We focused on the activation level of the 11 SR DU rescuer genes of the 4 drug targets of Taxane. We find that, as predicted, rescuer genes indeed become over-active in the relapsed resistant tumors of initially responsive patients (overall ranksum p-value<1.6E−5), and this increase is significant compared to random genes (empirical p-value<0.026, FIG. 15b). As in the previous breast cancer case, non-responders have initially higher levels of rescuers' activity than responders (ranksum p-value<3.8E−7) and this is significant compared to random genes (empirical p-value<4.0E−4, FIG. 8a). The activity of the 11 rescuers signature at the pretreatment stage enables us to predict the future emergence of resistance (AUC=0.75, FIG. 8b). Interestingly, the second strongest predictor of acquired resistance, FOXM1, is already known to play a role in resistance to Taxane³¹ and Cisplatin³² therapies in breast cancer, and a recent report demonstrated its role in Taxane resistance in ovarian cancer³³. The top and third most important rescuers, PLOD 1 and LOX, regulate extracellular matrix metabolism, contributing to metastasis³⁴. Notably, an analysis of multidrug resistance (MDR) genes' expression shows a marked inverse correlation between their activation and the level of rescue reprogramming occurring in Taxane resistant samples (Spearman correlation=−0.80 (p-value<0.021), FIG. 15C). This suggests an interesting complementary relation between these two different resistance mechanisms. An similar analysis of 155 primary breast cancer patients treated with Tamoxifen³⁵ shows that a binary classifier based on the activity states of 13 rescuers signature of Tamoxifen's drug targets can predict the patients whose tumor will relapse (AUC=0.74, FIG. 8d), identifying main SR rescuers invoking resistance to Tamoxifen in a clinical setting.

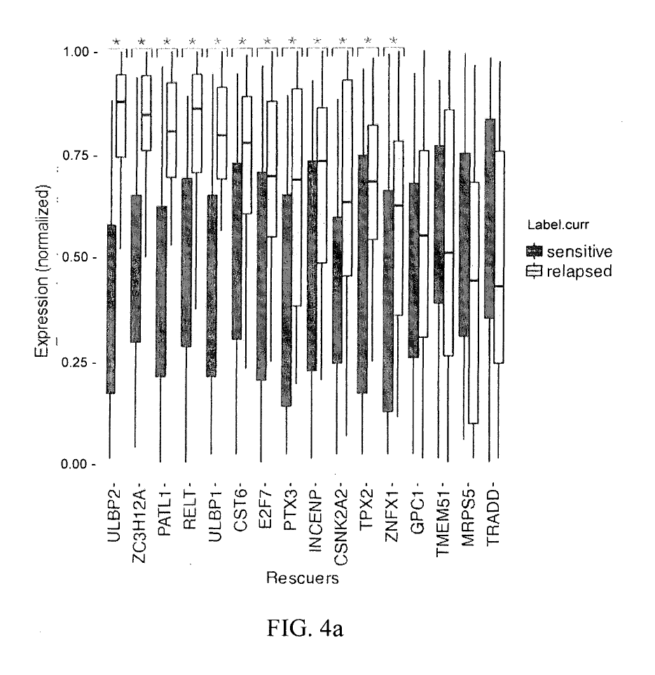

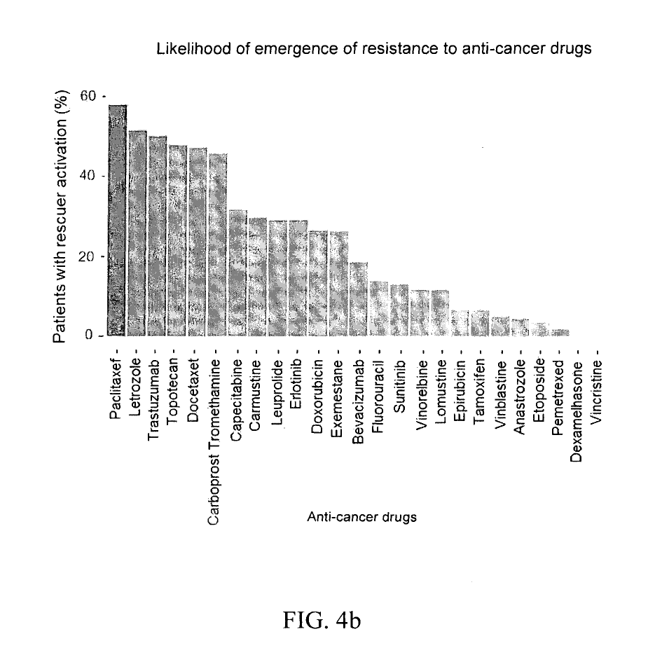

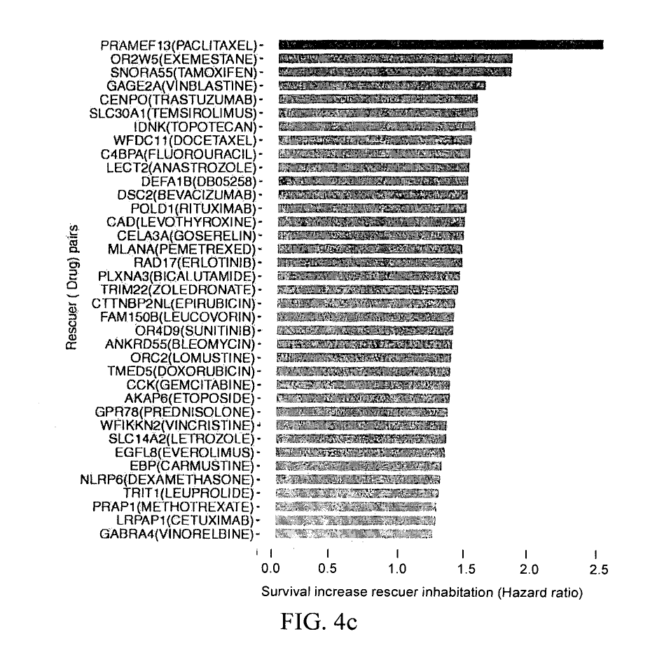

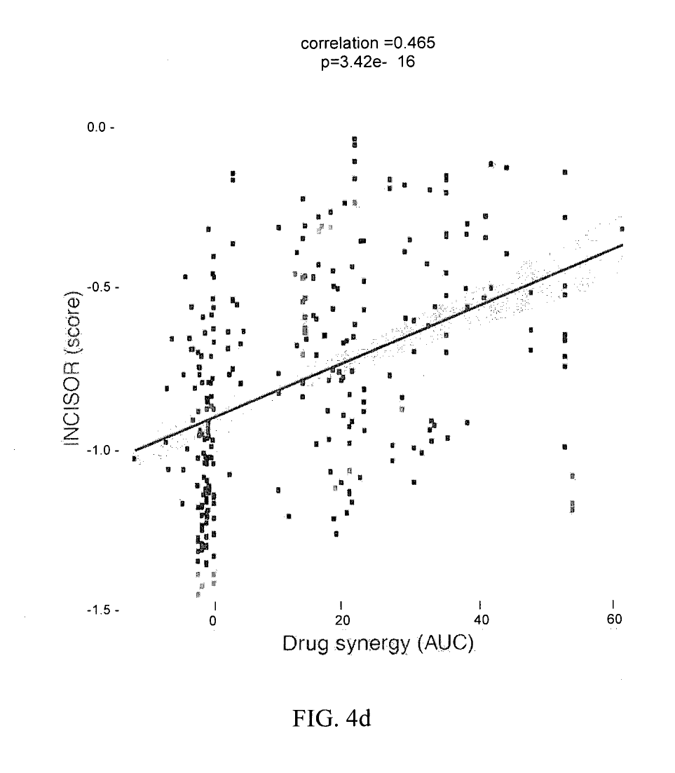

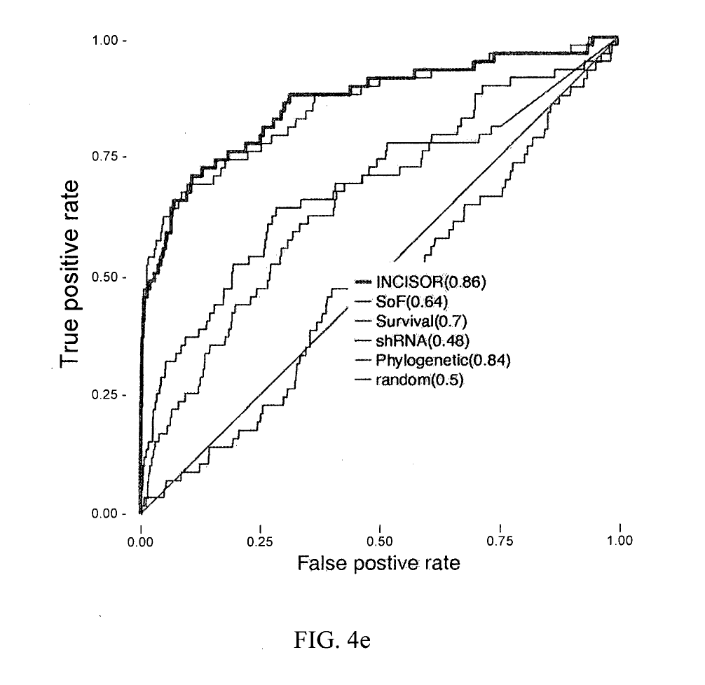

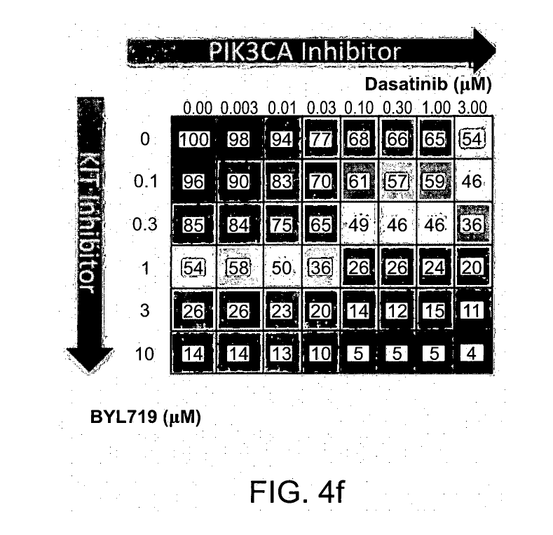

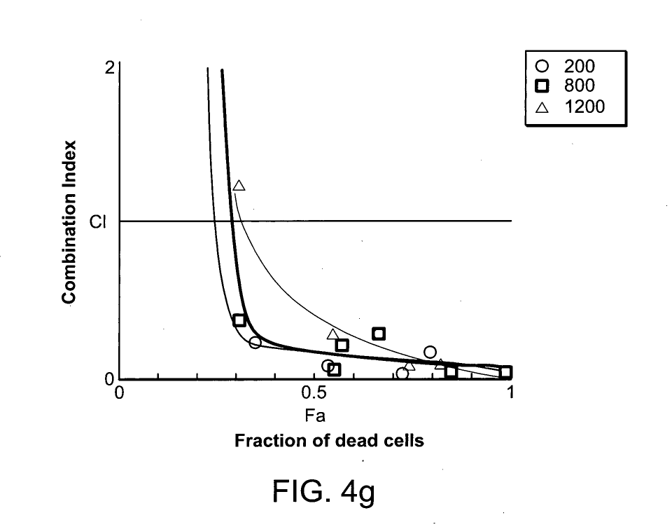

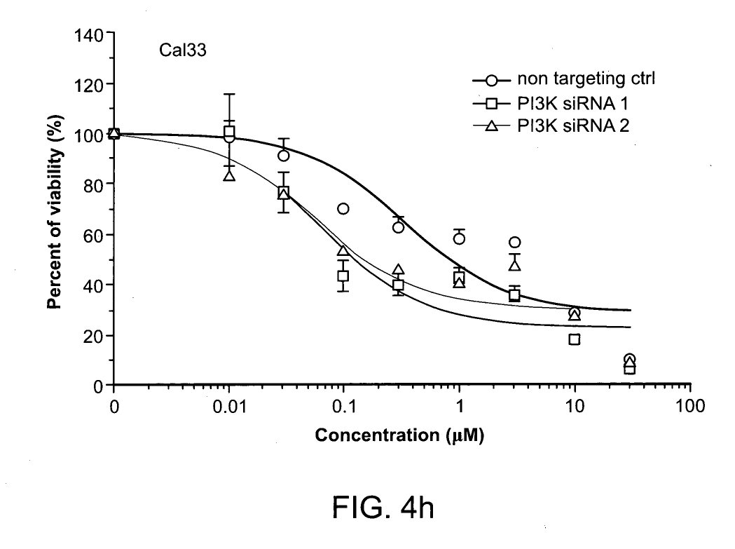

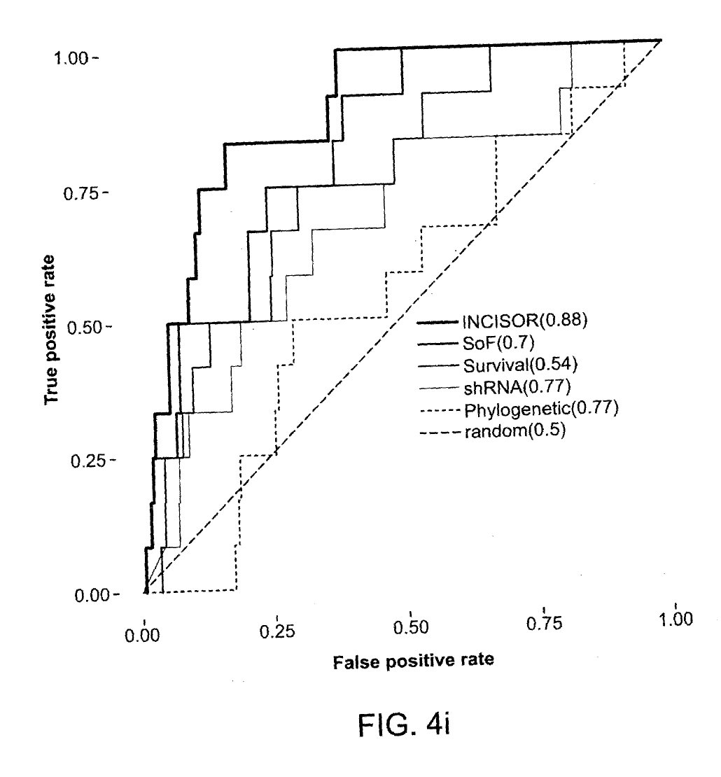

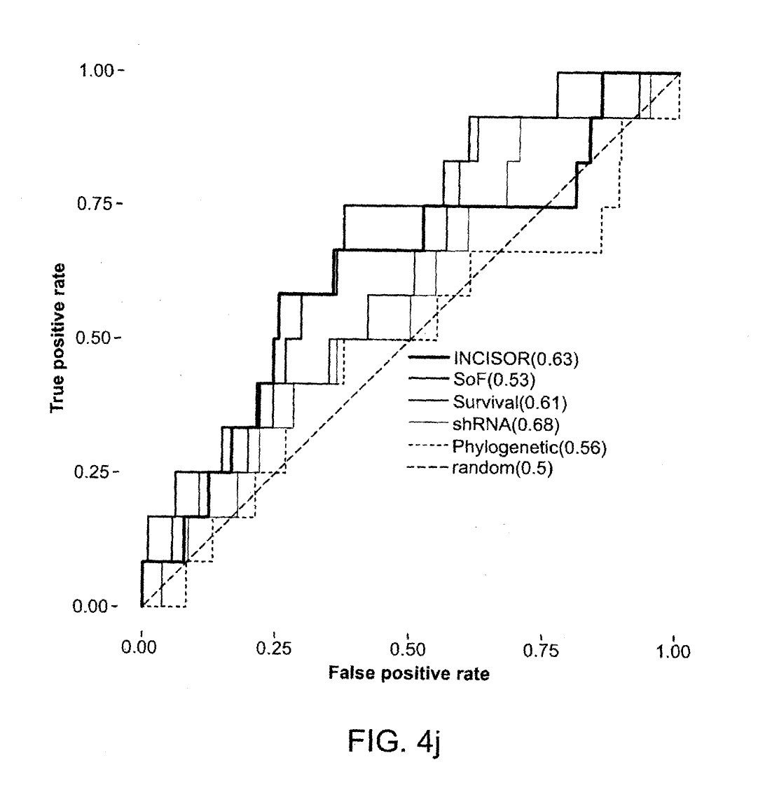

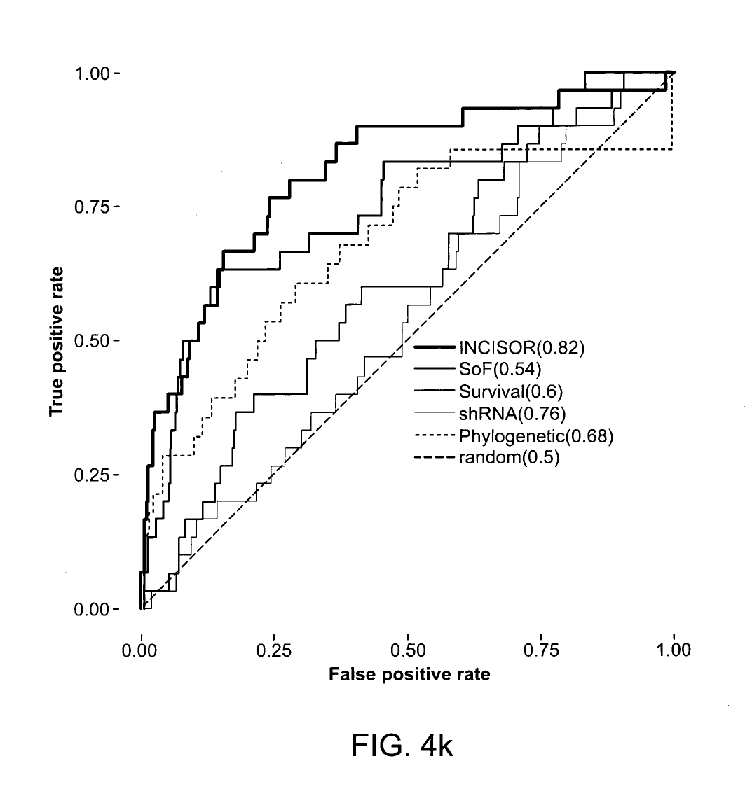

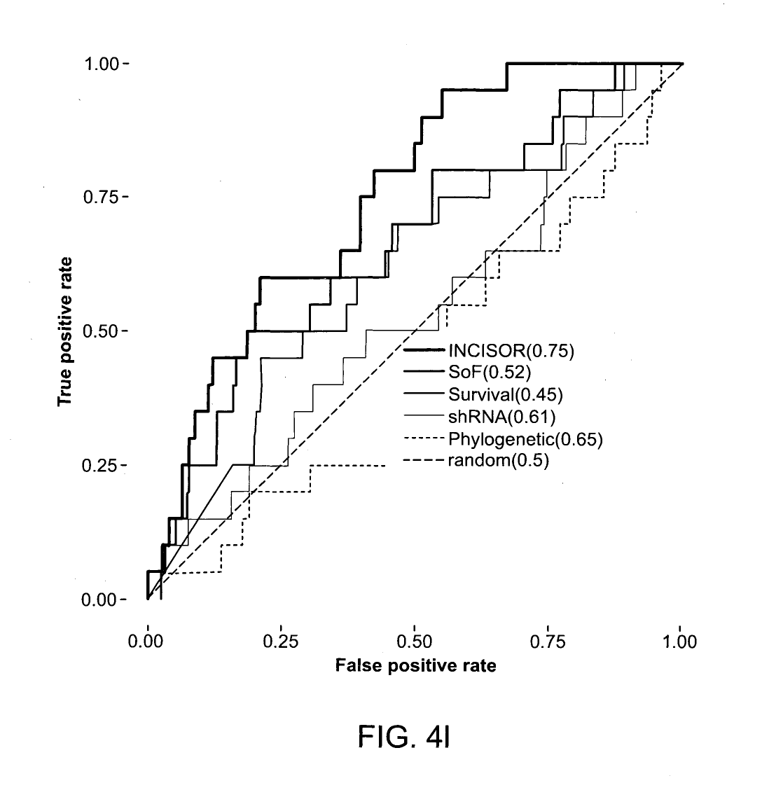

Our analysis naturally raises a new treatment opportunity, based on targeting the rescuer hubs to reduce likelihood of developing resistance that may serve as supplement to current chemotherapy. To this end, we provide a list of cancer type-specific main rescuer hubs, many of which have been already associated with resistance. Interestingly, none of rescuer hubs are targeted by current anti-cancer therapies. The expected clinical utility of targeting each of these key rescuer genes following treatment is shown in FIG. 4C, as estimated from its effects on patients' survival in the TCGA. Further, by quantifying the number of samples with functionally active rescuers among the patients that receive a specific drug we provide estimates of the likelihood that resistance via SR molecular pathways will emerge following their treatment (FIG. 4B).

In summary, this work presents and comprehensively studies a new concept of synthetic rescue reprogramming in cancer, and has developed INCISOR, a data-driven framework for inferring genome-wide SR networks. Our study reveals that the cellular reprogramming is prevalent across cancer types, of significant clinical importance and associated with patient survival, drug response and the emergence of resistance. Synthetic rescue is shown to serve as a universal platform that is capable of predicting and providing molecular insights to the response/resistance of many different cancers to a variety of treatments. SR reprogramming has considerable translational importance: (a) First and foremost, it lays the basis for assessing the likelihood that resistance will emerge due to SR reprogramming; this is relevant both to optimizing the treatment of individual patients and for prioritizing new drugs targets in specific cancer types. (b) Second, targeting key rescuer genes can offer a new class of treatments for adjuvant cancer therapies aimed at counteracting resistance and tumor heterogeneity. (c) Finally, a better characterization of SR reprogramming can help guide the rational design of combinatorial treatments targeting both vulnerable genes and their rescuers. Thus, combined with SL information, uncovering and utilizing cancer SR networks is likely to significantly advance future cancer treatment.

### Predicting Pan-Cancer Drug Response II

Using the drug-DU-SR, we analyzed 3,873 TCGA patient samples that have been treated⁶, including drugs that were used to treat at least 30 patients. For each drug tested, we divided the treated samples into rescued (predicted non-responders) and non-rescued (predicted responders) groups based on the number of over-active rescuers of the drug target genes in the drug-DU-SR network. That is, if a sample has many over-active rescuers of the specific targets of the given cancer drug given (deduced from their gene expression and SCNA values in that sample) we predict it to be a non-responder and vice versa, if it has very few (or none) active rescuers of the drug given we predict it to be responsive. We then analyzed patient survival data of treated patients to evaluate the predictive power of drug-DU-SR by comparing the decrease in survival in the rescued group compared to the non-rescued group using Cox regression analysis. As evident, SRs can be successfully used to predict drug response in an unsupervised manner (which is hence less prone to over-fitting) (FIG. 3g).

### Predicting Adjuvant Therapy Candidates for Counteracting the Emergence of Resistance Via DU-SR Interactions

Down-regulating DU-SR rescuers provide a unique opportunity to mitigate drug-resistance. For each drug in TCGA collection, we first identified all DU-SR rescuer partners of its drug targets. We then investigated the impact of the down-regulation of these rescuers by comparing the survival of patients whose rescuer activation is low vs. high (using a log-rank test) per each drug treatment. We selected the top rescuers of each drug that show the highest improvement in patient survival when inactivated and reported 19 drug-rescuer pairs that have significant clinical impacts. That is, we predict that targeting these major rescuers will significantly improve the response (in terms of survival) of patients receiving cancer treatments specifically rescued by these genes (FIG. 4C).

### Estimating the Likelihood of Developing Resistance to Anti-Cancer Drug Treatments Via DU-SR Interactions

The proportion of patients who have over-activated rescuers provides an estimate of the likelihood of developing SR-mediated resistance. For 25 anti-cancer drugs, whose response is predictable by SR network, we estimated the drug's likelihood to develop resistance by the fraction of patients whose tumors harbor significantly over-activated DU-SR rescuers of the drug targets. (See FIG. 4B)

### Example 3: Evaluating the Predictive Survival Signal of the Inferred SR Networks

To evaluate the aggregate survival predictive signal of the pan-cancer SRs we applied INCISOR™ to pan-cancer TCGA samples (training set) to identify the SR pairs and tested their clinical significance in a completely independent METABRIC dataset (test set) to avoid potential risk of over-fitting, which includes the gene expression, SCNA, and survival of 1981 breast cancer patients. Based on the number of functionally active SRs in each tumor sample, the top 10 percentile of samples were considered as rescued and the bottom 10 percentile as non-rescued. We then estimated the significance of improvement of survival in the rescued vs non-rescued samples using a log rank test. (FIG. 3a).

### Example 4: Tracing the Number of Functionally Active SR Pairs in Tumors During Cancer Progression

To study the functional activation of SRs as cancer progresses we divided the breast cancer patients in METABRIC dataset into 6 classes of cancer progression (removing censored data), by dividing them equally into 6 bins according to their survival times (N=627). First, in each bin, we counted the mean fraction of functionally active SRs. Such pairs are defined by the under-activation of the vulnerable gene and the over-activation of the rescuer gene, where the latter are determined based on their SCNA and gene expression values (FIG. 10g). Second, we defined a vulnerable gene as rescued if more than N number of rescuers are over-activated with the threshold N running from 0 to 4, and counted the mean fraction of rescued vulnerable genes in the six progression bins (FIG. 10h).

### Example 5: Identifying the Clinical Significance of Reprogrammed SR and Buffered SR

Using the cancer progression classes described above, we classified the DU SRs identified by INCISOR™ based on the relations of three frequency values: rescuer over-activation (for), vulnerable gene inactivation (fᵥ), and functional activation of SR (f_{SR}). An SR pair is defined as reprogrammed SR (rSR) if the inactivity of the vulnerable gene A occurs first (in an earlier stage) and is followed by the over-activation of rescuer gene B (i.e., occurring at a later stage). Accordingly, we classified an SR pair as an rSR if fₒᵣ and f_{SR} are highly correlated while fᵥ and f_{SR} are not, and f_{SR} increases as cancer progresses. Similarly, an SR was classified as buffered (bSR) when the over-activation of rescuer gene B precedes the inactivation of vulnerable gene A. We classified as an SR pair as a bSR if fᵥ and f_{SR} are highly correlated while fₒᵣ and f_{SR} are not, and f_{SR} increases as cancer progress.

### Example 6: Charting the Molecular Mechanisms Underlying Drug Resistance Using SR Networks

Resistance to therapy in cancer may arise due to diverse mechanisms including drug efflux, mutations altering drug targets and downstream adaptive responses in the molecular pathways targeted. The latter mainly involves reprogramming changes in the sequence, copy number, expression, epigenetics, and phosphorylation of proteins that buffer the disrupted function of the drug targets, Indeed, numerous recent transcriptomic and sequencing studies have identified molecular signatures underlying the emergence of resistance to specific drugs.

We analyzed multiple drug response and resistance datasets where gene expression (and SCNA for limited cases) was measured from the patients treated with targeted therapy^{26,30,36-38}. For each dataset we identified drug targets from Drugbank²⁴ and the rescuer genes were specifically inferred by applying the relaxed condition to the specific treatment of interest. To check the over-activation of rescuers in post-treatment samples (relative to pre-treatment), we performed a paired one-sided Wilcoxon rank-sum test. To associate the over-activation of rescuers in non-responders (compared to responders) we first divided samples into rescued and not-rescued groups based on the number of over-active rescuers, and performed a one-sided Wilcoxon rank-sum test between the two groups. When information on patient survival is available (instead of drug response) we performed a log rank test between the two groups using progression-free survival and/or overall survival. To predict the emergence of resistance based on pre-treatment gene-expression (and/or SCNA) in an unsupervised manner, we divided the samples into predicted resistant and sensitive groups based on the number of over-activated rescuers in pre-treatment samples and then performed a one-sided Wilcoxon rank-sum test. The supervised predictor was built using SVM with rescuer expression profile as input feature, and the accuracy of the supervised predictor was determined using cross-validation. To compare the resistance arising from multidrug resistance and synthetic rescues, we considered the post-treatment increase of gene activation level of the rescuer partners of the given drug targets with the gene expression levels of 12 MDR-associated genes³⁹ in relapsed tumors. To validate our SR network with the recent findings on pathways associated with the resistance of 4 different drug treatments (BET^{1,2}, AR³, EGFR⁴ and BRAF⁵ inhibitors), we first applied INCISOR™ to identify treatment-specific DU-SR rescuers. We then performed a pathway enrichment analysis of them and observed that there are significant overlaps in the cellular processes to which these rescuers belong and the resistance gene sets reported in these studies. The details and additional analysis for each such dataset are provided in Supplementary Information.

### Experimental Analyses

We next set out to experimentally test our SR predictions in vitro focusing on a subset of the predicted SRs involving mTOR, a major kinase regulating cancer growth and survival. We studied rSR and bSR predictions of the DD-SR type as they can be readily validated by in vitro knockdown (KD) experiments. Our investigation was performed in a head and neck squamous cell carcinoma (HNSC) cell-line, where mTOR is known to be essential for cancer progression and its inhibition by Rapamycin interferes with cancer progression (also confirmed in our analysis, Wilcoxon rank-sum P<4.5E−15, Supplementary Information). In difference from its overall effect, we hypothesized that when mTOR's predicted vulnerable DD-SR partners are knocked down, Rapamycin treatment will not inhibit but induce cancer progression as per the DD definition. To test this predicted reversal of effect, we tested 10 (pan-cancer) DD-rSR pairs where mTOR is the predicted rescuer gene via shRNA knockdowns of the vulnerable partner gene followed by Rapamycin treatment. The KD of mTOR's vulnerable partners hampers tumor proliferation both in an in vitro tissue culture (Paired Wilcoxon rank-sum P<1.3E−5) and in an in vivo mouse model (Paired Wilcoxon rank-sum P<6.5E−6, see Supplementary Information). We observed a significant reversal effect of Rapamycin treatment on proliferation in 6 out of 10 vulnerable gene KDs (FIG. 16a, aggregate Wilcoxon rank-sum P<2.1E−8). The experiments testing the shRNA KD of five different sets of control (non-vulnerable) genes followed by mTOR treatment reassuringly failed to produce a significant rescue signal. A similar but less marked rescue effect is observed when mTOR is the vulnerable gene in DD-bSR interactions (FIG. 16b, P<4.3E−4 across 9 predicted SR interactions), consistent with the observation of superior predictive power of rSR above. An experimental testing of the predicted HNSC-specific DD-type rescuers of mTOR yielded an additional validation of the predicted mTOR DD partners in an analogous manner (FIG. 8g).

We used Rapamycin because it is a highly specific mTOR inhibitor and hence enables targeting of a predicted rescuer gene by a highly specific drug, combined with the ability to knock down predicted vulnerable genes in a clinically-relevant lab setting. We used HNSC cell-line HN12, which, like most HNSC cells, is highly sensitive to Rapamycin⁴⁰. For this, we applied INCISOR™ to identify top 10 vulnerable partners and 9 rescuer partners of mTOR in a pan-cancer scale. We also identified HNSC-specific DD-type vulnerable partners of mTOR.

We performed the shRNA knockout and mTOR inhibition in the following steps (FIG. 8f). Each of these mTOR's vulnerable/rescuer partners together with the controls was knocked down in HN12 cell lines, after which mTOR was inactivated via Rapamycin treatment. HN12 cells were infected with a library of retroviral barcoded shRNAs at a representation of ˜1,000 and a multiplicity of infection (MOI) of ˜1, including at least 2 independent shRNAs for each gene of interest and controls. 25 genes were included as controls (71 shRNA in total; Table 6). At day 3 post infection cells were selected with puromycin for 3 days (1 μg/ml) to remove the minority of uninfected cells. After that, cells were expanded in culture for 3 days and then an initial population-doubling 0 (PDO) sample was taken. For in vitro testing, the cells were divided into 6 populations, 3 were kept as a control and 3 were treated with Rapamycin (100 nM). Cells were propagated in the presence or not of a drug for an additional 12 doublings before the final, PD13 sample was taken. For in vivo testing, cells were transplanted into the flanks of athymic nude mice (female, four to six weeks old, obtained from NCI/Frederick, Md.), and when the tumor volume reached approximately 1 cm³ (approximately 18 days after injection) tumors were isolated for genomic DNA extraction. Mice studies were carried out according to National Institutes of Health (NIH) approved protocols (ASP #10-569 and 13-695) in compliance with the NIH Guide for the Care and Use of Laboratory Mice. shRNA barcode was PCR-recovered from genomic samples and samples sequenced to calculate the abundance of the different shRNA probes. From these shRNA experiments, we obtained cell counts for each gene knock-down at the following three time points: (a) post shRNA infection (PDO, referred as initial count), (b) shRNA treatment followed by either Rapamycin treatment (PD13, referred as treated count, 3 replicates) or control (PD13, referred as untreated count, 3 replicates) (c) shRNA infected cell injected to mice (tumor, referred as in-vivo count, 2 replicates). To obtain normalized counts at each time point, cell counts of each shRNA at each time point were divided by corresponding a total number of cell count. To estimate cell growth rate at treated, untreated and in vivo time points for each gene X, normalized counts were divided by initial normalized count as follow:

$$growth rate ( X ) = \frac{normalized count ( X )}{initial normalized count ( X )}$$

Effect of Rapamycin treatment on cell growth on knockdown of gene X was calculated as:

$$rapamycin effect ( X ) = \frac{treated growth rate ( X )}{mean untreated growth rate ( X )}$$

To quantify the lethality of vulnerable knockdown, we performed a one-sided Wilcoxon rank-sum test between initial normalized count with in vivo normalized count for in vivo lethality (and with the untreated normalized count for in vitro lethality). To compare rescue effect of Rapamycin treatment between shRNA knockdown of mTOR's vulnerable gene partner and control gene knockdown, we performed a one-sided Wilcoxon rank-sum test between Rapamycin effects of mTOR partner vulnerable genes and control genes.

### Example 11: Using INCISOR™ for the Identification of SLs

In this section, we describe using INCISOR™ to predict SL interactions (SLi). INCISOR™ may be further modified along these lines to identify other types of genetic interactions in additional to SLs and SRs, e.g., for the identification of synthetic dosage lethal (SDL) interactions where the down regulation of one gene coupled with the up regulation of its SDL partner is lethal. We name the variant of INCISOR for identification of SLi and synthetic dosage lethality (SDL) interactions as ISLE (Identification of clinically relevant Synthetic Lethality). Specifically, it describes adopting different statistical screens in INCISOR™ to identify SLi that occurs in a patient's tumor and is likely to have a therapeutic value.
(1) Molecular survival of the fittest (SoF): A SoF-SLi-pattern between two genes (A and B) denotes that samples, where both gene A and B are inactive, are significantly less frequent than expected. Analogous to SR identification, we employ a simple binomial test to identify depletion of samples in the different activity bins followed by standard false discovery correction.
(2) Patient Survival screening: Co-inactivated of a SL gene pair (A and B) in a tumor is lethal, and hence patients with co-inactive SL gene pair will have better survival Accordingly, INCISOR™ employs a Cox multivariate regression analysis to identify candidate SL partners whose co-inactivation is associated with improved survival to a greater extent compared to the additive effect of the individual gene inactivation of the candidate SL partners. Similar to SR identification, we control for various confounding factors including cancer types, sex, race, and age.
(3) Phenotypic screening: By definition, it is expected that gene A will be essential only when its SL partner gene B is inactive in a given cancer cell line. Accordingly, INCISOR™ uses genome-wide shRNA screening to identify a gene pair A and B as candidate SL partners if both gene A and gene B shows conditional essentiality based on its partner's low gene expression/SCNA.
(4) Phylogenetic screening: Same as SR phylogenetic screen

### Example 12 Supplementary Information and Tables

1 Inscisor Pipeline->I Replaced it with the New Method Description

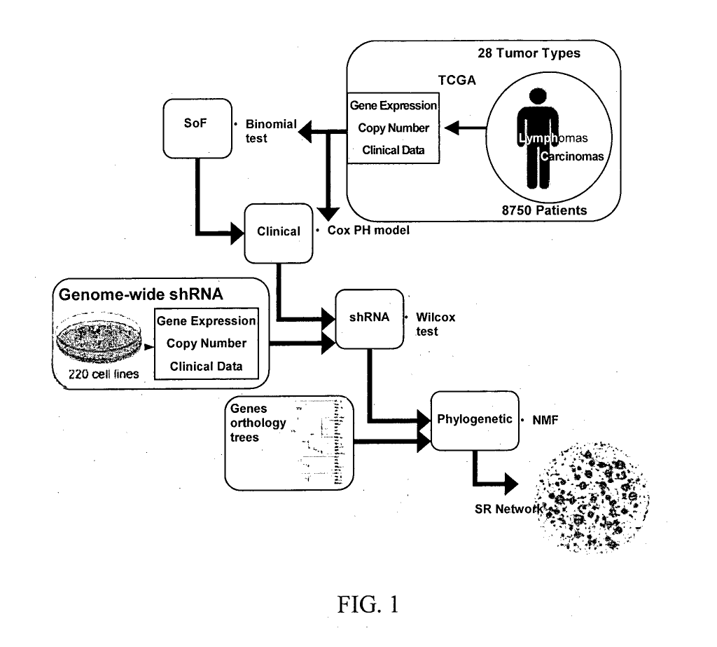

INCISOR identifies candidate SR interactions employing four independent statistical screens (FIG. 1), each tailored to test a distinct property of SR pairs. We describe here the identification process for the DU-type SR interactions (Down-Up interactions, where the up-regulation of rescuer genes compensates for the down-regulation of a vulnerable gene (e.g., by an inactivating drug), FIG. 6). Then we discuss how to modify DU-INCISOR to detect the other SR types (DD, UD, and UU). We identify pan-cancer SRs (those common across many cancer types) analyzing gene expression, somatic copy number alteration (SCNA), and patient survival data of The Cancer genome Atlas (TCGA) from 7,995 patients in 28 different cancer types and integrating genome-wide shRNA screens in around 220 cell lines composing in the total of 1.2 billion shRNA experiments. The same approach can be used to identify cancer type specific SRs, in an analogous manner. INCISOR is composed of four sequential steps:
(1) Molecular survival of the fittest (SoF): We mine gene expression and SCNA of 8450 TCGA tumor samples to identify vulnerable gene (V) and rescuer gene (R) pairs having the property that tumor samples in the non-rescued state (that is samples with underactive gene V and non-overactive gene R, activity states 3 in FIG. 6) are significantly less frequent than expected (due to lethality, activity states 1 and 2 in FIG. 6), whereas samples in the rescues state (that is samples with under-active gene V but over-active gene R) appear significantly more than expected (testifying to an explicit rescue from lethality). Specifically, we first divide tumor samples into the non-rescued and rescued states (activity states) and then we employ a binomial test to identify depletion or enrichment of samples in the different activity states followed by standard false discovery correction² as follows:
   To reliably identify the enrichment/depletion of an activity state, we used both gene expression (GE) and somatic copy number alteration (SCNA). We inferred enrichment/depletion of an activity state independently using gene expression and SCNA. We define the activity state as enriched/depleted only when the activity state is significantly enriched/depleted after FDR² correction for both gene expression and SCNA independently. We infer an activity state A of a rescuer R and vulnerable V gene pair as enriched/depleted using gene expression in the following manner: First, a gene is defined as inactive (respectively, overactive) if its expression level is less (greater) than the 33rd-percentile (67th-percentile) across samples. A gene has its normal activation level if its expression level is between the 33rd and 67th percentile (across samples). Out of total N tumor samples, if n1 (n2) is the number of samples in the activity state using gene R (V) independently and m is number of samples in the activity state, the significance of enrichment or depletion is determined using a Binomial

$$( N , \frac{n 1 * n 2}{{N}^{2}} ) .$$

Enrichment/depletion of the activity state using SCNA is inferred in an analogous fashion.
(2) Patient Survival screening: The next steps utilize patient survival data to narrow down which of the SR candidate pairs from step 1 are the most promising candidates. This step aims to selects vulnerable gene (V) and rescuer gene (R) pair having the property that tumor samples in rescued state (that is samples with underactive gene V and overactive gene R) exhibits significantly worse patient's survival as compared to non-rescued state tumors. Specifically, we perform a stratified Cox regression with an indicator variable indicating if a tumor is in rescued state for each patient. To infer an SR interaction, INCISOR checks association of the indicator variable with poor survival, controlling for individual gene effect on survival. The regression also controls for various confounding factors including, cancer types, sex, age, and race.
   Similar to SoF, to reliably estimate an effect of putative SR pair on patient's survival, we use both gene expression and SCNA. Clinical effect on survival is inferred independently for gene expression and SCNA data. We define the pair to have the significant effect on survival, only when both the gene-expression-based survival effect and the SCNA based survival effect are significant after multiple hypothesis corrections.
   Dividing TCGA tumor samples into the rescued and non-rescued states similar to the SoF step, INCISOR determines gene-expression based survival effect of an activity state A gene pair (rescuer R and vulnerable gene V) using the following stratified Cox proportional hazard model:

h_{g}(t,patient)˜h_{0g}(t)exp(β₁I(V,R)+β₂g(V)+β₃g(R)+β₄ age)

Where, g is a stratification of the all possible combinations of patients' stratifications based on cancer-type, age and sex. h_{g} is the hazard function (defined as risk of death of patients per unit time) and h_{0g} (t) is the baseline-hazard function at time t of the gth stratification. The model contains four covariates: (i) I(V, R): indicator variable if the patient's tumor is in the activity state A, (ii) g(V) and (iii) g(R): gene expression of V and R, (iv) age: age of the patient. βₛ are the unknown regression coefficient parameters of the covariates, which quantify the effect of covariates on the survival. All co-variates are quantile normalized to N(0,1) normal distribution. The βₛ are determined by standard likelihood maximization of the model using R-package “Survival”. The significance of β₁, which is coefficient for SR interactions term is determined by comparing the likelihood of the model with the NULL model without the interaction indicator I(V, R) followed by a Wald's test[Therneau, 2000 #341], i.e:

h_{null,g}(t,patient)˜h_{0g}(t)exp(β₂g(V)+β₃g(R)+β₄ age)

The p-value obtained by the Wald's test is corrected for multiple hypotheses assumptions. INCISOR determines the SCNA-based survival effect of the putative SR pair in an analogous fashion, by replacing gene-expression values in each bin with the corresponding SCNA values.
(3) shRNA screening: This screen is based on searching for candidate SR pairs (that have passed the first two screening steps) that fulfill the following two conditions in pertaining cancer cell-line screens: (i) the knockdown of a candidate vulnerable gene V is not essential in cell lines where its candidate rescuer gene R is over-active, and (ii) knockdown of the candidate rescuer gene R is lethal in cell lines where V is inactive. Using genome-wide shRNA screens, INCISOR examines the samples where V and R show the aforementioned conditional essentiality. Specifically, we perform two Wilcoxon rank sum tests to check for the conditional essentiality of V and R as follows:
   Using two genome-wide shRNA dataset, INCISOR determines the conditional essentiality of both V and R using gene-expression and SCNA independently. INCISOR infers the pair to have SR interactions based on shRNA screen, if the V and R both show (multiple hypotheses corrected) significant conditional essentiality in either of the datasets.
   Gene-expression-based conditional essentiality of V in a dataset is determined by first dividing the cell-lines into active and inactive groups using the expression of R (due to limited number of cell lines, cell lines were divided into active/inactive if they are greater/less than median expression R) from the dataset, and then comparing the essentiality of V in the two the groups. The significance of essentiality is determined by a standard Ranksum Wilcoxon test if V shows significantly lower essentiality in the active group is significantly compared to the inactive group. The conditional essentiality of R is determined in an analogous manner.
(4) Phylogenetic profiling screening: The final set of putative SRs is prioritized using an additional step of phylogenetic screening, which checks for phylogenetic similarity (presence or absence across an array of different species spanning the tree of life) between the genes composing the candidate interacting pair. This allows to further prioritize SR interactions that are more likely to be true SRs.
   We study if a gene pair (V and R) has co-evolved together by comparing the phylogenetic profiles of these individual genes in a diverse set of 87 divergent eukaryotic species by adopting the method from Tabasch et. al[Tabach, 2013 #336] [Tabach, 2013 #331]. In brief, this method quantifies the presence or absence of a gene in a continuous fashion (instead of a discrete presence/absence score) by comparing the sequence similarity and therefore retaining more evolutionary information[Tabach, 2013 #336][Tabach, 2013 #331]. Then the matrix of the continuous phylogenetic score of all genes is clustered using a non-negative matrix factorization (NMF)[Kim, 2007 #344], and a cluster membership score vector is determined by using the NMF encoding matrix. The similarity of the phylogenetic profiles of the two genes examined in a given candidate SR pair is then determined by calculating the Euclidian distance between the cluster membership vector of each genes in the pair. The top 5% of the candidate SR pairs examined at this step with the highest phylogenetic similarity are predicted as the final set of SR pairs.

To process half a billion gene pairs for around 9,000 patient tumor samples in a reasonable time, the most computationally intensive parts of INCISOR are coded in C++ and ported to R. Further; INCISOR uses open Multiprocessing (OpenMP) programming in C++ to use multiprocessor in large clusters. Also, INCISOR performs coarse-grained parallelization using R-packages “parallel” and “foreach”. Finally, INCISOR uses Terascale Open-source Resource and QUEue Manager (TORQUE) to uses more than 1000 cores in the large cluster to efficiently infer genome-wide SR interactions.

INCISOR to detect DD, UD and UU interactions: INCISOR identifies DD, UD and UU type interactions in an analogous manner as of DU identification with following additional modifications: (i) The statistical tests in SoF and Survival screening (i.e. Binomial test and Cox Regression) are modified so as to account for each type of SR interaction different activity states are rescued and not-rescued states occur in different activity states for various type of SR interactions (FIG. 6 b-d). (ii) Similarly, shRNA screen is only used DD (for UD and UU interaction lethality occurs due to over-expression of the vulnerable gene and hence the screen cannot be used). In DD interaction, knockdown of rescuer gene, which decreases the cell proliferation and hence is essential for the tumor cell, increase the cell proliferation due to activation of SR rescuer. A Wilcox test quantifying significance of increase of cell proliferation due to rescuer knockdown is used as shRNA screening. (iii) The phylogenetic screen remains same as the case of DU identification.

### 2 Pan-Cancer SR Network

### 2.1 DU Network

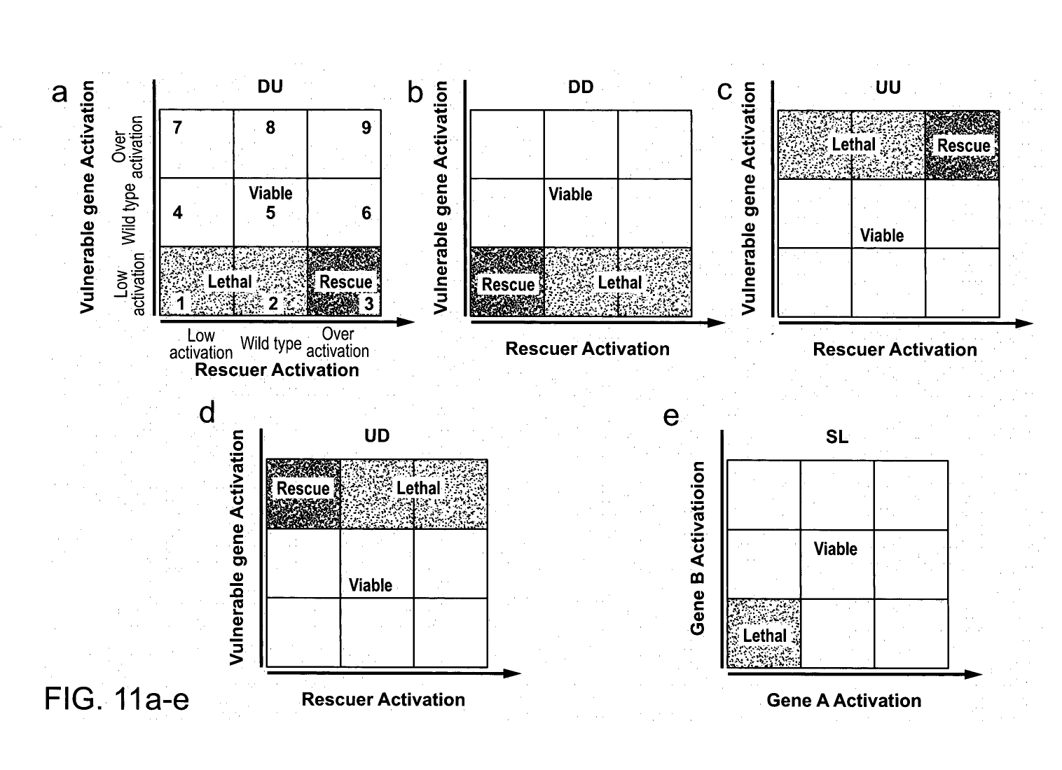

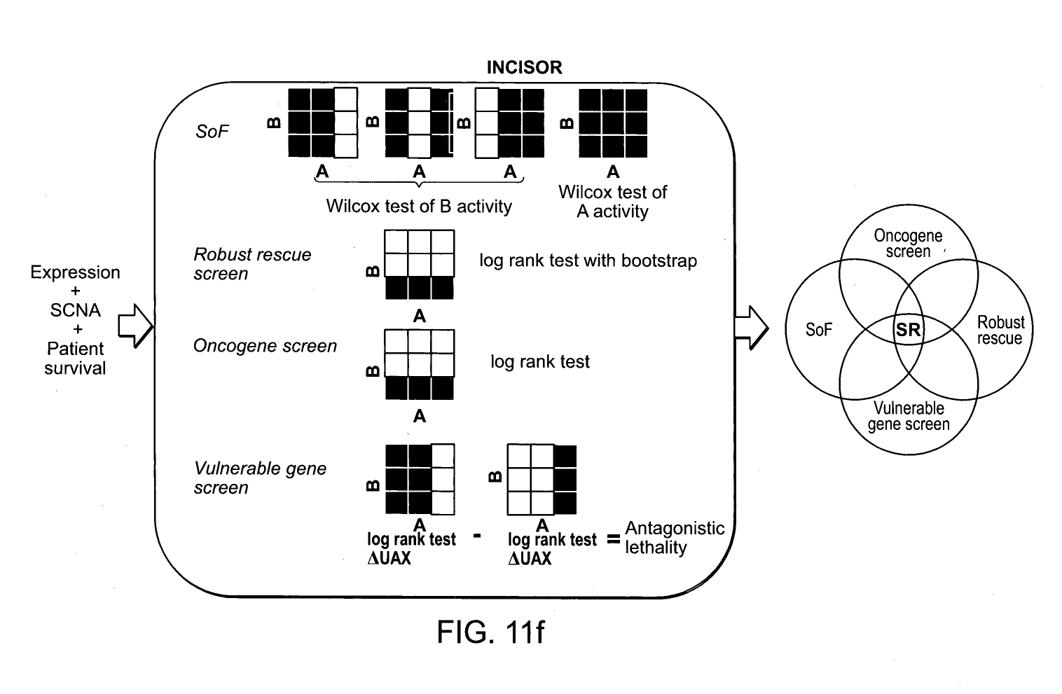

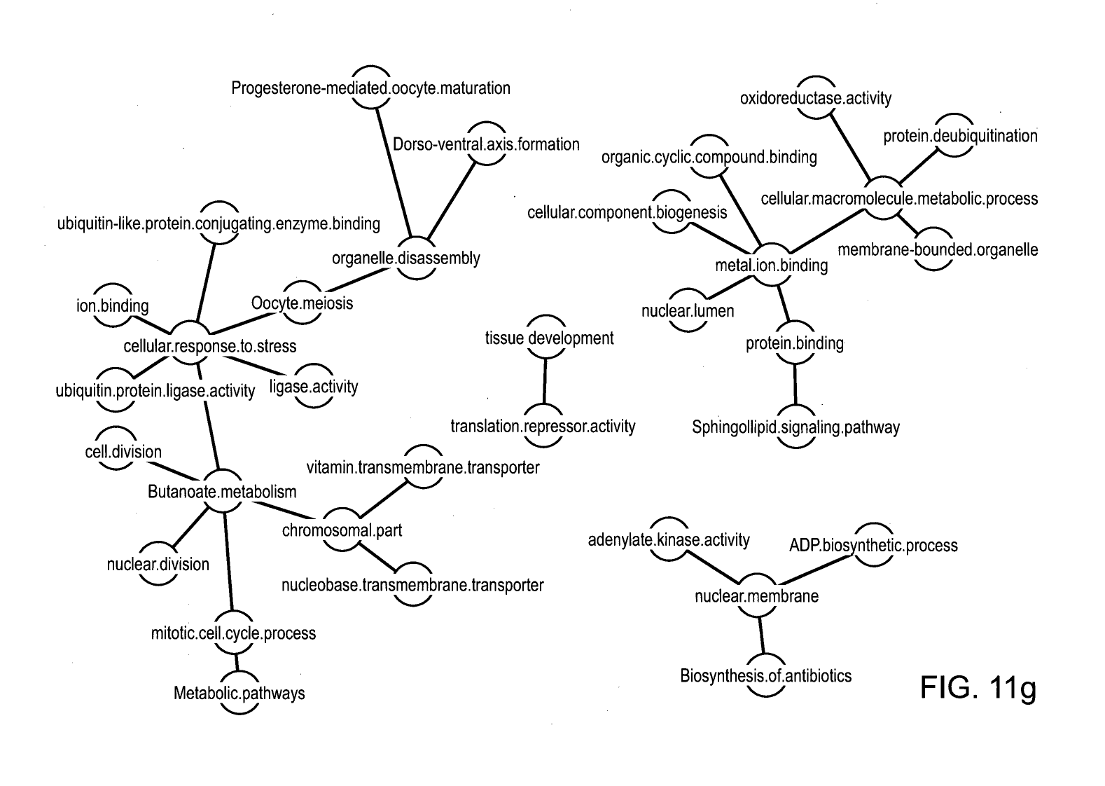

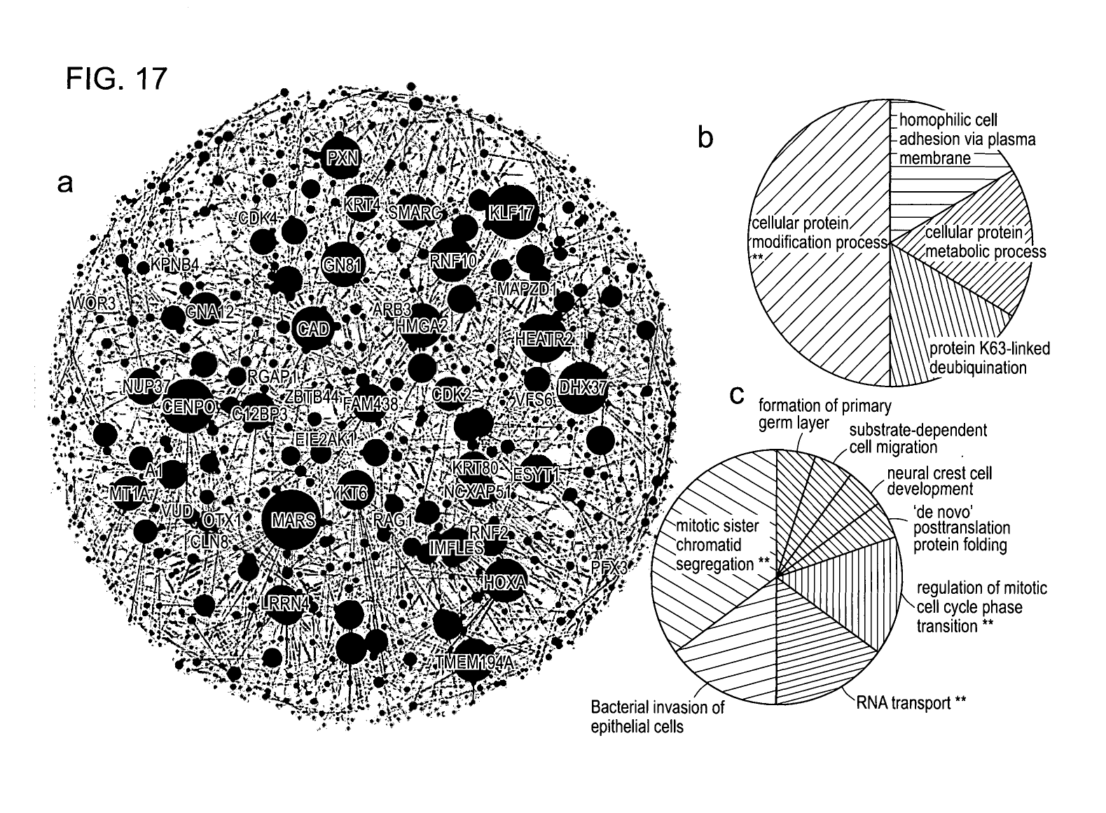

We applied INCISOR to the pan-cancer TCGA data spanning 7,995 samples across 28 different cancer types. SR interactions are overwhelmingly asymmetric, where only 10 genes (ARL2BP, FOXL1, GLDN, JAM2, MT1A, PLEKHM2, SLC19A3, TMEM39B, UACA, UBE3B) are both rescuers and vulnerable genes. The pan-cancer DU-SR network has 2,033 interactions involving 686 rescuer genes and 1,513 vulnerable genes (FIG. 17). We carried out gene enrichment analyses using ClueGO⁴². Vulnerable genes are enriched with cellular process regulation, protein metabolic and developmental processes and the rescuers are enriched with mitotic cellular, macromolecule metabolic and embryo development processes (FIG. 17b,c), and in pairwise the inactivation of genes involved in metabolism and adenylate kinase activity is rescued by genes in mitotic cell cycle, and nuclear membrane, respectively (FIG. 11h). To check whether SR interaction is mediated by physical contact of proteins, we compared a protein-protein interaction (PPI) network⁴³ and our SR network. We found a small fraction (2.5%) of SR-DU interactions (hypergeometric p-value=0.70) are mediated by physical protein interactions.

If a cellular response to the inhibition of a vulnerable gene results in overactivation of an oncogenic rescuer, such inhibition will be carcinogenic. Indeed, by mining the data of carcinogenic agents and their targets⁴⁴⁻⁴⁶ we found that drugs that inhibit vulnerable partners of known oncogenes⁴⁷ are known to be carcinogenic (hypergeometric P<0.03). We considered the DU-rescuer oncogenes that have more than 5 vulnerable partners, and identified their association with the drug targets of the carcinogenic agents identified above using DrugBank²⁴.

### 2.1.1 Clinical Significance of SR DU Network Across Cancer Types

To determine clinical significance of DU-type network across different cancer types, we divided the TCGA dataset by half for each cancer type into a training set and a testing set. We first identified SR pairs by applying INCISOR to the training set, and we tested the clinical significance of the pairs by the fraction of SR pairs that are individually significant in testing set. FIG. 7a shows the fraction of significant SR pairs in each different cancer types. This is a natural way to estimate the clinical significance in each cancer type because many of the cancer types have lower than 200 samples in TCGA.

TABLE S1
Survival Cox regression in METABRIC dataset with features as DU-SR network and other
confounding factors The table summarizes the Cox regression analysis of patient survival
based on DU-SR network and other factors in METABRIC dataset. DU-SR is significant
(p-value < 5E−15) even after controlling for other confounding factors.

| Factors | coef | exp(coef) | se(coef) | z | Pr(>\|z\|) | Significance |
|---|---|---|---|---|---|---|
| Synthetic rescue | 1.45E−01 | 1.16E+00 | 1.85E−02 | 7.826 | 5.00E−15 | *** |
| Age at diagnosis | 1.33E−02 | 1.01E+00 | 3.41E−03 | 3.908 | 9.30E−05 | *** |
| Size | 1.30E−02 | 1.01E+00 | 1.80E−03 | 7.182 | 6.87E−13 | *** |
| Lymph nodes | 6.65E−02 | 1.07E+00 | 5.50E−03 | 12.083 | <2.00E−16 | *** |
| positive | | | | | | |
| Genomic instability | 1.27E−05 | 1.00E+00 | 2.39E−05 | 0.53 | 0.5961 | |
| ERBB2 | −6.66E−01 | 5.14E−01 | 3.34E−01 | −1.992 | 0.0464 | * |
| ESR1 | 2.34E−01 | 1.26E+00 | 9.72E−02 | 2.402 | 0.0163 | * |
| ESR2 | −5.67E−02 | 9.45E−01 | 2.22E−01 | −0.256 | 0.7981 | |
| PGR | −4.71E−01 | 6.24E−01 | 2.97E−01 | −1.584 | 0.1132 | |

### 2.1.2 Clinical Significance of SR DU Network in Other Cancer Types

In the main text, we identified DU-SR network (and others) using TCGA data, and validated it in an independent METABRIC breast cancer cohort dataset²⁵. We compared the survival of patients whose tumors have many vs. few functionally active DU-SRs, and found that rescued tumor samples typically accompany worse patient survival (FIG. 3a). This collective clinical significant in METABRIC data is not simply due to lower expression or copy number of the vulnerable genes in the rescued samples. The mRNA expression and SCNA of the DU-SR vulnerable genes are in fact higher in non-rescued samples than rescued samples (overall ranksum P<2.2E−16 for both), and found 108 (166) of them are significantly up-regulated (amplified) and 700 (1,036) of them are significantly down-regulated (lost their copies) in rescued samples (ranksum p-value<0.05). This shows that the clinical rescue effect is not simply mediated by differential activation of the vulnerable partners.

We also tested the clinical significance of the pan-cancer DU-SR network in another independent dataset for an ovarian cancer patient cohort from International Cancer Genome Consortium (ICGC)⁴⁸. We analyzed copy number alteration, gene expression and patient survival data of 81 patients, and compared the survival of rescued vs non-rescued tumor samples. We observed rescued samples show worse survival compared to non-rescued samples (logrank p-value<0.017, ΔAUC=0.4) (FIG. 7b). We also observed 9.5% of the individual pan-cancer SR-DU pairs show significance (logrank p-value<0.05) in this dataset.

### 2.1.3 TCGA (Single Nucleotide) Mutation Analysis

We examined the TCGA mutation profile to infer causality of SR interaction (DU-type) in pancancer-scale. (The single nucleotide polymorphism mutation profile has not been used in the SR prediction pipeline and hence can serve for independently validating INCISOR predictions.). If the vulnerable gene's inactivation leads to selection for rescuer activation, we expect more rescuers will be active (over-expressed and/or increased copy number) when their vulnerable partner suffers deleterious mutation. We tested this hypothesis using TCGA mutation profile that spans 5,031 patients of 23 cancer types, and we considered SR interactions of 341 genes that have mutations in at least 30 patients. We identified the rescuers of the 341 genes by applying less conservative INCISOR. Using Wilcoxon test, we statistically compared the GE and SCNA of the rescuers in patients with and without vulnerable gene mutations. Indeed, we found that the copy number of rescuers were significantly higher in samples with mutated vulnerable genes than without such mutation (Wilcoxon P<1.2e−100). The expression of rescuer genes was also significantly higher in samples with mutations in vulnerable genes than in those where they are intact (Wilcoxon P<1.1E−17). Overall, 81% of 341 mutated vulnerable genes showed higher copy number of rescuers in the event they were mutated; with 33% of the genes having such a statistically significant increase in their rescuers' copy number (Wilcoxon p<0.05). Only 2.8% of the genes showed statistically significant decrease in rescuers' copy number. In terms of mRNA, 17% of the mutated vulnerable genes showed significant under-expression of corresponding rescuers. FIG. 7c shows the key vulnerable genes, when mutated, whose rescuers show significant increase both in copy number and gene-expression. Extended Data FIG. 7d shows the key rescuer genes that show significant increase both in copy number and gene-expression when their vulnerable gene partners are mutated.

Interestingly, we also identified 7 vulnerable genes whose rescuers have significantly lower copy number variation in mutated samples. We suspected that somatic mutations in these 7 genes might increase its activity. Indeed we found that 3 genes mutations are significantly associated with higher copy number variation or higher gene-expression. In particular, samples with mutations in GATA3 have both higher copy number and gene expression variance.

Our analysis revealed that CDH11, a membrane protein that mediates cell-cell adhesion and is related to ERK signaling pathways⁴⁹, is highly rescued when mutated. It was mutated in 2.1% of TCGA samples. INCISOR predicts IFT172 and MSH2 as DU rescuers of CDH11. MSH2 protein is part of mismatch repair complex (MutS), whose deregulation is associated with emergence of drug resistance. In samples where CHD11 is mutated, these rescuers shows significant increase in copy number (Wilcoxon P<2.6E−6) and expression (Wilcoxon P<0.03). To investigate whether the cells are indeed functionally rescued by over-expression of rescuers genes, we examined the patients with CDH11 mutation and compared the survival of these patients when rescuers of CDH11 are highly activated to their survival when they are not. As anticipated, patients whose inactivated CHD11 is rescued show much poorer survival (FIG. 7e). This analysis demonstrates that a somatic mutation that inactivates a key cancer driver gene can be buffered/rescued by activation of rescuer genes.

### 2.1.4 Cancer-Drug DU SR Network

In identifying the original genome-wide SR-DU network, we have applied a very conservative criterion (FDR<0.01 wherever applicable) at each steps of INCISOR. As a result, the network contained only 2033 interactions (6.2E−4% of all possible gene pairs), leaving out many potential rescuers of many drug targets. To capture DU-type rescuers of anti-cancer drug targets in a more comprehensive manner we modified INCISOR as follows: (i) Vulnerable gene screening was eliminated (because gene targets are by definition known to inhibit cancer progression) (ii) An FDR correction was applied only at the last step, and (iii) The SR significance P-value threshold were relaxed to accommodate weaker SR interactions. The resultant network cancer drug SR network (drug-DU-SR) includes the targets of the majority of 37 key cancer drugs administered to patients in TCGA. drug-DU-SR network includes 170 interactions that consists of 103 rescuers of 36 targets (vulnerable genes) of 37 anti-cancer drugs (FIG. 16c). A pathway enrichment analysis shows the rescuers are highly enriched with lipid storage/transport, thioester/fatty acid metabolism, and drug efflux transporters (FIG. 7g).

### 2.1.5 Drug Response Prediction in Breast Cancer Patients

To verify that DU rescue is an adaptive response of cancer (as opposed to occurring in some cells simply because there is higher basal expression of rescuer genes), we sought to determine if drug treatment stimulates a larger change in rescuer gene expression in clinical non-responder patients versus in responder patients. We used a dataset of 25 breast cancer patients (BC25 dataset) for which expression data was available before and after they were treated with a cocktail of three drugs (epirubicine, cyclophosphamide, and docetaxel), which collectively target four ‘vulnerable’ genes in our treatment-specific SR-DU network²⁶. Remarkably, we found a significantly higher expression fold change (pre-versus post-drug treatment) among the 19 predicted rescuer genes for clinical non-responders vs. responders (17 & 8 patients per group; ranksum p-value<1E−7 when pooling expression of all rescuers across all targets per group; see FIG. 12a,b for per-target breakdown). By next re-calculating this fold change metric on a per-rescuer-gene basis, we were able to rank DU pairs (there were 20 total, incorporating the 19 rescuers) by degree of potency (i.e., by their p-values). We found this ranking to be highly consistent with the rescue effect of the same DU pairs calculated using the BC-DU-SR network (as in step 3 of INCISOR) (Spearman p=0.54, p<1E−3; see FIG. 12c), a reassuring cross-check.

Identification of markers to predict drug response is a key challenge. To address this using our insights from the SR expression data, we built an SVM predictor of treatment response of the BC25 patients based on the pre-treatment expression of the 19 rescuer genes (AUC of 0.71, FIG. 12d). We specifically used the rescuer overexpression profile (a binary vector specifying whether the 19 rescuers are overexpressed or not) as input for the SVM classifier. Feature selection revealed two genes, ATAD2 and PBOV1, that are the most predictive of patient drug responsiveness. ATAD2 is required to induce the expression of a subset of target genes of estrogen receptor including MYC²⁷, and is also known to be associated with drug resistance to Tamoxifen and 5-Fluorouracil^{50,28}. PBOV1 is overexpressed in prostate and breast cancer, and its knockout was reported to disrupt the emergence of resistance to Taxane treatment in prostate cancer⁵¹.

### 2.1.6 Survival Prediction in Gastric Cancer Patients

We further studied pre-treatment and post-treatment expression from 22 gastric cancer patients that acquired resistance to chemotheraphy regiment of Cisplatin and Fluorouracil²⁹. INCISOR identified 15 rescuers of TYMS gene, a target of Fluorouracil using pancancer TCGA data. The expression of the rescuers was significantly over-expressed in post-treatment samples compared to the pre-treatment samples (Wilcoxon p<1.3e−12). Out of 15 rescuers, 11 were significantly over-expressed while the expression of only one rescuer was significantly down regulated (P<0.05, FIG. 12e). Next, we analyzed a larger cohort of 123 gastric cancer patients treated with Cisplatin and Fluorouracil for which we have the pre-treatment tumors gene expression and the patients' progression-free and overall survival rates. Based on the number of highly over-expressed rescuers in each sample, we divided the samples into predicted “rescued” samples and “not-rescued” samples. Indeed, we found that overall survival was significantly worse in predicted rescued samples compared with non-rescued samples (FIG. 12f), and the progression-free survival of the patients was significantly worse in rescued samples as compared to non-rescued samples (FIG. 12g). Reassuringly, overall-survival and progression-free survival were not associated with randomly chosen rescuer genes (FIG. 12h,i).

In order to benchmark the four steps of INCISOR, we identified SR pairs individually by each step of SR using TCGA and analyzed their molecular and clinical significance in the gastric cancer dataset. Specifically, for each INCISOR's step we ranked all possible DU rescuer of TYMS gene using TCGA pan-cancer data and identified the top 20 most significant DU rescuer genes of TYMS gene for each step separately. We then analyzed the over-expression of predicted rescuer in post-treatment (acquired resistant) samples of gastric cancer relative to pre-treatment samples (FIG. 12j). Rescuer genes identified by Robust rescue effect, Oncogene rescuer screening and SoF shows significant over-expression in post-treatment samples. Expectedly rescuer genes identified by Vulnerable gene screening and random genes does not show any over-expression. Next, in order to analyze clinical significance of each rescuer, we analyzed expression and progression-free survival of 123 gastric cancer patients. Analogous to FIG. 12f, we compute the decrease in patient's progression free survival (ΔAUC) in rescued samples over non-rescued samples separately for each step (FIG. 12k). The expression of rescuer genes identified by each of the 4 steps predicts progression free survival.

2.1.7 Predicting acquired resistance in breast and ovarian cancer patients Beyond initial drug response, our overarching hypothesis suggests that SR circuits might contribute to adaptive evolution in tumors after a drug insult, and thus to tumor relapse. To test this, we analyzed longitudinal expression and sequencing data of 81 stage-II, III ovarian cancer patients (OC81 dataset), who were treated with platinum-based therapy and Taxane³⁰ (FIG. 15a), focusing on the activation level of Taxane's 18 identified rescuer genes (of its 3 drug targets), which includes MYC known to play an important role in Taxane resistance in ovarian cancer⁵². Here, the gene activation is measured by the rank of gene expression (GE) or SCNA across all samples in the dataset. In line with our previous observations, we first found significantly higher expression of the 18 rescuer genes in initial non-responder versus responder patients (Wilcoxon rank-sum p-value<1.5E−4; expression and copy number were also significantly higher than for random genes, empirical p-value<0.045, FIG. 8a). Six out of 18 rescuers (respectively, none) showed significant higher (lower) activation in non-responders than in responders (individual Wilcoxon rank-sum p-value<0.05, which is not expected for 18 random genes, empirical p-value<0.036). We then went further and analyzed the patients that initially responded but then relapsed, and found remarkably that rescuer genes became over-active in these relapsed resistant tumors (overall ranksum p-value<5.8E−5), and to a significantly higher degree than 18 random genes (empirical p-value<4.0E−4, FIG. 15b). Five out of 18 rescuers (respectively, none) showed significant post-treatment increase in gene activation (decrease) compared to pre-treatment (individual Wilcoxon rank-sum p-value<0.05, which is not expected for 18 random genes, empirical p-value<0.05). Characteristically high expression profiles of the 18 rescuer genes at the pretreatment stage gave a clear predictive signal for future emergence of resistance (AUC=0.77 for SVM predictor, FIG. 8b).

To get more insight into the rescuer-relapse relationship in the OC81 dataset, we examined the rescuer genes that most contributed to the accuracy of our SVM relapse predictor. The most important rescuer, CLLU1OS is known to be up-regulated in chronic lymphocytic leukemia⁵³, and the second most predictive rescuer, XKR9, plays an important role in apoptosis⁵⁴, and the methylation of the third most predictive rescuer, NPBWR1, is a key prognostic factor for lung cancer patient survival⁵⁵.

Notably, an analysis of multidrug resistance (MDR) genes' expression shows a marked inverse correlation between their activation and the level of rescue reprogramming occurring in Taxane resistant samples (Spearman correlation=−0.63 (p-value<0.03)). Specifically, we considered the gene activation level of 12 MDR genes³⁹, and the gene expression level of 18 rescuers. Our analysis classifies two different groups of patients who develop resistance through either MDR activation or SR reprogramming (FIG. 15c).

We further analyzed the expression data of 155 primary breast cancer patients who were treated with Tamoxifen³⁵, where tumor relapsed in 52 patients within 5 years. With the activity states of 13 rescuers of Tamoxifen's 6 drug targets, our binary classifier was able to predict the patients whose tumor will recur (AUC=0.74, FIG. 8d). The strongest predictor of acquired resistance, RAN, associated with RAS oncogene and androgen receptor (AR), is known to play a role in the resistance to anti-androgen drugs⁵⁶. The third strongest predictor, MAN1C1, is known to be over-activated in cancer cell lines, which would later develop resistance⁵⁷. The function of the second strongest predictor, TMEM200B, a trans-membrane protein, is not known well, indicating its potential role in emerging drug resistance.

It is expected that the synthetic lethal partners of the drug targets will also become active in response to the drug treatment; however, our analysis shows that the activation profile of SL partners does not carry information on tumor relapse. To distinguish the predictive power of SR-DU partners versus SL partners, we built an SVM classifier based on the activity states of 18 SL partners of Taxane's 3 drug targets in ovarian cancer. The accuracy of our classifier was not higher at all compared to the accuracy of 18 random genes (AUC=0.52, FIG. 8c).

### Gene Ontology Distance and Moonlight Gene Analysis

In order to estimate functional relationship between a rescuer and its vulnerable gene partner, we used most common gene ontology (GO) distance measure⁵⁸, which quantifies semantic similarity between GO terms. When multiple GO terms were associated with a single gene similarity score, maximum similarity score was taken as combined similarity score (when we change the combining method to average we obtain similar significance). For each SR-DU pair (FIG. 11g), we computed the similarity measure. The significance of the similarity measure was determined with two set of controls: (a) SR-DU pairs were shuffled to break the original SR-DU interaction. (b) Random pairs. For each set of control we determined the similarity measure in analogous manner. Rank-Sum Wilcoxon test provided the significance of similarity. A particularly interesting case involves RPL23, which suppresses tumor progression by stabilizing P53 protein. It is a moonlighting gene⁵⁹, having two additional secondary functions as a ribosomal protein and an inhibitor of cell cycle arrest⁶⁰. A GO analysis of its 12 predicted rescuer partners shows that they include its secondary functions (Table S2).

TABLE S2
Synthetic rescue interaction of moonlight gene RPL23 The
table lists the 10 rescuer partners of moonlighting gene
RPL23, marking the similarity in their cellular processes.

| MOONLIGHTING GENE | | RESCUER GENES | |
|---|---|---|---|
| RPL23 | 1. Constructs part of 60S | ARNTL2 | circadian and hypoxia factors |
| | subunit, ribosomal | BCAT1 | enzyme catalyzes the reversible transamination of |
| | protein | | branched-chain alpha-keto acids to branched-chain L- |
| | 2. Binds to and inhibits a | | amino acids essential for cell growth |
| | ubiquitin ligase | BHLHE41 | control of circadian rhythm and cell differentiation. can |
| | HDM2, which | | interact with ARNTL |
| | stabilizes of tumor | CASC1 | Cancer Susceptibility Candidate 1 |
| | suppressor p53⁵⁹. | FGFR1OP2 | Signaling by FGFR |
| | 3. Binds nucleophosmin | LMRP | major histocompatibility complex (MHC) class I |
| | and sequesters it in the | | molecules |
| | nucleolus to block its | MRPS35 | Mitochondrial Ribosomal Protein |
| | binding to Miz1 (a | PPFIBP1 | axon guidance and mammary gland development, found to |
| | transcriptional | | interact with S100A4, a calcium-binding protein related to |
| | activator and | | tumor invasiveness and metastasis |
| | repressor), playing a | REP15 | Regulates transferrin receptor recycling from the endocytic |
| | role in inhibiting cell- | | recycling compartment |
| | cycle airest⁶⁰. | STK38L | regulation of structural processes in differentiating and |
| | | | mature neuronal cells. |

### Cancer-Specific Rescuer Hubs

Targeting the rescuer hubs, the rescuers that have a large number of vulnerable partners, will reduce likelihood of developing resistance and should supplement current chemotherapy. For each cancer type, we identified the rescuer hub whose activation was best associated with a decrease in survival of patients (in TCGA). The list of genes provided in Table S3, can serve as target whose inhibition will reduce the likelihood of developing resistance. ODCI is a rescuer hub in general across cancer types, and specifically kidney cancer, acute myeloid leukemia (AML), and prostate cancer. Its over-expression is known to cause chemoresistance by overcoming drug-induced apoptosis and promoting proliferation⁶¹. Similarly many other rescuer hubs are reported to be associated with resistance. Interestingly, none of the rescuer hubs are targeted by current anti-cancer therapies. This may be due to the fact that rescuers become critical for cell proliferation only after vulnerable gene knockdown in cells. This also underscores that targeting rescuers has not been harnessed and SR can provide an entirely new class of drugs.

TABLE S3
Cancer type-specific rescuer hubs. For pancancer, each cancer type,
and breast cancer subtype, we identified the rescuer gene that has largest
number of vulnerable partners. The number (hub size) and identities
of vulnerable partners are listed.

| Cancer | | Hub | |
|---|---|---|---|
| type | Rescuer | size | Vulnerable partner genes |
| pancancer | ODC1 | 16 | ATP6V0D1, BBS2, CCDC79, CETP, CMTM4, DDX19A, DHX38, GABARAPL2, |
| | | | GLG1, GNAO1, MT1E, PSMB10, RANBP10, TRADD, TSNAXIP1, VPS4A |
| CESC | BCL11A | 14 | CDH16, CES2, COTL1, DHX38, FTSJD1, FUK, KLHDC4, NOL3, PHKB, RNF166, |
| | | | SPATA2L, TK2, TMED6, TMEM208 |
| CHOL | C1orf122 | 7 | ANAPC16, ANK3, ARFGAP2, DNAJB12, GPRIN2, MYBPC3, OR13A1 |
| COAD | APITD1 | 1 | CLRN3 |
| DLBC | C2orf16 | 13 | ARL2BP, CDH5, CES2, CMTM2, DPEP2, FUK, GFOD2, HERPUD1, IL34, LCAT, |
| | | | NRN1L, TRADD, VPS4A |
| GBM | LRRC69 | 3 | CCDC151, EPOR, RGL3 |
| HNSC | PMFBP1 | 4 | ADAMTSL3, AP3B2, MRPL46, SNURF |
| KICH | BCL11A | 11 | CDH16, CES2, DHX38, FTSJD1, KLHDC4, NOL3, PHKB, RNF166, SPATA2L, |
| | | | TK2, TMEM208 |
| KIRC | C1orf122 | 8 | ANAPC16, ANK3, DNAJB12, ERCC6, GPRIN2, HKDC1, HNRNPH3, OR13A1 |
| KIRP | ODC1 | 16 | ATP6V0D1, BBS2, CCDC79, CETP, CMTM4, DDX19A, DHX38, GABARAPL2, |
| | | | GLG1, GNAO1, MT1E, PSMB10, RANBP10, TRADD, TSNAXIP1, VPS4A |
| LAML | ODC1 | 16 | ATP6V0D1, BBS2, CCDC79, CETP, CMTM4, DDX19A, DHX38, GABARAPL2, |
| | | | GLG1, GNAO1, MT1E, PSMB10, RANBP10, TRADD, TSNAXIP1, VPS4A |
| LGG | LY6K | 6 | HDHD2, PIAS2, SLC14A1, SLC14A2, SMAD7, ST8SIA5 |
| LIHC | CCDC30 | 7 | DCTN6, MTMR9, MTUS1, PCM1, PHYHIP, SLC18A1, SLC25A37 |
| LUAD | RLF | 14 | ADAMTSL1, ATP8B4, DENND4A, FAM96A, IGDCC4, INTS10, LIPC, MTMR9, |
| | | | RAB11A, RAB8B, SECISBP2L, SNX1, TLN2, TRIP4 |
| LUSC | GREB1 | 2 | HP, KLHL36 |
| OV | RLF | 11 | DENND4A, FAM96A, IGDCC4, INTS10, LIPC, MTMR9, RAB11A, RAB8B, |
| | | | SNX1, TLN2, TRIP4 |
| PAAD | C1orf122 | 7 | ANAPC16, DNAJB12, ERCC6, GPRIN2, HKDC1, HNRNPH3, OR13A1 |
| PRAD | ODC1 | 16 | ATP6V0D1, BBS2, CCDC79, CETP, CMTM4, DDX19A, DHX38, GABARAPL2, |
| | | | GLG1, GNAO1, MT1E, PSMB10, RANBP10, TRADD, TSNAXIP1, VPS4A |
| SARC | PEX14 | 5 | C10orf131, HPSE2, PDCD4, PIK3AP1, SFXN2 |
| SKCM | RLF | 11 | ATP8B4, DENND4A, FAM96A, IGDCC4, LIPC, RAB11A, RAB8B, SECISBP2L, |
| | | | SNX1, TLN2, TRIP4 |
| STAD | RDH16 | 5 | ACTR3B, KCNH2, PTN, TBXAS1, UBN2 |
| TGCT | CTNNBIP1 | 4 | C10orf131, FBXL15, LGI1, NDUFB8 |
| UCEC | SAMHD1 | 3 | COG4, NRN1L, SLC12A4 |
| UCS | ARHGEF10L | 5 | ANXA7, PRKG1, RUFY2, SEC24C, SLC25A16 |
| UVM | FAM136A | 3 | COG8, NFATC3, VPS4A |
| BRCA-all | NFYC | 3 | JAK2, NARG2, RAB27A |
| BRCA- | ACN9 | 2 | CDH5, DPEP2 |
| LuminalB | | | |
| BRCA- | BCL11A | 3 | FTSJD1, FUK, TMED6 |
| Basal | | | |
| BRCA- | POU3F1 | 6 | C10orf111, DNAJC24, FAM180B, JRKL, PTER, TRAF6 |
| Her2 | | | |

### 2.1.7.1 Second Line of Therapy Against Emergence of Resistance

Currently, there is no mechanistic approach to recommend a second line of therapy in case patients acquire resistance to a therapy. SR network provides a unique opportunity to recommend such therapy based on molecular mechanism. We provide a list of drug targets—rescuers that get over-expressed to bypass progression lethality of drug—that can serve as an effective second line of action to the relapsed tumors for each drug (FIG. 4c). For each drug, we identified a rescuer of the drug target that is most clinically significant.

### 2.1.7.2 Estimating the Likelihood of Emergence of Resistance to Anti-Cancer Drug Treatments

If resistance emerges for a drug through the mechanism of SR activation, then the proportion of patients who have rescuer over-activation will provide a conservative estimate of the likelihood of developing resistance. To that end, for the drug whose response is predicted by the SR network, we estimated the drug's likelihood to foster resistance. FIG. 4b shows the proportion of patients with an over-activated rescuer for each drug whose response was predicted by the SR network. For each drug this proportion provides the likelihood that a patient treated with the drug will acquire resistance.

### 2.1.7.3 SR Partners of Cancer Drivers and Metabolic Genes

Next, we provide a list of SR interactions that involve main oncogenic driver genes. A rescuer or vulnerable partner of a cancer driver gene can play an important role in cancer, specifically in resistance emergence or drug effectiveness. These partner genes might be a viable target for a drug to mitigate cancer progression or resistance. First we compiled a list of oncogenic driver genes from three sources (i) CancerQuest (http://www.cancerquest.org/), (ii) Tumor Portal⁶², and (iii) oncogenic drivers and associated genes⁴⁷, summing up to 327 genes, all of which are incorporated by reference in their entireties. Next, using the INCISOR pipeline, we identified rescuers of 33 cancer genes, and the vulnerable partners of 32 cancer genes (Table S4).

TABLE S4
SR interactions of cancer associated genes. The table lists
the vulnerable and rescuer partners of cancer associated genes.

| Cancer | | Cancer | |
|---|---|---|---|
| genes | Vulnerable partners | genes | Rescuer partners |
| ACVR1B | EWSR1 | ACVR1B | CCIN, HRCT1 |
| AKT2 | INSR | APOL2 | CSPP1, PVT1 |
| ARID1B | COL23A1, FAM153A, FLT4, | BCL2 | C8orf33, DYNLT1, FBXO30, PLAGL1, |
| | GJD3, KRT222, KRT27, NBR1, | | RNASET2, T, TFB1M, ZNF250, ZNF706 |
| | PTRF, WNK4 | | |
| ARID2 | PRODH | BMPR1A | C1orf94, FAM159A |
| ASXL1 | C22orf34, FA2H | CSF1R | C5orf28, HTR1E |
| CBFB | KLF13, SCG5 | CYLD | ATP6V0A2, BHLHE41, BRAP, CPSF7, CTDSP2, |
| | | | DDB1, EPYC, ERP27, FAM60A, LRRTM4, |
| | | | NUP107, OAS3, PAPOLG, RASSF9, RFC5, |
| | | | VPS37C |
| CCND1 | MT1L | EP300 | CPSF1, FOXH1, KCNV1, LRRC14, SARNP, |
| | | | TAC3 |
| CDH1 | CYP4X1, MRPS15, OSCP1, | EWSR1 | ACVR1B, RNF139 |
| | TRAPPC3 | | |
| CDK4 | CDH13 | FBXW7 | FUCA2, HBS1L, KLHL32 |
| CDKN2C | ARAP1, CACNB2, CXCL12, | FUS | STEAP1 |
| | FAM188A, IPMK, PTER, RHOD, | | |
| | SPAG6, SUV420H1, ZNF485 | | |
| CTCF | INSC, TRIM68 | GATA3 | HSPA13, NTNG1, OPRD1 |
| CYLD | ACSBG1, CTSH, TSPAN3 | JAK3 | SLC16A6 |
| EXT1 | CNDP2, GPR124, KIAA1328, | KEAP1 | C17orf64 |
| | KLB, RPL9, SLC14A1, | | |
| | SPATA18, TMX3, ZNF236, | | |
| | ZNF407 | | |
| EXT2 | BBS4, CALML4, CCPG1, | KIT | SALL4, SLPI |
| | DMXL2, IQCH, MAP2K5, | | |
| | MEGF11, RNF111, SLC24A1, | | |
| | TMOD2, TSPAN3 | | |
| FANCF | ARRDC4 | KLF4 | DPY19L4 |
| KRAS | BTNL9, ELF2, IQGAP2, SAP30L | LYL1 | HOXB8, KIAA0391 |
| MDM2 | ZNF253 | MAP3K1 | IRX4 |
| MSH6 | UMOD | MLLT1 | NT5C, RNF168 |
| MUTYH | GLB1L, IHH, OBSL1 | NPM1 | COL12A1, ZDHHC5 |
| MYB | ARL4D, LRRC41, PLEKHM1, | PDGFB | CS, RPS26, TAC3 |
| | TBX21 | | |
| MYC | CBLN2, CCDC102B, CHST9, | PDGFRA | CASC1 |
| | FAM69C, SALL3, SLC39A6, | | |
| | SMAD4, ZNF407 | | |
| MYCN | ACSF3, CBFA2T3, GGT5, | PRDM1 | RSPO2 |
| | KLHL36, NOL3, TRADD | | |
| PMS1 | CCL22, CDK10, CX3CL1, DEF8, | PTEN | FIZ1, NLRP11, ZNF580 |
| | GLG1, GNAO1, GPR56, TEPP, | | |
| | ZFP90 | | |
| POLE | ZNF676, ZNF91 | SETBP1 | EIF3H, EZR, FAM91A1, POU5F1B, RAET1E |
| PRDM1 | ARFIP1, NR3C2, RPS3A, TIGD4 | SMAD2 | C6orf70, TFB1M |
| RARA | CDH15, EPM2A, GCDH, JDP2, | SMAD4 | ANXA13, MYC, RAD21, UTP23 |
| | JUNB, OR7C1, RNF166, SNAI3, | | |
| | TCF21, TCF25, ZNF430 | | |
| RET | HMHA1 | SMARCB1 | PKHD1L1 |
| RPL5 | RASSF4 | SMO | CNGB1 |
| SRC | THUMPD1 | TET2 | GTF2H5, MTRF1L, PCMT1 |
| TAL1 | SVIL | TIAM1 | OSMR |
| TNFAIP3 | COL25A1, GUCY1A3, MGST2, | TSC1 | SLC25A32 |
| | MMAA, SH3RF1 | | |
| WT1 | ABHD2, PEX11A | XPC | CYP2B7P1, LYRM2 |
| ZHX2 | CARD10, HDAC10, TTC38 | | |

We also provide a list of SR interactions that involve metabolic genes. Deregulated metabolism is a hallmark of cancer, and their SR partners may play important roles in the process and offer key information on how to counteract cancer progression or resistance. We analyzed the DU-SR network of 1496 metabolic genes using INCISOR pipeline, and identified rescuers of 83 metabolic genes, and the vulnerable partners of 52 metabolic genes (FIG. 11g).

### 2.2 Pancancer DD, UD and UU Networks

Next, we applied INCISOR to pancaner TCGA to identify the genome-wide DD-SR network. The resultant network has 317 interactions that are composed of 159 vulnerable and 197 rescuer genes. Gene enrichment analysis revealed that the vulnerable genes are enriched with processes associated with Toll-like receptor signaling pathways and nerve development. These vulnerable genes are rescued by extracellular matrix disassembly, neuromuscular process and glutathione transferase activity.

In a similar manner, we identified and analyzed the UD and UU, SR networks. The UD SR network contains 505 vulnerable genes and 371 rescuer genes, encompassing 926 interactions. The UU SR network contains 169 vulnerable genes and 68 rescuer genes, encompassing 212 interactions. Gene enrichment of the UD network revealed that vulnerable genes were enriched with processes associated with ion transport and eNOS trafficking, which were rescued by the activation of regulators of biosynthesis process and CD4 T-cell differentiation. On the other hand, in the UU network vulnerable genes were associated with cell cycle (S-phase) and beta-catenin binding; the rescuers were associated with process associated with differentiation cell proliferation.

### 2.3 Pancancer SL Network and Combined Clinical Impact of SL and SR

We identified SL interactions in an analogous manner to SR with slight modifications. Since SL is a symmetric interaction, we performed the false positive control of step 3 for both genes, and eliminated step 2 in the INCISOR pipeline. The procedure led to 304 SL pairs with logrank p-value<1.23E−8.

The functional activity of SL and SR networks determines tumor aggressiveness and patient survival. We found that the clinical impact of the combined SR and SL networks is more significant than any of their individual impacts (FIG. 3f, compare FIG. 3a-d, FIG. 8e). We assigned a SL/SR score to each patient, which adds the number of functionally active SL/SRs. We confirmed that the patients (87 samples) with both higher SL score (>90 percentile) and low SR score (<10 percentile) have significantly better survival than the patients (158 samples) with both lower SL score (<10 percentile) and high SR score (>90 percentile) (logrank p-value<6.59E−6). This combined impact is stronger than any single interactions.

### 3 Breast Cancer SR Network

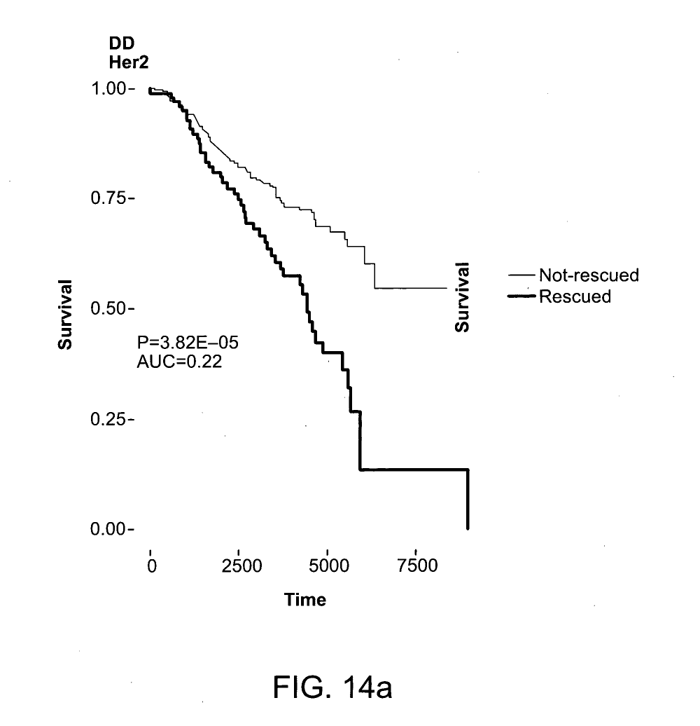

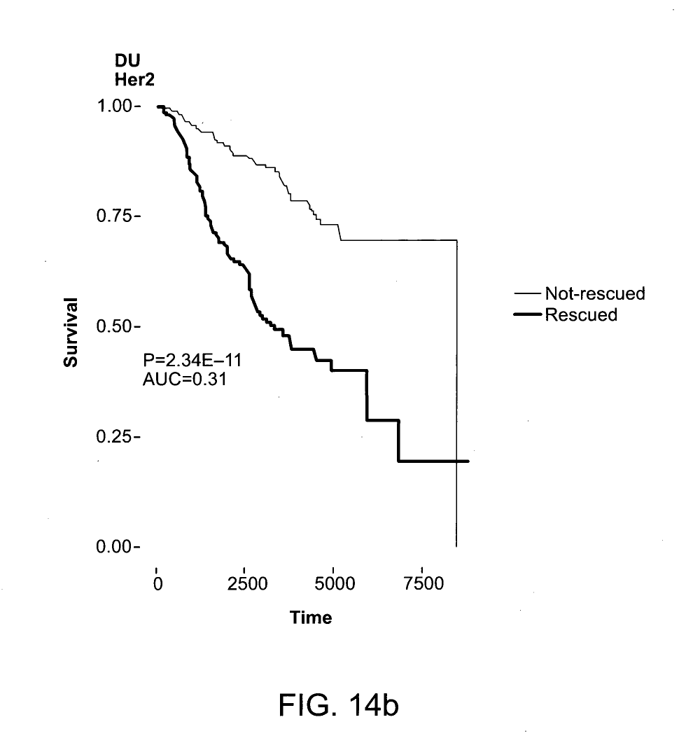

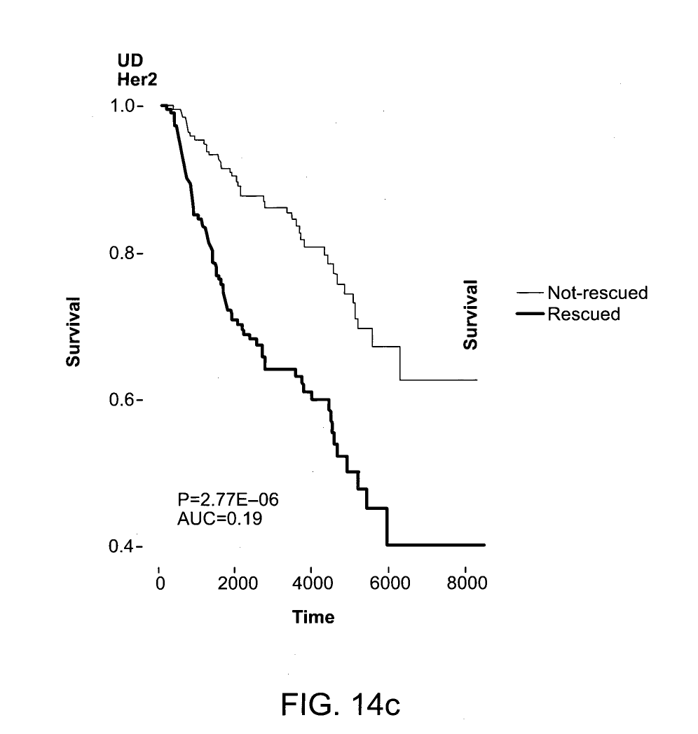

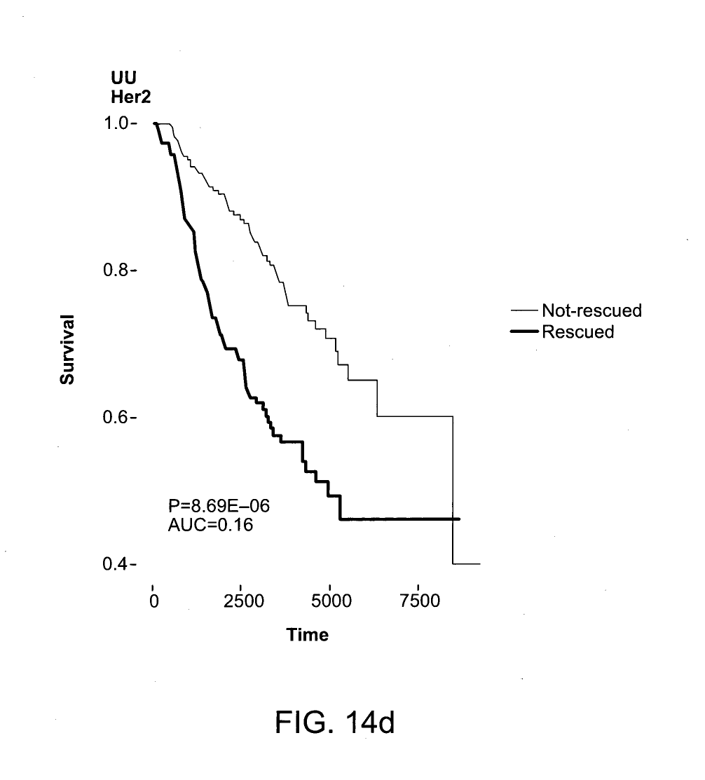

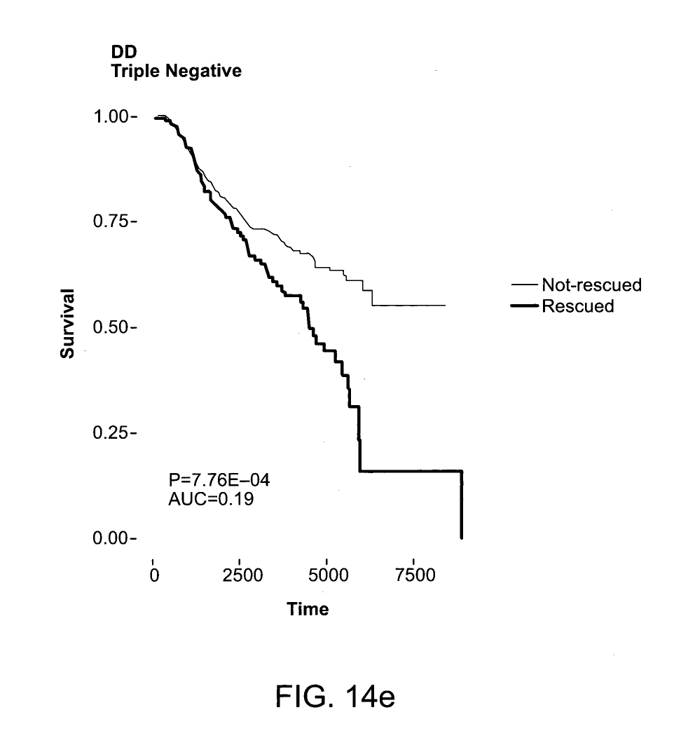

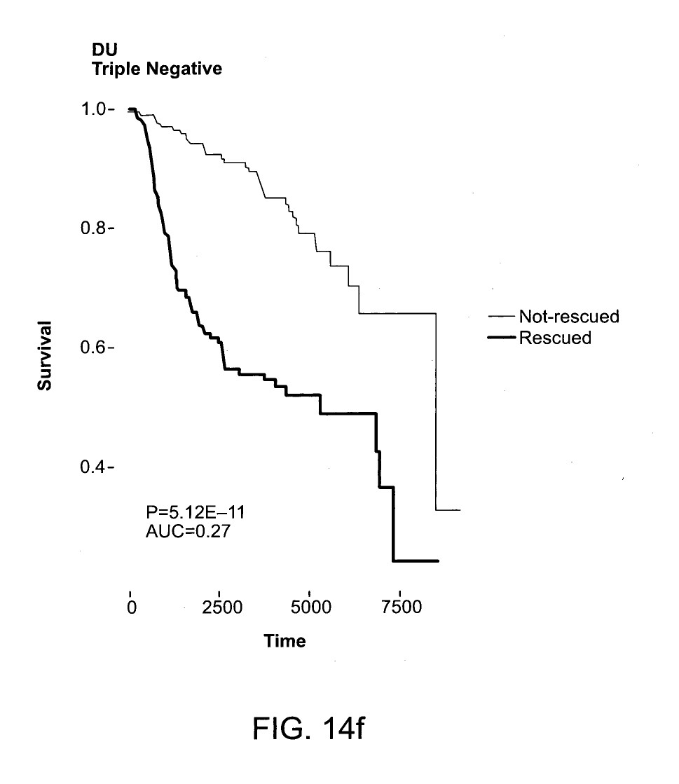

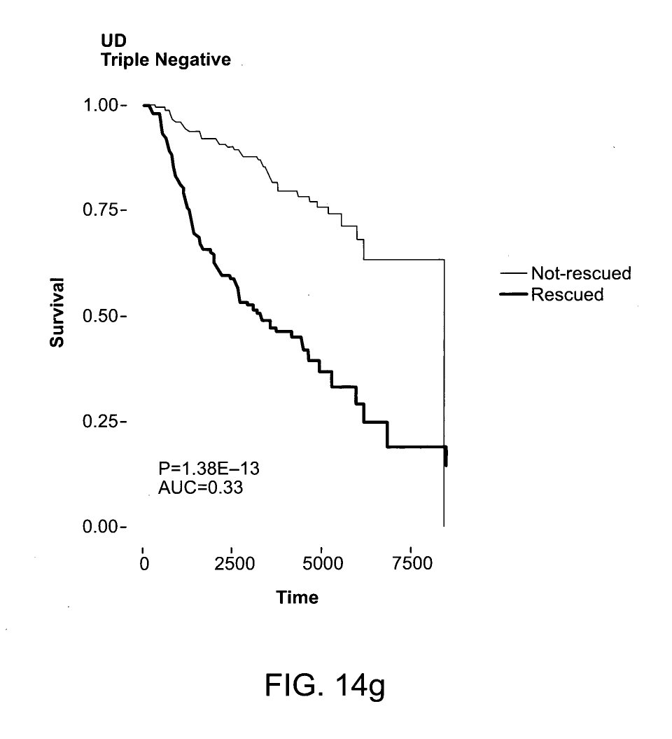

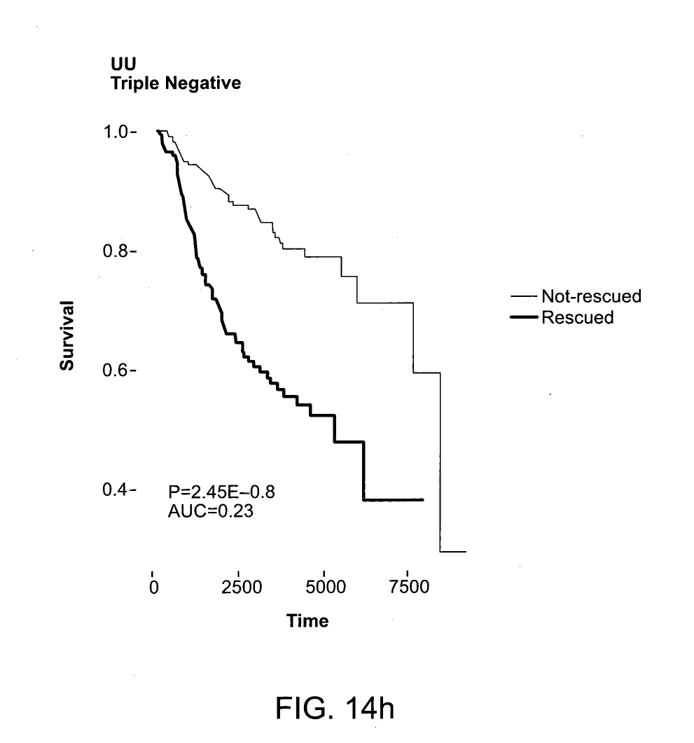

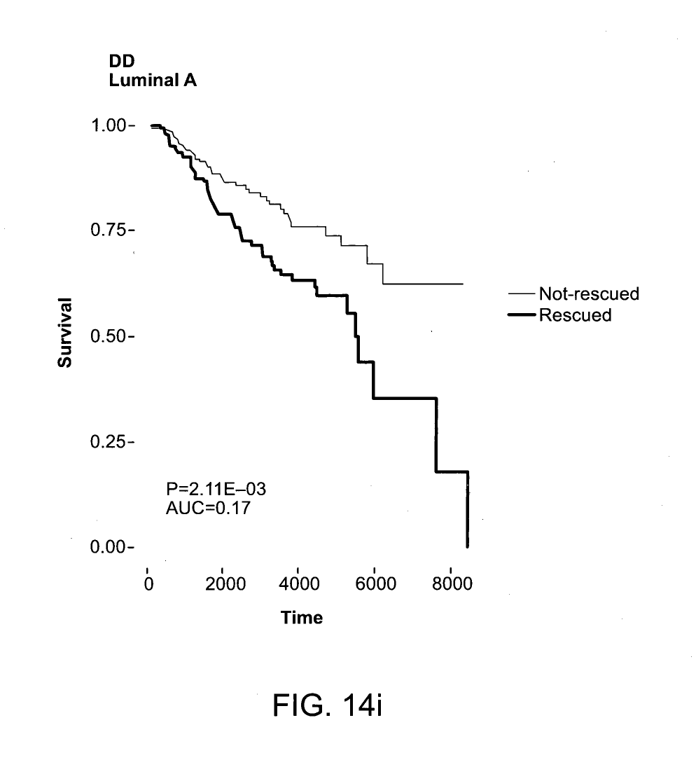

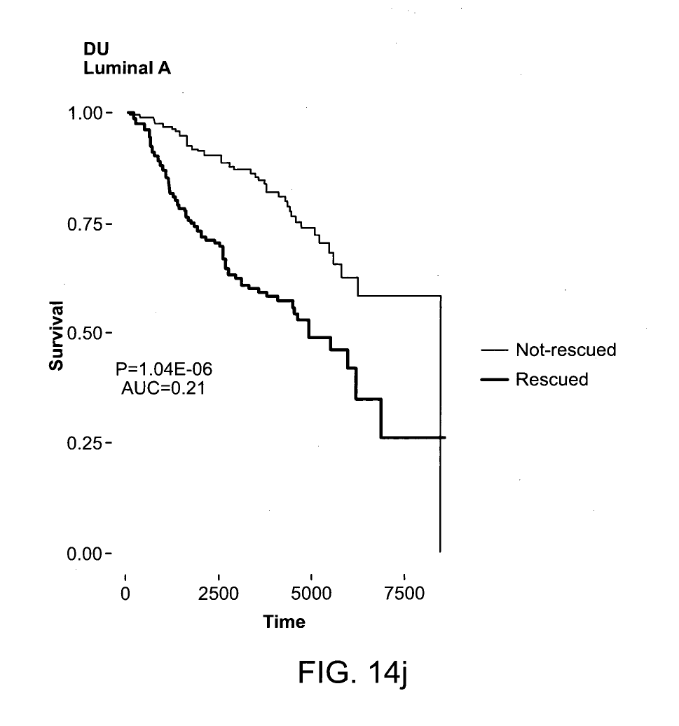

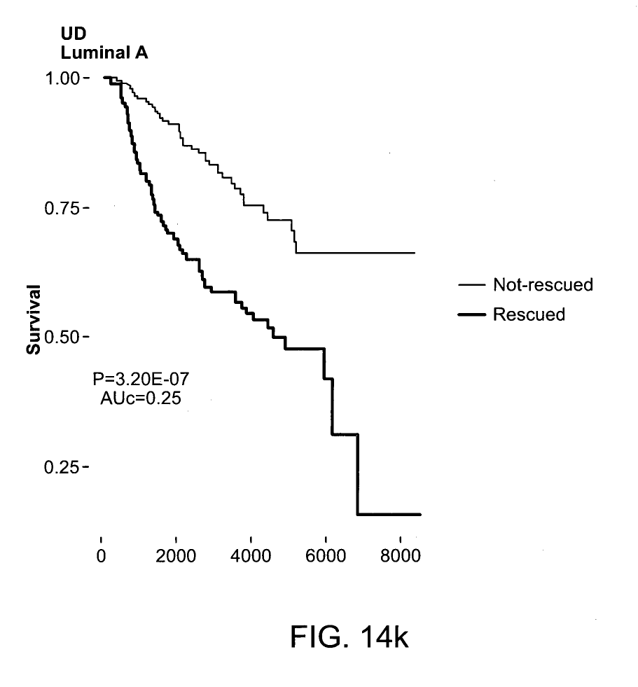

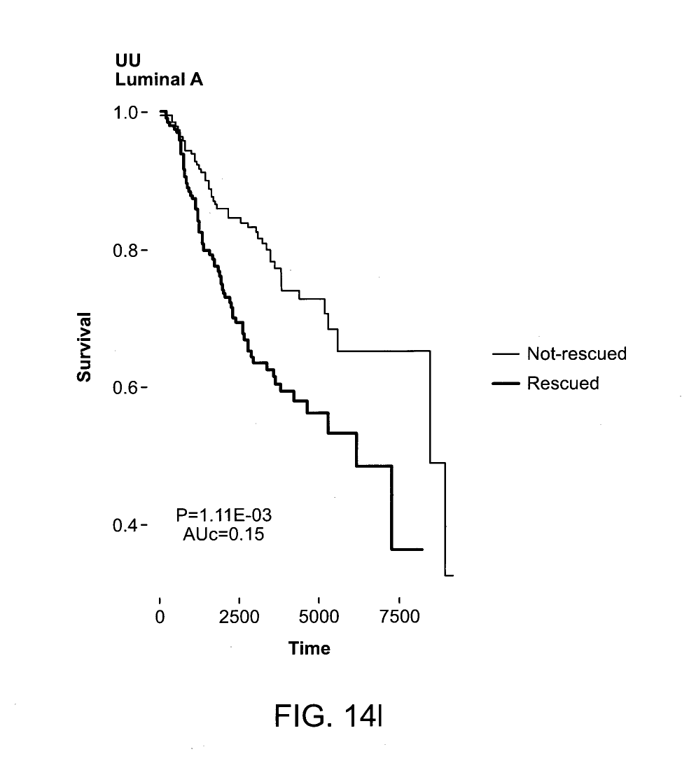

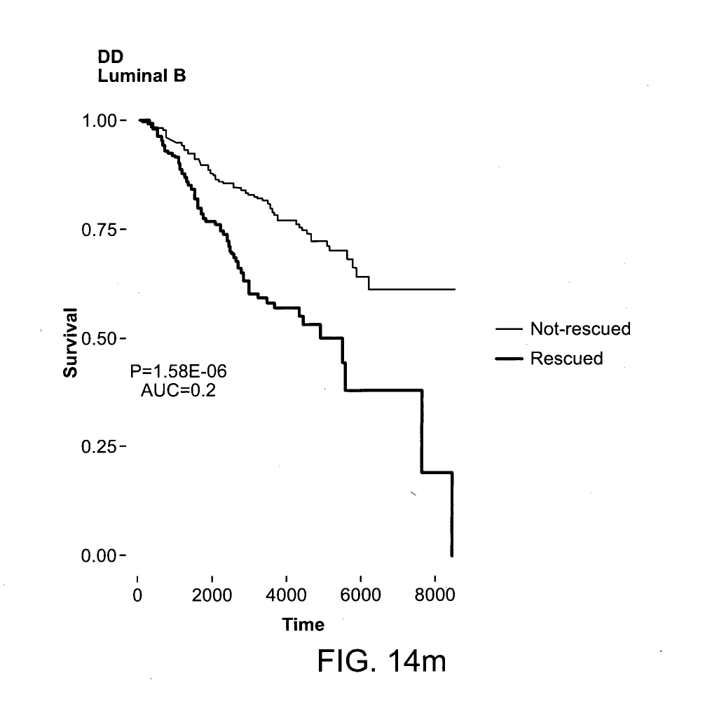

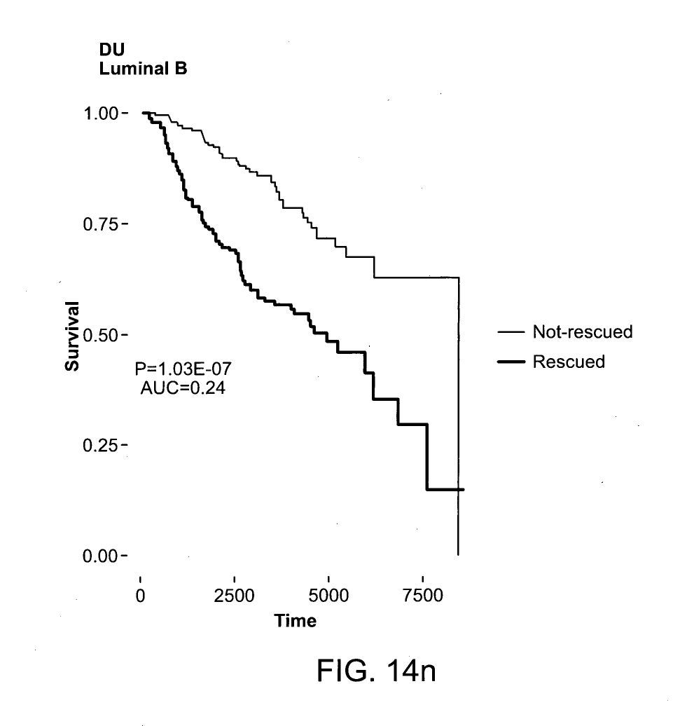

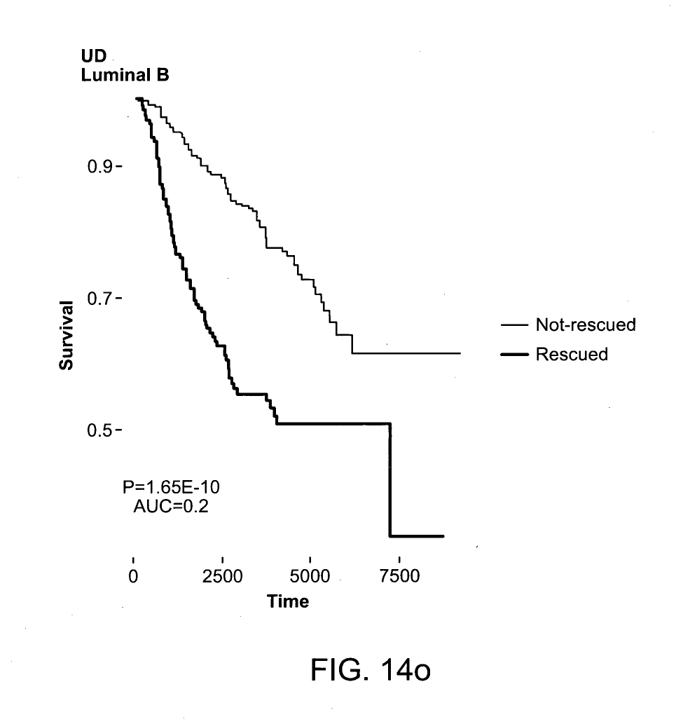

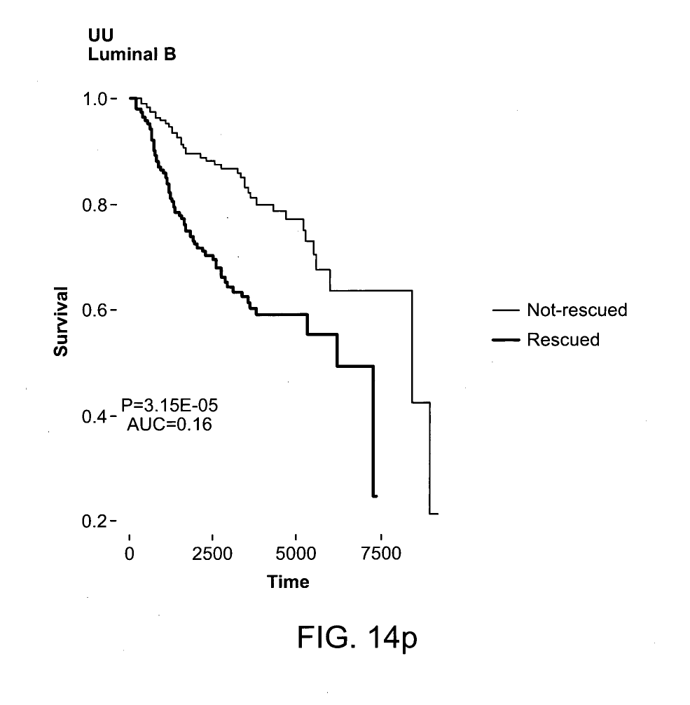

3.1 SR Networks We applied INCISOR to TCGA 1098 breast cancer (BC) patient data to identify the four different types of SR networks specific to breast cancer. We have chosen breast cancer as it has the largest numbers of samples in the TCGA collection, and also has a large independent cohort METABRIC on which we could test the emerging predictions in an independent manner. FIG. 14a shows the resulting BC-DU-SR cancer network, on which we focus most of the section, as it is probably the most intuitive one and, more importantly, it displays the strongest predictive signal, successfully predicting patients' survival in METABRIC BC cohort²⁵.

We next used TCGA BC data to identify DD, UD, and UU type SR networks that are specific to breast cancer. DD network contains 244 vulnerable genes and 110 rescuer genes, encompassing 781 interactions. UD network contains 635 vulnerable genes and 176 rescuer genes, encompassing 1189 interactions. Finally UU network contains 1056 vulnerable genes and 311 rescuer genes, encompassing 3096 interactions.

Interestingly, BC-DU-SR pairs are enriched with several immune processes: vulnerable genes are enriched for tolerance against natural killer cells (the inactivation of which will make cancer cells more susceptible to the immune system), while rescuer genes are enriched for negative regulation of cytokines (which could subsequently prevent cytokine-driven immune cell recruitment). UU rescuers are enriched with macromolecular metabolism, and the vulnerable genes are enriched with protein carboxylation (p-value<1E−4). DD vulnerable genes are enriched with zinc-ion response and negative regulation of growth (p-value<1E−5), and DD rescuers are enriched with nitrobenzene metabolism and detoxification (p-value<1E−7). DU vulnerable genes are enriched with chemokine receptor binding and DNA binding (p-value<1E−5), and DU rescuers are enriched with mitochondrial organization and metabolic process (p-value<1E−4). The UD network is associated with immune response: UD vulnerable genes are enriched with antigen processing (p-value<1E−5), and UD rescuers are enriched with T-cell receptor signaling pathway (p-value<1E−3). UU vulnerable genes are enriched with phosphatidylserine metabolism and antigen process (p-value<1E−3), and UU rescuers are enriched with post-translational protein folding and cell-cell adhesion (p-value<1E−3). Interestingly, BC SR-DU shows a strong involvement of immune-related processes (Table 5): while vulnerable SR-DU genes are enriched with tolerance against natural killer cells (the inactivation of which will increase the cancer cells' susceptibility to the immune system), the rescuer genes are enriched with negative regulation of cytokines (which may prevent immune cells from being recruited by cytokines).

### 3.2 Patient Survival Prediction Using SR Networks

To generate these SR-dependent survival predictions we quantified the number of functionally active SRs in each tumor sample—that is, the number of DU-SR pairs where a vulnerable gene is inactive and its rescuer partner is over-activated in the given sample. As expected, we find that breast cancer samples with a large number of functionally active pairs have significantly worse survival than samples with fewer active pairs, as the former are rescued (FIG. 10a-d). This finding is true for each of the other three SR types, albeit to a lesser extent than the DU-SR type. Combining SR with SL interactions slightly improves the survival predictive power further (logrank p-value<1E−300, ΔAUC=0.42).

The three inherent states of SR interaction—i.e. viable, non-rescued (lethal) and rescued states—display different effects on cancer progression and consequently on patient's clinical prognosis (FIG. 8e). For example, insofar as the SR-DU interaction between a vulnerable gene FGF10 and a rescuer EEA1: patients with either FGF10 WT (viable state) or EEA1 over-activation (rescued state) have lower survival than patients with non-rescued EEA1 knockdown (FIG. 10e). However, patients with the SR pair in rescued state have even lower survival than those patients in viable state. Similarly, patients whose tumor has many SR pairs in non-rescued state have better survival compared to those patients whose tumor has many SR pairs in viable state. As shown in the main text, patients harboring tumors with extensive SR reprogramming have collectively worse survival than the other two groups of patients (FIG. 8e), suggesting the three states of SR have distinct clinical prognoses and are significantly different from each other.

Impact of inactivation of a vulnerable gene can be estimated by comparing the survival of patients in whose tumors the gene is inactivated (‘non-rescued state’) to patients in whose tumors the gene is active (‘rescued state’) (using logrank test). In case a vulnerable gene has more than one rescuer, we collectively compared the patient survival of rescued vs. non-rescued samples. Our analysis shows that the vulnerable genes whose inactivation leads to much better patient survival are more highly rescued in breast cancer. In particular, they have a larger number of rescuer partners (Spearman p=0.11, p-value<0.02).

### 3.3 SR Levels Increase as Cancer Progresses

To study the dynamics of SR functional activity as cancer progresses, we stratified the BC patients in the METABRIC dataset into six different cancer progression bins by their survival times. As expected, cancer progression is accompanied by an increase in the number of functionally active SRs in the tumors (FIG. 10g) and by an increase in the number of inactive vulnerable genes that are rescued (FIG. 10h).

### 3.4 Reprogrammed and Buffered SRs:

We distinguished between reprogrammed SRs (rSR), where the rescuer gene over-activation occurs after the inactivation of its paired vulnerable gene, to buffered SR (bSR), where the rescuer gene over-activation precedes the inactivation of the vulnerable gene.

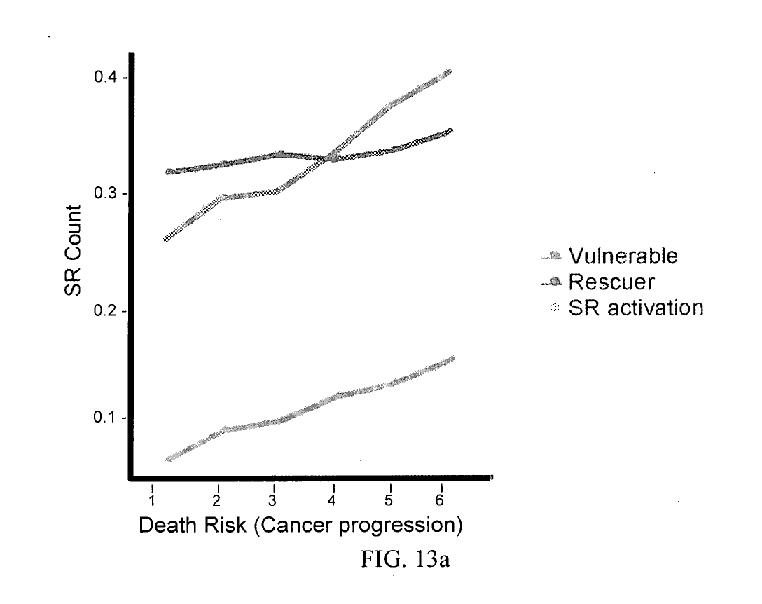

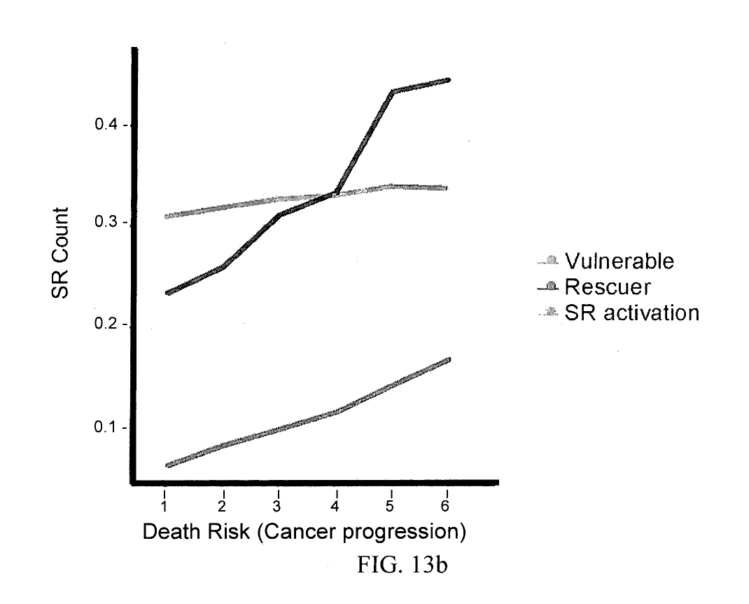

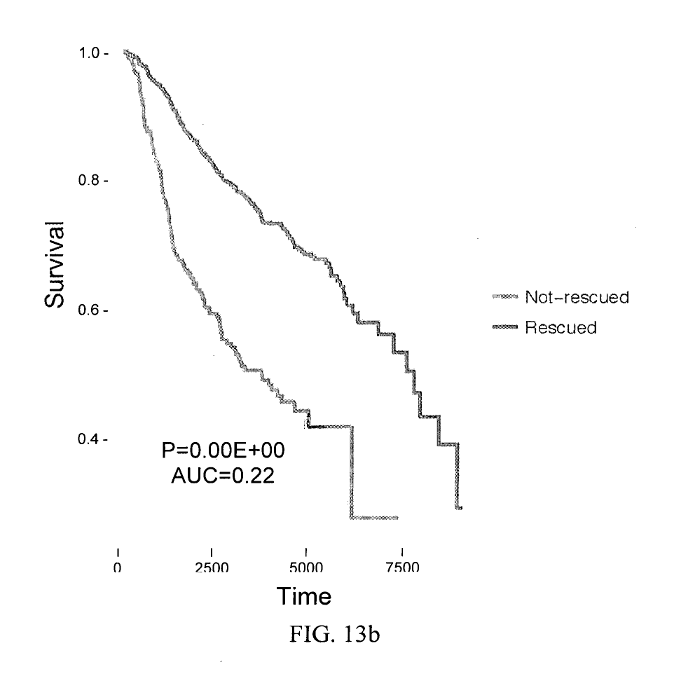

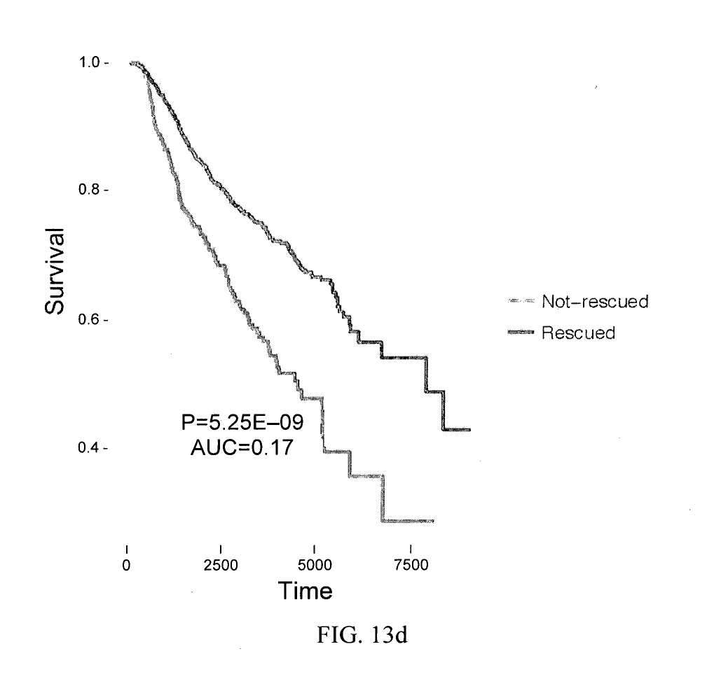

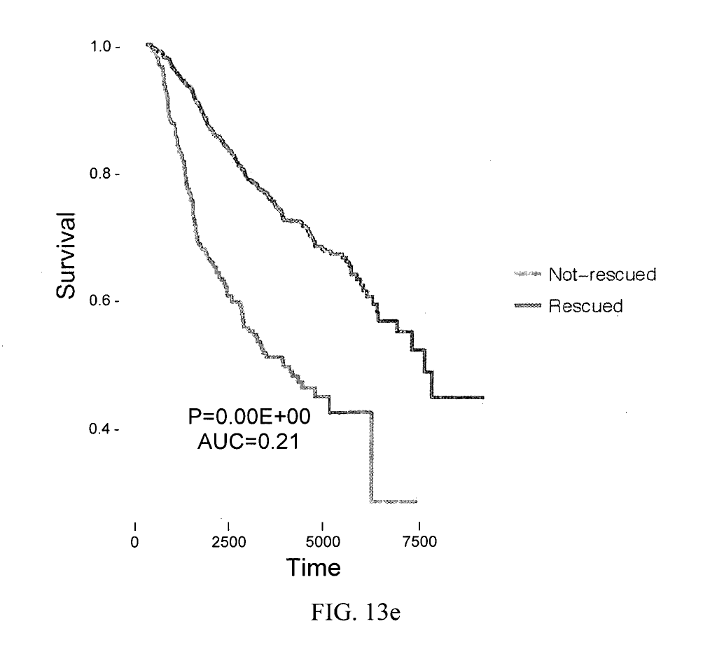

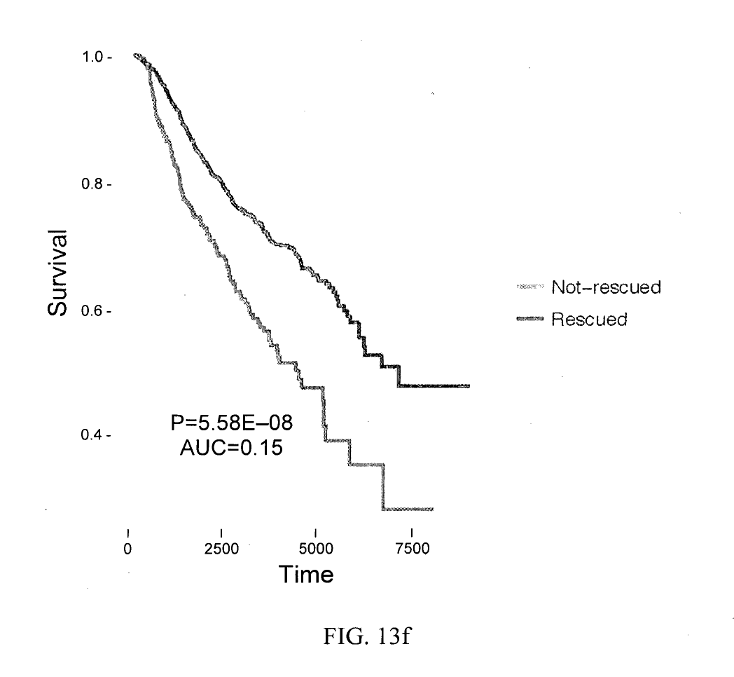

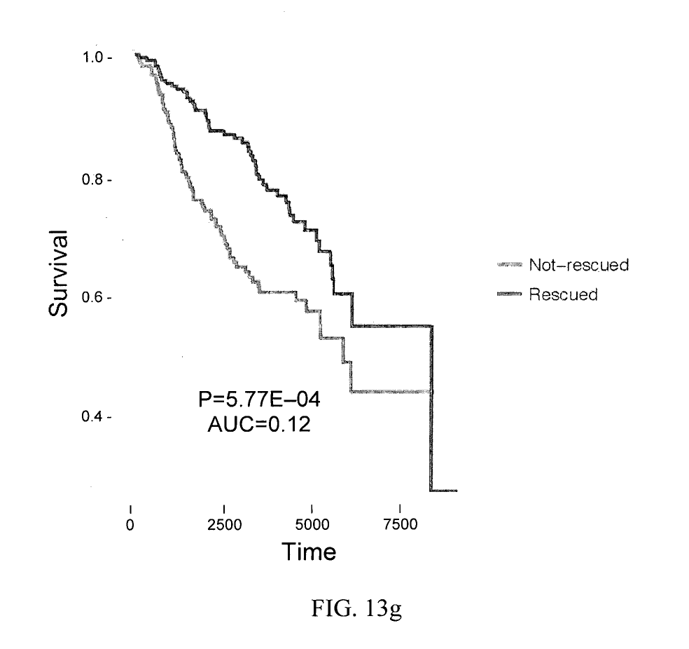

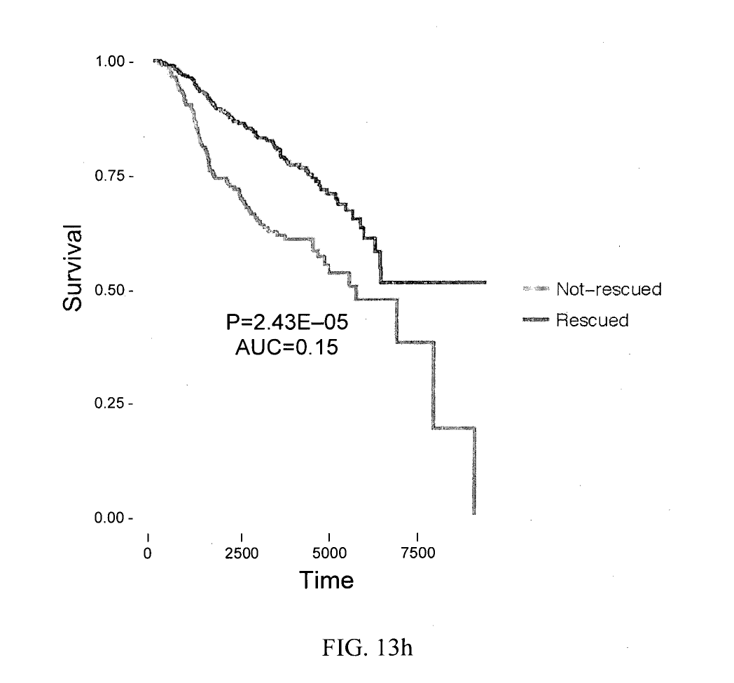

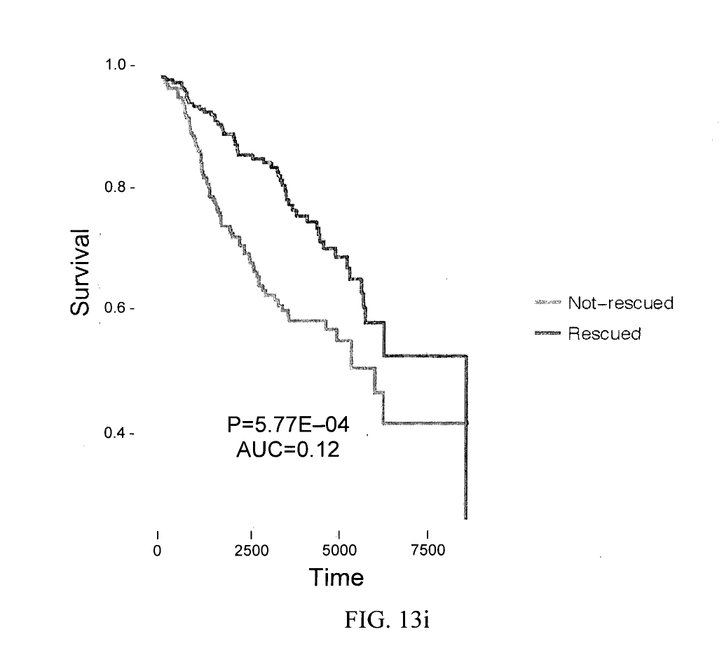

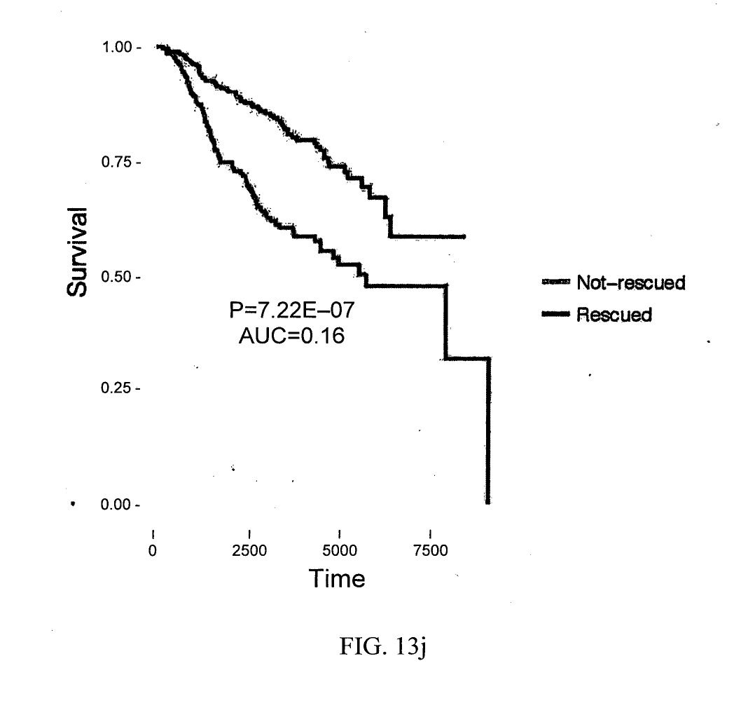

In order to infer if an SR pair is reprogrammed or buffered, we analyzed the fraction of samples with over-active rescuers (fᵣ), inactive vulnerable genes (fᵥ), and functional activation of SR (f_{SR}) at each of 6 cancer progression bins used in Supplementary Information Section 3.3. We classified an SR pairs as an rSR if fᵣ and f_{SR} are highly correlated (Spearman correlation>0.3, p-value<0.05) while fᵥ and f_{SR} are not (Spearman correlation<0 or Spearman correlation p-value>0.05), and f_{SR} is increasing as cancer progresses as shown in FIG. 13a. Similarly, an SR pair was classified as bSR if fᵥ and f_{SR} are highly correlated while fᵣ and f_{SR} are not (analogous to the conditions for rSR above), and f_{SR} is increasing as cancer progresses (FIG. 13b).

While in general SRs carry clinical significance irrespective of their order of occurrence (FIG. 3), rSRs have a significantly stronger survival predictive signal than bSRs (FIG. 13c-j). We first considered the clinical impact of rSR activation—the decrease in survival due to rescuer over-activation given its vulnerable partner is inactivated (which we define as rescue effect in the main text). We confirmed that rSRs have highly significant rescue effect (FIG. 13c), and this effect arises from the pairwise interaction rather than a consequence of single gene (rescuer) over-activation (FIG. 13g), demonstrated by much lower p-value and higher ΔAUC (Δ(ΔAUC)=0.22-0.12). The rescue effect of bSR, conversely, is not much more significant compared to the rescuer control (FIG. 13d,h).

We then considered the clinical impact of bSR activation—the decrease in survival due to vulnerable gene inactivation given its rescuer partner is already over-active. The inactivation of the bSR vulnerable gene is expected to be inconsequential because its rescuer partner is already over-active. We confirmed that the clinical impact of bSR is indeed minimal (FIG. 13f,j). However, we still observed a very strong impact of rSR even in this case (FIG. 13e,i). This means the compensating rescuer activation in response to the loss of the vulnerable gene drives the patient into an even worse state than before the loss. This is consistent with our observation in FIG. 10e, and points to the active role of SR in the emergence of drug resistance.

### 3.5 SR Networks Predict Drug Response of Cancer Cell Lines and Breast Cancer Patients (TCGA)

We next investigated the ability of the DU-SR network to predict the response of cancer cell lines to treatment with commonly used anticancer drugs. The predictions are obtained in a straightforward unsupervised manner (no training data is involved) by analyzing the cell-lines' transcriptomics data to determine cell-line specific gene activity and quantify how many of the SR rescuer partners of the inhibited target(s) of a specific drug tested are over-activated in a given cell line. We analyzed the response of 24 common anti-cancer drugs in 488 cancer cell lines in the CCLE database⁶³. The SR network accurately classifies the cell lines into responder and non-responders for 9 drugs (FIG. 10i). Next, we used breast cancer DU SR network to predict the clinical response of 3873 (pan cancer) patients in the TCGA dataset, focusing on 37 common anticancer drugs. Using the network and transcriptomics data of cancer patients we classified each patient to be a non-responder (or a responder) to a given drug if one or more of the rescuer partners of that drug target are over-active (and as a responder otherwise). We then compared the survival rates of predicted responders to those of non-responders, to examine how well our predictions separated true responders and non-responders. As demonstrated, we quite accurately classify patients into responder and non-responders for 15 of the drugs (FIG. 10j).

The SR network can be used to identify key genes, whose targeting will mitigate emergence of resistance in cancer therapies. To this end we provide a list of major rescuers and their expected clinical utility following treatment targeting their associated vulnerable genes (FIG. 10k), as estimated from their effects on patients' survival in the TCGA. Further, by quantifying the number of samples with functionally active rescuers among the patients that receive a specific drug we provide estimates of the likelihood that resistance will emerge following treatment if these rescuers are not targeted, too (FIG. 10l).

### 3.6 SR Buffers the Lethal Impact of Essential Genes

We identified the essential genes in breast cancer using the essentiality screening data of their knockdown in cancer cell lines^{17,18}. Specifically, we selected those genes that mark top 5% essentiality score in each cell line for more than 20 out of 30 breast cancer cell lines (N=304). We then checked if their inactivation leads to better patient survival using mRNA, SCNA and survival data of TCGA BC and METABRIC. We selected 118 nominal essential genes, which are essential in cell line screening but do not significantly improve patient survival when inactivated (logrank p-value>0.5). As control, we selected 124 actual essential genes, which show significance in patient samples (logrank p-value<0.05). A pathway enrichment analysis shows nominal essential genes are enriched with translation initiation and actual essential genes with cell-cycle regulation (hypergeometric p-value<1.3E−4).

We identified the SR-DU rescuers of the nominal and actual essential genes to compare the number of their rescuer partners and clinical significance. We observed nominal essential genes have a higher number of rescuers (t-test p-value<0.03) and higher collective clinical significance (nominal essential genes: logrank p-value<3.5E−10, control logrank p-value<1.2E−5).

We further tested if an advanced tumor shows higher prevalence of the SR pairs specific to the nominal essential genes than the control SR pairs. We selected aggressive breast cancer samples (N=103) from the most advanced progression step in the tumor evolution analysis. The SR pairs of nominal essential genes indeed show higher level of activation in advanced tumors than in the control (ranksum p-value<1.1E−9) in a more significant manner than three other groups of tumor samples: early stage breast cancer samples from the earliest progression step, all breast cancer samples in METABRIC, and all other cancer samples in TCGA (ranksum p-value>0.2). In particular, the difference between the clinical impact and essentiality in cell lines measured by the ratio of essentiality to clinical significance, positively correlates with the functional activity of SR in aggressive tumors (Spearman p=0.24, p-value<9.2E−4).

### 3.7 SR Partners of Cancer Associated Genes

We analyzed the DU-type rescuer partners of cancer driver genes. Cancer driver genes include the genes strongly associated with cancer that are reported in (http://www.cancerquest.org/) and Tumor Portal⁶², which is incorporated by reference in its entirety, and strongly clinically relevant genes whenover-active or under-active, based on Kaplan-Meier analysis—a total of 45 genes. Using INCISOR pipeline, we identified rescuers of 13 cancer genes in breast cancer (Table S5).

TABLE S5
DU-type rescuer partners of cancer genes in breast
cancer. The table lists the rescuer partners of 13 cancer
genes in breast cancer DU-SR network.

| Cancer Genes | Rescuers |
|---|---|
| CBFB | TNFRSF21 |
| CCNE2 | CYP20A1, DUSP18, PAX3, ZNF454 |
| CDKN1B | MDH1, NCOA7, ODC1, PTPRK, STX7, TRMT11, |
| | UGP2 |
| CTCF | TNFRSF21 |
| ESRP1 | CCDC89, PAX3, ZNF454 |
| FGF3 | BNIP2, MYO5A, NRP1, USP6NL |
| FGF4 | C6orf123, USP6NL |
| GATA3 | PIK3R4, TNFAIP1 |
| KRAS | AIM1, AMD1, AMIGO1, CLIC4, FAM101B, IRAK2, |
| | KCNA2, PARD3B, PAX6, RSC1A1, SLC22A25, |
| | SOS1, TAF13, TCEB3, TCP11L1 |
| NRAS | ABCE1, ACSL1, CASP3, KIAA0922, PAQR3, |
| | SLC10A6 |
| PIK3CA | ACSL1, ARHGAP10, MGST1, MID1, MRPL13, |
| | NDRG1, TMEM40 |
| BRCA1 | ANKRD40, ORMDL3, SPAG9 |
| HER2 | C6orf195, RABGAP1, RC3H2, UBXN2A, PRPSAP1 |

### 4 Breast Cancer—Subtypes SR Network

We applied our INCISOR pipeline to identify specific SR specific networks for four classical subtypes of breast cancer including Her2, triple-negative, luminal-A, and luminal-B, based on analyzing the TCGA BC data.

In Her2 subtype, DU vulnerable genes are enriched with cell migration and toll-like receptor pathway, and the rescuers are enriched with non-coding RNA metabolism, DNA recombination, and p53 binding.

In basal subtype, DU vulnerable genes are enriched with gamma-aminobutyric acid signaling, and the rescuers are enriched with phosphatidylglycerol metabolism. In luminal-A subtype, DU vulnerable genes are enriched with chemokine, cytokine, G-protein coupled receptor pathway, and the rescuers are enriched with lipoprotein receptor pathway and telomere maintenance. In luminal-B subtype, DU vulnerable genes are enriched with dicarboxylic acid catabolism, and rescuers are enriched with cell growth.

The sub-type specific networks derived show significant predictive signal in predicting patients' survival (FIG. 14), even though it is less than the predictive signal of all BC samples together (FIG. 14, due to the much smaller sample size). Comparing different type of SRs, DU has the highest predictive power in all cancer subtypes.

5 Identifying treatment-specific SR interactions

To capture DU-type rescuers of the drug targets of each drug treatment dataset, we modified INCISOR as follows: (i) Vulnerable gene screening was eliminated (because gene targets are, by definition, known to inhibit cancer progression) (ii) An FDR correction was applied only at the last step, and (iii) The SR significance P-value threshold was relaxed to accommodate weaker SR interactions. In case the survival data is available in the given drug treatment dataset, we then quantified the clinical significance of each of the candidate SR (e.g. in case of drug response, survival difference between responders and non-responders or in case of resistance, survival difference of resistant vs sensitive samples). In case survival data was not available, we used relaxed criteria as in the drug-DU-SR network without the cross-validation against METABRIC data. The intersection of clinically significant SR and the SR pairs from each of four steps of our pipeline constitute the final set of SR. If there were no overlaps, thresholds of each step were adjusted such that there was at least one SR in the intersection.

### Functional Enrichment

For the network level functional enrichment analysis, we used ClueGO⁴² (a Cytocscape plugin) with default settings except: (a) GO, KEGG and reactome ontologies were included, (b) network specificity was set to medium, (c) Bonferroni correction for multiple hypothesis correction, (d) Pathways with p-values<0.05 were included. To perform pairwise GO analysis for an SR network, we first identified GO terms that are enriched in rescuer genes (using standard parameters in GOFunction package⁶⁴). To determine GO processes rescued by a set of rescuers in an enriched GO term, we created a gene set composed of vulnerable partners of the rescuers. Finally, we identified GO terms significantly enriched in the vulnerable gene set (FDR<0.05).

6 In-vitro validation in HNSC

To test our ability to predict and experimentally validate a key rescuer gene, we studied the role of mTOR as a predicted rescuer gene in head and neck squamous cell carcinoma (HNSC), where is it thought to play an important role⁶⁵. Rapamycin is a highly specific mTOR inhibitor⁴⁰ and hence enables to target a predicted rescuer gene by a highly specific drug, combined with the ability to knock down predicted vulnerable genes in a clinically-relevant lab setting. To this end we studied SR-DD predictions in a HNSC cell-line HN12, which, like most HNSC cells, is highly sensitive to rapamycin⁶⁶. For this we applied INCISOR to identify top 10 vulnerable partners and 9 rescuer partners of mTOR in a pancancer scale. We also identified HNSC-specific DD-type vulnerable partners of mTOR. In addition to the pancancer SRs, we tested the 19 HNSC specific vulnerable DD-SR partners of mTOR. Detailed information on the shRNA sequence and cell counts are listed in Table 6.

FIG. 8f summarizes the experimental procedure. Each of the mTOR's vulnerable/rescuer partners together with the controls were knocked down in HN12 cell lines, after which mTOR was inactivated via Rapamycin treatment. HN12 cells were infected with a library of retroviral barcoded shRNAs at a representation of ˜1,000 and a multiplicity of infection (MOI) of ˜1, including at least 2 independent shRNAs for each gene of interest and controls. At day 3 post infection cells were selected with puromycin for 3 days (1 μg/ml) to remove the minority of uninfected cells. After that, cells where expanded in culture for 3 days and then an initial population-doubling 0 (PDO) sample was taken. For in vitro testing, the cells were divided into 6 populations, 3 were kept as a control and 3 where treated with rapamycin (100 nM). Cells where propagated in the presence or not of drug for an additional 12 doublings before the final, PD13 sample was taken. For in vivo testing, cells were transplanted into the flanks of athymic nude mice (female, four to six weeks old, obtained from NCI/Frederick, Md.), and when the tumor volume reached approximately 1 cm³ (approximately 18 days after injection) tumors where isolated for genomic DNA extraction. Mice studies were carried out according to National Institutes of Health (NIH) approved protocols (ASP #10-569 and 13-695) in compliance with the NIH Guide for the Care and Use of Laboratory Mice. shRNA barcode was PCR-recovered from genomic samples and samples sequenced to calculate abundance of the different shRNA probes. From these shRNA experiments, we obtained cell counts for each gene knock-down at the following three time points: (a) post shRNA infection (PDO, referred as initial count), (b) shRNA treatment followed by either Rapamycin treatment (PD13, referred as treated count, 3 replicates) or control (PD13, referred as untreated count, 3 replicates) (c) shRNA infected cell injected to mice (tumor, referred as in-vivo count, 2 replicates). To obtain normalized counts at each time point, cell counts of each shRNA at each time point were divided by corresponding total number of cell count.

Since our in vitro experimental analyses were carried out in HNSC cell lines, we also performed experimentally testing for HNSC specific SRs. Specifically, we studied rSR of the HNSC specific DD type as they can be readily validated by in vitro knockdown (KD) experiments. We obtained reversal of rapamycin treatment when vulnerable partner of mTOR is knocked out (FIG. 8g; paired Wilcoxon P<1.1E−06 for 19 pairings). This implies rapamycin treatment that is generally not beneficial for tumor progression but becomes beneficial when mTOR's vulnerable partners are knocked out.

### 7 SR Based Therapeutics Opportunities

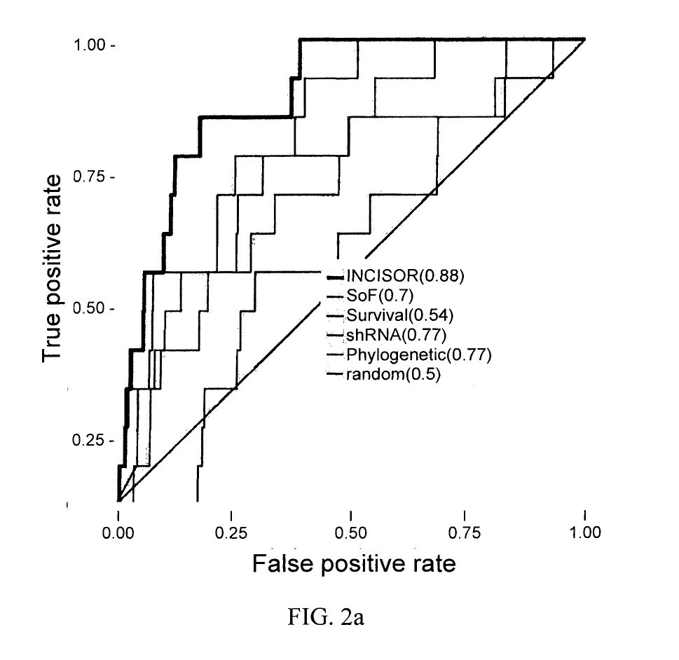

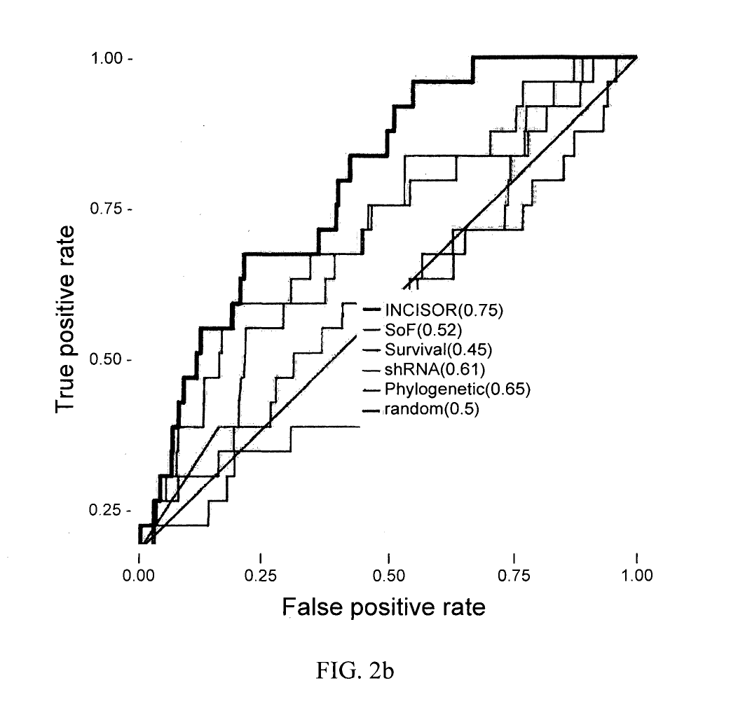

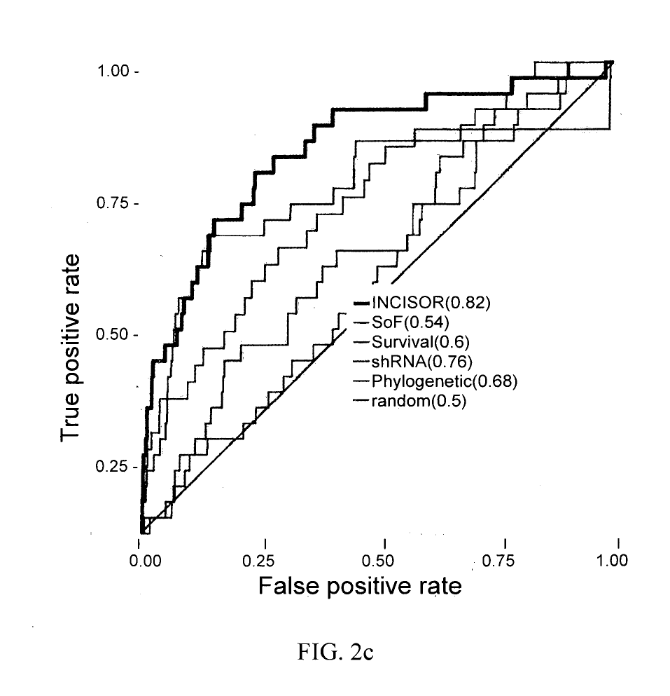

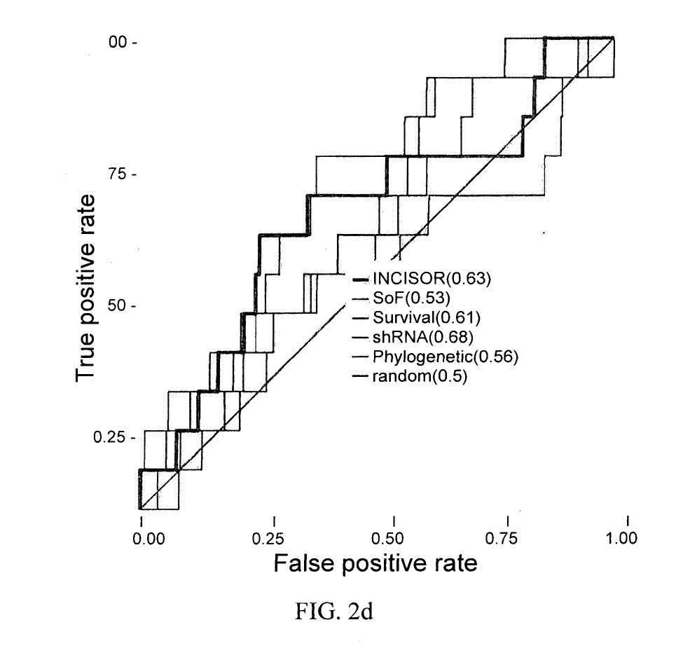

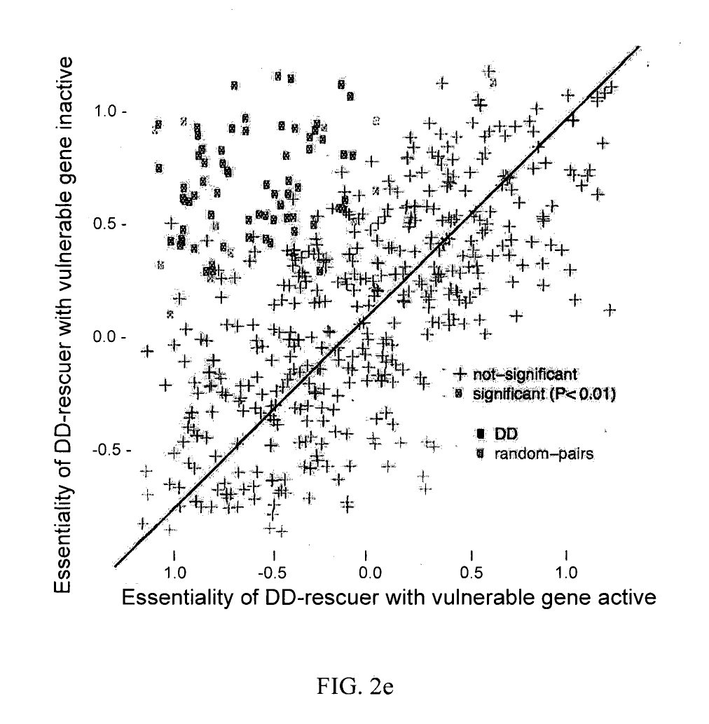

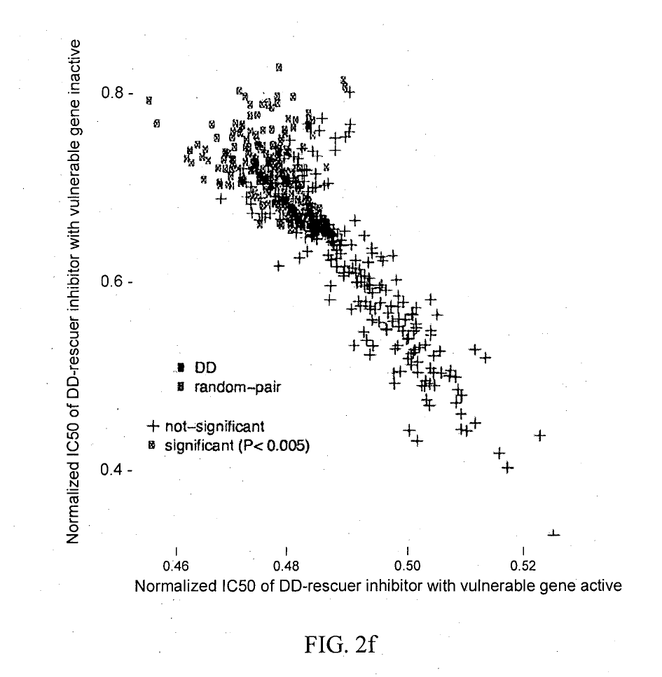

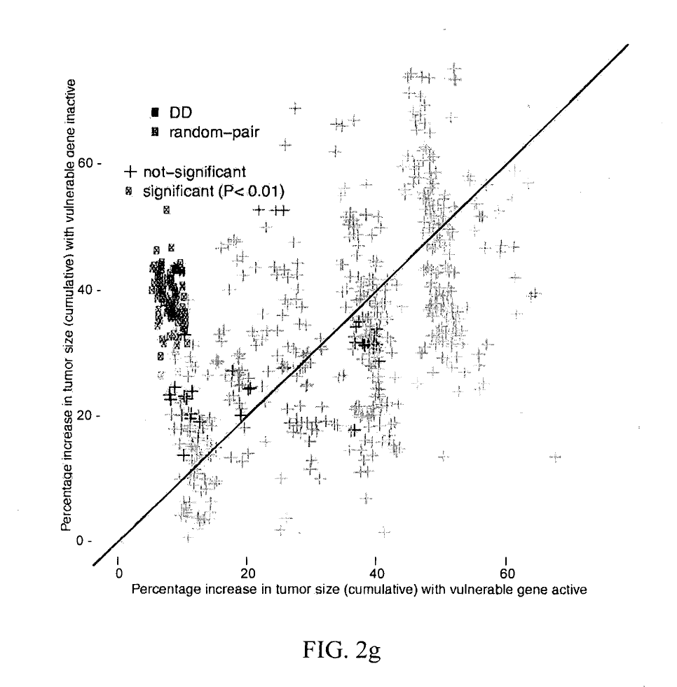

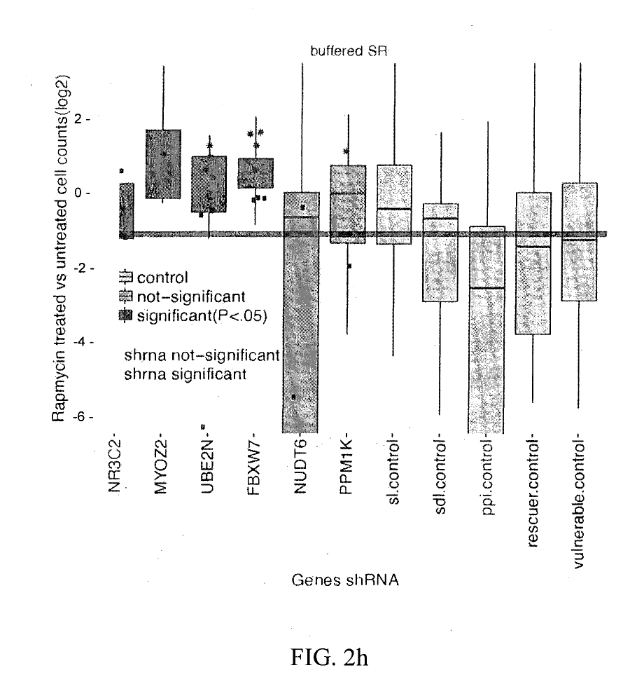

The functional activity of SL and SR networks determines tumor aggressiveness and patient survival. We demonstrate here that the clinical impact of the combined SR and SL networks is more significant than their individual impacts (FIG. 2f). The SL network provides information on the selectivity and efficacy of a given drug⁶⁷. As pointed out above, the SR network provides complementary information on the likelihood to incur resistance. Combining SL and SR networks, we can predict a drug that has the highest efficacy/selectivity and lowest chance of developing resistance.

SR reprogramming can be used to develop two novel classes of sequential treatment regimens of anticancer therapies. First, almost all cancer patients who initially respond to a drug, have the potential to develop resistance to the treatment and experience tumor relapse. Currently, we do not have the ability to access and prepare for the second line of treatment for the relapsed tumors, till it happens to the patients, which is often too late. SR provides a way to infer, together with pretreatment expression screening, whether resistance will emerge quickly and, more importantly, the possible mechanisms of the emergence of resistance and how they can be mitigated by subsequent treatments (as demonstrated in FIG. 4C). Therefore, SR can guide decisions on the second line of action without biopsies from the relapsed tumors. Second, some of the targeted anti-cancer therapies are known to be more efficient and effective in treating cancer (eg. kinase inhibitors) than other drugs, provided tumors are homogenously addicted to their target gene. Using SR interaction between the target gene (as rescuer) and its vulnerable partners, it is possible to make the tumor population homogeneous by targeting the vulnerable partners of the rescuer. In response to the vulnerable gene inactivation, cancer cells will over-activate the rescuer, which will lead to oncogenic (or non-oncogenic) addiction⁶⁸. In the second line of treatment, the rescuer can be targeted to eradicate the homogeneous tumor population, thus efficiently treating cancer.

Difference between SL and SR

It is necessary to be aware of the difference between SL and SR. First, as revealed in FIG. 6, their molecular states are different. In SR, the inactivation of the vulnerable gene is lethal, only over-activation of rescuers retains the cell viability under the condition (i.e. normal expression level is not enough to rescue the cell). However, in SL, the inactivation of one of the SL partners is not lethal unless the other partner is inactivated (i.e. normal expression level does not lead to a lethal state). In other words, the inactivation of a vulnerable gene is in general lethal in SR, unless it is rescued, but the inactivation of a single gene is not lethal in SL pairs. In our analysis we made a clear distinction between SL and SR. In ovarian and breast cancer analysis, the activation profile of SL partners of the drug target genes have poor predictive potential for tumor relapse (FIG. 8c), while over-activation profile of rescuers show great predictive potential (FIG. 8b,d). Also, the predictive power for drug response is significantly reduced if a vulnerable gene is defined rescued when its rescuer partner is not over-activated but only normally activated (FIG. 7f).

Second, in SL, if any two partner genes are both inactive, it will be lethal irrespective of activity of any other genes. But in SR, the inactivation of a rescuer partner of a vulnerable gene does not guarantee lethality because an alternative rescuer may have been over-activated to rescue the cell. Third, while SL has two cellular states of viable and lethal; SR have additional third state rescued, where cancer is often more aggressive than in both viable and lethal states (see FIG. 3e). Fourth, both SL and SR may play roles in determining effectiveness of cancer therapy. In SL, targeted treatments, which inactivate one of the SL partners, lead to the activation of the other partner from inactive state to escape conditional lethality. On the other hand in SR, in response to the inactivation of the vulnerable gene due to targeted therapies, a cancer cell rewires the pathways associated with the targeted cellular function by changing wild-type activity of its rescuer gene (to over-active or inactive state) to escape lethality. In sum, SL is an inherent property of the system, but SR is an adaptive cellular response, where cells reprogram their molecular activity state to evade lethality.

These differences have therapeutic implications. Unlike SL, therapy based on SR is likely to be used only in combination with other primary therapies. While SL-based therapy can selectively kill cancer cells, SR based therapy, on other hand, may not be selective. However, if the primary therapy is selective and SR interaction is highly synergistic (implying selectivity), then the combined therapy will be also selective.

### REFERENCES

1. Fong, C. Y. et al. BET inhibitor resistance emerges from leukaemia stem cells. Nature 525, 538-42 (2015).
2. Rathert, P. et al. Transcriptional plasticity promotes primary and acquired resistance to BET inhibition. Nature 525, 543-547 (2015).
3. Miyamoto, D. T. et al. RNA-Seq of single prostate CTCs implicates noncanonical Wnt signaling in antiandrogen resistance. Science 349, 1351-6 (2015).
4. Bertotti, A. et al. The genomic landscape of response to EGFR blockade in colorectal cancer. Nature 526, 263-7 (2015).
5. Sun, C. et al. Reversible and adaptive resistance to BRAF(V600E) inhibition in melanoma. Nature 508, 118-+(2014).
6. Cancer Genome Atlas Research, N. et al. The Cancer Genome Atlas Pan-Cancer analysis project. Nat Genet 45, 1113-20 (2013).
7. Mills, J. R. et al. RNAi screening uncovers Dhx9 as a modifier of ABT-737 resistance in an EI-myc/Bcl-2 mouse model. Blood 121, 3402-3412 (2013).
8. Falkenberg, K. J. et al. A genome scale RNAi screen identifies GLI1 as a novel gene regulating vorinostat sensitivity. Cell Death Differ 23, 1209-18 (2016).
9. Stuhlmiller, T. J. et al. Inhibition of Lapatinib-Induced Kinome Reprogramming in ERBB2-Positive Breast Cancer by Targeting BET Family Bromodomains. Cell Rep 11, 390-404 (2015).
10. Marcotte, R. et al. Functional Genomic Landscape of Human Breast Cancer Drivers, Vulnerabilities, and Resistance. Cell 164, 293-309 (2016).
11. Crystal, A. S. et al. Patient-derived models of acquired resistance can identify effective drug combinations for cancer. Science 346, 1480-6 (2014).
12. Chou, T. C. Drug combination studies and their synergy quantification using the Chou-Talalay method. Cancer Res 70, 440-6 (2010).
13. Wilson, F. H. et al. A functional landscape of resistance to ALK inhibition in lung cancer. Cancer Cell 27, 397-408 (2015).
14. Hugo, W. et al. Non-genomic and Immune Evolution of Melanoma Acquiring MAPKi Resistance. Cell 162, 1271-1285 (2015).
15. Garnett, M. J. et al. Systematic identification of genomic markers of drug sensitivity in cancer cells. Nature 483, 570-U87 (2012).
16. Iorio, F. et al. A Landscape of Pharmacogenomic Interactions in Cancer. Cell 166, 740-54 (2016).
17. Cheung, H. W. et al. Systematic investigation of genetic vulnerabilities across cancer cell lines reveals lineage-specific dependencies in ovarian cancer. Proc Natl Acad Sci USA 108, 12372-7 (2011).
18. Marcotte, R. et al. Essential gene profiles in breast, pancreatic, and ovarian cancer cells. Cancer Discov 2, 172-89 (2012).
19. Hartwell, L. H., Szankasi, P., Roberts, C. J., Murray, A. W. & Friend, S. H. Integrating genetic approaches into the discovery of anticancer drugs. Science 278, 1064-1068 (1997).
20. Kaelin, W. G. The concept of synthetic lethality in the context of anticancer therapy. Nature Reviews Cancer 5, 689-698 (2005).
21. Ashworth, A., Lord, C. J. & Reis, J. S. Genetic Interactions in Cancer Progression and Treatment. Cell 145, 30-38 (2011).
22. Costanzo, M. et al. The genetic landscape of a cell. Science 327, 425-31 (2010).
23. Motter, A. E., Gulbahce, N., Almaas, E. & Barabasi, A. L. Predicting synthetic rescues in metabolic networks. Molecular Systems Biology 4(2008).
24. Law, V. et al. Drug Bank 4.0: shedding new light on drug metabolism. Nucleic Acids Research 42, D1091-D1097 (2014).
25. Curtis, C. et al. The genomic and transcriptomic architecture of 2,000 breast tumours reveals novel subgroups. Nature 486, 346-52 (2012).
26. Stickeler, E. et al. Basal-like molecular subtype and HER4 up-regulation and response to neoadjuvant chemotherapy in breast cancer. Oncology Reports 26, 1037-1045 (2011).
27. Ciro, M. et al. ATAD2 Is a Novel Cofactor for MYC, Overexpressed and Amplified in Aggressive Tumors. Cancer Research 69, 8491-8498 (2009).
28. Zhang, N., Yin, Y., Xu, S. J. & Chen, W. S. 5-fluorouracil: Mechanisms of resistance and reversal strategies. Molecules 13, 1551-1569 (2008).
29. Kim, H. K. et al. A gene expression signature of acquired chemoresistance to cisplatin and fluorouracil combination chemotherapy in gastric cancer patients. PLoS One 6, e16694 (2011).
30. Patch, A. M. et al. Whole-genome characterization of chemoresistant ovarian cancer. Nature 521, 489-U458 (2015).
31. Carr, J. R., Park, H. J., Wang, Z. B., Kiefer, M. M. & Raychaudhuri, P. FoxM1 Mediates Resistance to Herceptin and Paclitaxel. Cancer Research 70, 5054-5063 (2010).
32. Kwok, J. M. et al. FOXM1 confers acquired cisplatin resistance in breast cancer cells. Mol Cancer Res 8, 24-34 (2010).
33. Zhao, F. et al. Overexpression of Forkhead Box Protein M1 (FOXM1) in Ovarian Cancer Correlates with Poor Patient Survival and Contributes to Paclitaxel Resistance. Plos One 9(2014).
34. Gilkes, D. M., Semenza, G. L. & Wirtz, D. Hypoxia and the extracellular matrix: drivers of tumour metastasis. Nature Reviews Cancer 14, 430-439 (2014).
35. Chanrion, M. et al. A gene expression signature that can predict the recurrence of tamoxifen-treated primary breast cancer. Clinical Cancer Research 14, 1744-1752 (2008).
36. Kim, H. K. et al. A Gene Expression Signature of Acquired Chemoresistance to Cisplatin and Fluorouracil Combination Chemotherapy in Gastric Cancer Patients. Plos One 6(2011).
37. Hatzis, C. et al. A Genomic Predictor of Response and Survival Following Taxane-Anthracycline Chemotherapy for Invasive Breast Cancer. Jama-Journal of the American Medical Association 305, 1873-1881 (2011).
38. Gonzalez-Malerva, L. et al. High-throughput ectopic expression screen for tamoxifen resistance identifies an atypical kinase that blocks autophagy. Proceedings of the National Academy of Sciences of the United States of America 108, 2058-2063 (2011).
39. Gottesman, M. M., Fojo, T. & Bates, S. E. Multidrug resistance in cancer: role of ATP-dependent transporters. Nat Rev Cancer 2, 48-58 (2002).
40. Amornphimoltham, P., Patel, V., Leelahavanichkul, K., Abraham, R. T. & Gutkind, J. S. A retroinhibition approach reveals a tumor cell-autonomous response to rapamycin in head and neck cancer. Cancer Res 68, 1144-53 (2008).
41. Efron, B. & Tibshirani, R. An introduction to the bootstrap, xvi, 436 p. (Chapman & Hall, New York, 1993).
42. Bindea, G. et al. ClueGO: a Cytoscape plug-in to decipher functionally grouped gene ontology and pathway annotation networks. Bioinformatics 25, 1091-3 (2009).
43. Szklarczyk, D. et al. STRING v10: protein-protein interaction networks, integrated over the tree of life. Nucleic Acids Res 43, D447-52 (2015).
44. US Department of Health and Human Services. Public Health Service, National Toxicology Program, Report on Carcinogens, Thirteenth Edition. (2014).
45. International Agency for Research on Cancer (IARC). Agents Classified by the IARC Monographs.

Vol 1-114. (2015).
46. Kuhn, M., Letunic, I., Jensen, L. J. & Bork, P. The SIDER database of drugs and side effects. Nucleic Acids Res (2015).
47. Vogelstein, B. et al. Cancer genome landscapes. Science 339, 1546-58 (2013).
48. Zhang, J. et al. International Cancer Genome Consortium Data Portal—a one-stop shop for cancer genomics data. Database (Oxford) 2011, bar026 (2011).
49. Marie, P. J. et al. Cadherin-mediated cell-cell adhesion and signaling in the skeleton. Calcif Tissue Int 94, 46-54 (2014).
50. Zou, J. X. et al. Kinesin Family Deregulation Coordinated by Bromodomain Protein ANCCA and Histone Methyltransferase MLL for Breast Cancer Cell Growth, Survival, and Tamoxifen Resistance. Molecular Cancer Research 12, 539-549 (2014).
51. Christudass, C., Sood, K., Yeater, D., Getzenberg, R. & Veltri, R. Taxol Resistance in Prostate Cancer: Rescue of Resistance and Expression of Prostate Cancer-Associated Genes Upon Treatment with Hdac Inhibitors. Journal of Urology 187, E323-E323 (2012).
52. Agarwal, R. & Kaye, S. B. Ovarian cancer: strategies for overcoming resistance to chemotherapy. Nat Rev Cancer 3, 502-16 (2003).
53. Buhl, A. M. et al. Identification of a gene on chromosome 12q22 uniquely overexpressed in chronic lymphocytic leukemia. Blood 107, 2904-11 (2006).
54. Suzuki, J., Imanishi, E. & Nagata, S. Exposure of phosphatidylserine by Xk-related protein family members during apoptosis. J Biol Chem 289, 30257-67 (2014).
55. Sandoval, J. et al. A prognostic DNA methylation signature for stage I non-small-cell lung cancer. J Clin Oncol 31, 4140-7 (2013).
56. Trendel, J. A. The hurdle of antiandrogen drug resistance: drug design strategies. Expert Opinion on Drug Discovery 8, 1491-1501 (2013).
57. Yague, E. et al. Ability to acquire drug resistance arises early during the tumorigenesis process. Cancer Research 67, 1130-1137 (2007).
58. Yu, G. et al. GOSemSim: an R package for measuring semantic similarity among GO terms and gene products. Bioinformatics 26, 976-8 (2010).
59. Dai, M. S. et al. Ribosomal protein L23 activates p53 by inhibiting MDM2 function in response to ribosomal perturbation but not to translation inhibition. Mol Cell Biol 24, 7654-68 (2004).
60. Wanzel, M. et al. A ribosomal protein L23-nucleophosmin circuit coordinates Mizl function with cell growth. Nat Cell Biol 10, 1051-61 (2008).
61. Pegg, A. E. Regulation of ornithine decarboxylase. Journal of Biological Chemistry 281, 14529-14532 (2006).
62. Lawrence, M. S. et al. Discovery and saturation analysis of cancer genes across 21 tumour types. Nature 505, 495-501 (2014).
63. Barretina, J. et al. The Cancer Cell Line Encyclopedia enables predictive modelling of anticancer drug sensitivity. Nature 483, 603-7 (2012).
64. Wang, J. et al. GO-function: deriving biologically relevant functions from statistically significant functions. Briefings in Bioinformatics 13, 216-227 (2012).
65. Iglesias-Bartolome, R., Martin, D. & Gutkind, J. S. Exploiting the head and neck cancer oncogenome: widespread PI3K-mTOR pathway alterations and novel molecular targets. Cancer Discov 3, 722-5 (2013).
66. Amornphimoltham, P. et al. Mammalian target of rapamycin, a molecular target in squamous cell carcinomas of the head and neck. Cancer Res 65, 9953-61 (2005).
67. Jerby-Arnon, L. et al. Predicting cancer-specific vulnerability via data-driven detection of synthetic lethality. Cell 158, 1199-209 (2014).
68. Weinstein, I. B. Cancer. Addiction to oncogenes—the Achilles heal of cancer. Science 297, 63-4 (2002).

Table 1. Experimental data of the genes screened in the mTOR experimental analysis

The table lists the sequence for shRNA knockout for each gene, and the measured cell counts of the genes in the mTOR experimental analysis

The following component of the Table 1 includes the names of the genes that correspond (in vertical sequential order from SEQ ID NO: 1-121) to the above-identified shRNAs designed for inhibition:

TABLE 2
Gene Sequences for Genetic Interactions.
DU Interactions
UBXN2A (SEQ ID NO: 121)

| | |
|---|---|
| 1 | agcggcgcgg ccgcggaacc tgaggcggtc tggggcggcg gcgctccggc tctgaagggc |
| 61 | tccagccaaa cggagcccgc ggccaaacgg tgcctgcggt gcctgagctg agtgaggccg |
| 121 | aggccgggag gccgtgcccg gagtaaggcg aaagagaatg aaagacgtag ataacctcaa |
| 181 | aagtataaaa gaagaatggg tttgtgaaac aggatctgat aatcaacctc ttggtaataa |
| 241 | tcaacaatca aattgtgaat attttgttga tagccttttt gaggaagctc agaaggttag |
| 301 | ttccaaatgt gtgtctcccg ctgaacagaa gaaacaggta gatgtaaata taaaattatg |
| 361 | gaaaaacgga ttcaccgtca acgacgattt cagaagttat tccgatggtg ccagtcagca |
| 421 | gtttttgaac tccatcaaaa agggggaatt accttcagaa ttacagggaa tttttgataa |
| 481 | agaagaggtg gacgttaaag ttgaagacaa gaaaaatgaa atatgtttgt ctacgaagcc |
| 541 | tgtgttccag cccttttcag gacagggtca cagactagga agtgccacac caaaaattgt |
| 601 | ttctaaagca aagaatattg aagttgaaaa taaaaataat ttgtctgctg ttccactgaa |
| 661 | caacttggaa cccattacta atatacagat ctggttggcc aatggaaaaa ggattgtcca |
| 721 | gaaatttaac attactcata gagtaagcca tatcaaagac ttcattgaaa aataccaagg |
| 781 | atctcaaaga agtcctccgt tttccctggc aacagctctt cctgtcctca ggttgctaga |
| 841 | tgagacactc acactggaag aagcagattt acagaatgct gtcatcattc agagactcca |
| 901 | aaaaactgca tcttttagag aactttcaga gcactgattt ttgatagact aagtggaaaa |
| 961 | tttgcagaga aatgatggtt gtaagtggac atgcaaacca aaattgggga ttggagaagt |
| 1021 | cagactcact agacttttgg ttcgagtact attgaactct ctcctgatga gaagatgttt |
| 1081 | agataagtac aagttaagaa agtagcatat gactggaaac tatattcagt gcactttctc |
| 1141 | caaaagacta cccagaaaaa tagacttatt ttcaaatacc agttatcaag atatattaaa |
| 1201 | tagctgtatt gtttagaatc ttaatatggt ataaattagc atatgtattc acaatattca |
| 1261 | ttcagacatc attcccagac agcagggatt tatttaaatg ttagctgtct gagtttttaa |
| 1321 | atagctaata cgaccgggta cagtggttca tgcctgtaat cccagaactt cgggaggccg |
| 1381 | agacaggcag atcacgaggt caacagattg agaccatcct ggcaaacatg gtgaaacccc |
| 1441 | atctctagta aaaatacaaa aattagctgg gcgtggcggt gcgcaactgt agtcccagct |
| 1501 | actcgggagg ctgaggcagg agaatctctt gaacctggca agtgtaggtt gcagtgagct |
| 1561 | gagattgagc aactgtactc cagcttggcg acagagcaag accccctctc aaaaataaat |
| 1621 | aaaataaagt aaaataaata taaataattg tggccgggtg caatggctca tgcctgtaat |
| 1681 | cccagcactt tgggaggctg agatgggagg atcacttgaa gccaggagtt taaaaccaga |
| 1741 | atgatcaaca gagtgagacc cctgtctata tattttttta atttaaaaaa taaaagaata |
| 1801 | aaattgtgta gctcagtata gtatcaagat taatctgcct actcacattt ctacacttta |
| 1861 | taaaaatgta ataaaagaaa attatctttc taaaaaaaaa aaaaaaaaa |

FAM43B (SEQ ID NO: 122)

| | |
|---|---|
| 1 | agcctgcgtg gggggagggg agaagagggc aaggggaggg gacaagagag ctagcggtcc |
| 61 | cgcccggtga tgtaggcagc ccggggaggt ggagccgcga cgcctgaagg agtccccacc |
| 121 | gcagccgcgc tctcggtctg ccccactaag cagccgccag cggctccggc gacccaaatt |
| 181 | gcggcggcag ggaccgcgga aatcccaccg tttgggcttg gtggacgtcc agcccacctc |
| 241 | acccccagcc ccggcccctc ctcgcttccc agacggctgg agacactccc gggaaaagcg |
| 301 | gtcctcagcc actcggccgc cgtccgcacc tcggctgctg gcccggctgg gcaccgggca |
| 361 | tctgcgaagc tagccctgcc tggcactggg catctccagg caacgactgt ccccggccct |
| 421 | gcccagcttc tcgcgactcc agggcggtgg acttctgcgc gccttccctc ccccggtctc |
| 481 | ccgacaggac gccggtgagc tccctgcgcc cccagcccct ttcgccgccg ccgcgatgct |
| 541 | gccctggaga cgtaacaaat tcgtgctggt ggaggacgag gccaagtgca aggcgaagag |
| 601 | cctgagtccg gggctcgcct acacgtcgct gctctccagc ttcctgcgct cctgcccgga |
| 661 | cctgctgccc gactggccgc tggagcgctt gggccgtgtg ttccgcagcc ggcgccagaa |
| 721 | agtggagctc aacaaggagg acccgaccta caccgtgtgg tacctgggca acgccgtcac |
| 781 | cctgcacgcc aagggcgacg gctgcaccga cgacgccgtg ggcaagatct gggctcgctg |
| 841 | cgggcctggc gggggcacta agatgaagct gacgctgggg ccgcacggca tccgcatgca |
| 901 | gccgtgcgag cgcagcgccg ccgggggttc ggggggccgc aggccggcgc acgcctacct |
| 961 | gctgccgcgc atcacctact gcacggcgga cgggcgccac ccgcgcgtct tcgcctgggt |
| 1021 | ctaccgccac caggcgcgcc acaaggccgt ggtgctgcgc tgccacgctg tgctgctggc |
| 1081 | gcgggcgcac aaggcgcgcg ccctggcccg cctgctccgc cagaccgcgc tggcggcctt |
| 1141 | cagcgacttc aagcgcctgc agcgccagag cgacgcgcgc cacgtgcgcc agcagcatct |
| 1201 | ccgcgctggg ggcgccgccg cctcggtgcc ccgcgcccca ctgcgccgcc tgctcaatgc |
| 1261 | caagtgcgcc taccggccgc cgccgagcga gcgcagccgc ggggcgccgc gcctcagcag |
| 1321 | catccaggag gaggacgagg aggaggagga ggacgacgcg gaggagcaag agggaggagt |
| 1381 | cccccagcgc gagcggccgg aggtgctcag cctggcccgg gagctgagga cgtgcagcct |
| 1441 | gcggggcgcc ccggcgcccc cgccgcccgc gcagccccgc cgctggaagg ccggccccag |
| 1501 | ggagcgggcg ggccaggcgc gctgagagcc gaaggacagg actcgcagcc ccaggcccga |
| 1561 | cccgccagac tcacagcctc caaccccggc cctgcccgct tcggctgccc cggcccccgg |
| 1621 | cccgtgtctc ccccgtggtc tccgtgttgt ccgccccgcc gcctcatttt ggctcagggt |
| 1681 | gatgcctgat acgcccttgg ttattggggg gtgttcctct ctccccacac ccggagtttc |
| 1741 | ccgggcctgc cattgtggac ccgcccccta tgctttacac ctagtctctt tgcccacaga |
| 1801 | cctcctcatt ccctcccaaa acatcctctc aagagaaggg aggagaagtt tcaagaaatc |
| 1861 | aggaggggtg ggtttggacc ctgggcaggg tggaggcagt gaccttgccc ttggtccctc |
| 1921 | tagccttctt ccctgtgcaa aaaaaaatga ccctggagag gcattcttgt aggagaagaa |
| 1981 | tctagcggcc ggggagaatt ggggccgggc cggcggtggg cagagtccgc tgctatacac |
| 2041 | acagggagga attctcacgc ccaagccccg cctctctacg ccttggagga ctcctgtgac |
| 2101 | ttcactgctc tgcctctgga gaacactggg agagtcctac cgacgttcaa acaacaggtt |
| 2161 | aggccaggta acagccctgc accaggccgc tgcccacgcc tctgccctgg cacccccagg |
| 2221 | ggattccttg cccatcccat ctctctgcag acggatgtgt gtggccccct cctaggtgcc |
| 2281 | ccacaaccag gaccaagatg gggctcccaa aggaggtaag gagaaccttt ggcaggtgct |
| 2341 | taggacactg actacctaga aagtagacgc agcagagttg ctcccaagtc gaggctcctc |
| 2401 | agagcaggtg ggtcctgaca gcagtggatt ctcccagcag gatgaggaag gagggtgtgt |
| 2461 | taaccaacca agggagtggg ccccccaccc aggtgtctcc gcaagaccac aaaaagccca |
| 2521 | aagatctatg tgtcactgat cattgtaaat aaagtggacc tgcttttaca gccctgtcac |
| 2581 | taaaaaaaaa aaaaaaaaaa aaaaaaaaaa aaaaaaaaaa aaaaaaaaaa aaaaaaaaaa |

CAD (SEQ ID NO: 123)

| | |
|---|---|
| 1 | gcgcgcccga ggctcctacg ctgccgcgcc cggcttctct ccagcgcccc gcgccgttag |
| 61 | ccacgtggac cgactccggc gcgccgtcct cacgtggttc cagtggagtt tgcagtcctt |
| 121 | cccgcttctc cgtactcgcc cccgcctctg agctcccttc ccatggcggc cctagtgttg |
| 181 | gaggacgggt cggtcctgcg gggccagccc tttggggccg ccgtgtcgac tgccggggaa |
| 241 | gtggtgtttc aaaccggcat ggtcggctac cccgaggccc tcactgatcc ctcctacaag |
| 301 | gcacagatct tagtgctcac ctatcctctg atcggcaact atggcatccc cccagatgaa |
| 361 | atggatgagt tcggtctctg caagtggttt gaatcctcgg gcatccacgt agcagcactg |
| 421 | gtagtgggag agtgctgtcc tactcccagc cactggagtg ccacccgcac cctgcatgag |
| 481 | tggctgcagc agcatggcat ccctggcttg caaggagtag acactcggga gctgaccaag |
| 541 | aagttgcggg aacaggggtc tctgctgggg aagctggtcc agaatggaac agaaccttca |
| 601 | tccctgccat tcttggaccc caatgcccgc cccctggtac cagaggtctc cattaagact |
| 661 | ccacgggtat tcaatacagg gggtgcccct cggatccttg ctttggactg tggcctcaag |
| 721 | tataatcaga tccgatgcct ctgccagcgt ggggctgagg tcactgtggt accctgggac |
| 781 | catgcactag acagccaaga gtatgagggt ctcttcttaa gtaatgggcc tggtgaccct |
| 841 | gcctcctatc ccagtgtcgt atccacactg agccgtgttt tatctgagcc taatccccga |
| 901 | cctgtctttg ggatctgcct gggacaccag ctattggcct tagccattgg ggccaagact |
| 961 | tacaagatga gatatgggaa ccgaggccat aaccagccct gcttgttggt gggctctggg |
| 1021 | cgctgctttc tgacatccca gaaccatggg tttgctgtgg agacagactc actgccagca |
| 1081 | gactgggctc ctctcttcac caacgccaat gatggttcca atgaaggcat tgtgcacaac |
| 1141 | agcttgcctt tcttcagtgt ccagtttcac ccagagcacc aagctggccc ttcagatatg |
| 1201 | gaactgcttt tcgatatctt tctggaaact gtgaaagagg ccacagctgg gaaccctggg |
| 1261 | ggccagacag ttagagagcg gctgactgag cgcctctgtc cccctgggat tcccactccc |
| 1321 | ggctctggac ttccaccacc acgaaaggtt ctgatcctgg gctcaggggg cctctccatt |
| 1381 | ggccaagctg gagaatttga ctactcgggc tctcaggcaa ttaaggccct gaaggaggaa |
| 1441 | aacatccaga cgttgctgat caaccccaat attgccacag tgcagacctc ccaggggctg |
| 1501 | gccgacaagg tctattttct tcccataaca cctcattatg taacccaggt gatacgtaat |
| 1561 | gaacgccccg atggtgtgtt actgactttt gggggccaga ctgctctgaa ctgtggtgtg |
| 1621 | gagctgacca aggccggggt gctggctcgg tatggggtcc gggtcctggg cacaccagtg |
| 1681 | gagaccattg agctgaccga ggatcgacgg gcctttgctg ccagaatggc agagatcgga |
| 1741 | gagcatgtgg ccccgagcga ggcagcaaat tctcttgaac aggcccaggc agccgctgaa |
| 1801 | cggctggggt accctgtgct agtgcgtgca gcctttgccc tgggtggcct gggctctggc |
| 1861 | tttgcctcta acagggagga gctctctgct ctcgtggccc cagcttttgc ccataccagc |
| 1921 | caagtgctag tagacaagtc tctgaaggga tggaaggaga ttgagtacga ggtggtgaga |
| 1981 | gacgcctatg gcaactgtgt cacgtattac atcattgaag tgaatgccag gctctctcgc |
| 2041 | agctctgccc tggccagtaa ggccacaggt tatccactgg cttatgtggc agccaagcta |
| 2101 | gcattgggca tccctttgcc tgagctcagg aactctgtga cagggggtac agcagccttt |
| 2161 | gaacccagcg tggattattg tgtggtgaag attcctcgat gggaccttag caagttcctg |
| 2221 | cgagtcagca caaagattgg gagctgcatg aagagcgttg gtgaagtcat gggcattggg |
| 2281 | cgttcatttg aggaggcctt ccagaaggcc ctgcgcatgg tggatgagaa ctgtgtgggc |
| 2341 | tttgatcaca cagtgaaacc agtcagcgat atggagttgg agactccaac agataagcgg |
| 2401 | atttttgtgg tggcagctgc tttgtgggct ggttattcag tggaccgcct gtatgagctc |
| 2461 | acacgcatcg accgctggtt cctgcaccga atgaagcgta tcatcgcaca tgcccagctg |
| 2521 | ctagaacaac accgtggaca gcctttgccg ccagacctgc tgcaacaggc caagtgtctt |
| 2581 | ggcttctcag acaaacagat tgcccttgca gttctgagca cagagctggc tgttcgcaag |
| 2641 | ctgcgtcagg aactggggat ctgtccagca gtgaaacaga ttgacacagt tgcagctgag |
| 2701 | tggccagccc agacaaatta cctataccta acgtattggg gcaccaccca tgacctcacc |
| 2761 | tttcgaacac ctcatgtcct agtccttggc tctggcgtct accgtattgg ctctagcgtt |
| 2821 | gaatttgact ggtgtgctgt aggctgcatc cagcagctcc gaaagatggg atataagacc |
| 2881 | atcatggtga actataaccc agagacagtc agcaccgact atgacatgtg tgatcgactc |
| 2941 | tactttgatg agatctcttt tgaggtggtg atggacatct atgagctcga gaaccctgaa |
| 3001 | ggtgtgatcc tatccatggg tggacagctg cccaacaaca tggccatggc gttgcatcgg |
| 3061 | cagcagtgcc gggtgctggg cacctcccct gaagccattg actcggctga gaaccgtttc |
| 3121 | aagttttccc ggctccttga caccattggt atcagccagc ctcagtggag ggagctcagt |
| 3181 | gacctcgagt ctgctcgcca attctgccag accgtggggt acccctgtgt ggtgcgcccc |
| 3241 | tcctatgtgc tgagcggtgc tgctatgaat gtggcctaca cggatggaga cctggagcgc |
| 3301 | ttcctgagca gcgcagcagc cgtctccaaa gagcatcccg tggtcatctc caagttcatc |
| 3361 | caggaggcta aggagattga cgtggatgcc gtggcctctg atggtgtggt ggcagccatc |
| 3421 | gccatctctg agcatgtgga gaatgcaggt gtgcattcag gtgatgcgac gctggtgacc |
| 3481 | cccccacaag atatcactgc caaaaccctg gagcggatca aagccattgt gcatgctgtg |
| 3541 | ggccaggagc tacaggtcac aggacccttc aatctgcagc tcattgccaa ggatgaccag |
| 3601 | ctgaaagtta ttgaatgcaa cgtacgtgtc tctcgctcct tccccttcgt ttccaagaca |
| 3661 | ctgggtgtgg acctagtagc cttggccacg cgggtcatca tgggggaaga agtggaacct |
| 3721 | gtggggctaa tgactggttc tggagtcgtg ggagtaaagg tgcctcagtt ctccttctcc |
| 3781 | cgcttggcgg gtgctgacgt ggtgttgggt gtggaaatga ccagtactgg ggaggtggcc |
| 3841 | ggctttgggg agagccgctg tgaggcatac ctcaaggcca tgctaagcac tggctttaag |
| 3901 | atccccaaga agaatatcct gctgaccatt ggcagctata agaacaaaag cgagctgctc |
| 3961 | ccaactgtgc ggctactgga gagcctgggc tacagcctct atgccagtct cggcacagct |
| 4021 | gacttctaca ctgagcatgg cgtcaaggta acagctgtgg actggcactt tgaggaggct |
| 4081 | gtggatggtg agtgcccacc acagcggagc atcctggagc agctagctga gaaaaacttt |
| 4141 | gagctggtga ttaacctgtc aatgcgtgga gctgggggcc ggcgtctctc ttcctttgtc |
| 4201 | accaagggct accgcacccg acgcttggcc gctgacttct ccgtgcccct aatcatcgat |
| 4261 | atcaagtgca ccaaactctt tgtggaggcc ctaggccaga tcgggccagc ccctcctttg |
| 4321 | aaggtgcatg ttgactgtat gacctcccaa aagcttgtgc gactgccggg attgattgat |
| 4381 | gtccatgtgc acctgcggga accaggtggg acacataagg aggactttgc ttcaggcaca |
| 4441 | gccgctgccc tggctggggg tatcaccatg gtgtgtgcca tgcctaatac ccggcccccc |
| 4501 | atcattgacg cccctgctct ggccctggcc cagaagctgg cagaggctgg cgcccggtgc |
| 4561 | gactttgcgc tattccttgg ggcctcgtct gaaaatgcag gaaccttggg caccgtggcc |
| 4621 | gggtctgcag ccgggctgaa gctttacctc aatgagacct tctctgagct gcggctggac |
| 4681 | agcgtggtcc agtggatgga gcatttcgag acatggccct cccacctccc cattgtggct |
| 4741 | cacgcagagc agcaaaccgt ggctgctgtc ctcatggtgg ctcagctcac tcagcgctca |
| 4801 | gtgcacatat gtcacgtggc acggaaggag gagatcctgc taattaaagc tgcaaaggca |
| 4861 | cggggcttgc cagtgacctg cgaggtggct ccccaccacc tgttcctaag ccatgatgac |
| 4921 | ctggagcgcc tggggcctgg gaagggggag gtccggcctg agcttggctc ccgccaggat |
| 4981 | gtggaagccc tgtgggagaa catggctgtc atcgactgct ttgcctcaga ccatgctccc |
| 5041 | cataccttgg aggagaagtg tgggtccagg cccccacctg ggttcccagg gttagagacc |
| 5101 | atgctgccac tactcctgac ggctgtaagc gagggccggc tcagcctgga cgacctgctg |
| 5161 | cagcgattgc accacaatcc tcggcgcatc tttcacctgc ccccgcagga ggacacctat |
| 5221 | gtggaggtgg atctggagca tgagtggaca attcccagcc acatgccctt ctccaaggcc |
| 5281 | cactggacac cttttgaagg gcagaaagtg aagggcaccg tccgccgtgt ggtcctgcga |
| 5341 | ggggaggttg cctatatcga tgggcaggtt ctggtacccc cgggctatgg acaggatgta |
| 5401 | cggaagtggc cacagggggc tgttcctcag ctcccaccct cagcccctgc cactagtgag |
| 5461 | atgaccacga cacctgaaag accccgccgt ggcatcccag ggcttcctga tggccgcttc |
| 5521 | catctgccgc cccgaatcca tcgagcctcc gacccaggtt tgccagctga ggagccaaag |
| 5581 | gagaagtcct ctcggaaggt agccgagcca gagctgatgg gaacccctga tggcacctgc |
| 5641 | taccctccac caccagtacc gagacaggca tctccccaga acctggggac ccctggcttg |
| 5701 | ctgcaccccc agacctcacc cctgctgcac tcattagtgg gccaacatat cctgtccgtc |
| 5761 | cagcagttca ccaaggatca gatgtctcac ctgttcaatg tggcacacac actgcgtatg |
| 5821 | atggtgcaga aggagcggag cctcgacatc ctgaagggga aggtcatggc ctccatgttc |
| 5881 | tatgaagtga gcacacggac cagcagctcc tttgcagcag ccatggcccg gctgggaggt |
| 5941 | gctgtgctca gcttctcgga agccacatcg tccgtccaga agggcgaatc cctggctgac |
| 6001 | tccgtgcaga ccatgagctg ctatgccgac gtcgtcgtgc tccggcaccc ccagcctgga |
| 6061 | gcagtggagc tggccgccaa gcactgccgg aggccagtga tcaatgctgg ggatggggtc |
| 6121 | ggagagcacc ccacccaggc cctgctggac atcttcacca tccgtgagga gctgggaact |
| 6181 | gtcaatggca tgacgatcac gatggtgggt gacctgaagc acggacgcac agtacattcc |
| 6241 | ctggcctgcc tgctcaccca gtatcgtgtc agcctgcgct acgtggcacc tcccagcctg |
| 6301 | cgcatgccac ccactgtgcg ggccttcgtg gcctcccgcg gcaccaagca ggaggaattc |
| 6361 | gagagcattg aggaggcgct gcctgacact gatgtgctct acatgactcg aatccagaag |
| 6421 | gaacgatttg gctctaccca ggagtacgaa gcttgctttg gtcagttcat cctcactccc |
| 6481 | cacatcatga cccgggccaa gaagaagatg gtggtgatgc acccgatgcc ccgtgtcaac |
| 6541 | gagataagcg tggaagtgga ctcggatccc cgcgcagcct acttccgcca ggctgagaac |
| 6601 | ggcatgtaca tccgcatggc tctgttagcc accgtgctgg gccgtttcta gggcctggct |
| 6661 | tcctcagcct cttctcttta ggcccagctg ctgggcaagg aattccagtg cctcctacgg |
| 6721 | gggcagcaca cttagatatt cctggacatc cagatagctc acatgtgctg accacacttc |
| 6781 | aggctctgga ctggagctct ctggcatggg ggtggggcct cagatgctgg ggcccagtct |
| 6841 | gccccatctt cattcctgca ccttaaacct gtacagtcat ttttctactg acttaataaa |
| 6901 | cagccgagct gtcccttgat gctgaaaaaa aaaaaaaaaa aa |

CENPO (SEQ ID NO: 124)

| | |
|---|---|
| 1 | gagtgcctca cctcgaggac cactttgcgc atgcgcccca gctcttggag gtaagcggct |
| 61 | gtgtgcgggt ggtcgcggtg agtgtgcaag gccgcggtgg ccgcgtgaca agcctgcgct |
| 121 | accagtgcgc ccgccggcca ggagaacgga gcttgtgata gatcctttcg taacaccaag |
| 181 | tattgtacca ggacctgcgg ctccgcccca gaggccgcca tcttcctgac cacccgaaag |
| 241 | gccggaccta ctccccggtg catcttggga tcagggcggg gccctgagcg ccgccatgct |
| 301 | tttgtacggc aggatcgcaa agcacgccgg gaccggttgg tttggttttg aagacgtgga |
| 361 | tggcgggaat tctcgcttct ggcctgggtg ttttagctca cttggaaagg ctagagaccc |
| 421 | aagtgagcag atcccgtaaa cagtctgaag agctgcagag cgtgcaggcc caggaaggtg |
| 481 | ctcttggaac caagattcat aaactaaggc gtctgcgaga tgagctgagg gctgtggtgc |
| 541 | ggcaccggcg agccagcgtg aaagcatgta ttgccaatgt agaacccaac caaacagtgg |
| 601 | agatcaatga gcaagaagca ttggaagaga aattggaaaa tgtgaaagcc attctgcagg |
| 661 | catatcattt tacaggcctc agtggtaaac tgaccagccg aggagtttgt gtctgcatca |
| 721 | gtactgcttt tgaggggaac ctattggatt cctattttgt ggaccttgtc atacagaaac |
| 781 | cactccggat acatcaccat tcagtcccag tcttcattcc cctggaagag atagctgcaa |
| 841 | aatatttaca gaccaacatc cagcacttcc tgttcagtct ctgcgagtac ctgaatgctt |
| 901 | actctgggag gaagtaccag gcagaccggc ttcagagtga ctttgcagcc ctcctgactg |
| 961 | ggcccttgca gagaaaccca ctgtgtaact tgctgtcatt tacttacaaa ctggatccag |
| 1021 | ggggtcagtc cttcccgttc tgtgctagat tgctgtataa ggacctcaca gcaactcttc |
| 1081 | ccactgacgt caccgtgaca tgtcaaggag tggaagtatt atccacttca tgggaggagc |
| 1141 | aacgagcatc tcatgaaact ctgttctgta cgaagccctt gcatcaagtg tttgcctcat |
| 1201 | ttacaagaaa aggagaaaag ttggatatga gtctggtctc ctaatagatt gttttcactg |
| 1261 | cactgggagc acatcagaga aataaatccc ccctcccctg ccaggtgaaa ggaaatattg |
| 1321 | cactttctgt tctcatgact aaggggacag gagttccaga agaacctttc aagatgatca |
| 1381 | ggaacaccag gacgagggcc gtctcacctc actcggacca catggagacc tcccttcaaa |
| 1441 | atgggagcca tgtcctgccc caccaagccc tgtctgaagt ggagcttccc cgcctgtgct |
| 1501 | ccctccacag tcccggaaag cccagcggca aaggcagctt tgtcccagct ctgccaccct |
| 1561 | cctgctcaca gtggtcaggg cccctcaggg gcaaggacgg cagggattgg aacgagggct |
| 1621 | ctggaaggac tgttcagccc tatgcctaag acccctatgc tggggacact acaggcacac |
| 1681 | acaggaatag cagggccacc ctcagagctc acacatccac gaacaaatga aggctgagga |
| 1741 | ggtttctaaa cctaaagtcc atgagtgtgc acttcaatcc aggaaggtcg ggacttcctt |
| 1801 | cagtttcaaa aaataaattc tcccttccgg tttggactgt tgcaggctcg aggccattca |
| 1861 | ggagttgtcc accacctggt ggggcagtgt gacagagggg ccattgggga aggtggctag |
| 1921 | cttatcccgc cccttcaaga agaaggtcag cagctccccc ttccccttca caaagatggg |
| 1981 | gcctcgcctc acaaagcgga agccgtactc tcggaggatg acttgggttt cttctaccac |
| 2041 | ctggagaggg agggggagca agaacgtggc gttacggggg gagcctagac tgagggcggg |
| 2101 | tgggggcttt gggtggttgg agccgagcac tgatccatgg gtcccaagca gtacgggaca |
| 2161 | ctccccaaac ctcccagggc caagcccttc cacccgtggc gagcagcggg tgggaaggag |
| 2221 | aaccctggag tgactggctg ggggcctcct ctcatccaga gacttctctc ctaggatggc |
| 2281 | catggtcacc tgggtggcag cactgttacc tggaaactgc cactgcctgc tcttctgtcc |
| 2341 | ctttgcccct ttcgtggagc ttttctgcca gacgccactg agacagatca caaggtatta |
| 2401 | gaaggttcat acccaaaggt aggccatatg catctagaac ttcagcccag attttgtgga |
| 2461 | tgggtggaag tgtttcttcc tgtgctgagg ctagctattg cagagattct tttccacttg |
| 2521 | ccccacgtct ctgcctctgg acttactgtt cagggccagg gtgggaggca ggggcacgtg |
| 2581 | ggaaagcact gttccggttt tgttctcatg ccgagtctga gcacgtgcca gctgtgccac |
| 2641 | tggacatacc tgaatgttgc ccatgacccc cgtggactcc atcctgctgg ctacattgac |
| 2701 | tgtattgccc cagatgtcgt agtgtggttt ccgggctccg atgaccccag ccagaacccc |
| 2761 | gcctttgttc atgcctaggg tagaggcata aagttcagca cagccacagg ccacaccttg |
| 2821 | ttatgggcct cagaagccat ctcctctcca gacctgtacc acaaagctcc taatgtaaca |
| 2881 | catcattgtc ctcattcaac ttggctgtat gctattggag ggtggaaatc acatctcctg |
| 2941 | tttatccgtg tgcttgttag gtgtcagccg ccaccccccc cccatatgca gatttactcg |
| 3001 | gcatggtagt ggccagcttc taacacagct ggtatttcaa gtctcctggg acctcactca |
| 3061 | ggaatgatac cccctcagta gaagcagcag gtgatcttaa ctcctttcaa agagcaggcc |
| 3121 | tgtctgggaa gccatgtcct cagcaggcac agcaacccct ctggaaatgg atcacaaact |
| 3181 | cacttctcag ccaggcaggc caagcttcta ttgtaacagt aggcacagta tagtcggatc |
| 3241 | atcacatcag ctgggttttt ggtttagtca tctagagtcg tctggactaa aggtctttca |
| 3301 | ggtctccttg ccctgtgagt gcgtgaacct ccccacccga attgcctcag ttgtcctgag |
| 3361 | cctcatgtct ctcctggtgg tgggccaggc ccctgcatgg gaagggagcc tgctgcgggg |
| 3421 | caggccagct gggggtgctc acctatgcgc agcatgaagt tattgaagga ctggttgttg |
| 3481 | atgttggtga gcgtatcctt catggccagc gcgaagtcgg ccaggtcagc caggtgctgc |
| 3541 | cagcgctctc tctcggactt gtcttcctgt gccaggggac cgtggagaaa gtgtcagggg |
| 3601 | ccgctcactg cagcagcctg ctctgctgcc ttccctggca gtgttctggg ggtggattcc |
| 3661 | ctacacctag atgttcaagg ccttactttt cctcccacaa aggagtcgca gccacgctag |
| 3721 | ctctgacttg ccactgtgac aaagttcacg tagcaggtct aggcaaagac tgggcaattg |
| 3781 | agcagaggag acggacctgt gagtctgacc acgaggcgga ccccttcacc ttggctgggc |
| 3841 | ctggtcctgg tccttaggtt ttgtcaggtt gtccttgttt ggatccctca actaggtgat |
| 3901 | aagcactgga gggggatgac ccgccttgga cgtgtttctt taacctcatc catataatag |
| 3961 | ggccgtggga tggttgtaga ggtaaagcag gatgatggtg ttttaagacc agagcttggg |
| 4021 | accagggctc ctacacctaa ttttctctcc tggtagctga acaaaggtct aaattagctt |
| 4081 | aacaaaagaa caggctgccg tcagccagag ttctgaaggc catgctttca gtttcccttg |
| 4141 | ttgacaattg ctctccagtt cctatgaaag cacagagcct tagggggcct ggccacagaa |
| 4201 | cacaaccatc ttaggcctga gctgtgaaca gcagggggtt gtgtgtctgt tctgtttctc |
| 4261 | tgcttgccga actttctcaa taaaccctat ttcttattta taaaaaaaaa aaaaaa |

TOP1MT (SEQ ID NO: 125)

| | |
|---|---|
| 1 | gctcgggcct tcccggcgtc tccgcgcagg cctcggggaa gcggggtccg ggggagccgt |
| 61 | ggtgcggtgg gaccgcgtgg gtcctggaag agctgcagag gagagtgacg gctttggatg |
| 121 | cgctttgccc cagggccttt cttcccggag ttggcctttt ccctgccctt ctcttctcct |
| 181 | ggcgtggtga cctgcctccc ttctcctgga tcgctttgct ggcagccacc ttgtaacacc |
| 241 | tcaggtggga gaaggagaag cacgaagacg gggtgaagtg gagacagctg gagcacaagg |
| 301 | gcccgtactt cgcaccccca tacgagcccc ttcccgacgg agtgcgtttc ttctatgaag |
| 361 | gaaggcctgt gagattgagc gtggcagcgg aggaggtcgc cactttttat gggaggatgt |
| 421 | tagatcatga atacacaaca aaggaggttt tccggaagaa cttcttcaat gactggcgaa |
| 481 | aggaaatggc ggtggaagag agggaagtca tcaagagcct ggacaagtgt gacttcacgg |
| 541 | agatccacag atactttgtg gacaaggccg cagcccggaa agtcctgagc agggaggaga |
| 601 | agcagaagct aaaagaagag gcagaaaaac ttcagcaaga gttcggctac tgtattttag |
| 661 | atggtcacca agaaaaaata ggcaacttca agattgagcc gcctggcttg ttccgtggcc |
| 721 | gtggcgacca tcccaagatg gggatgctga agagaaggat cacgccagag gatgtggtta |
| 781 | tcaactgcag cagggactcg aagatccccg agccgccggc ggggcaccag tggaaggagg |
| 841 | tgcgctccga taacaccgtc acgtggctgg cagcttggac cgagagcgtt cagaactcca |
| 901 | tcaagtacat catgctgaac ccttgctcga agctgaaggg ggagacagct tggcagaagt |
| 961 | ttgaaacagc tcgacgcctg cggggatttg tggacgagat ccgctcccag taccgggctg |
| 1021 | actggaagtc tcgggaaatg aagacgagac agcgggcggt ggccctgtat ttcatcgata |
| 1081 | agctggcact gagagcagga aatgagaagg aggacggtga ggcggccgac accgtgggct |
| 1141 | gctgttccct ccgcgtggag cacgtccagc tgcacccgga ggccgatggc tgccaacacg |
| 1201 | tggtggaatt tgacttcctg gggaaggact gcatccgcta ctacaacaga gtgccggtgg |
| 1261 | agaagccggt gtacaagaac ttacagctct ttatggagaa caaggacccc cgggacgacc |
| 1321 | tcttcgacag gctgaccacg accagcctga acaagcacct ccaggagctg atggacgggc |
| 1381 | tgacggccaa ggtgttccgg acctacaacg cctccatcac tctgcaggag cagctgcggg |
| 1441 | ccctgacgcg cgccgaggac agcatagcag ctaagatctt atcctacaac cgagccaacc |
| 1501 | gagtcgtggc cattctctgc aaccatcagc gagcaacccc cagtacgttc gagaagtcga |
| 1561 | tgcagaatct ccagacgaag atccaggcaa agaaggagca ggtggctgag gccagggcag |
| 1621 | agctgaggag ggcgagggct gagcacaaag cccaagggga tggcaagtcc aggagtgtcc |
| 1681 | tggagaagaa gaggcggctc ctggagaagc tgcaggagca gctggcgcag ctgagtgtgc |
| 1741 | aggccacgga caaggaggag aacaagcagg tggccctggg cacgtccaag ctcaactacc |
| 1801 | tggaccccag gatcagcatt gcctggtgca agcggttcag ggtgccagtg gagaagatct |
| 1861 | acagcaaaac acagcgggag aggttcgcct gggctctcgc catggcagga gaagactttg |
| 1921 | aattctaacg acgagccgtg ttgaaacttc ttttgtatgt gtgtgtgttt ttttcactat |
| 1981 | taaagcagta ctggggaatt ttgtacaata aaatgtgtgc aagtgcttgt acatcactag |
| 2041 | aaaaa |

IL34 (SEQ ID NO: 126)

| | |
|---|---|
| 1 | catcagacgg gaagcctgga ctgtgggttg ggggcagcct cagcctctcc aacctggcac |
| 61 | ccactgcccg tggcccttag gcacctgctt ggggtcctgg agccccttaa ggccaccagc |
| 121 | aaatcctagg agaccgagtc ttggcacgtg aacagagcca gatttcacac tgagcagctg |
| 181 | cagtcggaga aatcagagaa agcgtcaccc agccccagat tccgaggggc ctgccaggga |
| 241 | ctctctcctc ctgctccttg gaaaggaaga ccccgaaaga cccccaagcc accggctcag |
| 301 | acctgcttct gggctgccat gggacttgcg gccaccgccc cccggctgtc ctccacgctg |
| 361 | ccgggcagat aagggcagct gctgcccttg gggcacctgc tcactcccgc agcccagcca |
| 421 | ctcctccagg gccagccctt ccctgactga gtgaccacct ctgctgcccc gaggccatgt |
| 481 | aggccgtgct taggcctctg tggacacact gctggggacg gcgcctgagc tctcaggggg |
| 541 | acgaggaaca ccaccatgcc ccggggcttc acctggctgc gctatcttgg gatcttcctt |
| 601 | ggcgtggcct tggggaatga gcctttggag atgtggccct tgacgcagaa tgaggagtgc |
| 661 | actgtcacgg gttttctgcg ggacaagctg cagtacagga gccgacttca gtacatgaaa |
| 721 | cactacttcc ccatcaacta caagatcagt gtgccttacg agggggtgtt cagaatcgcc |
| 781 | aacgtcacca ggctgagggc ccaggtgagc gagcgggagc tgcggtatct gtgggtcttg |
| 841 | gtgagcctca gtgccactga gtcggtgcag gacgtgctgc tcgagggcca cccatcctgg |
| 901 | aagtacctgc aggaggtgga gacgctgctg ctgaatgtcc agcagggcct cacggatgtg |
| 961 | gaggtcagcc ccaaggtgga atccgtgttg tccctcttga atgccccagg gccaaacctg |
| 1021 | aagctggtgc ggcccaaagc cctgctggac aactgcttcc gggtcatgga gctgctgtac |
| 1081 | tgctcctgct gtaaacaaag ctccgtccta aactggcagg actgtgaggt gccaagtcct |
| 1141 | cagtcttgca gcccagagcc ctcattgcag tatgcggcca cccagctgta ccctccgccc |
| 1201 | ccgtggtccc ccagctcccc gcctcactcc acgggctcgg tgaggccggt cagggcacag |
| 1261 | ggcgagggcc tcttgccctg agcaccctgg atggtgactg cggatagggg cagccagacc |
| 1321 | agctcccaca ggagttcaac tgggtctgag acttcaaggg gtggtggtgg gagcccccct |
| 1381 | tgggagagga cccctgggaa gggtgttttt cctttgaggg ggattctgtg ccacagcagg |
| 1441 | gctcagcttc ctgccttcca tagctgtcat ggcctcacct ggagcggagg ggacctgggg |
| 1501 | acctgaaggt ggatggggac acagctcctg gcttctcctg gtgctgccct cactgtcccc |
| 1561 | ccgcctaaag ggggtactga gcctcctgtg gcccgcagca gtgagggcac agctgtgggt |
| 1621 | tgcaggggag acagccagca cggcgtggcc attctatgac cccccagcct ggcagactgg |
| 1681 | ggagctgggg gcagagggcg gtgccaagtg ccacatcttg ccatagtgga tgctcttcca |
| 1741 | gtttcttttt tctattaaac accccacttc ctttggaaaa aaaaaaaaaa aaa |

NEBL (SEQ ID NO: 127)

| | |
|---|---|
| 1 | cctgcgcggc ggcggcggcg aggcggggga gcgagtgagc gcgaggggcg ggcgcgagtg |
| 61 | actgtgtgag tcacccgtac ctggagtgcg agcgacgcag agccagcggc gcggagccgg |
| 121 | agccggagcc gagacccagc gcctgcgagc ccgagagcgc ggccggcccc aggcgccagg |
| 181 | ccccgtcgcc ctccccgtgc actcacccgt ggcccggcgc cgactcccta cccggcgccc |
| 241 | gccgcccgca gccctcccgc ctgccaggag gcggtgcggg gctcgccggg ggatgtcaca |
| 301 | gcggctcctg ggagccagca gccgccgccg ccgccgcccc cgggaaccgc gatcatgaac |
| 361 | ccccagtgcg cccgttgcgg aaaagtcgtg tatcccaccg agaaagtcaa ctgcctggat |
| 421 | aagtattggc ataaaggatg tttccattgt gaggtctgca agatggcact caacatgaac |
| 481 | aactacaaag gctatgaaaa gaagccctat tgtaatgcac actacccgaa gcagtccttc |
| 541 | accacggtgg cagatacacc tgaaaatctt cgcctgaagc agcaaagtga attgcagagt |
| 601 | caggtcaagt acaaaagaga ttttgaagaa agcaaaggga ggggcttcag catcgtcacg |
| 661 | gacactcctg agctacagag actgaagagg actcaggagc aaatcagtaa tgtaaaatac |
| 721 | catgaagatt ttgaaaaaac aaaggggaga ggctttactc ccgtcgtgga cgatcctgtg |
| 781 | acagagagag tgaggaagaa cacccaggtg gtcagcgatg ctgcctataa aggggtccac |
| 841 | cctcacatcg tggagatgga caggagacct ggaatcattg ttgcacctgt tcttcccgga |
| 901 | gcctatcagc aaagccattc ccaaggctat ggctacatgc accagaccag tgtgtcatcc |
| 961 | atgagatcaa tgcagcattc accaaatcta gacctaccga gccatgtacg attacagtgc |
| 1021 | ccaggatgaa gacgaggtct cctttagaga cggcgactac atcgtcaacg tgcagcctat |
| 1081 | tgacgatggc tggatgtacg gcacagtgca gagaacaggg agaacaggaa tgctcccagc |
| 1141 | gaattacatt gagtttgtta attaattatt tctccctgcc ctttgagctt tattctaatg |
| 1201 | tatcccaaac ctaatctttt taaaagatag aagatacttt taagacaact tggccattat |
| 1261 | tttacaatga tgtatccttc ctttgacaat tagacacaca ggtaccagga agaaggaatg |
| 1321 | acctctgggc tgaaaacagc agcattttca gtaattccta caaacaaaaa tctttgtgtc |
| 1381 | tggacacctg gtgctgctaa ttgtgttcat ggtttccttt gattggctat tgaacccttc |
| 1441 | tgggaaatgt atttttgtag actttaatag agaagttgat tgtcccttaa atgtagtgtg |
| 1501 | tgtttgaaac ttcttagctg tcactttgga atcaccccaa gccaattctc ttaactctgt |
| 1561 | aatgcagcca ataatttcaa acccgttttg cttttgagtc atgaggcaat ttccaatatt |
| 1621 | agtgaaaatt gcccaatata ataagtgtaa acagtggcag aaggacagtc tggttaaaat |
| 1681 | tatattgact ggtggcctta gggatctaga aacttctact aaacagagaa atttccttgt |
| 1741 | tccctaggct gactggtatc tatttatttc tcatttgtac caaggcatct cctactctcc |
| 1801 | atttatattc tatggaccca agtctatgct cagttccaca gaatgtcagg accaaataac |
| 1861 | ttcacagcta ctctgcaaag ggcaaattat aatgtcattg atataatttc cctagtagca |
| 1921 | tttaccctgt tgcatgtcat gtagattcaa gcttctgtaa cataggcagc tgcactgcgc |
| 1981 | gttcctatta ttgaagcaaa aagggtgact gatacctaaa agccctttct tcctctagtc |
| 2041 | gccagctcat cagaaaaaca tactttgaaa agatgcttga gattttcctg ctgcatcgca |
| 2101 | ctctagtttt gaaggattta catcttagga aataacatgt atactctagt aaataagcga |
| 2161 | tttaggtgtt ccattgaaca gctttgatta acttaatgcc accattgatt tcaaagtgaa |
| 2221 | gaaaatgtaa cagaagccag tgaagcaatg gaagctggag tgtgactgga aaaatactca |
| 2281 | gcaaacaaag ttaccaattc catacagaga tgatctggta tcttcttttg gaaaatggta |
| 2341 | ttcaaattct ggaatggaaa tctagccacc aaaacgggtt aatcaaaaga cgtccttttc |
| 2401 | catttttttt tgcttttatt ttctaaatca tttttaaggg aatgaaacag gaatgtcatc |
| 2461 | agagattttt tagtacaggc ccaagagcct gttctctaag aaagaaattg ttgccatgtt |
| 2521 | ttgattttcg aataagtgac tttgcaggct ttatgctagc ccttgctggt gggtcttgaa |
| 2581 | atttcatcca gagtctgcag tccaggtcac caagccagcg gcacccgtcg gcaaccctgt |
| 2641 | gtttttctga ttgtgccgtt tactgtgacc tgcaacgggg tggcattcac ttagggtctg |
| 2701 | acttcacagc tatgacaaaa ccgaaaaagc aaaactgcaa aaaagtacta agatgtacgg |
| 2761 | gtcttgggga tatctgcctt atatgttata ttcaaggaaa ttaacaaaac atcctgtaaa |
| 2821 | acatcgttta aggaaacgtt tactagtcca aaggccaaag ctaatttatt tccactttag |
| 2881 | aaaagttagc acatgctttt gaaaatctgt gatttcattt tattaggcta aaagggtaaa |
| 2941 | taggctttat tacactgaag ctgcatctat atgtcactga cataaagttg aaaaaataaa |
| 3001 | tgcaggcaaa taactagaga cttcttttaa gggggtttgg ctggttttct ctcactgaaa |
| 3061 | tggccagtcg tgattaaagt gataaaaccc catatctgtt ttggtatatt gtacacaaac |
| 3121 | ctacaaaaat aaactgaact tgcaatattt ttgcaaaaaa atctgtcgtt aaaactgagg |
| 3181 | ataaaatacc tgctcaattt tattttacta agtatatatt tacatttcac ccaggcaggc |
| 3241 | cattttcttt tgtgattata agaaagagta gttgttgatt aaattttcag actaaatata |
| 3301 | ggacaggtac aattttggat aaatagcaca tttataagaa ccgcaatgaa aactgacttg |
| 3361 | aaataatgct tgtaatcagg aaagtaattt catccaccga tttcaaaacc agattcactg |
| 3421 | agcataaaag tcaatacata tttgaggaat aagtctccta aaattttaag cttcacgtaa |
| 3481 | taatgtttgc atagcaaaat atttctgctt caagccttta ggaattaaga tctgatcaga |
| 3541 | atttaactaa agggtagttg ttttacaatg aagactaaaa ctgaacaaga tgttgcatgc |
| 3601 | tcttgaggcc ataatttggt agtgttggca gttgttaata aagcttgtca ggatgttaag |
| 3661 | catctcagga gaaatattgg aaaattatat gtataaaacc aaagtgctat ttttaaaagc |
| 3721 | atcatttaaa aaaaaatgac atgcctgaac aacttttcca ctttccacgt gcttccctcc |
| 3781 | cacctttggt ttggcaacag gtatctcgtg catgaagctg acagctaaag aagattttaa |
| 3841 | aaattgagtt aaagatgact gtgtaaatgt ccaagcacag agagcatgca cctgactttc |
| 3901 | taaagtttga tgtgttctca agcctgacag aagcacaagg aacagtttga tacactttta |
| 3961 | aaaggttctg aaaacaaagc tgtataggga tcctctctct cttgagcaaa gtatagcaac |
| 4021 | agaatatatt gcttttgttg taagcttttg tagtacatgt ttttactaat aattcttgtt |
| 4081 | ctctagaaag ctttctattt ctaacctatg gcaaaatgaa tccttcatgt cttcttgtta |
| 4141 | ttgtttacac acttgcagtg tagcccagtt tgaaatattt atttggttat caactgccca |
| 4201 | tggaggaggc tcttgatgat cccaggtctc ctcgacctcc atacaccaca caggcatttg |
| 4261 | taagcacagt ttccacaagc accttgtagg aatatggata agattagacc agcccctctc |
| 4321 | tgtccactgg gtttatttct tgaagaagat gcagatctgg tttttccaat gtgccacagt |
| 4381 | ctttccttat cctctccatg ctgagcttga caacactctg ggaatgagga acaagacttt |
| 4441 | ttctaaaaag atagtggaag ttcaagggat gtacctcgtt ttcaggttca tccatctcca |
| 4501 | gtggaatgtt ttcaataaaa gatgaagaaa atgtgtgtga tctttaataa cacatcccta |
| 4561 | tagaaagtgg ataaaagata taccaaaact gtaatacaga tatatacaaa tataggtgcc |
| 4621 | tttttgatta ctcttgtttg tctagtatgc tcttggaaag aaaaccaagc aagcaagttg |
| 4681 | ctgcctattc tatagtaata ttttattaca catgattgat atttttgtgg tagggaagtg |
| 4741 | ggatgctcct cagatattaa aggtgttagc tgattgtatt ttatctctaa agatttagaa |
| 4801 | ctttagaaaa tgccgacttc ttccatctat ttctgaaagg ttctttgtgg atttatatag |
| 4861 | agttgagcta tataaacatt aactttagat ttgggattta aaatgcctat tgtaagatag |
| 4921 | aataattgtg aggctggatt cactacacaa gatgaacttc acttcataaa ttaattatac |
| 4981 | cttagcgatt tgcttctgat aatctaaaag tggctagatt gtggttgttt tggttaaggt |
| 5041 | gatatggagg tgggagagct tttagttaag taagaagcta tgtaaactga caaggatgct |
| 5101 | aaaataaaag tctctgaagt attccatgcc ttttggaccc tttcctcgca actaactgtc |
| 5161 | aactgttgat caaaaaagtc aaggcattgt atgttgcttc tgtggttatt attctgtgat |
| 5221 | gcttagacta cttgaaccca taaacttgga agaatctttg agcaaatttt ctcagttgtc |
| 5281 | tgtatgactt cagtatattc ctgggaatgc cataggattt tttgtgcttg atacatggta |
| 5341 | tccagtttgc atagtatcac ttctttgtaa tccagttgct gttaagaatg atgtacttta |
| 5401 | aaggaaaaga gaaaactgca tcacagtccc attctccagt gtccatgcaa tgaattgctg |
| 5461 | agcatttagg aagcagcacc aagtctatta caggcatggt gtgaaacttg atgtttgacc |
| 5521 | tgtgatcaaa attgaaccat tgtacagttt ggcttctgtt tgcttcaaaa tatgtagaat |
| 5581 | tgtggttgat gattaatttg cgagactaac tttgagagtg taacagtttt gaagaaaaca |
| 5641 | ttgaatgttt tgcaaatgaa ggggcttcac ggaatgttac aatgttacta atataatttg |
| 5701 | gcttttgtta tgcaaattgt taacaccagc tattaaaata tattttagta gaaatgcttt |
| 5761 | aattcatatt tttttcctct acactgtgaa tctttaagcc ttggtggact agagcaacat |
| 5821 | cgtgctgccc aaaggactaa cctatgcaaa ctagttcaca ttttagtgga tgtcgcagtt |
| 5881 | aatgtgtaat aagacattat ttcccctgca taatgtacaa cagcattgaa atgacacatt |
| 5941 | aagcctagca tcacattgta tagtacagtc actcacaaac ccttcaaggc taccctaatc |
| 6001 | attaacatta atatttgttt aaaagcaaat caccgattta tctattgaaa ctacttaaat |
| 6061 | gacggcaaac caggaatgac agatggctgt gtcagcaatg gctttaatgt gttccctgca |
| 6121 | agtggtctcc tatgatagaa ctgcgttctc aaatgcactc tcttcagggt cttaatattc |
| 6181 | tgtgttttct ctctgtattt gtaaaacatt ataacacatt aatttcctat ctctacacat |
| 6241 | ttggtttgct taaataaatg caggatataa aaaaaatggt tcacttcttg gctctcaccg |
| 6301 | tggtttcttg gagcatgggt tgttagatgc aagcaatgca ccctaataat accccgggtc |
| 6361 | tgagatttaa catgacaact cacatcaaat cgcatcagag gtgtgtgctg ccttcagtgc |
| 6421 | atttacattg gtgaatcagt caagatattt tcctccccca aataaactta gttgtaagtg |
| 6481 | ataacaatat tatgcttctc caagctcagt atctttctga ttttatatca aagtaccgca |
| 6541 | acaatgcatc attgtagtta atttatttca agaataaatt cctcatatgt cctcaatagt |
| 6601 | acaattctaa ttttcttcta ttcataagat gaaagaaatg gtttggagca tagaatagaa |
| 6661 | agtgcacaaa ttgagtacat aaaatgggaa gcaactgatt tctcagctaa gaaaggctca |
| 6721 | tttatcacag aacacaattg cttttctccc cccactacgc ttcccataat tgaaaaagtg |
| 6781 | agtccctatt tttcacactc atataaatct atgcgatttg gatgctagtc ttattgtatt |
| 6841 | attttgtaaa actttctctt tggctcataa tccttcctaa ttgtaaattg ataaactttg |
| 6901 | cggatgacat ctgctcgtag aataaacact tcttccaaaa aaaaaaaaaa aaaa |

FTSJD1 (SEQ ID NO: 128)

| | |
|---|---|
| 1 | agtgggactt gagtgcctcc tggtccctgt ctgccggcat tcgcggctgc ggggcccgga |
| 61 | ggtgggactg gcttcccggt gccgcgaggg cgggtccgga cagccttccc cccagtccgg |
| 121 | cgcaccatct ccctgccttg tggctggagg cgccgcggac ccaaagggag ggaccatccc |
| 181 | gggaagcagc cccgagagcg gaagtgcaga atggcttcct cgagagagta aagtgcagcc |
| 241 | tctccagaca ctggggcccc agtgggcgtg ggcgaaggta atccaggcct gggtacgatt |
| 301 | ccgggccctc cttcgacttc ccagcggttg ctggtaggag gagttggcgg aagcacttgg |
| 361 | aactccttta taagtgtcag ctgtgagatt ttaatttgat ttgaaaatga gtaagtgcag |
| 421 | aaagacacca gttcagcagc tagcaagtcc cgcgtcattc agcccagata ttcttgctga |
| 481 | catttttgaa ctctttgcca agaacttttc ttatggcaag ccacttaata atgagtggca |
| 541 | gttaccagat cccagtgaga ttttcacctg tgaccacact gaacttaatg catttcttga |
| 601 | tttgaagaac tccctaaatg aagtaaaaaa cctactgagt gataagaaac tggatgagtg |
| 661 | gcatgagcac actgctttca ctaataaagc ggggaaaatc atttctcatg ttagaaaatc |
| 721 | tgtgaatgct gaactttgta ctcaagcatg gtgtaagttc catgagattt tgtgcagctt |
| 781 | tccacttatt ccacaggaag cttttcagaa tggaaaactg aattctctac acctttgtga |
| 841 | agctccagga gcttttatag ctagtctcaa ccactactta aaatcccatc ggtttccttg |
| 901 | tcattggagt tgggtagcga atactctgaa tccataccat gaagcaaatg acgacctcat |
| 961 | gatgattatg gatgaccggc ttattgcaaa taccttgcac tggtggtact ttggtccaga |
| 1021 | taacactggt gatatcatga ccctgaaatt cttgactgga cttcagaatt tcataagcag |
| 1081 | catggctact gttcacttgg tcactgcaga tgggagtttt gattgccaag gaaacccagg |
| 1141 | tgaacaagaa gctttagttt cttctttgca ttactgtgaa gttgtcactg ctctgaccac |
| 1201 | tcttggaaac ggtggctctt ttgttctaaa gatgtttact atgtttgaac attgttccat |
| 1261 | aaacttgatg tacctgctaa actgttgttt tgaccaagtc catgttttca aacctgctac |
| 1321 | tagcaaggca ggaaactccg aagtctatgt ggtttgcctc cactataagg ggagagaggc |
| 1381 | catccatcct ctgttatcta agatgacctt gaattttggg actgaaatga aaaggaaagc |
| 1441 | cctttttccc catcatgtga ttcctgattc ttttcttaag agacatgaag aatgttgtgt |
| 1501 | gttctttcat aaatatcagc tagagactat ttctgaaaac attcgtctat ttgagtgcat |
| 1561 | gggaaaggcg gaacaagaaa agctgaataa tttaagggat tgtgctatac aatattttat |
| 1621 | gcaaaaattt caactgaaac atctttccag aaataattgg ctagtaaaaa aatctagtat |
| 1681 | tggttgtagt acaaatacaa aatggtttgg gcagaggaac aaatatttta aaacttataa |
| 1741 | tgaaaggaag atgctagaag ccctttcatg gaaagataaa gtagccaaag gatactttaa |
| 1801 | tagttgggct gaagaacatg gtgtatatca tcctgggcag agttctattt tagaaggaac |
| 1861 | agcttccaat cttgagtgtc acttatggca tattttggag ggaaagaaac tgccaaaggt |
| 1921 | aaaatgttct cctttttgca atggtgaaat tttaaaaact cttaatgaag caattgaaaa |
| 1981 | gtcattagga ggagctttta atttggattc caagtttagg ccaaaacagc agtattcttg |
| 2041 | ttcttgtcat gttttttctg aagaactgat attttccgag ttgtgtagcc ttactgagtg |
| 2101 | ccttcaggat gagcaggttg tagtacccag caatcaaata aagtgcctgc tggtgggctt |
| 2161 | ttcgactctc cgtaatatca aaatgcatat accgttggaa gttcgactcc tagaatcagc |
| 2221 | tgaactcaca acttttagct gttcattgct tcatgatgga gatccaactt accagcgttt |
| 2281 | atttttggac tgccttctac attcattgcg ggagcttcat acaggagatg ttatgatttt |
| 2341 | gcctgtactt tcttgcttca caagatttat ggctggtttg atctttgtac tccacagttg |
| 2401 | ttttagattc atcacttttg tttgtcccac atcctctgat cccctgagga cctgcgcagt |
| 2461 | cctgctatgt gttggttatc aggaccttcc aaatccagtt ttccgatatt tgcagagtgt |
| 2521 | gaatgaattg ttgagcactt tgctcaactc tgactcaccc cagcaggttt tacagtttgt |
| 2581 | gccaatggag gtactcctta agggggccct gcttgatttt ttgtgggatt tgaatgctgc |
| 2641 | cattgctaaa aggcatttgc atttcattat tcaaagagag agagaagaaa ttatcaacag |
| 2701 | ccttcagtta caaaactgaa catatgcttt ctgagattca actttatgat ttcttataat |
| 2761 | ttgcccagta tttgcatcct gttgctctat taatttaaaa accttttatt ttggggaaag |
| 2821 | gccaacattt gcatcattca aagtctcatt aattctggaa aaccatccat tctgatctct |
| 2881 | agggtatata cacccacagg catagagctc ttccacgtgg tggaatctat gcaatgatag |
| 2941 | atattcacac tctaaatatg aggtgtgtgt atgtgtatgg gtggccacag ccatgcttac |
| 3001 | ctatgccatt tagttggtct tacttaatct gcttaagatt tgcatctgtg tacctttgtt |
| 3061 | cagattagtt ttttttttcc agccgatttc ctcttagtgg ctaatgctgt tagtgaattt |
| 3121 | tccaactaat ttcctctcat tggttaatgt tgttaatgaa ttgagagagg taattgagga |
| 3181 | aaggaaatga gtaaatcact gttcagcaac actgatttcc gttaacacat cagttatgaa |
| 3241 | tttcagggaa ttcatctcgc cagattcttg ataacatgcc attcattgcc cttaggtgat |
| 3301 | tgaccctatt ttcttacatg gctcaaataa aactagtatg ctgttgtatg aatcttttac |
| 3361 | tgaccacacc atccaactat aaaaatataa cgggacagct ttaaaccaaa gatcatgttt |
| 3421 | agaacaatga aaaattattt gttgtatcta atacacgcct gtattgtgaa aagcttcatt |
| 3481 | tagcaatgat gtaataattt ttaacttcca ggaaataatc tgtgaatgga aagatttttt |
| 3541 | aagattttga gatagtgttt agtctcatgt tgggaacaca tgaatgtgat gaacatagtg |
| 3601 | aatactaaag aaaacgcttc agactttcag aatgatggtt cagaatttaa aatttttaat |
| 3661 | cttttctaat ttcttttttt cagtgtgaaa atagcacttt accaaaagat tagccatgaa |
| 3721 | atggttattt tgccagttac atttgatttc ttttgtatct gcaatgtaat gagttatttt |
| 3781 | atttcttctg tatttgcagt gtaatgagtt tttgtggcaa agtgtattaa gcaatttttc |
| 3841 | attatcttga agttccacaa agtggagaat atttatattc tcacatgcat tttaggcact |
| 3901 | tttgatatgt gaaaatagat gtattttctg atgcatttgg ttaataaata ttaatctgaa |
| 3961 | cattttcatg ttctttgcta ttttgaattc cattatagat tcatgaataa agtcattact |
| 4021 | agagagaaaa aaaaaaaaaa |

DRC7 (SEQ ID NO: 129)

| | |
|---|---|
| 1 | aggttgttac catggagatg gctaacagct agagcaggct gtcctcggag ggaaccgggt |
| 61 | cacatcgcag ggccacctct agctgcaaga gaatctggga agctgagcaa ttcaaaccag |
| 121 | gcacactgct gccccccaca caactggggt tctgccgtat agaagaggag actggatctt |
| 181 | tggagacatt ccatctccag acacccagag acgctccaga atggaggtcc tgagggagaa |
| 241 | ggtggaggag gaggaggagg ccgagcggga ggaggcggcc gagtgggctg aatgggcgag |
| 301 | gatggagaaa atgatgaggc cagttgaggt gcggaaggag gaaatcacct taaagcagga |
| 361 | gacgctcaga gacctggaga agaagctgtc agagatccag atcactgtct cagcggagct |
| 421 | cccggccttt accaaggaca ctattgacat ctccaagctg cccatttcct acaaaaccaa |
| 481 | cacacccaag gaggaacacc tgctgcaggt ggcagacaac ttctcccgcc agtacagcca |
| 541 | tctgtgcccg gaccgcgtgc ccctcttcct gcaccccctg aacgagtgtg aagtgcccaa |
| 601 | gttcgtgagc acaaccctcc ggcccacact gatgccctac cccgagctct acaactggga |
| 661 | cagctgtgcc cagtttgtct ccgacttcct caccatggtg cccctgcctg accctctcaa |
| 721 | gccgccctcg cacctgtact cctcgaccac tgtgctcaag taccagaagg ggaactgctt |
| 781 | tgacttcagt acgctgctct gctccatgct tatcggctct ggctatgatg cttactgcgt |
| 841 | caacggctac ggctcgctgg acctgtgcca catggacctg acgcgggagg tgtgcccact |
| 901 | cactgtgaag cccaaggaga ccatcaagaa ggaggaaaag gtgctgccta agaagtatac |
| 961 | catcaaaccc cccagggacc tgtgcagcag gtttgagcag gagcaagagg tgaagaagca |
| 1021 | gcaggagatc agagcccagg agaagaagcg gctgagggag gaggaggagc gcctcatgga |
| 1081 | agcggagaag gcaaagccgg atgccctgca cggcctgcgg gtgcactcct gggtccttgt |
| 1141 | gctatcgggg aagcgcgagg tgcctgagaa cttcttcatc gacccattca caggacatag |
| 1201 | ctacagcacc caggatgagc acttcctggg catcgaaagc ctgtggaacc acaagaacta |
| 1261 | ctggatcaac atgcaggatt gctggaactg ctgcaaggac ttgatctttg acctgggtga |
| 1321 | ccctgtgaga tgggagtaca tgctcctggg gactgataag tctcagctgt ccttgactga |
| 1381 | agaagacgac agtgggataa acgatgagga tgatgtggaa aatctgggca aggaggatga |
| 1441 | ggataagagc ttcgacatgc cccactcgtg ggtggagcag attgagatct ccccggaagc |
| 1501 | atttgagacc cgctgcccga acgggaagaa ggtgattcag tacaagaggg caaagctgga |
| 1561 | gaagtgggcc ccgtacctca atagcaatgg ccttgtgagc cgcctcacca cctatgagga |
| 1621 | cttgcagtgt accaatattt tggagataaa ggagtggtac cagaaccggg aagacatgct |
| 1681 | ggagctgaaa cacataaaca agaccacaga cctgaagaca gactacttca agcctggcca |
| 1741 | cccccaggct ctgcgcgtgc actcgtacaa gtccatgcaa cctgagatgg accgtgtcat |
| 1801 | tgagttttat gaaacggccc gtgtggatgg cctgatgaag cgggaggaga cacccaggac |
| 1861 | aatgacagag tactatcaag gacgcccaga cttcctctcc taccgccatg ccagcttcgg |
| 1921 | accccgagtc aagaagctca ctctgagcag tgcagagtca aacccccggc ccattgtgaa |
| 1981 | aatcacagag cggttcttcc gcaacccagc gaagcccgcg gaggaggacg tggcagagcg |
| 2041 | cgtgtttctg gtcgcggagg agcgcatcca gctgcgctac cactgccgtg aggaccacat |
| 2101 | cacggcctcc aagcgcgagt tcctgcggcg caccgaggtg gacagcaaag gcaacaagat |
| 2161 | catcatgacg cccgacatgt gcatcagctt cgaggtggag cccatggagc acaccaagaa |
| 2221 | gctgctctac cagtacgagg ccatgatgca cctgaagagg gaggagaagc tgtccagaca |
| 2281 | tcaggtctgg gagtcagagc tggaggtgct ggagattctg aagcttcgag aggaagagga |
| 2341 | ggcggcgcac acactgacca tctccatcta tgacaccaag cggaatgaga agagcaagga |
| 2401 | atatcgggag gccatggagc gcatgatgca cgaagagcac ctgcggcagg tggagaccca |
| 2461 | gctggactac ctggccccat tcctggccca gctcccgcca ggagagaaac taacatgctg |
| 2521 | gcaggcggtg cgcctcaagg atgagtgcct cagcgacttc aagcagcggc tcatcaacaa |
| 2581 | ggccaacctc atccaggccc gctttgagaa ggagacccag gagctgcaaa agaagcagca |
| 2641 | gtggtaccag gagaaccagg tgacgctgac acccgaggat gaagacctgt acctgagtta |
| 2701 | ctgctctcag gccatgttcc gcatccgcat cctggagcag cgcctcaatc gacacaagga |
| 2761 | actggcccca ctgaagtacc tggctctgga ggaaaagctc tacaaggacc cacgcctggg |
| 2821 | ggagctccag aaaatattcg cttgatgtcc ctcctggggc ctcagccaga gctgccagag |
| 2881 | aaaggaaacc tcttcccgca gcctggctcc tgtgttccct ctatccagcc aatgcctgtt |
| 2941 | tacacagaca cctggcctca ctgccagccc acctccccta cagccctgtt tgttcctgct |
| 3001 | tctcatgatt ttcctgtaaa taaacacact cttaatttgc caaaaaaaaa aaaaaaa |

ZCCHC2 (SEQ ID NO: 130)

| | |
|---|---|
| 1 | atgctgagga tgaagctgcc gctgaagcca acgcaccccg cggagccgcc gcccgaggcg |
| 61 | gaggagcccg aggcggacgc gcggccgggc gcgaaggcgc cttcgcgccg ccgccgcgac |
| 121 | tgccgccccc cgccgccgcc gccgccgccc gcgggcccgt cgcggggccc tctgccgccg |
| 181 | ccgccgccgc cccggggact cgggccgcct gttgctggtg gagcggcggc gggggcgggt |
| 241 | atgccgggcg gcggcggggg gccctcggcg gcgctgcgcg agcaggagcg ggtatacgag |
| 301 | tggttcgggc tggtgctggg ctcggcgcag cgcctggagt tcatgtgcgg gctgctggac |
| 361 | ctgtgcaacc cgctggagct gcgcttcctt ggctcgtgcc tggaggacct ggcgcgcaag |
| 421 | gactaccact acctgcgcga ctcggaggcc aaggccaacg gcctctcgga cccggggccg |
| 481 | ctggccgact tccgagagcc cgcggtgcgc tcgcgcctca tcgtctacct ggcgctgctg |
| 541 | ggctcggaga accgggaggc cgctggccgt ctgcaccgcc tgctacccca ggtggactcg |
| 601 | gtgctcaaaa gcctgcgcgc ggcccggggc gagggctcgc ggggcggcgc ggaggacgag |
| 661 | cgcggcgagg acggcgacgg cgagcaggac gccgagaagg acggctcagg cccggaaggc |
| 721 | ggcattgtgg agccccgggt cggcggcggg cttggctcca gggcccagga ggaactgctg |
| 781 | ctgctcttca ccatggcctc gctgcacccg gctttctcct tccaccagcg ggtcaccctg |
| 841 | agggaacact tggagaggct ccgcgccgcg ctccgcgggg gccccgagga cgcggaggtg |
| 901 | gaggtagagc cgtgcaagtt tgccggcccc agggcccaga acaactctgc tcatggtgat |
| 961 | tacatgcaaa ataacgagag cagcttaata gagcaagctc caatacctca ggacggactt |
| 1021 | accgtggcac ctcacagagc tcagcgagaa gctgtacaca ttgagaagat aatgttgaaa |
| 1081 | ggagtccaga gaaaaagagc tgacaaatac tgggagtaca ctttcaaagt aaattggtct |
| 1141 | gatctttcag tcacaacagt aacaaaaacc caccaagaac tacaggaatt tctactgaag |
| 1201 | cttccaaagg aactgtcttc agagactttt gacaagacca tcttaagagc cctgaatcag |
| 1261 | ggttccttga aaagggagga acggcgacat cctgacctag agcccatcct aaggcagcta |
| 1321 | ttttcaagtt catcacaagc ttttctacaa agtcagaaag tacacagctt ctttcagtcc |
| 1381 | atatcatcag actccctaca cagtatcaat aacttacaat cctctctgaa gacttctaag |
| 1441 | atattagaac acttaaaaga agacagctct gaagcttcaa gtcaagaaga agatgtgttg |
| 1501 | cagcatgcca taatccacaa gaagcatact gggaaaagtc ccattgtgaa caatattggt |
| 1561 | acaagttgtt ctccattgga tgggcttacc atgcaatatt ctgaacagaa tggaattgtg |
| 1621 | gattggagga agcaaagctg taccaccatt caacacccag agcactgtgt gacctcggct |
| 1681 | gaccagcatt ctgctgaaaa acggagttta tcttcaataa ataagaagaa aggaaagcca |
| 1741 | caaacagaaa aggagaaaat taagaaaact gacaacagat tgaatagtag aataaatggt |
| 1801 | attagactct ccactcctca gcatgcccat ggtggtactg tgaaagatgt gaatttggac |
| 1861 | attggctctg gacatgacac atgtggagaa acatcttcag agagttacag ttctccatct |
| 1921 | agtccccgac atgatggaag agaaagtttt gaaagtgaag aagagaaaga cagagacaca |
| 1981 | gacagcaatt ctgaggattc tgggaatcca tcaacaacta ggtttacagg ttacggttct |
| 2041 | gtcaaccaga ctgtcactgt caagccacct gttcaaattg cttcactagg aaatgagaat |
| 2101 | ggaaaccttt tagaagatcc cttaaactca cccaagtatc agcatatttc ttttatgcca |
| 2161 | acgttacact gtgtcatgca caatggtgcc cagaagtctg aagttgtcgt tcctgcaccc |
| 2221 | aaacccgctg atggcaaaac catagggatg cttgttccta gtcctgttgc tatttctgca |
| 2281 | ataagggagt ctgcaaattc aacccctgtt ggaatactag ggccaacagc ttgcactgga |
| 2341 | gaatcggaaa agcaccttga gttactggct tcccctttac ctattccatc aaccttcctt |
| 2401 | ccacacagta gtactcccgc tttgcatctt acagttcaga ggctaaagtt gccaccacca |
| 2461 | cagggatctt ctgagagctg cacagttaac atcccacaac aaccacccgg aagcctgagc |
| 2521 | atcgcatcac caaacactgc ctttattcct atccataacc caggtagttt cccaggctct |
| 2581 | cctgttgcta ccacggaccc catcacaaaa tctgcatccc aagtggtagg actcaatcaa |
| 2641 | atggtgcctc aaattgaggg aaacacaggg acagtccctc agcctaccaa tgtgaaggta |
| 2701 | gttcttccag cagctggcct ctcagctgct cagccaccag cttcctaccc cttaccaggc |
| 2761 | tctccccttg ctgccggcgt gttacccagc cagaactcca gtgtgctcag cacagcagca |
| 2821 | acttctcccc agccagcgag cgcaggtatc agccaggccc aggcaactgt tcctcctgca |
| 2881 | gttcctaccc acaccccagg ccctgccccg agcccaagcc ctgccttgac acacagtacc |
| 2941 | gcgcagagtg acagcacctc ttacatcagt gctgtgggga acacgaacgc taatgggaca |
| 3001 | gtagtgccac cgcagcagat gggctcaggt ccttgtggtt cttgtgggcg aaggtgcagc |
| 3061 | tgtgggacca atggaaacct tcagctaaat agttactatt atcctaatcc aatgcctgga |
| 3121 | ccaatgtacc gagtcccttc attctttact ctgccatcca tttgcaatgg cagctacctc |
| 3181 | aaccaagcac atcagagcaa tggaaaccaa cttccttttt ttctgcctca gactccatat |
| 3241 | gcaaatggac tggtacatga cccagtcatg gggagccaag ccaactatgg catgcagcag |
| 3301 | atggcaggat ttgggagatt ctatcctgta tatccagcac ctaacgtagt tgccaacacc |
| 3361 | agtggttcgg ggcccaagaa gaatgggaat gtctcatgtt acaattgtgg tgtaagcgga |
| 3421 | cactatgcac aggactgtaa gcagtcgtcc atggaggcca atcaacaagg cacttacaga |
| 3481 | ctgagatacg cacctcccct ccccccttct aatgatacgt tggattctgc agactgaaac |
| 3541 | gagtaaagct tgcctactta atacactcaa gtgtggggag tcatggggtg tggaggggag |
| 3601 | gaaaggaaag gtattttgtt tctttgtcta tacatttcct agatttctat gcagttggga |
| 3661 | tttttcattt ctcttgtacc aatgtccaaa acaagaaaga atgcaatgct tttgagcctc |
| 3721 | tggtctcctg gttcaacaac aggcttatat gtatgataca tgtaatttaa accttcagac |
| 3781 | aaacttaaat gttggtgcgt gctttttttt ttttttttac actgaatact tgctgtgtgc |
| 3841 | aatgtttact gaatctttaa aactgtgtat ttgacctttt ttttacaaca ctggtgacag |
| 3901 | tcatatggtt ttgaaaaaaa aaagaaattt tgcttcttcc cagcttttct cactttcacc |
| 3961 | ctaaacgaca cttcctcccc agccagcctc actctgtctc cggcccgcag caggagcagc |
| 4021 | cagcagtgca ttcaccccac ttttgtaaac tgctctgcat ataaaccaag ggcagaatgt |
| 4081 | ttcaccctga tcttatggga ggaatcaaac tcccaaaata gtgtgtatat atgtaataaa |
| 4141 | cagcgtcacg taaatacata tatgcagtgc ttgttgtcca aatagaaatg aaaataagtg |
| 4201 | gaagagagag gaagaagtca aaccatatga aactgaaaaa atatgacgta cgaaatggac |
| 4261 | aaaaagcttt ttctgaaacc aactttttac ttccatcatc cttttttagc ctgttgcttc |
| 4321 | agagagacac aaagtgaaca cactggtgtg aatgtcgctc tctgtgtgct tgtgtttgta |
| 4381 | atgaaagtct acagccaatt ttacttgtct accaccgtgt tgtgctcaaa gagacactac |
| 4441 | ttgagtgaag atttcttctt tccctgtacc agctgttaca gtgttacgtt gtgtttaaaa |
| 4501 | tgtgtatggt ttattgcaat ctgaacagag ctatgggttt ctaccataag tcaggttgtt |
| 4561 | tgttccctaa cctgtctctc atagcaaagt cacttttata acagtttacc actatgcttg |
| 4621 | attataatgt gaaaggcgga attctgagtg tgttaagatg gtattaatca tgtcggtgtc |
| 4681 | atgtcactaa gtttaatgct gctgttttta aaaaaaaaaa aaagtttttt taaaaagcca |
| 4741 | atctatgtac taaattgctt ccaggtaatt tttgatttcc taaagtgcac tgaggttatc |
| 4801 | tggaagattg ggtgtatttt ttggtgactg ctgcattcat cagcaatgaa cagtttccac |
| 4861 | tgtatagtcc taggggtcag ggggtggggg tttcattttc cattcctcag cacagagcag |
| 4921 | aaatgataga tttttattgt ttggagtaac gttggtatgc agcagaggaa cgtaaacatt |
| 4981 | tggtcttggt tcagaagcct aacagattgc tagacaagag aaaaaacttg aagaaaaaag |
| 5041 | aagcttaatt tcatgcttca taagtagcat ttatatttat agcaccaatg tacattttga |
| 5101 | aactttcttt caggggtggg agttatgggg aaggggtggg tgtgaagggg tagatgaaag |
| 5161 | ctttaattta gaaagaaagt tcaagtaaag gaaattattt tgattaaata tattttattt |
| 5221 | gatctgggta tttttggacc acattattaa attaattgtt aagctgcagt tgagttgttc |
| 5281 | aagtgagagt tttgataagc cacttatggg ccgcgttgtg aatcacttgc cagttgtact |
| 5341 | ttatggagct tattttatga tttaaaatac tgtactgtac ataggaggta tgttaccttc |
| 5401 | tccttatttg tatgtttacc atatactttg atatttgaaa tgttatgtac tggaaaggcc |
| 5461 | acttatattt ctagaacaga ttggatttta tgcaaccttt tttccttgaa ttaacagcaa |
| 5521 | taaaaaaatg aaaaacagct taaaaaaaaa aaaaaaaaa |

SL Gene interactions
ARHGDIA (SEQ ID NO: 131)

| | |
|---|---|
| 1 | cgcgtggggc ccgggccaga cctgagggcc cctccttggg gacgcggggg gcgccgggcc |
| 61 | ggcagccgcg gtccatcgcg ttcgggggcg acgcggggat tggggcgcgg cctcccccag |
| 121 | cgcccgggcc acgcccggca cggattgcgg gccctgcgga agtgcgggcc gcgccctagg |
| 181 | atcccggcgc ctacggctat cctcgcgcgg cgcggaggcc ccagcccctg gaggaagcag |
| 241 | ggcggcctgg accccggcct gggtgtcccg ggtgtgctgc tccctgaccc acctcccacg |
| 301 | ctgccgggaa ggatctgagc ctgacagatc ccctgccggg tgtcccgacc caggctaagc |
| 361 | ttgagcatgg ctgagcagga gcccacagcc gagcagctgg cccagattgc agcggagaac |
| 421 | gaggaggatg agcactcggt caactacaag cccccggccc agaagagcat ccaggagatc |
| 481 | caggagctgg acaaggacga cgagagcctg cgaaagtaca aggaggccct gctgggccgc |
| 541 | gtggccgttt ccgcagaccc caacgtcccc aacgtcgtgg tgactggcct gaccctggtg |
| 601 | tgcagctcgg ccccgggccc cctggagctg gacctgacgg gcgacctgga gagcttcaag |
| 661 | aagcagtcgt ttgtgctgaa ggagggtgtg gagtaccgga taaaaatctc tttccgggtt |
| 721 | aaccgagaga tagtgtccgg catgaagtac atccagcata cgtacaggaa aggcgtcaag |
| 781 | attgacaaga ctgactacat ggtaggcagc tatgggcccc gggccgagga gtacgagttc |
| 841 | ctgacccccg tggaggaggc acccaagggt atgctggccc ggggcagcta cagcatcaag |
| 901 | tcccgcttca cagacgacga caagaccgac cacctgtcct gggagtggaa tctcaccatc |
| 961 | aagaaggact ggaaggactg agcccagcca gaggcgggca gggcagactg acggacggac |
| 1021 | gacggacagg cggatgtgtc ccccccagcc cctcccctcc ccataccaaa gtgctgacag |
| 1081 | gccctccgtg cccctcccac cctggtccgc ctccctggcc tggctcaacc gagtgcctcc |
| 1141 | gacccccctc ctcagccctc ccccacccac aggcccagcc tcctcggtct cctgtctcgt |
| 1201 | tgctgcttct gcctgtgctg tgggggagag aggccgcagc caggcctctg ctgccctttc |
| 1261 | tgtgcccccc aggttctatc tccccgtcac acccgaggcc tggcttcagg agggagcgga |
| 1321 | gcagccattc tccaggcccc gtggttgccc ctggacgtgt gcgtctgctg ctccggggtg |
| 1381 | gagctggggt gtgggatgca cggcctcgtg ggggccgggc cgtcctccag ccccgctgct |
| 1441 | ccctggccag cccccttgtc gctgtcggtc ccgtctaacc atgatgcctt aacatgtgga |
| 1501 | gtgtaccgtg gggcctcact agcctctaac tccctgtgtc tgcatgagca tgtggcctcc |
| 1561 | ccgtcccttc cccggtggcg aacccagtga cccagggaca cgtggggtgt gctgctgctg |
| 1621 | ctccccagcc caccagtgcc tggccagcct gcccccttcc ctggacaggg ctgtggagat |
| 1681 | ggctccggcg gcttggggaa agccaaattg ccaaaactca agtcacctca gtaccatcca |
| 1741 | ggaggctggg tattgtcctg cctctgcctt ttctgtctca gcgggcagtg cccagagccc |
| 1801 | acaccccccc aagagccctc gatggacagc ctcactcacc ccacctgggc ccagccagga |
| 1861 | gccccgcctg gccatcagta tttattgcct ccgtccgtgc cgtccctggg ccactggcct |
| 1921 | ggcgcctgtt cccccaggct ctcagtgcca ccacccccgg caggccttcc ctgacccagc |
| 1981 | caggaacaaa caagggacca agtgcacaca ttgctgagag ccgtctcctg tgcctccccc |
| 2041 | gccccatccc cggtcttcgt gttgtgtctg ccaggctcag gcagaggcgc ctgtccctgc |
| 2101 | ttcttttctg accgggaaat aaatgcccct gaaggagcaa aaaaaaaaaa |

FAM63B (SEQ ID NO: 132)

| | |
|---|---|
| 1 | gcagtcaggc ggaggcaagc tcagagcgca cggacagagc ggtagcgcgc gcccgcgcgc |
| 61 | gttcttagta ctctccccgg tgacgtgcct gaccgaggcc gcgccagggc gctgttgctg |
| 121 | ccaatacagc tgtcatggcg tccaaggcgc tggctgcgga gaagtggccg cggtctccat |
| 181 | agagctgggg gcgggcggcc cggtatggag agcagccccg agagcctgca gccgctagaa |
| 241 | cacggggtgg cggccgggcc agcgtcaggg acaggttctt cgcaggaagg gctacaggag |
| 301 | accaggctcg ccgctggtga tggtcctggg gtatgggcgg cggagaccag cggcgggaat |
| 361 | gggctggggg cggcggccgc caggaggagc ctcccggact cggcttctcc cgcgggctct |
| 421 | cctgaggttc ccggaccctg cagctcctcc gcgggtttgg acttgaagga cagtggtttg |
| 481 | gagagtcctg ctgccgccga ggcgcctctg agagggcagt acaaggtgac cgcctccccg |
| 541 | gagacagccg tggccggagt gggtcatgag ttgggtaccg ccggagacgc gggagcccgc |
| 601 | ccggatctcg ccggcacctg ccaagcagaa ctgaccgccg ccggctccga agagcccagc |
| 661 | agcgccggcg gcctcagcag cagttgcagc gacccgagcc ctcctgggga atctccgagc |
| 721 | ctggactctc tggagtcgtt ctctaacctg cattcttttc ccagtagctg cgagttcaat |
| 781 | agtgaggagg gagcggagaa cagggtccct gaggaggagg agggcgcggc ggtgttgccc |
| 841 | ggggctgttc ctctgtgcaa ggaggaggag ggggaggaga ccgctcaggt gctggcggcc |
| 901 | tccaaggaac gcttcccggg acaatctgtg tatcacatca agtggatcca gtggaaggaa |
| 961 | gagaacacac ccatcatcac ccagaatgag aacggaccct gccccttgct ggccatcctc |
| 1021 | aatgttttgc tcctggcctg gaaggtgaaa cttccaccga tgatggaaat cataactgct |
| 1081 | gagcagctga tggaatattt aggagattac atgcttgatg caaagccaaa agaaatttca |
| 1141 | gaaattcaac gtttaaatta tgaacagaat atgagtgatg ccatggcaat tttgcacaaa |
| 1201 | ctacagacag gcctggatgt aaatgtaaga ttcactggtg ttcgagtgtt tgaatataca |
| 1261 | ccagaatgca tagtatttga tcttcttgat attcctttgt accatgggtg gttagtagac |
| 1321 | cctcagattg atgacattgt aaaagctgtt ggtaactgca gctacaacca actagtggag |
| 1381 | aagatcatct cttgtaaaca gtcagacaat agtgagctgg ttagtgaagg ctttgtagct |
| 1441 | gagcagtttc taaataacac agccactcaa ctgacatacc atggattatg tgaactaact |
| 1501 | tcaacggttc aggaaggaga actttgtgtg ttctttcgga ataatcattt tagcaccatg |
| 1561 | accaaataca agggtcaact gtatttgttg gtaacggacc aggggtact tactgaagag |
| 1621 | aaagttgttt gggaaagcct acacaacgta gatggtgatg gaaatttctg tgactcagaa |
| 1681 | tttcatcttc gacctccttc agatcctgaa actgtataca aaggacaaca agatcagata |
| 1741 | gatcaggatt atcttatggc attatctcta caacaagaac agcagagcca agagatcaat |
| 1801 | tgggaacaaa tcccggaagg aatcagtgat ttggaactag caaagaaact ccaagaggaa |
| 1861 | gaggacagac gggcttctca atactatcag gaacaggaac aagcagcagc tgctgctgct |
| 1921 | gctgcttcta cacaggctca gcagggccag ccagcacaag cctctccatc aagtggaaga |
| 1981 | caatctggga atagtgaacg taaacggaag gaaccacgag aaaaagataa agaaaaagaa |
| 2041 | aaggaaaaaa atagctgtgt tattttgtaa caagtgttgg cttctgttgg aaccacctat |
| 2101 | atgtcttgag aaacaaaacc acaggaggaa aggaagaaaa accgatcaat accgtctgtg |
| 2161 | cctgatttcc taatggattt tgttcgtttt ttcaggggaa cggttgttac ttagttacaa |
| 2221 | tcagactttt tcaagtcaca caatacactc tttatgagct ggagtttcat gttacaagtt |
| 2281 | ggaaatgctg tgtgttgaca ttcatgaaaa atactgcact tgtagccaga ttagcaaatc |
| 2341 | acagcaaatt ttgtgtcata gtgacattca taactcatat cagttagtaa gctattatat |
| 2401 | cttctgttct aacaatgaat ggaggtaatt gatttagtct gattccttcc tgaaatctaa |
| 2461 | atattagcac aatagtttct gaaattttac aatgttaaat tatgatctaa ttcatgagaa |
| 2521 | accacgggtt taacataggg attcaaaaaa acaaaaacaa aagaatagga ataaataacc |
| 2581 | cttaattgta tattggacta gttcagccct taaacagctt tacctttatt taggaatgta |
| 2641 | cattttaggt attatcttga tcatggagct tagttttaat ttagatagca aaaataaaga |
| 2701 | tttgtatttc ttttccaata gcaaaaagtt acataacact aatacttata acctatcaat |
| 2761 | atcagatatt aatgactttg tagtgttgta aaattttgag gaattttgga gtctttatca |
| 2821 | taggtaacct ggaccacagt tactatttat tgacaatgtg attgagtgta tggaggaaag |
| 2881 | cacagtggat gctaggcttt gtaaatatgg ggatgtagaa aagcagatag ttcagtgtct |
| 2941 | acctttttct agaactacct tgaaccttaa attttaagtc atgttcattg ctagaaaatt |
| 3001 | aaatgtactt attaaaacca atgaaaaagc acatttctga aatgaagtta gagataatct |
| 3061 | ctgtgtctta taaaaagaca ttaataaaaa tctgaaaggg ccgggcgcag tggctcacgc |
| 3121 | ctgtaatccc aacattttgg gaggccaagg tgggcggatc atctgaggcc aggagttcga |
| 3181 | gaccagcctg gccagcatgg tgaaaccctg tctctactaa aaatacaaaa aatcagcctg |
| 3241 | gcatggtggt gcgtgcctgt agtcccagct actcagggct gaggcaggag aattgcttga |
| 3301 | acccggcagg cagaggttgc agtgagccga gatcgccctg ctgcactcca gcctgggtga |
| 3361 | cagagggaga ctccgtctca aaaaaaaaaa agtctgagag tagctaagaa tttatgtaaa |
| 3421 | agcaatcaga gtttttaatt tatgggaacc aaataaaact ataacctcat agtgtttata |
| 3481 | agaactcaga aataatattt atttaacttt attatgaggc cacacatatt ttcctgtgtt |
| 3541 | tctatatata gtttggaaaa ctatccttaa tagtctgttt tatatgcctt atatttaaaa |
| 3601 | gtttgtttta gttattttga aagactattg ctgctgcaaa tagttgtgtg ctttacattc |
| 3661 | taagcttcag tacatttatt taagagcatc ataatctgac ctgagcatcc acttggagag |
| 3721 | tgtttttttt gtgtgtggtc tggggtgaca aaagaccaca aaaatgtgtg gtctggattt |
| 3781 | tttcaactat gtcattaact ttatgatcca agaccagtta taggatgaat ctgtatgtaa |
| 3841 | aaatagagtc ttatttatgg aaggaattat tctaagggaa aaatccaggg tcaagctgta |
| 3901 | tcttttatgt cctttatatt gcatgtctat ttctgttaca caatttgtta tttcttcaaa |
| 3961 | tttcctatgg tagcatgata aatcatcaaa gaacctgttt gggatataaa actctgatag |
| 4021 | aaaatattta atgagtatct tgattataac ctagaatatg tatacgttag taaaataacc |
| 4081 | agatatacta cagaactctc tattggctca aacaggttga cctcaatcca agtttactct |
| 4141 | tgatatcact ctgttggctg aaggaggtaa ctcaaacctc agggtttgtt tttcccggga |
| 4201 | cagatagtag tgatagtgca ttatatttga ataagaaaaa caaaccagta taccttgaga |
| 4261 | aattttaaaa agcatagttg aggcatattt tttcataatt atatacttat ctgtttattg |
| 4321 | cccatggaaa atatatgtgt agaagtattt cttctgttat ttgttactat cttcttaatt |
| 4381 | tgttccaaag aaaatgctgc catactgcat tccctctgga aggaaacaaa acaaaacaaa |
| 4441 | actcactcaa aaccagcagt gctgctatca gataagtaga tgtcaatgta tacttacaag |
| 4501 | gaaaaactaa aaaatgtaat gtgttaattc agcctttttc tatgtaatat ttccaagtca |
| 4561 | gactttctta cattcctgga atttactttg atataccaag aataataatg ataaaatgtt |
| 4621 | tgctttgatt actgtggggg gaaagatgaa atgttcaatt gtattaaaac aaacaagctt |
| 4681 | ttcagagata ctggtttcct gcccttgaag ggtataaaga atttagatca tgcctgtaat |
| 4741 | cccagtactt tgggaggccg aggcaggtgg atcacctgag atcaggagtt cgagaccagc |
| 4801 | ctggccaaca tggcaaaacc ctgtctctac taaaaataca ataattagcc aggcatggtg |
| 4861 | gcgggcacct gtcatcccag ctacttggga ggctgaggca ggagaatcgc ttgaacccag |
| 4921 | gaggcagtga ttgcagtgag ctgagatagc accactgcat gcaagcctgg gcaatagagc |
| 4981 | gagactccgt ctcaaaaaaa aaaaaaaaaa aaaaattaga gctattgtgt ctttattttc |
| 5041 | ttaaattttg cccaaggtaa cgttatatat cccaccactt cattgctggt ttgggtacat |
| 5101 | aggattttga aagtggtata ttaaagtctt tccttccaag tattttgtaa tacttgaaaa |
| 5161 | ttcttagatg tatactgcta acaaaagtta gaacttaaac atttttgttt ttatcattta |
| 5221 | tagcctagat tagggacata tttgcatcaa ccaaatcatc attagatttg aaaataggca |
| 5281 | gatgaatgaa caaatatggt cattgcactt tccttttact ttcagagtct aagtatattc |
| 5341 | cttaaggtta gtaaccagtc tttattaaaa atataaaatt tttcttcatg tctaatccca |
| 5401 | ttgcatccac aatgctgtga tttatagtac atgatcaaca cttaaaagta ctttacatat |
| 5461 | gtgtgtttct gaagcaagtt ttcatgacct ctgttagatt ctcaaaagaa ttcagaactt |
| 5521 | caatttaaga atcaccattt taagaataca tgtgtacata tacacattaa gcagtataaa |
| 5581 | gcagctaaaa ttggcattgg ttttacactg gtgcagtgtg cttaggtaaa gtaacttctt |
| 5641 | ccatgtttca aggtcaggtt cagagttgaa tgaagtgtag atttaaattt aggattaggc |
| 5701 | tttggaatat atcttgtttt tattgtctca catttctgat attgactact tatcccatat |
| 5761 | tctgtttcaa attctttatc atatttcaag ttctttctca tacttcttga tcttggctta |
| 5821 | actaagcaag ttagtatcag agactagttg actgaaccca agattaaaca ttttgcactt |
| 5881 | gcacaaaacc ttcttagcat tttgctttca atgaatcaga aagtcaattc actaagagac |
| 5941 | agatcatgag aggaaagaga actagaggcc aataaataaa ataattgttc atatattaat |
| 6001 | gttcacatgt gaactacata tctaaaatct tggagaaaaa tcaaggcaag aatttccaga |
| 6061 | actgtcctca aatagctcat ttatttaagt tttgttaaaa agcaaaagcg aattgattac |
| 6121 | atttgattaa cttttcctat tccatgcaca agttacctta aaacatgata aaaaccttat |
| 6181 | gggcattacc tatcacacag tacttatgca taaacttata atagtaaaat tactaatgtt |
| 6241 | tgataaaata agatggaggc attacaaata gtctacagtt tgtattttaa ggaattggac |
| 6301 | atgaagaatt ctagatcatt ttgtgtctat aaacccgact ttctatcttg ccttgggcaa |
| 6361 | actttctgtg cctcaatgta ctctttaaat atgtgaagga tgctcttttt gattaagtgt |
| 6421 | tttgcactcc tgaataaagg gcatagtata agcacaaagt atgacttaat ttatcacaaa |
| 6481 | tattacacat cctatgttct tgaatgtgca cacttttttc tcaataacaa aatatatctt |
| 6541 | aagtcagttt ttttaatgct gtcaaaattt gtagaatttt ctttgagtat ggcgtgatct |
| 6601 | cttcccaaat gcattttaca gttttttgtg tgttctatag actatagagt caaaatcaag |
| 6661 | agtattttga gaggatcaga agcatttaaa aatctatttt tttctagtat ctttcacaga |
| 6721 | tctaaatatt tagattctct ttgccttttt ctccatggaa tacggtggta tcaaattact |
| 6781 | aatacagtat ataaacttcg tttgcattgg tggaattcat ttagatctct caagtaatat |
| 6841 | tattttaggg ctatataaat tgtgttttta gtgtaaaatg ttatttgata atgtgaagtt |
| 6901 | aaatcccttt tagaaagtga ctgaaaatgg taaaggaact catcagaatc ttagcgttct |
| 6961 | taagttctct gataatttag tatattttat taatgatgtc caacacctct aagattgttg |
| 7021 | agaaaacatg aagaattgag gttactcttc tcaggtgaca ctttaaatat taaaatcaga |
| 7081 | ggcttcctga acaaaacaaa ttgcaaaata gcgataatgg catgggagag gccagatgca |
| 7141 | ggactctggt aaatttaact tactttgaat atctatctaa attttagttc atgcatgttc |
| 7201 | ttacttaatc ctggtgtttt tgctcttaga tgttagagtt taataaattg tgatacgcat |
| 7261 | atattttttt acatgaagga ttctactttc taattttact tttctgatct caagaaaatt |
| 7321 | aaacttgaaa aacggggtaa aattcttcaa ctattgcctc aagttcagtt ttgtcctatt |
| 7381 | gtcctgagaa aggagattta gacttgtctg cctaacacag gtatttttta gggcatcgta |
| 7441 | ctatcccaga gaaagtgttg agataccatg gcagaaatat aaaacctaag ctttgaaccc |
| 7501 | cagtagactt cttcttctgc cattaagtct ctctttatct gatattctaa ggatttcttc |
| 7561 | aaactactta ataatttgtc accattaact ttaatatcca gttttaatct gcactgtaat |
| 7621 | atcctgcttt gagaagaaag aatgcctcat aaattagaga aggacaaaac aaaatgtttt |
| 7681 | ggaaggtgat cctggctcct ttggctctca taattgtttt atagctgaaa ataaaaagtc |
| 7741 | aggaaactgg cccggtgcgg tggctcatgc ctgtaatccc agcactttgg gaggctgagg |
| 7801 | tgggtggatc acctgaagtc aggagttcga gaccagcctg gccaacatgg tgaaaccctg |
| 7861 | tctctactaa aaatacaaaa aaaattagct gggtgtggtg gcacatgcct gtaatcccag |
| 7921 | ccactgggga ggttgaggcg caagaatcgc ttgaacccgg aaggcagagg ttgcagtgag |
| 7981 | ccaagattgc cccactgcac tccagcctgg gcgactgagc gagactctta tatctcaaaa |
| 8041 | aaaaaaaaaa aaaaaagtca agaaactgaa attcccattt aagttctcaa atcagtgatc |
| 8101 | tgtcaaaata ggccttgtaa ctgaaatacc ttacaaagca gttctaacta atgcaatgtg |
| 8161 | ttttttaaaa atttttaatg aaccttacat tgtgaacata attgcaacat gttttaagac |
| 8221 | aaacagtatt taatccttga agacctgtct tgtatgtctc tcaattttgt cagaattttt |
| 8281 | attattgttt ttcacatatg tgaaataagc agttttttca gggtacatag ggtatctttg |
| 8341 | ttttacagat ttttaaagat gaggttttga aaagccctca gaggtttttg ttaaaagact |
| 8401 | atcttgctta ataaatgaca gcttgttaca gattcacaca ttacaagtag gacagtataa |
| 8461 | caggagattg gtgtgtgaat gctacaaaac agtcagcaaa aggaatcatg tttgcttgtg |
| 8521 | aaacttcaga ggtaccctga aagtcatttc ctaaagctag tgcgtgtgaa tcttttcctt |
| 8581 | gaattgtgca gaataattgg attgaggcac atattttgag gagtagcaag tggaatggta |
| 8641 | taatgactac agagaaaatt atcttgaaat atagcaagga agagaaacaa gttttctttc |
| 8701 | tccactttat tgttggacta attgggtcaa tttgctgtga catatcaaag atctctttgt |
| 8761 | gccaggccaa gactggctac tgagttctca aagcgtttta atatatagat tacgtatgag |
| 8821 | tgcctatttt ttcctcctcc tttcattttt tatcttaata cccattttac ttctgaaata |
| 8881 | attcatctgt tttgctttat gaccagcttt aatttcaatt gaggaataat aacaacccta |
| 8941 | gagattcata ggaaagagca ttgaaataca ttttttgcat aaagatacct aaaaccatct |
| 9001 | acccagctta gggttgaact gaatttctgt gaaataaatt tgttttaaat actaattatt |
| 9061 | ttaaaactac ttaattctta aaaacaatgt catcagtttc aaaactttca ctttgggagg |
| 9121 | atattcctta aaaggcatac atagatggta aagtataaaa tatttctgac agaattattc |
| 9181 | agtattattc aacatttact ttcatgtttg ttattgtacc acaaagatag tgtcattgtt |
| 9241 | gggttaaaat gttggctgtt tttgttaata tacttaaaac tgtaaccagt gaataacacc |
| 9301 | tgtagtattt tttattatag attatatttt atttcaataa actttgatat ttagaccaaa |
| 9361 | aaaaaaaaaa aaaaa |

HMGCS2 (SEQ ID NO: 133)

| | |
|---|---|
| 1 | ataaagtcct gccgggcacc actgggcatc tctttcaagg tttctgctgg gtttctgaac |
| 61 | tgctgggttt ctgcttgctc ctctggagat gcagcgtctg ttgactccag tgaagcgcat |
| 121 | tctgcaactg acaagagcgg tgcaggaaac ctccctcaca cctgctcgcc tgctcccagt |
| 181 | agcccaccaa aggttttcta cagcctctgc tgtccccctg gccaaaacag atacttggcc |
| 241 | aaaggacgtg ggcatcctgg ccctggaggt ctacttccca gcccaatatg tggaccaaac |
| 301 | tgacctggag aagtataaca atgtggaagc aggaaagtat acagtgggct tgggccagac |
| 361 | ccgtatgggc ttctgctcag tccaagagga catcaactcc ctgtgcctga cggtggtgca |
| 421 | acggctgatg gagcgcatac agctcccatg ggactctgtg ggcaggctgg aagtaggcac |
| 481 | tgagaccatc attgacaagt ccaaagctgt caaaacagtg ctcatggaac tcttccagga |
| 541 | ttcaggcaat actgatattg agggcataga taccaccaat gcctgctacg gtggtactgc |
| 601 | ctccctcttc aatgctgcca actggatgga gtccagttcc tgggatgggc tgaggggaac |
| 661 | ccatatggag aatgtgtatg acttctacaa accaaatttg gcctcggagt acccaatagt |
| 721 | ggatgggaag ctttccatcc agtgctactt gcgggccttg gatcgatgtt acacatcata |
| 781 | ccgtaaaaaa atccagaatc agtggaagca agctggcagc gatcgaccct tcacccttga |
| 841 | cgatttacag tacatgatct ttcatacacc cttttgcaag atggtccaga agtctctggc |
| 901 | tcgcctgatg ttcaatgact tcctgtcagc cagcagtgac acacaaacca gcttatataa |
| 961 | ggggctggag gctttcgggg ggctaaagct ggaagacacc tacaccaaca aggacctgga |
| 1021 | taaagcactt ctaaaggcct ctcaggacat gttcgacaag aaaaccaagg cttcccttta |
| 1081 | cctctccact cacaatggga acatgtacac ctcatccctg tacgggtgcc tggcctcgct |
| 1141 | tctgtcccac cactctgccc aagaactggc tggctccagg attggtgcct tctcttatgg |
| 1201 | ctctggttta gcagcaagtt tcttttcatt tcgagtatcc caggatgctg ctccaggctc |
| 1261 | tcccctggac aagttggtgt ccagcacatc agacctgcca aaacgcctag cctcccgaaa |
| 1321 | gtgtgtgtct cctgaggagt tcacagaaat aatgaaccaa agagagcaat tctaccataa |
| 1381 | ggtgaatttc tccccacctg gtgacacaaa cagccttttc ccaggtactt ggtacctgga |
| 1441 | gcgagtggac gagcagcatc gccgaaagta tgcccggcgt cccgtctaaa ggtgttctgc |
| 1501 | agatccatgg aaagcttcct gggaaacgta tgctagcaga gcttctcccc gtgaatcata |
| 1561 | tttttaagat cccactctta gctggtaaat gaatttgaat cgacatagta gccccataag |
| 1621 | catcagccct gtagagtgag gagccatctc tagcgggccc ttcattcctc tccatgctgc |
| 1681 | aatcactgtc ctgggcttat ggtgctatgg actaggggtc ctttgtgaaa gagcaagatg |
| 1741 | gagcaatgga gagaagacct cttcctgaat cactggactc cagaaatgtg catgcagatc |
| 1801 | agctgttgcc ttcaagatcc agataaactt tcctgtcatg tgttagaact ttattattat |
| 1861 | taatattgtt aaacttctgt gctgttcctg tgaatctcca aattttgtac cttgttctaa |
| 1921 | gctaatatat agcaattaaa aagagagaaa gaggaaatga ttcctgcgtt tcttggaacc |
| 1981 | cagaatacaa acccagccta acatgcagca agcctgctag accttgtggg tcagagggct |
| 2041 | gggtccttgc ctcacaggct gcctctgtcc ccttgcaatt ccattctatt tctgccacat |
| 2101 | gccaagtgct atgacaggta caaggcaaat aagaacggta gaacacagct tcccccagcc |
| 2161 | cacttccctg ttctaaagac accacataga cagagagcag cagacagggg ccagcaggag |
| 2221 | ctgtagttca gatcttcttg gtcattcctt gccgctgtta tttgaacaaa taaacacagc |
| 2281 | gcaaaggtta acaagttttt gccttctata gccaaaaata aaaaaataaa taaattttga |
| 2341 | aaaaaaaaaa a |

IQGAP1 (SEQ ID NO: 134)

| | |
|---|---|
| 1 | ggaccccggc aagcccgcgc acttggcagg agctgtagct accgccgtcc gcgcctccaa |
| 61 | ggtttcacgg cttcctcagc agagactcgg gctcgtccgc catgtccgcc gcagacgagg |
| 121 | ttgacgggct gggcgtggcc cggccgcact atggctctgt cctggataat gaaagactta |
| 181 | ctgcagagga gatggatgaa aggagacgtc agaacgtggc ttatgagtac ctttgtcatt |
| 241 | tggaagaagc gaagaggtgg atggaagcat gcctagggga agatctgcct cccaccacag |
| 301 | aactggagga ggggcttagg aatggggtct accttgccaa actggggaac ttcttctctc |
| 361 | ccaaagtagt gtccctgaaa aaaatctatg atcgagaaca gaccagatac aaggcgactg |
| 421 | gcctccactt tagacacact gataatgtga ttcagtggtt gaatgccatg gatgagattg |
| 481 | gattgcctaa gattttttac ccagaaacta cagatatcta tgatcgaaag aacatgccaa |
| 541 | gatgtatcta ctgtatccat gcactcagtt tgtacctgtt caagctaggc ctggcccctc |
| 601 | agattcaaga cctatatgga aaggttgact tcacagaaga agaaatcaac aacatgaaga |
| 661 | ctgagttgga gaagtatggc atccagatgc ctgcctttag caagattggg ggcatcttgg |
| 721 | ctaatgaact gtcagtggat gaagccgcat tacatgctgc tgttattgct attaatgaag |
| 781 | ctattgaccg tagaattcca gccgacacat ttgcagcttt gaaaaatccg aatgccatgc |
| 841 | ttgtaaatct tgaagagccc ttggcatcca cttaccagga tatactttac caggctaagc |
| 901 | aggacaaaat gacaaatgct aaaaacagga cagaaaactc agagagagaa agagatgttt |
| 961 | atgaggagct gctcacgcaa gctgaaattc aaggcaatat aaacaaagtc aatacatttt |
| 1021 | ctgcattagc aaatatcgac ctggctttag aacaaggaga tgcactggcc ttgttcaggg |
| 1081 | ctctgcagtc accagccctg gggcttcgag gactgcagca acagaatagc gactggtact |
| 1141 | tgaagcagct cctgagtgat aaacagcaga agagacagag tggtcagact gaccccctgc |
| 1201 | agaaggagga gctgcagtct ggagtggatg ctgcaaacag tgctgcccag caatatcaga |
| 1261 | gaagattggc agcagtagca ctgattaatg ctgcaatcca gaagggtgtt gctgagaaga |
| 1321 | ctgttttgga actgatgaat cccgaagccc agctgcccca ggtgtatcca tttgccgccg |
| 1381 | atctctatca gaaggagctg gctaccctgc agcgacaaag tcctgaacat aatctcaccc |
| 1441 | acccagagct ctctgtcgca gtggagatgt tgtcatcggt ggccctgatc aacagggcat |
| 1501 | tggaatcagg agatgtgaat acagtgtgga agcaattgag cagttcagtt actggtctta |
| 1561 | ccaatattga ggaagaaaac tgtcagaggt atctcgatga gttgatgaaa ctgaaggctc |
| 1621 | aggcacatgc agagaataat gaattcatta catggaatga tatccaagct tgcgtggacc |
| 1681 | atgtgaacct ggtggtgcaa gaggaacatg agaggatttt agccattggt ttaattaatg |
| 1741 | aagccctgga tgaaggtgat gcccaaaaga ctctgcaggc cctacagatt cctgcagcta |
| 1801 | aacttgaggg agtccttgca gaagtggccc agcattacca agacacgctg attagagcga |
| 1861 | agagagagaa agcccaggaa atccaggatg agtcagctgt gttatggttg gatgaaattc |
| 1921 | aaggtggaat ctggcagtcc aacaaagaca cccaagaagc acagaagttt gccttaggaa |
| 1981 | tctttgccat taatgaggca gtagaaagtg gtgatgttgg caaaacactg agtgcccttc |
| 2041 | gctcccctga tgttggcttg tatggagtca tccctgagtg tggtgaaact taccacagtg |
| 2101 | atcttgctga agccaagaag aaaaaactgg cagtaggaga taataacagc aagtgggtga |
| 2161 | agcactgggt aaaaggtgga tattattatt accacaatct ggagacccag gaaggaggat |
| 2221 | gggatgaacc tccaaatttt gtgcaaaatt ctatgcagct ttctcgggag gagatccaga |
| 2281 | gttctatctc tggggtgact gccgcatata accgagaaca gctgtggctg gccaatgaag |
| 2341 | gcctgatcac caggctgcag gctcgctgcc gtggatactt agttcgacag gaattccgat |
| 2401 | ccaggatgaa tttcctgaag aaacaaatcc ctgccatcac ctgcattcag tcacagtgga |
| 2461 | gaggatacaa gcagaagaag gcatatcaag atcggttagc ttacctgcgc tcccacaaag |
| 2521 | atgaagttgt aaagattcag tccctggcaa ggatgcacca agctcgaaag cgctatcgag |
| 2581 | atcgcctgca gtacttccgg gaccatataa atgacattat caaaatccag gcttttattc |
| 2641 | gggcaaacaa agctcgggat gactacaaga ctctcatcaa tgctgaggat cctcctatgg |
| 2701 | ttgtggtccg aaaatttgtc cacctgctgg accaaagtga ccaggatttt caggaggagc |
| 2761 | ttgaccttat gaagatgcgg gaagaggtta tcaccctcat tcgttctaac cagcagctgg |
| 2821 | agaatgacct caatctcatg gatatcaaaa ttggactgct agtgaaaaat aagattacgt |
| 2881 | tgcaggatgt ggtttcccac agtaaaaaac ttaccaaaaa aaataaggaa cagttgtctg |
| 2941 | atatgatgat gataaataaa cagaagggag gtctcaaggc tttgagcaag gagaagagag |
| 3001 | agaagttgga agcttaccag cacctgtttt atttattgca aaccaatccc acctatctgg |
| 3061 | ccaagctcat ttttcagatg ccccagaaca agtccaccaa gttcatggac tctgtaatct |
| 3121 | tcacactcta caactacgcg tccaaccagc gagaggagta cctgctcctg cggctcttta |
| 3181 | agacagcact ccaagaggaa atcaagtcga aggtagatca gattcaagag attgtgacag |
| 3241 | gaaatcctac ggttattaaa atggttgtaa gtttcaaccg tggtgcccgt ggccagaatg |
| 3301 | ccctgagaca gatcttggcc ccagtcgtga aggaaattat ggatgacaaa tctctcaaca |
| 3361 | tcaaaactga ccctgtggat atttacaaat cttgggttaa tcagatggag tctcagacag |
| 3421 | gagaggcaag caaactgccc tatgatgtga cccctgagca ggcgctagct catgaagaag |
| 3481 | tgaagacacg gctagacagc tccatcagga acatgcgggc tgtgacagac aagtttctct |
| 3541 | cagccattgt cagctctgtg gacaaaatcc cttatgggat gcgcttcatt gccaaagtgc |
| 3601 | tgaaggactc gttgcatgag aagttccctg atgctggtga ggatgagctg ctgaagatta |
| 3661 | ttggtaactt gctttattat cgatacatga atccagccat tgttgctcct gatgcctttg |
| 3721 | acatcattga cctgtcagca ggaggccagc ttaccacaga ccaacgccga aatctgggct |
| 3781 | ccattgcaaa aatgcttcag catgctgctt ccaataagat gtttctggga gataatgccc |
| 3841 | acttaagcat cattaatgaa tatctttccc agtcctacca gaaattcaga cggtttttcc |
| 3901 | aaactgcttg tgatgtccca gagcttcagg ataaatttaa tgtggatgag tactctgatt |
| 3961 | tagtaaccct caccaaacca gtaatctaca tttccattgg tgaaatcatc aacacccaca |
| 4021 | ctctcctgtt ggatcaccag gatgccattg ctccggagca caatgatcca atccacgaac |
| 4081 | tgctggacga cctcggcgag gtgcccacca tcgagtccct gataggggaa agctctggca |
| 4141 | atttaaatga cccaaataag gaggcactgg ctaagacgga agtgtctctc accctgacca |
| 4201 | acaagttcga cgtgcctgga gatgagaatg cagaaatgga tgctcgaacc atcttactga |
| 4261 | atacaaaacg tttaattgtg gatgtcatcc ggttccagcc aggagagacc ttgactgaaa |
| 4321 | tcctagaaac accagccacc agtgaacagg aagcagaaca tcagagagcc atgcagagac |
| 4381 | gtgctatccg tgatgccaaa acacctgaca agatgaaaaa gtcaaaatct gtaaaggaag |
| 4441 | acagcaacct cactcttcaa gagaagaaag agaagatcca gacaggttta aagaagctaa |
| 4501 | cagagcttgg aaccgtggac ccaaagaaca aataccagga actgatcaac gacattgcca |
| 4561 | gggatattcg gaatcagcgg aggtaccgac agaggagaaa ggccgaacta gtgaaactgc |
| 4621 | aacagacata cgctgctctg aactctaagg ccacctttta tggggagcag gtggattact |
| 4681 | ataaaagcta tatcaaaacc tgcttggata acttagccag caagggcaaa gtctccaaaa |
| 4741 | agcctaggga aatgaaagga aagaaaagca aaaagatttc tctgaaatat acagcagcaa |
| 4801 | gactacatga aaaaggagtt cttctggaaa ttgaggacct gcaagtgaat cagtttaaaa |
| 4861 | atgttatatt tgaaatcagt ccaacagaag aagttggaga cttcgaagtg aaagccaaat |
| 4921 | tcatgggagt tcaaatggag acttttatgt tacattatca ggacctgctg cagctacagt |
| 4981 | atgaaggagt tgcagtcatg aaattatttg atagagctaa agtaaatgtc aacctcctga |
| 5041 | tcttccttct caacaaaaag ttctacggga agtaattgat cgtttgctgc cagcccagaa |
| 5101 | ggatgaagga aagaagcacc tcacagctcc tttctaggtc cttctttcct cattggaagc |
| 5161 | aaagacctag ccaacaacag cacctcaatc tgatacactc ccgatgccac atttttaact |
| 5221 | cctctcgctc tgatgggaca tttgttaccc ttttttcata gtgaaattgt gtttcaggct |
| 5281 | tagtctgacc tttctggttt cttcattttc ttccattact taggaaagag tggaaactcc |
| 5341 | actaaaattt ctctgtgttg ttacagtctt agaggttgca gtactatatt gtaagctttg |
| 5401 | gtgtttgttt aattagcaat agggatggta ggattcaaat gtgtgtcatt tagaagtgga |
| 5461 | agctattagc accaatgaca taaatacata caagacacac aactaaaatg tcatgttatt |
| 5521 | aacagttatt aggttgtcat ttaaaaataa agttccttta tatttctgtc ccatcaggaa |
| 5581 | aactgaagga tatggggaat cattggttat cttccattgt gtttttcttt atggacagga |
| 5641 | gctaatggaa gtgacagtca tgttcaaagg aagcatttct agaaaaaagg agataatgtt |
| 5701 | tttaaatttc attatcaaac ttgggcaatt ctgtttgtgt aactccccga ctagtggatg |
| 5761 | ggagagtccc attgctaaaa ttcagctact cagataaatt cagaatgggt caaggcacct |
| 5821 | gcctgttttt gttggtgcac agagattgac ttgattcaga gagacaattc actccatccc |
| 5881 | tatggcagag gaatgggtta gccctaatgt agaatgtcat tgtttttaaa actgttttat |
| 5941 | atcttaagag tgccttatta aagtatagat gtatgtctta aaatgtgggt gataggaatt |
| 6001 | ttaaagattt atataatgca tcaaaagcct tagaataaga aaagcttttt ttaaattgct |
| 6061 | ttatctgtat atctgaactc ttgaaactta tagctaaaac actaggattt atctgcagtg |
| 6121 | ttcagggaga taattctgcc tttaattgtc taaaacaaaa acaaaaccag ccaacctatg |
| 6181 | ttacacgtga gattaaaacc aattttttcc ccattttttc tccttttttc tcttgctgcc |
| 6241 | cacattgtgc ctttatttta tgagccccag ttttctgggc ttagtttaaa aaaaaaatca |
| 6301 | agtctaaaca ttgcatttag aaagcttttg ttcttggata aaaagtcata cactttaaaa |
| 6361 | aaaaaaaaaa ctttttccag gaaaatatat tgaaatcatg ctgctgagcc tctattttct |
| 6421 | ttctttgatg ttttgattca gtattctttt atcataaatt tttagcattt aaaaattcac |
| 6481 | tgatgtacat taagccaata aactgcttta atgaataaca aactatgtag tgtgtcccta |
| 6541 | ttataaatgc attggagaag tatttttatg agactcttta ctcaggtgca tggttacagc |
| 6601 | ccacagggag gcatggagtg ccatggaagg attcgccact acccagacct tgttttttgt |
| 6661 | tgtattttgg aagacaggtt ttttaaagaa acattttcct cagattaaaa gatgatgcta |
| 6721 | ttacaactag cattgcctca aaaactggga ccaaccaaag tgtgtcaacc ctgtttcctt |
| 6781 | aaaagaggct atgaatccca aaggccacat ccaagacagg caataatgag cagagtttac |
| 6841 | agctccttta ataaaatgtg tcagtaattt taaggtttat agttccctca acacaattgc |
| 6901 | taatgcagaa tagtgtaaaa tgcgcttcaa gaatgttgat gatgatgata tagaattgtg |
| 6961 | gctttagtag cacagaggat gccccaacaa actcatggcg ttgaaaccac acagttctca |
| 7021 | ttactgttat ttattagctg tagcattctc tgtctcctct ctctcctcct ttgaccttct |
| 7081 | cctcgaccag ccatcatgac atttaccatg aatttacttc ctcccaagag tttggactgc |
| 7141 | ccgtcagatt gttgctgcac atagttgcct ttgtatctct gtatgaaata aaaggtcatt |
| 7201 | tgttcatgtt aaaaaaaaa |

MAGT1 (SEQ ID NO: 135)

| | |
|---|---|
| 1 | gtgtagcgcc agcgcgctgt gacgtaatgt gaggggtctc ccggcagggc tgagctggac |
| 61 | caatgaggaa aggcaagggg ccgatttgcc tgttctcacg ccccaccctc agacctagcc |
| 121 | ggagcaaagt ttcacttata gaagggagag gagcgaacat ggcagcgcgt tggcggtttt |
| 181 | ggtgtgtctc tgtgaccatg gtggtggcgc tgctcatcgt ttgcgacgtt ccctcagcct |
| 241 | ctgcccaaag aaagaaggag atggtgttat ctgaaaaggt tagtcagctg atggaatgga |
| 301 | ctaacaaaag acctgtaata agaatgaatg gagacaagtt ccgtcgcctt gtgaaagccc |
| 361 | caccgagaaa ttactccgtt atcgtcatgt tcactgctct ccaactgcat agacagtgtg |
| 421 | tcgtttgcaa gcaagctgat gaagaattcc agatcctggc aaactcctgg cgatactcca |
| 481 | gtgcattcac caacaggata ttttttgcca tggtggattt tgatgaaggc tctgatgtat |
| 541 | ttcagatgct aaacatgaat tcagctccaa ctttcatcaa ctttcctgca aaagggaaac |
| 601 | ccaaacgggg tgatacatat gagttacagg tgcggggttt ttcagctgag cagattgccc |
| 661 | ggtggatcgc cgacagaact gatgtcaata ttagagtgat tagaccccca aattatgctg |
| 721 | gtccccttat gttgggattg cttttggctg ttattggtgg acttgtgtat cttcgaagaa |
| 781 | gtaatatgga atttctcttt aataaaactg gatgggcttt tgcagctttg tgttttgtgc |
| 841 | ttgctatgac atctggtcaa atgtggaacc atataagagg accaccatat gcccataaga |
| 901 | atccccacac gggacatgtg aattatatcc atggaagcag tcaagcccag tttgtagctg |
| 961 | aaacacacat tgttcttctg tttaatggtg gagttacctt aggaatggtg cttttatgtg |
| 1021 | aagctgctac ctctgacatg gatattggaa agcgaaagat aatgtgtgtg gctggtattg |
| 1081 | gacttgttgt attattcttc agttggatgc tctctatttt tagatctaaa tatcatggct |
| 1141 | acccatacag ctttctgatg agttaaaaag gtcccagaga tatatagaca ctggagtact |
| 1201 | ggaaattgaa aaacgaaaat cgtgtgtgtt tgaaaagaag aatgcaactt gtatattttg |
| 1261 | tattacctct ttttttcaag tgatttaaat agttaatcat ttaaccaaag aagatgtgta |
| 1321 | gtgccttaac aagcaatcct ctgtcaaaat ctgaggtatt tgaaaataat tatcctctta |
| 1381 | accttctctt cccagtgaac tttatggaac atttaattta gtacaattaa gtatattata |
| 1441 | aaaattgtaa aactactact ttgttttagt tagaacaaag ctcaaaacta ctttagttaa |
| 1501 | cttggtcatc tgattttata ttgccttatc caaagatggg gaaagtaagt cctgaccagg |
| 1561 | tgttcccaca tatgcctgtt acagataact acattaggaa ttcattctta gcttcttcat |
| 1621 | ctttgtgtgg atgtgtatac tttacgcatc tttccttttg agtagagaaa ttatgtgtgt |
| 1681 | catgtggtct tctgaaaatg gaacaccatt cttcagagca cacgtctagc cctcagcaag |
| 1741 | acagttgttt ctcctcctcc ttgcatattt cctactgaaa tacagtgctg tctatgattg |
| 1801 | tttttgtttt gttgtttttt tgagacggtc tcgctgtgtc acacaggctg gagtgcagtg |
| 1861 | gcgtgagctc ggctgactgc aaactctgcc tcccaggttt aagcgattct cctgtcacag |
| 1921 | cttcccaagt agctgggatt tacaggtgtg caccgccatg ccaggctaat ttttgtgttt |
| 1981 | ttagtagaga cagggtttcg ccaagttgtc caggctggtc ttgaactcct gggctcaagt |
| 2041 | gatccgcccg cctcagtctc ccaaagtgcg aggatgacat gtgtgagcta ccacaccagc |
| 2101 | aatgtctatg cttctcgata gctgtgaaca tgaaaagaca tctattggga gtccgaggca |
| 2161 | ggtggattgc ttgaggccag gagttagaga ccagcctggc caacaaggca aaaccccgtc |
| 2221 | tctactaaaa atatgaaaat tagctgggct tggtggctca tgcctataat cctagctact |
| 2281 | tgggaggctg aggcacgaga cttgcttaat acctgggagg cggagattgc agtgagccga |
| 2341 | gatcacgcta ctgcgctcca gcctgagtga tagagtgaga ctctgtctca aaaaaaagta |
| 2401 | tctctaaata caggattata atttctgctt gagtatggtg ttaactacct tgtatttaga |
| 2461 | aagatttcag attcattcca tctccttagt tttcttttaa ggtgacccat ctgtgataaa |
| 2521 | aatatagctt agtgctaaaa tcagtgtaac ttatacatgg cctaaaatgt ttctacaaat |
| 2581 | tagagtttgt cacttattcc atttgtacct aagagaaaaa taggctcagt tagaaaagga |
| 2641 | ctccctggcc aggcgcagtg acttacgcct gtaatctcag cactttggga ggccaaggca |
| 2701 | ggcagatcac gaggtcagga gttcgagacc atcctggcca acatggtgaa accccgtctc |
| 2761 | tactaaaaat ataaaaatta gctgggtgtg gtggcaggag cctgtaatcc cagctacaca |
| 2821 | ggaggctgag gcacgagaat cacttgaact caggagatgg aggatcagt gagccaagat |
| 2881 | cacaccactg cactccagcc tggcaacaga gcgagactcc atctcaaaaa aaaaaaaaaa |
| 2941 | agtaagaaag aaaaggactc ccttagaatg ggaaagaaaa atcataaaat attgagctga |
| 3001 | tgcctgtata tagaaattaa gcgtttctcg aaagctgttc tatgttttgc tgttatttta |
| 3061 | gtctttattc tcttccttta ggtggagaaa caaagtacca atttgaaggg atttttttta |
| 3121 | ttttgtcttt tggtttctgt cagtagaaat aaccatatgt gctaaccaaa tttctgtgaa |
| 3181 | gaatgttttc atggttatca ttatatctaa ctataacctc ccccatagtt atgaagagta |
| 3241 | acctgaaatg ccactattgt ggaaatagga taattgtaat tgtgaaaaaa taattttaag |
| 3301 | gaaatcttac aagtattaca ttaaaaagat actatgactg ccacctgcca tttaccttct |
| 3361 | aataaccctg ccatgtggtt tgcagaaaga gatggatata gtagcctcag aagaaatatt |
| 3421 | ttatgtgggt tttttgtttt tcgttactag atttcatgga tgaggggata tggttgacct |
| 3481 | tttacttttt aatggagcag ccagtttttg ttaattactc acttgtaaat tgtgagattc |
| 3541 | tgaattcctt acctgctatt cttgtacttg tctcaggcca aatctatgct gtggttctta |
| 3601 | tgagacttgt atgaagatgc cctgatttgt acagattgac cacgggaata ctactgccat |
| 3661 | gtaatctgta tagttccaga taatttgtca tgaacattga cagaatgaca attttttgta |
| 3721 | tttgcttttt ctccctttaa gagcacattc ttctgtaagg agaaaggcag cattctggct |
| 3781 | aaaatgtgta gaaggtaatt tactacactt ataaaatagt gtgacttttg tgaaaatttt |
| 3841 | gaattagctt tcatatgaag tgccttaagt agactcttca tttacttttc tggtaatggt |
| 3901 | ttaaatatca tttgttatgc atttttaaga tacagttcag aatgacacat tgtagtggca |
| 3961 | aagataacca aatgtctggc tgtttgcttt ttgaccatat caataaactt ttacaatcta |
| 4021 | aaaaaaaaaa aaaaa |

ZIM2 (SEQ ID NO: 136)

| | |
|---|---|
| 1 | ggtgcagaag tctgggcagc tgcgggagga gaggtttggg aggcgcggga gatgtccacc |
| 61 | ctgggctggt ggcgccgccg ggcgccgggc gccatgaggg tgcgctaggc ggctgttcgt |
| 121 | gcccgaggct gcgcagcact gagctttgcc ttcttgatct tccgtccttc ttggagacga |
| 181 | ctggcgagag gaagagggac taggtccaaa cgctaggtgg ctgggtccag atacctgtgt |
| 241 | tttgactctg ttcctgtgga tagctgcttg gtctgaagtt ccagaaagga tcctgttccc |
| 301 | agacagccgg agacccgcac caaggaggag atcatcgagc tcttggtcct tgagcagtac |
| 361 | ctgaccatca tccctgaaaa gctcaagcct tgggtgcgag caaaaaagcc ggagaactgt |
| 421 | gagaagctcg tcactctgct ggagaattac aaggagatgt accaaccaga agacgacaac |
| 481 | aacagtgacg tgaccagcga cgacgacatg acccggaaca gaagagagtc ctcaccacct |
| 541 | cactcagtcc attctttcag tggtgaccgg gactgggacc ggaggggcag aagcagagac |
| 601 | atggagccac gagaccgctg gtcccacacc aggaacccaa gaagcaggat gcctccgcgg |
| 661 | gatctttccc ttcctgtggt ggcgaaaaca agctttgaaa tggacagaga ggacgacagg |
| 721 | gactccaggg cttatgagtc ccgatctcag gatgctgaat cataccaaaa tgtggtggac |
| 781 | ctcgctgagg acaggaaacc tcacaacaca atccaggaca acatggaaaa ctacaggaag |
| 841 | ctgctctccc tcggtttcct tgctcaggac tctgtccctg cagaaaagag gaacacagag |
| 901 | atgttagaca atctgccatc tgctgggtcc cagttcccgg acttcaaaca cttaggaaca |
| 961 | tttctggtgt ttgaggagtt ggtgaccttc gaggatgtgc ttgtggactt cagcccagag |
| 1021 | gaacttagtt cccttagtgc tgctcagaga aacctctaca gggaggtgat gctggagaat |
| 1081 | taccggaacc tggtctccct ggggcaccag ttctctaaac ctgacattat ctcacgcctg |
| 1141 | gaagaggagg aatcatatgc aatggagaca gacagcagac atacagtgat ttgtcaagga |
| 1201 | gagtctcatg atgatccatt ggaaccacac cagggcaacc aagagaaact tttgactcct |
| 1261 | ataacaatga atgaccccaa gaccctcact ccggaaagaa gctatggcag tgatgaattt |
| 1321 | gagagaagct ctaatcttag taaacaatca aaggatcctc taggaaagga tccccaggaa |
| 1381 | ggcactgctc ctggaatatg tacgagtccc cagtcagcat cccaagagaa caaacacaac |
| 1441 | agatgtgaat tttgcaaacg aacctttagt acgcaagtag cccttaggag acacgaacgg |
| 1501 | atccatactg ggaagaaacc ctatgaatgt aaacagtgtg ctgaagcctt ctatctcatg |
| 1561 | ccacacctca acagacatca gaagacccat tctggtagga agacttctgg ctgcaatgaa |
| 1621 | ggtagaaagc cttccgtcca gtgtgcgaat ctctgtgaac gtgtaagaat tcacagtcag |
| 1681 | gaggactact ttgaatgttt tcagtgcggc aaagcttttc tccagaatgt gcatcttctt |
| 1741 | caacatctca aagcccatga ggcagcaaga gtccttcctc ctgggttgtc ccacagcaag |
| 1801 | acatacttaa ttcgttatca gcggaaacat gactacgttg gagagagagc ctgccagtgt |
| 1861 | tgtgactgtg gcagagtctt cagtcggaat tcatatctca ttcagcatta tagaactcac |
| 1921 | actcaagaga ggccttacca gtgtcagcta tgtgggaaat gtttcggccg accctcatac |
| 1981 | ctcactcaac attatcaact ccattctcaa gagaaaactg ttgagtgcga tcactgttga |
| 2041 | gaaaccttta gtcacagcac acacttttct caacattatt ggcttcctcc tagagtgttg |
| 2101 | tgagtgtgag aaggcctttc actagcccca ccttgttaac aacttgaaca ttcatcaaag |
| 2161 | tgtggtaaaa aaaaaaaaaa aaaaaaaaa |

RPS19 (SEQ ID NO: 137)

| | |
|---|---|
| 1 | gtactttcgc catcatagta ttctccacca ctgttccttc cagccacgaa cgacgcaaac |
| 61 | gaagccaagt tcccccagct ccgaacagga gctctctatc ctctctctat tacactccgg |
| 121 | gagaaggaaa cgcgggagga aacccaggcc tccacgcgcg accccttggc cctccccttt |
| 181 | acctctccac ccctcactag acaccctccc ctctaggcgg ggacgaactt tcgccctgag |
| 241 | agaggcggag cctcagcgtc taccctcgct ctcgcgagct ttcggaactc tcgcgagacc |
| 301 | ctacgcccga cttgtgcgcc cgggaaaccc cgtcgttccc tttcccctgg ctggcagcgc |
| 361 | ggaggccgca cgatgcctgg agttactgta aaagacgtga accagcagga gttcgtcaga |
| 421 | gctctggcag ccttcctcaa aaagtccggg aagctgaaag tccccgaatg ggtggatacc |
| 481 | gtcaagctgg ccaagcacaa agagcttgct ccctacgatg agaactggtt ctacacgcga |
| 541 | gctgcttcca cagcgcggca cctgtacctc cggggtggcg ctggggttgg ctccatgacc |
| 601 | aagatctatg ggggacgtca gagaaacggc gtcatgccca gccacttcag ccgaggctcc |
| 661 | aagagtgtgg cccgccgggt cctccaagcc ctggaggggc tgaaaatggt ggaaaaggac |
| 721 | caagatggcg gccgcaaact gacacctcag ggacaaagag atctggacag aatcgccgga |
| 781 | caggtggcag ctgccaacaa gaagcattag aacaaaccat gctgggttaa taaattgcct |
| 841 | cattcgtaaa aaaaaaaaaa aaaaaaaaaa aa |

IQGAP3 (SEQ ID NO: 138)

| | |
|---|---|
| 1 | gtcctgtctg gcggtgccga cggtgagggg cggtggccca acggcgggag attcaaacct |
| 61 | ggaagaagga ggaacatgga gaggagagca gcgggcccag gctgggcagc ctatgaacgc |
| 121 | ctcacagctg aggagatgga tgagcagagg cggcagaatg ttgcctatca gtacctgtgc |
| 181 | cggctggagg aggccaagcg ctggatggag gcctgcctga aggaggagct tccttccccg |
| 241 | gtggagctgg aggagagcct tcggaatgga gtgctgctgg ccaagctagg ccactgtttt |
| 301 | gcaccctccg tggttccctt gaagaagatc tacgatgtgg agcagctgcg gtaccaggca |
| 361 | actggcttac atttccgtca cacagacaac atcaactttt ggctatctgc aatagcccac |
| 421 | atcggtctgc cttcgacctt cttcccagag accacggaca tctatgacaa aaagaacatg |
| 481 | ccccgggtag tctactgcat ccatgctctc agtctcttcc tcttccggct gggattggcc |
| 541 | cctcagatac atgatctata cgggaaagtg aaattcacag ctgaggaact cagcaacatg |
| 601 | gcgtccgaac tggccaaata tggcctccag ctgcctgcct tcagcaagat cgggggcatc |
| 661 | ttggccaatg agctctcggt ggatgaggct gcagtccatg cagctgttct tgccatcaat |
| 721 | gaagcagtgg agcgaggggt ggtggaggac accctggctg ccttgcagaa tcccagtgct |
| 781 | cttctggaga atctccgaga gcctctggca gccgtctacc aagagatgct ggcccaggcc |
| 841 | aagatggaga aggcagccaa tgccaggaac catgatgaca gagaaagcca ggacatctat |
| 901 | gaccactacc taactcaggc tgaaatccag ggcaatatca accatgtcaa cgtccatggg |
| 961 | gctctagaag ttgttgatga tgccctggaa agacagagcc ctgaagcctt gctcaaggcc |
| 1021 | cttcaagacc ctgccctggc cctgcgaggg gtgaggagag actttgctga ctggtacctg |
| 1081 | gagcagctga actcagacag agagcagaag gcacaggagc tgggcctggt ggagcttctg |
| 1141 | gaaaaggagg aagtccaggc tggtgtggct gcagccaaca caaagggtga tcaggaacaa |
| 1201 | gccatgctcc acgctgtgca gcggatcaac aaagccatcc ggaggggagt ggcggctgac |
| 1261 | actgtgaagg agctgatgtg ccctgaggcc cagctgcctc cagtgtaccc tgttgcatcg |
| 1321 | tctatgtacc agctggagct ggcagtgctc cagcagcagc agggggagct tggccaggag |
| 1381 | gagctcttcg tggctgtgga gatgctctca gctgtggtcc tgattaaccg ggccctggag |
| 1441 | gcccgggatg ccagtggctt ctggagcagc ctggtgaacc ctgccacagg cctggctgag |
| 1501 | gtggaaggag aaaatgccca gcgttacttc gatgccctgc tgaaattgcg acaggagcgt |
| 1561 | gggatgggtg aggacttcct gagctggaat gacctgcagg ccaccgtgag ccaggtcaat |
| 1621 | gcacagaccc aggaagagac tgaccgggtc cttgcagtca gcctcatcaa tgaggctctg |
| 1681 | gacaaaggca gccctgagaa gactctgtct gccctactgc ttcctgcagc tggcctagat |
| 1741 | gatgtcagcc tccctgtcgc ccctcggtac catctcctcc ttgtggcagc caaaaggcag |
| 1801 | aaggcccagg tgacagggga tcctggagct gtgctgtggc ttgaggagat ccgccaggga |
| 1861 | gtggtcagag ccaaccagga cactaataca gctcagagaa tggctcttgg tgtggctgcc |
| 1921 | atcaatcaag ccatcaagga gggcaaggca gcccagactg agcgggtgtt gaggaacccc |
| 1981 | gcagtggccc ttcgaggggt agttcccgac tgtgccaacg gctaccagcg agccctggaa |
| 2041 | agtgccatgg caaagaaaca gcgtccagca gacacagctt tctgggttca acatgacatg |
| 2101 | aaggatggca ctgcctacta cttccatctg cagaccttcc aggggatctg ggagcaacct |
| 2161 | cctggctgcc ccctcaacac ctctcacctg acccgggagg agatccagtc agctgtcacc |
| 2221 | aaggtcactg ctgcctatga ccgccaacag ctctggaaag ccaacgtcgg ctttgttatc |
| 2281 | cagctccagg cccgcctccg tggcttccta gttcggcaga agtttgctga gcattcccac |
| 2341 | tttctgagga cctggctccc agcagtcatc aagatccagg ctcattggcg gggttatagg |
| 2401 | cagcggaaga tttacctgga gtggttgcag tattttaaag caaacctgga tgccataatc |
| 2461 | aagatccagg cctgggcccg gatgtgggca gctcggaggc aatacctgag gcgtctgcac |
| 2521 | tacttccaga agaatgttaa ctccattgtg aagatccagg catttttccg agccaggaaa |
| 2581 | gcccaagatg actacaggat attagtgcat gcaccccacc ctcctctcag tgtggtacgc |
| 2641 | agatttgccc atctcttgaa tcaaagccag caagacttct tggctgaggc agagctgctg |
| 2701 | aagctccagg aagaggtagt taggaagatc cgatccaatc agcagctgga gcaggacctc |
| 2761 | aacatcatgg acatcaagat tggcctgctg gtgaagaacc ggatcactct gcaggaagtg |
| 2821 | gtctcccact gcaagaagct gaccaagagg aataaggaac agctgtcaga tatgatggtt |
| 2881 | ctggacaagc agaagggttt aaagtcgctg agcaaagaga aacggcagaa actagaagca |
| 2941 | taccaacacc tcttctacct gctccagact cagcccatct acctggccaa gctgatcttt |
| 3001 | cagatgccac agaacaaaac caccaagttc atggaggcag tgattttcag cctgtacaac |
| 3061 | tatgcctcca gccgccgaga ggcctatctc ctgctccagc tgttcaagac agcactccag |
| 3121 | gaggaaatca agtcaaaggt ggagcagccc caggacgtgg tgacaggcaa cccaacagtg |
| 3181 | gtgaggctgg tggtgagatt ctaccgtaat gggcggggac agagtgccct gcaggagatt |
| 3241 | ctgggcaagg ttatccagga tgtgctagaa gacaaagtgc tcagcgtcca cacagaccct |
| 3301 | gtccacctct ataagaactg gatcaaccag actgaggccc agacagggca gcgcagccat |
| 3361 | ctcccatatg atgtcacccc ggagcaggcc ttgagccacc ccgaggtcca gagacgactg |
| 3421 | gacatcgccc tacgcaacct cctcgccatg actgataagt tccttttagc catcacctca |
| 3481 | tctgtggacc aaattccgta tgggatgcga tatgtggcca aagtcctgaa ggcaactctg |
| 3541 | gcagagaaat tccctgacgc cacagacagc gaggtctata aggtggtcgg gaacctcctg |
| 3601 | tactaccgct tcctgaaccc agctgtggtg gctcctgacg ccttcgacat tgtggccatg |
| 3661 | gcagctggtg gagccctggc tgccccccag cgccatgccc tgggggctgt ggctcagctc |
| 3721 | ctacagcacg ctgcggctgg caaggccttc tctgggcaga gccagcacct acgggtcctg |
| 3781 | aatgactatc tggaggaaac acacctcaag ttcaggaagt tcatccatag agcctgccag |
| 3841 | gtgccagagc cagaggagcg ttttgcagtg gacgagtact cagacatggt ggctgtggcc |
| 3901 | aaacccatgg tgtacatcac cgtgggggag ctggtcaaca cgcacaggct gttgctggag |
| 3961 | caccaggact gcattgcccc tgatcaccaa gaccccctgc atgagctcct ggaggatctt |
| 4021 | ggggagctgc ccaccatccc tgaccttatt ggtgagagca tcgctgcaga tgggcacacg |
| 4081 | gacctgagca agctagaagt gtccctgacg ctgaccaaca agtttgaagg actagaggca |
| 4141 | gatgctgatg actccaacac ccgtagcctg cttctgagca ccaagcagct gttggccgat |
| 4201 | atcatacagt tccatcctgg ggacaccctc aaggagatcc tgtccctctc ggcttccaga |
| 4261 | gagcaagaag cagcccacaa gcagctgatg agccgacgcc aggcctgtac agcccagaca |
| 4321 | ccggagccac tgcgacgaca ccgctcactg acagctcact ccctcctgcc actggcagag |
| 4381 | aagcagcggc gcgtcctgcg gaacctacgc cgacttgaag ccctggggtt ggtcagcgcc |
| 4441 | agaaatggct accaggggct agtggacgag ctggccaagg acatccgcaa ccagcacaga |
| 4501 | cacaggcaca ggcggaaggc agagctggtg aagctgcagg ccacattaca gggcctgagc |
| 4561 | actaagacca ccttctatga ggagcagggt gactactaca gccagtacat ccgggcctgc |
| 4621 | ctggaccacc tggcccccga ctccaagagt tctgggaagg ggaagaagca gccttctctt |
| 4681 | cattacactg ctgctcagct cctggaaaag ggtgtcttgg tggaaattga agatcttccc |
| 4741 | gcctctcact tcagaaacgt catctttgac atcacgccgg gagatgaggc aggaaagttt |
| 4801 | gaagtaaatg ccaagttcct gggtgtggac atggagcgat ttcagcttca ctatcaggat |
| 4861 | ctcctgcagc tccagtatga gggtgtggct gtcatgaaac tcttcaacaa ggccaaagtc |
| 4921 | aatgtcaacc ttctcatctt cctcctcaac aagaagtttt tgcggaagtg acagaggcaa |
| 4981 | agggtgctac ccaagcccct cttacctctc tggatgcttt ctttaacact aactcaccac |
| 5041 | tgtgcttccc tgcagacacc cagagctcag gactgggcaa ggcccaggga ttctcacccc |
| 5101 | ttccccagct gggaggagct tgcctgcctg gccacagaca gtgtatcttc taattggcta |
| 5161 | aagtgggcct tgcccagagt ccagctgtgt ggcttttatc atgcatgaca aacccctggc |
| 5221 | tttcctgcca gatggtagga catggacctt gacctgggaa agccattact cttgtgtctg |
| 5281 | ctactgccct cccacagtca ccccaatatt acaagcactg ccccagcggc ttgatttccc |
| 5341 | ctctgccttc cttctctctg cactcccaca aagccagggc caggctcccc atccctacct |
| 5401 | cccactgcat cagcagtggg tgttcctgcc cttcctgagt ctaggcagct ctgctgctgt |
| 5461 | gatctgcaca ccctccaacc tgggcaggga ctggggggat gcagtgtgtg ttagtgccca |
| 5521 | tgtggcattg tggcactgtt gccccccatg gcggcatggg caagatgacc ttccattagc |
| 5581 | ttcaagtctt gttctcttgt ctgtggtctg tttaatatgt gggtcactag ggtatttatt |
| 5641 | ctttctccca tccttacact ctggatcatt gtgcagactt aatcagggtt ttaacgcttt |
| 5701 | catttttttt tttttttttt tttttttgag ctcaaagaga gttctcattt tccctattca |
| 5761 | aactaatacc catgccgtgt tttttacctt ggatttaaag tcaccttagg ttggggcaac |
| 5821 | agattctcac tcatgtttaa gatcttgtta tttcagcttc ataagatcaa agaggagtct |
| 5881 | ttcccttttc tcttttaccc tcaggattct catcccttac agctgactct tccaggcaat |
| 5941 | ttccatagat ctgcagtcct gcctctgcca cagtctctct gttgtcccca catctaccca |
| 6001 | acttcctgta ctgttgccct tctgatgtta ataaaagcag ctgttactcc caaaaaaaaa |
| 6061 | aaaaaaaaa |

XRCC3 (SEQ ID NO: 139)

| | |
|---|---|
| 1 | ctattggagg agaaggccga gaggagcagg acggcgggaa gaggagtgcg gaacccgcgg |
| 61 | gagagtcccc agggagacac ttaagggaaa ttaaactgca gagtgcaaga gatgcctcag |
| 121 | tcaagtcagc caaaaacacg cgggtcatcc ccaagcccca gagagtgaca gagccccgat |
| 181 | gacacggaca cctcggctgc tgtcacttcc ctggttcggg cctcccacag gctttgaatt |
| 241 | gaaggcgagt gcctcagaat ttgcatccat tgttctgtct ttcctgggaa gttattcatc |
| 301 | ctggtggcca gcccaccgac aaaatggatt tggatctact ggacctgaat cccagaatta |
| 361 | ttgctgcaat taagaaagcc aaactgaaat cggtaaagga ggttttacac ttttctggac |
| 421 | cagacttgaa gagactgacc aacctctcca gccccgaggt ctggcacttg ctgagaacgg |
| 481 | cctccttaca cttgcgggga agcagcatcc ttacagcact gcagctgcac cagcagaagg |
| 541 | agcggttccc cacgcagcac cagcgcctga gcctgggctg cccggtgctg gacgcgctgc |
| 601 | tccgcggtgg cctgcccctg gacggcatca ctgagctggc cggacgcagc tcggcaggga |
| 661 | agacccagct ggcgctgcag ctctgcctgg ctgtgcagtt cccgcggcag cacggaggcc |
| 721 | tggaggctgg agccgtctac atctgcacgg aagacgcctt cccgcacaag cgcctgcagc |
| 781 | agctcatggc ccagcagccg cggctgcgca ctgacgttcc aggagagctg cttcagaagc |
| 841 | tccgatttgg cagccagatc ttcatcgagc acgtggccga tgtggacacc ttgttggagt |
| 901 | gtgtgaataa gaaggtcccc gtactgctgt ctcggggcat ggctcgcctg gtggtcatcg |
| 961 | actcggtggc agccccattc cgctgtgaat ttgacagcca ggcctccgcc cccagggcca |
| 1021 | ggcatctgca gtccctgggg gccacgctgc gtgagctgag cagtgccttc cagagccctg |
| 1081 | tgctgtgcat caaccaggtg acagaggcca tggaggagca gggcgcagca cacgggccgc |
| 1141 | tggggttctg ggacgaacgt gtttccccag cccttggcat aacctgggct aaccagctcc |
| 1201 | tggtgagact gctggctgac cggctccgcg aggaagaggc tgccctcggc tgcccagccc |
| 1261 | ggaccctgcg ggtgctctct gccccccacc tgcccccctc ctcctgttcc tacacgatca |
| 1321 | gtgccgaagg ggtgcgaggg acacctggga cccagtccca ctgacacggt ggcggctgca |
| 1381 | caacagccct gcctgagaag ccccgacaca cggggctcgg gcctttaaaa cgcgtctgcc |
| 1441 | tgggccgtgg cacagctggg agcctggttc agacacagct cttccagggc agcggctcca |
| 1501 | ctttctcatc cgaagatggt ggccacagac tgacccccat ctgagctggg gggatgttct |
| 1561 | gcctctccct gggtctgggg acaggcccgc ttgctgggta cctggtcccc actgctgagc |
| 1621 | tggcccttgg ggagaggtga ttctcagggc tggagcctgg ggtgtcctac agtgactccc |
| 1681 | tgggagccgc ctgcttcttc tctccacatg gaagcccaac tggggttgcg tctgaggcct |
| 1741 | gccccctggg ctggggcctc agaccccctc agccttggga ccgtgcccac gagggtctcc |
| 1801 | cctcctgcac acagggcagt ccttactccc ccaccactca ggccacagtg gggctgcagg |
| 1861 | caggcggctc ctcctcaccc acctctgggt ccttggctcc cgggggcccc acctcggcac |
| 1921 | acactgtgcc ccacaaaact tcagtgtggt acaaggtgga gaaagcatat cccaccaacc |
| 1981 | tccagtgtca gggtccagga gagcctgggg gtggggggac tgccttgtct ctagtagtgt |
| 2041 | ggcctgtgcc agcaccacag ccggtcagag gagcgcaggc agcgcagggc tggcacgtga |
| 2101 | caggctcgtc agccacctgg gaacacagtt ctgggcaaag aggatccgag gttgagagga |
| 2161 | aggagggtcc cggtgtatcc tggccctggg ggtctgggcg tccagctcag ccctggcctg |
| 2221 | gctgggtggt attctggtag ggatatggca ggactcctgg cagggccacc tgcaggaccc |
| 2281 | tgtcctgcag tcccacactg tgcagaccca gtcccacact gtggccaggc cttacatctg |
| 2341 | gctggaaagc agagcctcct gggaacacat ctggctgcac aggctgaaat atccacccag |
| 2401 | caggcagagt ggcgtggcct ccccatgggc acagtggtga cccccttgat tcccaccgta |
| 2461 | caaccccctc caccccccac tcagtgcctc cacatgctgc ctggcacaga ccaggccttt |
| 2521 | gacaaataaa tgttcaatgg atgcaaaaaa aaaaaaaaaa aaa |

RPL13A (SEQ ID NO: 140)

| | |
|---|---|
| 1 | cacttctgcc gcccctgttt caagggataa gaaaccctgc gacaaaacct cctccttttc |
| 61 | caagcggctg ccgaagatgg cggaggtgca ggtcctggtg cttgatggtc gaggccatct |
| 121 | cctgggccgc ctggcggcca tcgtggctaa acagtgaagt acctggcttt cctccgcaag |
| 181 | cggatgaaca ccaacccttc ccgaggcccc taccacttcc gggcccccag ccgcatcttc |
| 241 | tggcggaccg tgcgaggtat gctgccccac aaaaccaagc gaggccaggc cgctctggac |
| 301 | cgtctcaagg tgtttgacgg catcccaccg ccctacgaca agaaaaagcg gatggtggtt |
| 361 | cctgctgccc tcaaggtcgt gcgtctgaag cctacaagaa agtttgccta tctggggcgc |
| 421 | ctggctcacg aggttggctg gaagtaccag gcagtgacag ccaccctgga ggagaagagg |
| 481 | aaagagaaag ccaagatcca ctaccggaag aagaaacagc tcatgaggct acggaaacag |
| 541 | gccgagaaga acgtggagaa gaaaattgac aaatacacag aggtcctcaa gacccacgga |
| 601 | ctcctggtct gagcccaata aagactgtta attcctcatg cgttgcctgc ccttcctcca |
| 661 | ttgttgccct ggaatgtacg ggacccaggg gcagcagcag tccaggtgcc acaggcagcc |
| 721 | ctgggacata ggaagctggg agcaaggaaa gggtcttagt cactgcctcc cgaagttgct |
| 781 | tgaaagcact cggagaattg tgcaggtgtc atttatctat gaccaatagg aagagcaacc |
| 841 | agttactatg agtgaaaggg agccagaaga ctgattggag ggccctatct tgtgagtggg |
| 901 | gcatctgttg gactttccac ctggtcatat actctgcagc tgttagaatg tgcaagcact |
| 961 | tggggacagc atgagcttgc tgttgtacac agggtatttc tagaagcaga aatagactgg |
| 1021 | gaagatgcac aaccaagggg ttacaggcat cgcccatgct cctcacctgt attttgtaat |
| 1081 | cagaaataaa ttgcttttaa agaaaaaaaa aaaaaaaaaa |

1. A method of identifying a genetic interaction in a subject or population of subjects comprising:
(a) selecting at least a first pair of nucleic acids comprising a first and second nucleic acid from a dataset of a subject or population of subjects, wherein either:
(i) expression or somatic copy number alteration (SCNA) of the first nucleic acid contributes to susceptibility of a disease or disorder and expression or SCNA of the second nucleic acid at least partially modulates or reverses the susceptibility caused by expression of the first nucleic acid; or
(ii) expression or somatic copy number alteration (SCNA) of both the first and second nucleic acids contribute to susceptibility of a disease or disorder greater than expression or SCNA in a control subject or control population of subjects; and
(b) correlating expression of the first pair of genes with a survival rate associated with a disease or disorder in the subject or the population of subjects;
(c) assigning a probability score to the first pair of genes based upon the survival rate;
(d) identifying the first pair of nucleic acid sequences as being in a genetic interaction if the probability score of step (c) is about or within the top twenty percent of a set of pairs of nucleic acid sequences correlated in step (c).

2. The method of claim 1 further comprising:
(i) calculating an essentiality value associated with the first pair of nucleic acids from an in vitro or in vivo dataset;
(ii) correlating the essentiality value with a likelihood that the first pair of nucleic acids is associated with the disease or disorder;
wherein both steps (i) and (ii) are performed sequentially after step (b); and
wherein the probability score of step (c) is based upon step (ii).

3. The method of claim 1, further comprising:
(iii) conducting a phylogenetic analysis of the first pair of nucleic acids across one or a plurality of data from a species which is not the species of the subject or population of the subjects; and
wherein step (iii) is performed after step (b) and before step (c); and
wherein the probability score of step (c) is based upon the phylogenetic analysis of step (iii).

4. The method of claim 1, wherein the step of selecting at least a first pair of nucleic acids comprises performing a binomial test to predict whether: (i) expression of the second nucleic acid at least partially reverses a biological effect of the expression of the first nucleic acid; or (ii) expression of the first and second nucleic acid sequences causes a biological effect the magnitude or phenotypic result of which exceeds a biological effect or phenotypic result caused by individual expression the first or second nucleic acid sequence.

5. The method of claim 1, wherein correlating expression of the first pair of nucleic acid sequences with a survival rate associated with a disease or disorder in the subject or the population of subjects comprises comparing expression of the first pair of nucleic acid sequences in a subject or population of subjects with the disease or disorder with expression of the first pair of nucleic acid sequences in a control subject or control population of subjects.

6.-7. (canceled)

8. The method of claim 2, wherein calculating an essentiality value is calculated by: exposing a cell expressing the first nucleic acid to a quantity of short hairpin ribonucleic acid (shRNA) complementary to the first nucleic acid sufficient to disrupt expression of the first nucleic acid in the cell, such that loss of function of the first nucleic acid causes susceptibility of the cell to die and monitoring lethality of the cell in the presence and absence of the second nucleic acid expressed at a quantity sufficient to rescue the cell from lethality; and quantifying the extent to which any cells die or survive in the presence and absence of the second nucleic acid.

9. The method of claim 2, calculating an essentiality value is calculated by performing a Wilcoxon rank-sum test.

10. The method of claim 3, wherein the phylogenetic analysis is performed using a non-negative matrix factorization test.

11. The method of claim 1, wherein the subject or population of subjects comprises data collected in the presence and absence of: an environmental stimulus or chemical substance.

12.-13. (canceled)

14. The method of claim 1, wherein the method is a computer-implemented method, the method comprising: in a system configured to perform statistical analysis comprising at least one processor and a memory, performing statistical analysis or calculating a probability score of any of steps (a), (b), or (c).

15. The method of claim 14, wherein the step of calculating the probability score or performing the statistical analysis, by the at least one processor, comprises:
setting, by the at least one processor, a predetermined value, stored in the memory, that corresponds to a probability score above which a nucleic acid sequence pair is correlated the subject or population survival rate;
calculating, by the at least one processor, the probability score, wherein calculating the probability score comprises receiving subject or population information associated with a disease or disorder, conducting one or a plurality of statistical tests from the information associated with a disease or disorder, and assigning a probability score based upon a comparison of an outcome of the statistical tests and the predetermined value.

16. (canceled)

17. A method of predicting responsiveness of a subject or population of subjects to a therapy comprising:
(a) selecting, from the subject or the population on the therapy, at least a first pair of nucleic acid sequences comprising a first and second sequence, wherein the first nucleic acid sequence is targeted by the therapy and expression of the second nucleic acid sequence at least partially contributes to the development of the resistance or at least partially enhances the responsiveness of the therapy targeting the first gene;
(b) correlating expression of the first pair of nucleic acid sequences with a survival rate associated with a disease or disorder in the subject or the population of subjects;
(c) assigning a probability score to the first pair of nucleic acid sequences based upon the survival rate;
(d) predicting the subject or population's responsiveness to a therapy based upon expression of the second nucleic acid sequence if the probability score of step (c) is about or within the top twenty percent of a set of pairs of nucleic acid sequences correlated in step (c).

18-23. (canceled)

24. The method of claim 17, further comprising:
(i) calculating an essentiality value associated with the first pair of nucleic acids from an in vitro and/or in vivo dataset;
(ii) correlating the essentiality value with a likelihood that the first pair of nucleic acid sequences is associated with responsiveness to a therapy for treatment of the disease or disorder;
wherein both steps (i) and (ii) are performed sequentially after step (b); and
wherein the probability score of step (c) is based upon step (ii)
wherein calculating an essentiality value is calculated by:
exposing a cell expressing the first nucleic acid to a quantity of short hairpin ribonucleic acid (shRNA) complementary to the first nucleic acid sufficient to disrupt expression of the first nucleic acid in the cell, such that either: (i) loss of function of the first nucleic acid causes susceptibility of the cell to die and monitoring lethality of the cell in the presence and absence of the second nucleic acid expressed at a quantity sufficient to rescue the cell from lethality; or (ii) the loss of function of the first nucleic acid alone does not have a phenotypic consequence, but the presence and absence of the second nucleic acid expressed at a quantity sufficient to lead the cell to lethality; and
quantifying the extent to which any cells die or survive in the presence and/or absence of the second nucleic acid and/or the therapy.

25.-26. (canceled)

27. The method of claim 17, wherein the subject or population of subjects comprises data collected while the subject or population of subjects is exposed to cancer therapy.

28. (canceled)

29. The method of claim 27, wherein the cancer therapy is Tamoxifin® or Herceptin®.

30.-32. (canceled)

33. A method of predicting a likelihood of a subject or population of subjects develops a resistance to a therapy comprising:
(a) selecting, from the subject or the population of subjects administered the therapy, at least a first pair of nucleic acid sequences comprising a first and second nucleic acid sequence, wherein the first nucleic acid sequence is targeted by the therapy and alteration in the expression of the second nucleic acid sequence at least partially contributes to the emergence of resistance reducing the effectiveness of the therapy targeting the first nucleic acid sequence;
(b) correlating expression of the first pair of nucleic acid sequences with a survival rate associated with a disease or disorder in the subject or the population of subjects;
(c) assigning a probability score to the first pair of nucleic acid sequences based upon the survival rate;
(d) predicting the subject or population's likelihood of developing resistance to a therapy based upon expression of the second nucleic acid sequence if the probability score of step (c) is about or within the top twenty percent of a set of pairs of nucleic acid sequences correlated in step (c).

34.-48. (canceled)

49. A method of predicting a prognosis and/or a clinical outcome of a subject or population of subjects suffering from a disease or disorder comprising:
(a) selecting at least a first pair of nucleic acids comprising a first and second nucleic acid, wherein either
(i) expression or SCNA of the first nucleic acid contributes to severity of a disease or disorder and expression of the second nucleic acid at least partially modulates the severity of the disease or disorder caused by expression of the first nucleic acid; or
(ii) expression or SCNA of both the nucleic acids contribute to susceptibility of a disease or disorder greater than a control subjects or population;
(b) correlating expression of the first pair of nucleic acid sequences with a survival rate associated with a disease or disorder in the subject or the population of subjects;
(c) assigning a probability score to the first pair of nucleic acid sequences based upon the survival rate;
(d) prognosing the clinical outcome of the subject or the population of subjects based upon the expression of the first pair of nucleic acid sequences if the probability score of step (c) is about or within the top twenty percent of a set of pairs of nucleic acid sequences correlated in step (c).

50. The method of claim 1 further comprising:
(i) calculating an essentiality value associated with the first pair of nucleic acids from an in vitro or in vivo dataset;
(ii) correlating the essentiality value with a likelihood that expression of the first pair of nucleic acids is associated with the prognosis of the disease or disorder in the subject or population of subjects;
wherein both steps (i) and (ii) are performed sequentially after step (b); and
wherein the probability score of step (c) is based at least partially upon step (ii).

51.-65. (canceled)

66. A method of selecting or optimizing a therapy for treatment of a disease or disorder in a subject or population of subjects, the method comprising:
(a) analyzing information from a subject or population of subjects associated with a disease or disorder comprising a step selecting at least a first pair of nucleic acids comprising a first and second nucleic acid,
(i) wherein expression of the first nucleic acid contributes to severity of a disease or disorder and expression of the second nucleic acid at least partially modulates the severity of the disease or disorder caused by expression of the first nucleic acid; or (ii) wherein expression of both nucleic acid contributes at least partially to severity of a disease or disorder and this has greater than control subject or control population; and
(b) comparing expression of the first pair of nucleic acid sequences with a survival rate associated with a disease or disorder in a control population of subjects; and
(c) assigning a probability score to the expression of the first pair of nucleic acid sequences based upon the survival rate of the subject or population of subjects associated with a disease or disorder;
(d) selecting a therapy useful for treatment of the disease or disorder based upon the expression of the first pair of nucleic acid sequences.

67.-78. (canceled)

79. A computer program product encoded on a computer-readable storage medium comprising instructions for:
(a) analyzing information from a subject or population of subjects associated with a disease or disorder comprising a step selecting at least a first pair of nucleic acids comprising a first and second nucleic acid, wherein expression of the first nucleic acid contributes to severity of a disease or disorder and expression of the second nucleic acid at least partially modulates the severity of the disease or disorder caused by expression of the first nucleic acid;
(b) comparing expression of the first pair of nucleic acid sequences with a survival rate associated with a disease or disorder in a control population of subjects; and
(c) assigning a probability score to the expression of the first pair of nucleic acid sequences based upon the survival rate of the subject or population of subjects associated with a disease or disorder.

80. The computer program product of claim 79 further comprising instructions for:
setting a predetermined value that corresponds to a probability score above which the first pair of nucleic acid sequence is correlated to effectiveness of or resistance to a therapy;
calculating the probability score, wherein calculating the probability score comprises analyzing information associated with a disease or disorder of the subject or the population of subjects; and
conducting one or a plurality of statistical tests from the information associated with a disease or disorder;
and assigning a probability score related to effectiveness of or resistance to a therapy based upon a comparison of outcomes from the statistical tests.

81. A system comprising the computer program product of claim 79.

82.-83. (canceled)
